(19) United States
(12) Patent Application Publication
(10) Pub. No.: US 2007/0203155 A1
(43) Pub. Date: Aug. 30, 2007

(54) Compounds And Compositions As Ppar Modulators

(75) Inventors: Robert Epple, San Diego, CA (US); Christopher Cow, San Diego, CA (US); Yongping Xie, San Diego, CA (US); Xing Wang, San Diego, CA (US); Ross Russo, San Diego, CA (US); Mihai Azimioara, La Jolla, CA (US); Enrique Saez, San Diego, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton (BM)

(21) Appl. No.: 11/597,282

(22) PCT Filed: May 24, 2005
(86) PCT No.: PCT/US05/18167

§ 371 Date: Nov. 21, 2006

Related U.S. Application Data
(60) Provisional application No. 60/574,137, filed on May 24, 2004.
(60) Provisional application No. 60/648,985, filed on Jan. 31, 2005.

(51) Int. Cl. A61K 31/506; A61K 31/4709; A61K 31/4439; A61K 31/426; A61K 31/427; C07D 417/02

(52) U.S. Cl. 514/256; 514/314; 514/366; 514/365; 514/369; 514/342; 544/333; 546/169; 546/269.7; 548/181

(57) ABSTRACT

The invention provides compounds, pharmaceutical compositions comprising such compounds and methods of using such compounds to treat or prevent diseases or disorders associated with the activity of the Peroxisome Proliferator-Activated Receptor (PPAR) families, particularly the activity of PPAR.

### CROSS-REFERENCE TO RELATED APPLICATIONS

This application claims the benefit of priority to U.S. Provisional Patent Application No. 60/574,137, filed 24 May 2004, and U.S. Provisional Patent Application No. 60/648,985, filed 31 Jan. 2005. The full disclosures of these applications are incorporated herein by reference in their entirety and for all purposes.

### BACKGROUND OF THE INVENTION

1. Field of the Invention

The invention provides compounds, pharmaceutical compositions comprising such compounds and methods of using such compounds to treat or prevent diseases or disorders associated with the activity of the Peroxisome Proliferator-Activated Receptor (PPAR) families, particularly the activity of PPARδ.

2. Background

Peroxisome Proliferator Activated Receptors (PPARs) are members of the nuclear hormone receptor super family, which are ligand-activated transcription factors regulating gene expression. Certain PPARs are associated with a number of disease states including dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, IBDs (irritable bowel disease), ulcerative colitis and Crohn's disease. Accordingly, molecules that modulate the activity of PPARs, particularly PPARδ, are useful as therapeutic agents in the treatment of such diseases.

### SUMMARY OF THE INVENTION

In one aspect, the present invention provides compounds of Formula I:

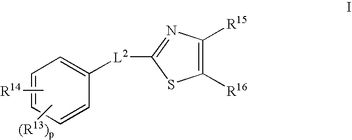

in which

p is an integer selected from 0 to 3;

L² is selected from —XOX—, —XS(O)₀₋₂X— and —XS(O)₀₋₂XO—; wherein X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-substituted-C₁₋₆alkoxy;

R¹³ is selected from halo, C₁₋₆alkyl, C₁₋₆alkoxy, hydroxy-C₁₋₆alkyl, halo-substituted-C₁₋₆alkyl, halo-substituted-C₁₋₆alkoxy, C₆₋₁₀aryl, C₅₋₁₀heteroaryl, C₃₋₁₂cycloalkyl and C₃₋₈heterocycloalkyl; wherein any aryl, heteroaryl, cycloalkyl and heterocycloalkyl of R¹³ is optionally substituted with 1 to 3 radicals independently selected from halo, nitro, cyano, C₁₋₆alkyl, C₁₋₆alkoxy, hydroxy-C₁₋₆alkyl, halo-substituted-C₁₋₆alkyl and halo-substituted-C₁₋₆alkoxy;

R¹⁴ is selected from —XOXC(O)OR¹⁷ and —XC(O)OR¹⁷; wherein X is a bond or C₁₋₄alkylene; and R¹⁷ is selected from hydrogen and C₁₋₆alkyl;

R¹⁵ and R¹⁶ are independently selected from —R¹⁸ and —YR¹⁸; wherein Y is a selected from C₁₋₆alkylene, C₂₋₆alkenylene, C₂₋₆alkynylene, —C(O)NR¹⁷— and —OX—; X is a bond or C₁₋₄alkylene; R¹⁷ is selected from hydrogen and C₁₋₆alkyl; and R¹⁸ is selected from C₃₋₁₂cycloalkyl, C₃₋₈heterocycloalkyl, C₆₋₁₀aryl and C₅₋₁₃heteroaryl; or R¹⁵ and R¹⁶ together with the atoms to which R¹⁵ and R¹⁶ are attached form fused bicyclic or tricyclic C₅₋₁₄heteroaryl;

wherein any aryl, heteroaryl, cycloalkyl and heterocycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈-heterocycloalkyl, C₆₋₁₀aryl, C₅₋₁₃heteroaryl, —XS(O)₀₋₂R¹⁷, —XS(O)₀₋₂XR¹⁹, —XNR¹⁷R¹⁷, —XNR¹⁷S(O)₀₋₂R¹⁷, —XNR¹⁷C(O)R¹⁷, —XC(O)NR¹⁷R¹⁷, —NR¹⁷C(O)R¹⁹, —XC(O)NR¹⁷R¹⁹, —XC(O)R¹⁹, —XNR¹⁷XR¹⁹ and —XOXR¹⁹; wherein any aryl, heteroaryl, cycloalkyl or heterocycloalkyl substituent is further optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl and halo-substituted-C₁₋₆alkoxy; wherein X is a bond or C₁₋₄alkylene; R¹⁷ is selected from hydrogen and C₁₋₆alkyl; and R¹⁹ is selected from C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition that contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which modulation of PPAR activity, particularly PPARδ, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which PPAR activity, particularly PPARδ, activity contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

Definitions

“Alkyl” as a group and as a structural element of other groups, for example halo-substituted-alkyl and alkoxy, can be either straight-chained or branched. C₁₋₆alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl can be phenyl or naphthyl, preferably phenyl. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzoimidazolyl, pyrimidinyl, furanyl, oxazolyl, isoxazolyl, triazolyl, tetrazolyl, pyrazolyl, thienyl, etc. “C₆₋₁₀arylC₀₋₄alkyl” means an aryl as described above connected via a alkylene grouping. For example, C₆₋₁₀arylC₀₋₄alkyl includes phenethyl, benzyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 1,4-dioxa-8-aza-spiro[4.5]dec-8-yl, etc.

“Halogen” (or halo) preferably represents chloro or fluoro, but can also be bromo or iodo.

“Treat”, “treating” and “treatment” refer to a method of alleviating or abating a disease and/or its attendant symptoms.

### DESCRIPTION OF THE PREFERRED EMBODIMENTS

The present invention provides compounds, compositions and methods for the treatment of diseases in which modulation of PPARδ activity can prevent, inhibit or ameliorate the pathology and/or symptomology of the diseases, which method comprises administering to the animal a therapeutically effective amount of a compound of Formula I.

In one embodiment, with reference to compounds of Formula I, p is an integer selected from 0 to 3; L² is selected from —XOX—, —XS(O)₀₋₂X— and —XS(O)₀₋₂XO—; wherein X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-substituted-C₁₋₆alkoxy; and R¹³ is C₁₋₆alkyl, C₁₋₆alkoxy and halo.

In a further embodiment, R¹⁴ is selected from —XOXC(O)OR¹⁷ and —XC(O)OR¹⁷; wherein X is a bond or C₁₋₄alkylene; and R¹⁷ is selected from hydrogen and C₁₋₆alkyl; R¹⁵ and R¹⁶ are independently selected from —R¹⁸ and —YR¹⁸; wherein Y is a selected from C₁₋₆alkylene, C₂₋₆alkenylene, —C(O)NR¹⁷— and —OX—; X is a bond or C₁₋₄alkylene; R¹⁷ is selected from hydrogen and C₁₋₆alkyl; and R¹⁸ is selected from C₆₋₁₀aryl, C₃₋₁₂cycloalkyl and C₅₋₁₃heteroaryl; or R¹⁵ and R¹⁶ together with the atoms to which R¹⁵ and R¹⁶ are attached form fused bicyclic or tricyclic C₅₋₁₄heteroaryl; wherein any aryl, heteroaryl and cycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl optionally substituted with C₁₋₆alkoxy, C₅₋₁₃heteroaryl, —XS(O)₀₋₂R¹⁷, —XS(O)₀₋₂XR¹⁹, —XNR¹⁷R¹⁷, —XNR¹⁷S(O)₀₋₂R¹⁷, —XNR¹⁷C(O)R¹⁷, —XC(O)NR¹⁷R¹⁷, —XNR¹⁷C(O)R¹⁹, —XC(O)NR¹⁷R¹⁹, —XC(O)R¹⁹, —XNR¹⁷XR¹⁹ and —XOXR¹⁹; wherein X is a bond or C₁₋₄alkylene; R¹⁷ is selected from hydrogen and C₁₋₆alkyl; and R¹⁹ is selected from C₆₋₁₀aryl, C₅₋₁₀heteroaryl, C₃₋₈heterocycloalkyl and C₃₋₁₂cycloalkyl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy.

In a further embodiment, the invention provides a compound of Formula Ia:

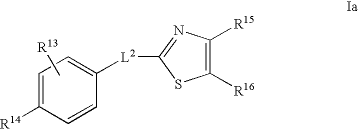

in which: L² is selected from —S(O)₀₋₂(CH₂)₁₋₄O—, —O(CH₂)₁₋₄S(O)₀₋₂—, —CH₂S(O)O₀₋₂—, —S(O)₀₋₂CH₂—, —S(O)₀₋₂—, —CH₂O and —OCH₂—; R¹³ is selected from C₁₋₆alkyl, C₁₋₆alkoxy and halo; R¹⁴ is selected from —OCH₂C(O)OH and —CH₂C(O)OH; R¹⁵ and R¹⁶ are independently selected from —R¹⁸ and —YR¹⁸; wherein Y is selected from C₁₋₆alkylene, C₂₋₆alkenylene, —C(O)NH— and —O(CH₂)₁₋₃—; and R¹⁸ is selected from phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]dioxepin-7-yl and quinolinyl; or R¹⁵ and R¹⁶ together with the atoms to which R¹⁵ and R¹⁶ are attached form 4,5-dihydro-naphtho[1,2-d]thiazol-2-yl, 4H-chromeno[4,3-d]thiazol-2-yl, 5,6-dihydro-4H-3-thia-1-aza-benzo[e]azulen-2-yl, benzthiazolyl, benzoxazolyl and 1-oxa-3-aza-cyclopenta[a]naphthalen-2-yl;

wherein any aryl, heteroaryl, cycloalkyl and heterocycloalkyl of R¹⁵, R¹⁶ or the combination of R¹⁵ and R¹⁶, is optionally substituted with 1 to 3 radicals independently selected from halo, cyano, nitro, methyl, isopropyl, isopropyl-sulfanyl, isopropyloxy, hydroxy-methyl, methyl-sulfanyl, methoxy, ethoxy, pentafluoroethoxy, trifluoromethyl, trifluoromethoxy, trifluoromethyl-sulfonyl, morpholino, phenoxy, benzoxy, ethyl-sulfonyl, dimethylamino, methyl-sulfonyl-amino, ethyl-sulfonyl, propyl, vinyl, propyloxy, sec-butoxy, trifluoromethyl-sulfanyl, dimethyl-amino-carbonyl, diethyl-amino-carbonyl, methyl-carbonyl-amino, methyl-carbonyl, cyclopentyl-oxy, isopropyl-methylamino-carbonyl, cyclopropyl-amino-carbonyl, cyclohexyl, morpholino, piperidinyl, indolyl, pyrrolidinyl, pyrrolidinyl-carbonyl, 2,3-dihydro-benzofuran-5-yl piperidinyl-carbonyl, morpholino-carbonyl, isopropyl-methyl-amino, isopropyl-methyl-amino-carbonyl, diethyl-amino, and phenyl optionally substituted with methoxy.

In a further embodiment are compounds of Formula Ib:

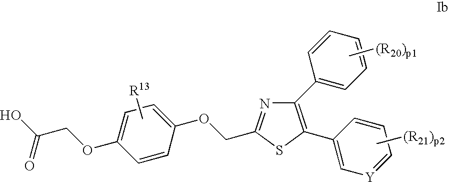

in which: p1 and p2 are independently selected from 0, 1 and 2; Y is selected from N and CH; R¹³ is selected from C₁₋₆alkyl, C₁₋₆alkoxy and halo; R²⁰ is selected from trifluoromethyl and trifluoromethoxy; and R²¹ is selected from isopropyloxy and methoxy.

Preferred compounds of Formula I are detailed in the Examples, infra. More preferred compounds of the invention are selected from: {4-[4-(4-isopropoxy-phenyl)-5-(4-trifluoromethoxy-phenyl)-thiazol-2-ylmethoxy]-2-methyl-phenoxy}-acetic acid; {4-[4-(4-isopropoxy-phenyl)-5-(4-trifluoromethyl-phenyl)-thiazol-2-ylmethoxy]-2-methyl-phenoxy}-acetic acid; and {4-[4-(6-methoxy-pyridin-3-yl)-5-(4-trifluoromethoxy-phenyl)-thiazol-2-ylmethoxy]-2-methyl-phenoxy}-acetic acid.

Pharmacology and Utility

Compounds of the invention modulate the activity of PPARs and, as such, are useful for treating diseases or disorders in which PPARs contributes to the pathology and/or symptomology of the disease. This invention further provides compounds of this invention for use in the preparation of medicaments for the treatment of diseases or disorders in which PPARs, particularly PPARδ, contributes to the pathology and/or symptomology of the disease.

Such compounds may therefore be employed for the treatment of prophylaxis, dyslipidemia, hyperlipidemia, hypercholesteremia, atherosclerosis, atherogenesis, hypertriglyceridemia, heart failure, hyper cholesteremia, myocardial infarction, vascular diseases, cardiovascular diseases, hypertension, obesity, cachexia, HIV wasting syndrome, inflammation, arthritis, cancer, Alzheimer's disease, anorexia, anorexia nervosa, bulimia, skin disorders, respiratory diseases, ophthalmic disorders, IBDs (irritable bowel disease), ulcerative colitis and Crohn's disease. Preferably for the treatment of prophylaxis, dyslipidemia, hyperlipidemia, hypercholesteremia, atherosclerosis, atherogenesis, hypertriglyceridemia, cardiovascular diseases, hypertension, obesity, inflammation, cancer, skin disorders, IBDs (irritable bowel disease), ulcerative colitis and Crohn's disease.

Compounds of the invention can also be employed to treat long term critical illness, increase muscle mass and/or muscle strength, increase lean body mass, maintain muscle strength and function in the elderly, enhance muscle endurance and muscle function, and reverse or prevent frailty in the elderly.

Further, the compounds of the present invention may be employed in mammals as hypoglycemic agents for the treatment and prevention of conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome X. Preferably type-1 and type-2 diabetes, Impaired Glucose Metabolism (IGM), Impaired Glucose Tolerance (IGT) and Impaired Fasting Glucose (IFG).

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of the invention or a pharmaceutically acceptable salt thereof. For any of the above uses, the required dosage will vary depending on the mode of administration, the particular condition to be treated and the effect desired. The present invention also concerns: i) a compound of the invention or a pharmaceutically acceptable salt thereof for use as a medicament; and ii) the use of a compound of the invention or a pharmaceutically acceptable salt thereof for the manufacture of a medicament for preventing or treating any of the diseases or disorders described above.

Administration and Pharmaceutical Compositions

In general, compounds of the invention will be administered in therapeutically effective amounts via any of the usual and acceptable modes known in the art, either singly or in combination with one or more therapeutic agents. A therapeutically effective amount can vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrollidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions can be sterilized and/or contain adjuvants, such as preserving, stabilizing, wetting or emulsifying agents, solution promoters, salts for regulating the osmotic pressure and/or buffers. In addition, they can also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations can also be used. Suitable formulations for topical application, e.g., to the skin and eyes, are preferably aqueous solutions, ointments, creams or gels well-known in the art. Such can contain solubilizers, stabilizers, tonicity enhancing agents, buffers and preservatives.

This invention also concerns a pharmaceutical composition comprising a therapeutically effective amount of a compound as described herein in combination with one or more pharmaceutically acceptable carriers.

Compounds of the invention can be administered in therapeutically effective amounts in combination with one or more therapeutic agents (pharmaceutical combinations).

Thus, the present invention also relates to pharmaceutical combinations, such as a combined preparation or pharmaceutical composition (fixed combination), comprising: 1) a compound of the invention as defined above or a pharmaceutical acceptable salt thereof; and 2) at least one active ingredient selected from:

a) anti-diabetic agents such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; DPPIV (dipeptidyl peptidase IV) inhibitors such as DPP728, LAF237 (vildagliptin—Example 1 of WO 00/34241), MK-0431, saxagliptin, GSK23A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid described in the patent application WO 03/043985, as compound 19 of Example 4, a non-glitazone type PPARγ agonist e.g. GI-262570;

b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;

c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;

d) anti-hypertensive agents, e.g., loop diuretics such as ethacrynic acid, furosemide and torsemide; diuretics such as thiazide derivatives, chlorothiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamnil; aldosterone receptor antagonists; and aldosterone synthase inhibitors;

e) a HDL increasing compound;

f) Cholesterol absorption modulator such as Zetia® and KT6-971;

g) Apo-A1 analogues and mimetics;

h) thrombin inhibitors such as Ximelagatran;

i) aldosterone inhibitors such as anastrazole, fadrazole, eplerenone;

j) Inhibitors of platelet aggregation such as aspirin, clopidogrel bisulfate;

k) estrogen, testosterone, a selective estrogen receptor modulator, a selective androgen receptor modulator;

l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib ({N-{5-[4-(4-methyl-piperazino-methyl)-benzoylamido]-2-methylphenyl}-4-(3-pyridyl)-2-pyrimidine-amine}) described in the European patent application EP-A-0 564 409 as example 21 or 4-Methyl-N-[3-(4-methyl-imidazol-1-yl)-5-trifluoromethyl-phenyl]-3-(4-pyridin-3-yl-pyrimidin-2-ylamino)-benzamide described in the patent application WO 04/005281 as example 92; and

m) an agent interacting with a 5-HT₃ receptor and/or an agent interacting with 5-HT₄ receptor such as tegaserod described in the U.S. Pat. No. 5,510,353 as example 13, tegaserod hydrogen maleate, cisapride, cilansetron;

or, in each case a pharmaceutically acceptable salt thereof; and optionally a pharmaceutically acceptable carrier.

Most preferred combination partners are tegaserod, imatinib, vildagliptin, metformin, a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid, a sulfonylurea receptor ligand, aliskiren, valsartan, orlistat or a statin such as pitavastatin, simvastatin, fluvastatin or pravastatin.

Preferably the pharmaceutical combinations contains a therapeutically effective amount of a compound of the invention as defined above, in a combination with a therapeutically effective amount of another therapeutic agent as described above, e.g., each at an effective therapeutic dose as reported in the art. Combination partners (1) and (2) can be administered together, one after the other or separately in one combined unit dosage form or in two separate unit dosage forms. The unit dosage form may also be a fixed combination.

The structure of the active agents identified by generic or trade names may be taken from the actual edition of the standard compendium “The Merck Index” or the Physician's Desk Reference or from databases, e.g. Patents International (e.g. IMS World Publications) or Current Drugs. The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

In another preferred aspect the invention concerns a pharmaceutical composition (fixed combination) comprising a therapeutically effective amount of a compound as described herein, in combination with a therapeutically effective amount of at least one active ingredient selected from the above described group a) to m), or, in each case a pharmaceutically acceptable salt thereof.

A pharmaceutical composition or combination as described herein for the manufacture of a medicament for the treatment of for the treatment of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.

Such therapeutic agents include estrogen, testosterone, a selective estrogen receptor modulator, a selective androgen receptor modulator, insulin, insulin derivatives and mimetics; insulin secretagogues such as the sulfonylureas, e.g., Glipizide and Amaryl; insulinotropic sulfonylurea receptor ligands, such as meglitinides, e.g., nateglinide and repaglinide; insulin sensitizers, such as protein tyrosine phosphatase-1B (PTP-1B) inhibitors, GSK3 (glycogen synthase kinase-3) inhibitors or RXR ligands; biguanides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors, e.g. isoleucin-thiazolidide; DPP728 and LAF237, hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (liver X receptor) and LXR (farnesoid X receptor) ligands, cholestyramine, fibrates, nicotinic acid and aspirin. A compound of the present invention may be administered either simultaneously, before or after the other active ingredient, either separately by the same or different route of administration or together in the same pharmaceutical formulation.

The invention also provides for pharmaceutical combinations, e.g. a kit, comprising: a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, in which R¹⁵ is cyclic (e.g. cycloalkyl, heterocycloalkyl, aryl and heteroaryl), can be prepared by proceeding as in reaction scheme Ia:

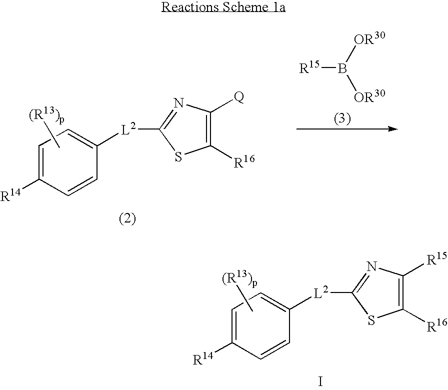

in which p, R¹³, R¹⁴, R¹⁶ and L² are as defined for Formula I in the Summary of the Invention. Q is a halogen, preferably Cl or Br; and R³⁰ is independently selected from hydrogen, C₁₋₆alkyl or the R³⁰ radicals can be cyclized. Compounds of Formula I are prepared by reacting a compound of formula 2 with a compound of formula 3 in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 120 to about 200° C. (microwave) and takes up to about 20 minutes to complete.

Compounds of Formula I, in which R¹⁶ is cyclic (e.g. cycloalkyl, heterocycloalkyl, aryl and heteroaryl), can be prepared by proceeding as in reaction scheme Ib:

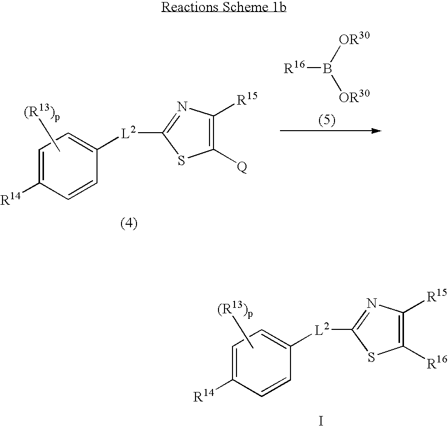

in which p, R¹³, R¹⁴, R¹⁶ and L² are as defined for Formula I in the Summary of the Invention. Q is a halogen, preferably Cl or Br; and R³⁰ is independently selected from hydrogen, C₁₋₆alkyl or the R³⁰ radicals can be cyclized. Compounds of Formula I are prepared by reacting a compound of formula 4 with a compound of formula 5 in the presence of a suitable catalyst (e.g., Pd(Ph₃)₄, or the like), a suitable base (e.g., Na₂CO₃, or the like) and a suitable solvent (e.g., water, ethanol, DME or the like). The reaction is carried out in the temperature range of about 120 to about 200° C. (microwave) and takes up to about 20 minutes to complete.

Compounds of Formula I, in which R¹⁴ is defined by —Y—COOR³¹, can be prepared by proceeding as in reaction scheme 2:

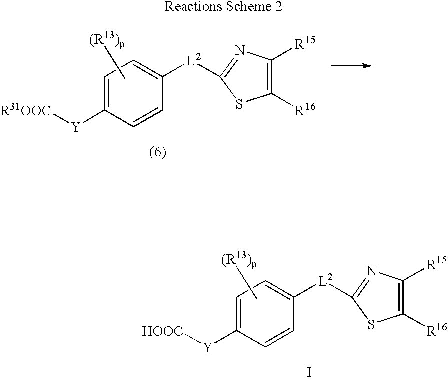

in which p, R¹³, R¹⁵, R¹⁶ and L² are as defined for Formula I in the Summary of the Invention; Y is —XOX— or —X— (wherein X is independently selected from a bond or C₁₋₄alkylene as defined in the Summary of the Invention) and R³¹ is an alkyl group, for example, methyl. Compounds of Formula I are prepared by reacting a compound of formula 4 in the presence of a suitable base (e.g., lithium hydroxide, or the like) and a suitable solvent (e.g., THF, water or the like). The reaction is carried out in the temperature range of about 0° C. to about 50° C. and takes up to about 30 hours to complete.

Compounds of Formula 9, in which R³ is —CH₃, —SH, —C(O)OC₂H₅, —CH₂OC(O)C(CH₃)₃ or a group defined by:

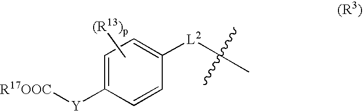

wherein Y is —XOX— or —X—; and p, R¹³, L², X and R¹⁷ are as defined in the Summary of the Invention), can be prepared by proceeding as in reaction scheme 3:

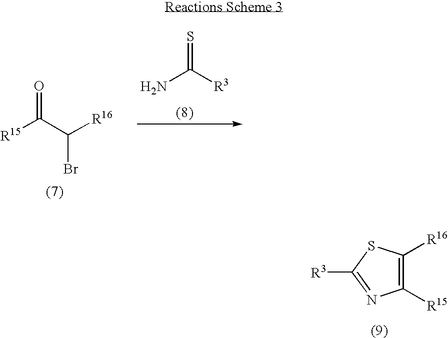

in which p, R¹³, R¹⁷ and L² are as defined for Formula I in the Summary of the Invention; R¹⁵ and R¹⁶ independently are selected from hydrogen, alkyl or any cyclic radical (cycloalkyl, heterocycloalkyl, aryl and heteroaryl as defined in the Summary of the Invention). Compounds of Formula 9 are prepared by reacting a compound of formula 7 with a compound of formula 8 optionally in the presence of a solvent (e.g., ethanol, or the like). The reaction is carried out in the temperature range of about 10 to about 200° C. and takes up to about 30 hours to complete.

Compounds of Formula I can be prepared by proceeding as in reaction scheme 4a and 4b:

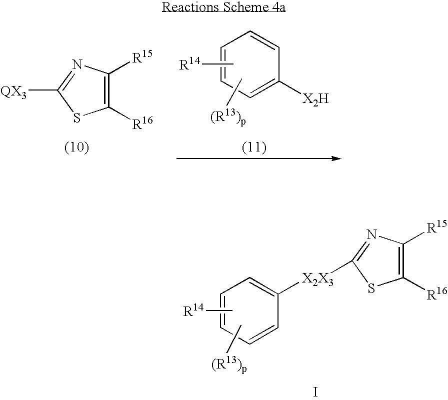

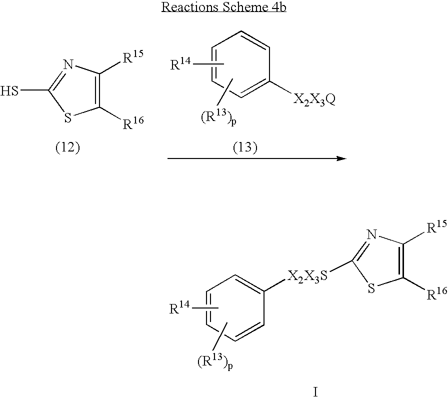

in which p, R¹³, R¹⁴, R¹⁵ and R¹⁶ are as defined for Formula I in the Summary of the Invention; X₂ is S or O; X₃ is a bond or C₁₋₄alkylene; and Q is a halo group, preferably Br or Cl. Compounds of Formula I are prepared by reacting a compound of formula 10 with a compound of formula 11 or a compound of formula 12 with a compound of formula 13 in the presence of a suitable solvent (e.g., cyanomethyl, ethanol or the like). The reaction is carried out in the temperature range of about 10 to about 80° C. and takes up to about 24 hours to complete.

Compounds of Formula I can be prepared by proceeding as in reaction scheme 5:

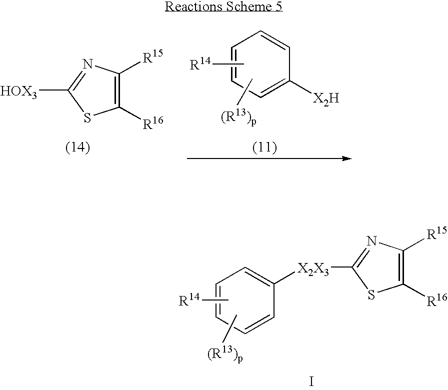

in which p, R¹³, R¹⁴, R¹⁵ and R¹⁶ are as defined for Formula I in the Summary of the Invention; X₂ is S or O; and X₃ is a bond or C₁₋₄alkylene. Compounds of Formula I are prepared by reacting a compound of formula 14 with a compound of formula 11 in the presence of a suitable solvent (e.g., DCM, THF or the like) and a suitable activating reagent (e.g., triphenylphosphine, diethylazodicarboxylate or the like). The reaction is carried out in the temperature range of about 0 to about 50° C. and takes up to about 24 hours to complete.

Detailed reaction conditions are described in the examples, infra.

Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme 1a, 1b, 2, 3, 4a, 4b or 5; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following intermediates and examples that illustrate the preparation of compounds of Formula I according to the invention.

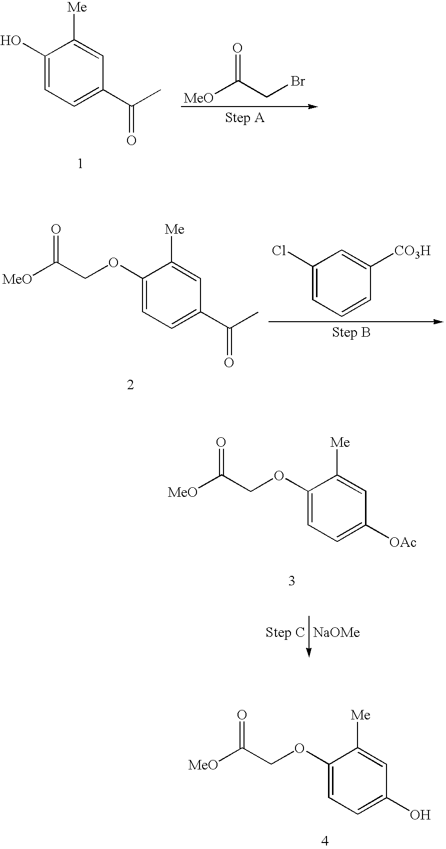

Intermediate 4. (4-Hydroxy-2-methyl-phenoxy)-acetic Acid Methyl Ester.

Step A: 4′-Hydroxy-3′-methylacetophenone 1 (25 g, 166.4 mmol) and methyl-bromoacetate (25.5 g, 166.4 mmol) is dissolved in MeCN (600 mL). Cs₂CO₃ (117.8 g, 332.9 mmol) is added and the mixture is stirred overnight at room temperature. After insoluble salts are filtered and washed with MeCN, the solvent is removed and the remainder is taken up in EtOAc and washed subsequently with 1 M HCl (3×500 mL) and H₂O (2×500 mL). The organic layer is dried (MgSO₄), filtered and concentrated to afford 2 as a white solid.

Step B: (4-Acetyl-2-methyl-phenoxy)-acetic acid methyl ester 2 (33 g, 151.3 mmol), 77% mCPBA (54.9 g, 264.8 mmol) and p-TsOH (2.9 g, 15.1 mmol) in DCM (650 mL) are heated under reflux for 48 hours. The reaction mixture is then washed with 1 M KI (2×500 mL) and NaHSO₃ (2×500 mL). The organic layer is dried (MgSO₄), filtered and concentrated to afford 3 as a brown syrup.

Step C: A solution of (4-acetoxy-2-methyl-phenoxy)-acetic acid methyl ester 3 (25 g, 105.0 mmol) in dry MeOH (400 mL) is combined with a 0.5 M solution of NaOMe in MeOH (210 mL, 105.0 mmol) and stirred for 1 hour at room temperature. The solution is neutralized with 1 M HCl and washed with H₂O (2×500 mL). The organic layer is dried (MgSO₄), filtered and concentrated to afford 4 as a brown solid: ¹H-NMR (400 MHz, CD₃OD) δ=6.65-6.51 (m, 3H), 4.60 (s, 2H), 3.75 (s, 3H), 2.19 (s, 3H). MS calculated for C₁₀H₁₃O₄ (M+H⁺) 197.1, found 197.2.

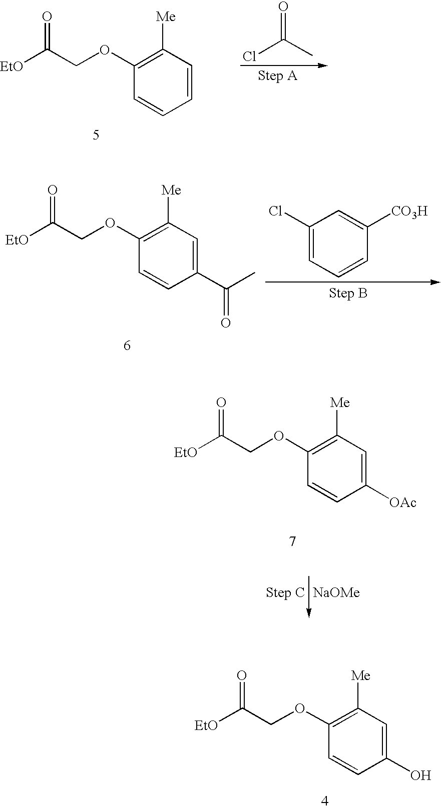

Intermediate 4 (Alternative Route). (4-Hydroxy-2-methyl-phenoxy)-acetic Acid Methyl Ester.

Step A: (2-Methylphenoxy)acetic acid ethyl ester 5 (66.03 g, 340 mmol) is dissolved in dichloroethane (400 mL). Aluminum chloride (100.02 g, 750 mmol) is added and the light-brown mixture is stirred for 10 minutes at room temperature until homogenous. Acetyl chloride (35 mL, 493 mmol) is added dropwise using an addition funnel. The rate of addition is adjusted to maintain a relatively slow emission of hydrogen chloride gas. The resulting dark brown solution is allowed to cool to room temperature, then is poured over 300 g of crushed ice. The mixture is diluted with dichloromethane (300 mL) and is washed successively with water, saturated NaHCO₃ solution, water, saturated NH₄Cl solution, and brine. The organic layer is dried over Na₂SO₄, filtered and concentrated to afford 6 as a brown oil that solidified as a crystalline mass. ¹H-NMR (400 MHz, CDCl₃) δ=7.79 (d, J=2.0 Hz, 1H), 7.77 (dd, J=2.0, 8.4 Hz, 1H), 6.69 (d, J=8.4 Hz, 1H), 4.71 (s, 2H), 4.26 (q, J=7.2 Hz, 2H), 2.54 (s, 3H), 2.32 (s, 2H), 1.29 (t, J=7.2 Hz, 3H).

Step B: (4-Acetyl-2-methyl-phenoxy)-acetic acid ethyl ester 6 (76.54 g, 324 mmol), 77% mCPBA (100.31 g, 407 mmol, 1.26 equiv.) and p-TsOH (13 g, 68 mmol, 21 mol %) in dichloroethane (450 mL) are heated to 50° C. for 30 hours. The reaction mixture is then washed with 1 M KI (2×500 mL) and NaHSO₃ (2×500 mL). The organic layer is dried (MgSO₄), filtered and concentrated to afford 7 as a brown syrup.

Step C: A solution of (4-Acetoxy-2-methyl-phenoxy)-acetic acid ethyl ester 7 (from step B above) in dry MeOH (400 mL) is combined with a 0.5 M solution of NaOMe in MeOH (650 mL, 325 mmol) and stirred for 2 h at room temperature. The solution is neutralized with 1 M HCl and washed with H₂O (2×500 mL). The organic layer is dried (Na₂SO₄), filtered and concentrated to afford 4 (21.7 g, 111 mmol, 34%, two steps) as a light-brown solid: ¹H-NMR (400 MHz, CDCl₃) δ=6.58 (d, J=2.8 Hz, 1H), 6.54 (d, J=8.4 Hz), 6.50 (dd, J=2.8, 8.4 Hz, 1H), 4.7 (br. s, 1H), 4.54 (s, 2H), 3.73 (s, 3H), 2.17 (s, 3H). MS calculated for C₁₀H₁₃O₄ (M+H⁺) 197.1, found 197.4.

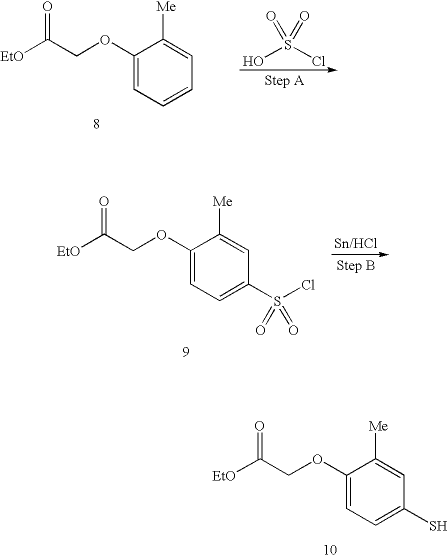

Intermediate 10. (4-Mercapto-2-methyl-phenoxy)-acetic Acid Ethyl Ester.

Step A: A 500 mL three-necked round bottom flasked is charged with chlorosulfonic acid (25 mL, 373.9 mmol), flushed with nitrogen and cooled to 0° C. Under nitrogen and vigorous stirring, ethyl(2-methylphenoxy)acetate 8 (40 g, 206.2 mmol) is added dropwise. The mixture is stirred for 90 minutes at 0° C., then poured on ice-water (200 mL). After the mixture is stirred for an additional 45 min at room temperature, the white precipitate is filtered, washed with ice-water and dried in vacuo to afford 9 as a white solid.

Step B: (4-Chlorosulfonyl-2-methyl-phenoxy)-acetic acid ethyl ester 9 (25 g, 85.4 mmol) and tin (50.8 g, 427 mmol) are suspended in EtOH and cooled to 0° C. After a solution of 4 N HCl in dioxane (107 mL, 427 mmol) is added dropwise, the resulting mixture is heated to reflux for 3 hours. Then the mixture is concentrated in vacuo, the remainder taken up in chloroform and filtered. The filtrate is concentrated in vacuo to a yellow oil, which is purified by chromatography (silica, Hex/EtOAc gradient) to afford 10 as a colorless oil: ¹H-NMR (400 MHz, CDCl₃) δ=7.14 (m, 1H), 7.07-7.10 (m, 1H), 6.59 (m, 1H), 4.60 (s, 2H), 4.25 (q, J=7.1 Hz, 2H), 3.33 (s, 1H), 2.24 (s, 3H), 1.29 (t, J=7.1 Hz, 3H). MS calculated for C₁₁H₁₄O₃S (M+H⁺) 227.1, found 227.4.

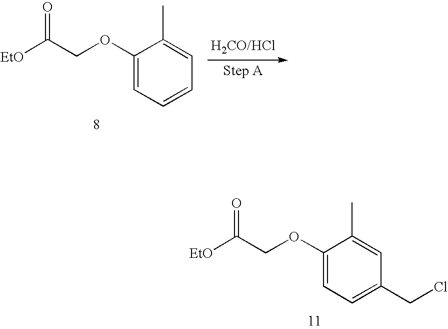

Intermediate 11. (4-Chloromethyl-2-methyl-phenoxy)-acetic Acid Ethyl Ester.

Step A: To a solution of ethyl(2-methylphenoxy)acetate 8 (20.0 g, 103 mmol) in petroleum ether (50 mL, b.p. 40-55° C.), HCl (120 mL, 12M) and formaldehyde (8.4 mL, 37%) are added, then the mixture is stirred for 25 h at room temperature. The mixture is diluted with EtOAc and the organic layer is washed with water three times, dried (MgSO₄), filtered and concentrated to afford crude product, which is purified by flash chromatography with 20% EtOAc/hexane to give (4-chloromethyl-2-methyl-phenoxy)-acetic acid ethyl ester 11 as a liquid: ¹H-NMR (400 MHz, CDCl₃) δ=7.19 (d, J=2.0 Hz, 1H), 7.14 (dd, J=2.0 Hz, J=8.0 Hz, 1H), 6.55 (d, J=8.0 Hz, 1H), 4.64 (s, 2H), 4.53 (s, 2H), 4.26 (q, J=7.2 Hz, 2H), 2.29 (s, 3H), 1.30 (t, J=7.2 Hz, 3H). MS calculated for C₁₂H₁₅ClO₃ (M−Cl⁺) 207.2, found 207.10.

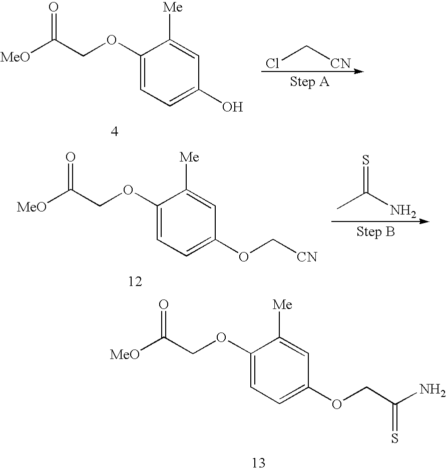

Intermediate 13. (2-Methyl-4-thiocarbamoylmethoxy-phenoxy)-acetic Acid Methyl Ester.

Step A: (4-Hydroxy-2-methyl-phenoxy)-acetic acid methyl ester 4 (1.64 g, 8.3 mmol) and chloroacetonitrile (0.553 mL, 8.7 mmol) are dissolved in acetonitrile (30 mL). Cs₂CO₃ (5.4 g, 16.7 mmol) is added and the mixture is stirred for 2 h at room temperature. Insoluble salts are filtered and washed with EtOAc, the solvent is removed to give an oil which crystallized under vacuum to give 12 (1.84 g, 7.83 mmol, 94%) as a pale yellow solid. ¹H-NMR (400 MHz, CDCl₃) δ=6.76 (s, 1H), 6.67 (s, 1H), 6.60 (d, J=8.5 Hz, 1H), 4.62 (s, 2H), 4.54 (s, 2H), 3.73 (s, 3H), 2.21 (s, 3H). MS calculated for C₁₂H₁₄NO₄ (M+H⁺) 236.1, found 236.3.

Step B: (4-Cyanomethoxy-2-methyl-phenoxy)-acetic acid methyl ester 12 (1.75 g, 7.45 mmol) and thioacetamide (1.12 g, 14.9 mmol) are dissolved in DMF (120 mL). HCl (4.0 N in 1,4-dioxane, 20 mL) is added and the mixture is stirred at 100° C. overnight. The mixture is diluted with saturated NaHCO₃, extracted with EtOAc and washed subsequently with H₂O (4×100 mL) and brine (100 mL). The organic layer is dried (MgSO₄), filtered and concentrated. The residue is triturated with DCM (5 mL) and hexanes (5 mL) and collected by filtration to afford 13 as a beige solid: ¹H-NMR (400 MHz, DMSO-d6) δ=6.84 (d, J=2.9 Hz, 1H), 6.78 (d, J=8.9 Hz, 1H), 6.71 (dd, J=3.0, 8.9 Hz, 1H), 4.75 (s, 2H), 4.67 (s, 2H), 4.04 (s, 1H), 3.69 (s, 3H), 2.18 (s, 3H). MS calculated for C₁₂H₁₆NO₄S (M+H⁺) 270.1, found 270.3.

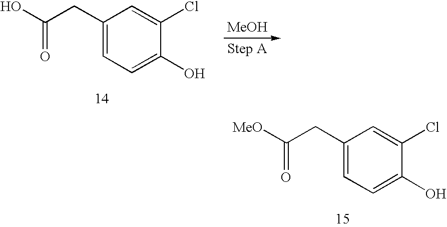

Intermediate 15. (3-Chloro-4-hydroxy-phenyl)-acetic Acid Methyl Ester.

Step A: 3-Chloro-4-hydroxy-phenyl)-acetic acid 14 (20 g, 107 mmol) is dissolved in MeOH (250 mL) containing catalytic amounts of conc. H₂SO₄ (2.5 mL). The solution is heated to reflux overnight. The solvent is evaporated, the remainder is dissolved in DCM and washed with H₂O (3×200 mL). The organic layer is dried (MgSO₄), filtered and concentrated to afford 15 as a light yellow solid: ¹H-NMR (400 MHz, CD₃OD) δ=7.21 (d, J=2.1 Hz, 1H), 7.01 (dd, J=2.1 Hz, J=8.3, 1H), 6.84 (d, J=8.3 Hz, 1H), 3.67 (s, 3H), 3.54 (s, 2H). MS calculated for C₉H₁₀ClO₃ (M+H⁺) 201.0, found 201.2.

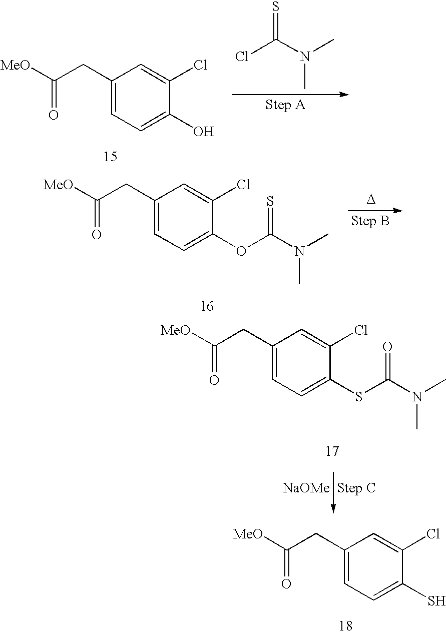

Intermediate 18. (3-Chloro-4-mercapto-phenyl)-acetic Acid Methyl Ester.

Step A: 3-(Chloro-4-hydroxy-phenyl)-acetic acid methyl ester 15 (4.1 g, 21.4 mmol), dimethyl thiocarbamoylchloride (3.2 g, 25.6 mmol), Et₃N (5.9 mL, 42.8 mmol) and DMAP (261 mg, 2.14 mmol) are dissolved in dry dioxane (30 mL) and heated to reflux for 16 h under nitrogen. The reaction mixture is cooled to room temperature, diluted with EtOAc and washed with H₂O (3×50 mL). The organic layer is dried (MgSO₄), filtered and concentrated to afford 16 as a colorless oil.

Step B: (3-Chloro-4-dimethylthiocarbamoyloxy-phenyl)-acetic acid methyl ester 16 (5.2 g, 18.1 mmol) is transferred to a 250 mL three-necked round bottom flask equipped with a thermometer. Tetradecane (45 mL) is added and the mixture is heated to reflux (250° C.) overnight. After cooling to room temperature the solvent is decanted, the remaining oil is washed several times with hexanes and purified by chromatography (silica, Hex/EtOAc gradient) to afford 17 as a brown oil.

Step C: (3-Chloro-4-dimethylcarbamoylsulfanyl-phenyl)-acetic acid methyl ester 17 (3.1 g, 10.8 mmol) is dissolved in 0.5 M NaOMe in MeOH. The mixture is heated to reflux for 4 h, then acidified with 1 M HCl. The organic solvent is evaporated, the remainder is extracted into EtOAc (50 mL) and washed with H₂O (2×50 mL). The organic layer is dried (MgSO₄), filtered, concentrated and purified (silica, hexanes/EtOAc gradient) to afford 18 as a pale yellow oil: ¹H-NMR (400 MHz, CDCl₃) δ=7.30-7.26 (m, 2H), 7.06-7.03 (m, 1H) 3.87 (s, 1H), 3.69 (s, 3H), 3.55 (s, 2H). MS calculated for C₉H₁₀ClO₂S (M+H⁺) 217.0, found 217.3.

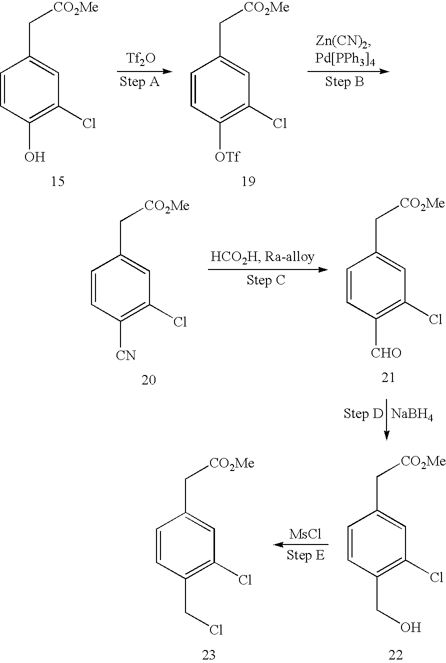

Intermediate 23. (3-Chloro-4-chloromethyl-phenyl)-acetic Acid Methyl Ester.

Step A: To a solution of (3-Chloro-4-hydroxy-phenyl)-acetic acid methyl ester 15 (15.9 g, 79.25 mmol) in CH₂Cl₂ (160 mL), triethylamine (11.04 mL, 79.25 mmol) and trifilic anhydride (13.33 mL, 79.25 mmol) are added at 0° C. and stirred for 1 hour. The reaction mixture is then diluted with EtOAc (300 mL) and washed with NaHCO₃, brine and water. The organic layer is dried (MgSO₄), filtered and concentrated to afford (3-chloro-4-trifluoromethanesulfonyloxy-phenyl)-acetic acid methyl ester 19 as an oil. MS calculated for C₁₀H₉ClF₃O₅S (N+H⁺) 333, found 333.95.

Step B: To a solution of (3-chloro-4-trifluoromethanesulfonyloxy-phenyl)-acetic acid methyl ester 19 (24.5 g, 73.64 mmol) in dry DMF (45 mL) is added zinc cyanide (8.91 g, 75.9 mmol) and tetrakis(triphenylphosphine)palladium (8.50 g, 7.36 mmol). The mixture is stirred for 34 h at 80° C. and then cooled to room temperature, diluted with EtOAc (150 mL) and poured into a saturated NaHCO₃ solution (150 mL). A white precipitate is removed by vacuum filtration. The filtrate is washed with H₂O, dried (MgSO₄), filtered and concentrated to afford crude product, which is purified by silic gel chromatography with 20% EtOAc/hexane to give (3-chloro-4-cyano-phenyl)acetic acid methyl ester 20 (11.6 g, 75.13 mmol) as a wax-like solid: ¹H-NMR (400 MHz, CDCl₃) δ=7.63 (d, J=8.0 Hz, 1H), 7.47 (d, J=1.2 Hz, 1H), 7.30 (dd, J=1.2 Hz, J=8.0 Hz, 1H), 3.72 (s, 3H), 3.69 (s, 2H). MS calculated for C₁₀H₉O₂ClN (M+H⁺) 210.0, found 210.0.

Step C: A solution of (3-chloro-4-cyano-phenyl)acetic acid methyl ester 20 (7.4 g, 35.3 mmol) in formic acid (100 mL, 88%) is combined with Raney-alloy (9.0 g) and heated to reflux (110° C.) overnight. Then the mixture is cooled to room temperature. The alloy is filtered off by Celite pad, washed with EtOAc and the filtrate is concentrated in vacuo. The remainder is diluted with EtOAc (250 mL) and washed with H₂O (2×) and NaHCO₃ (2×). The organic layer is dried (MgSO₄), filtered and concentrated to afford crude product, which is purified by silic gel chromatography with EtOAc/hexane to give (3-Chloro-4-formyl-phenyl)-acetic acid methyl ester 21 as a wax-like solid: ¹H-NMR (400 MHz, CDCl₃) δ=10.31 (s, 1H), 7.84 (d, J=8.0 Hz, 1H), 7.58 (s, 1H), 7.45 (d, J=8.0 Hz, 1H), 3.86 (s, 2H), 3.64 (s, 3H).

Step D: A solution of (3-chloro-4-formyl-phenyl)-acetic acid methyl ester 21 (0.6 g, 2.82 mmol) in MeOH (4.0 mL) is added dropwise to a solution of NaBH₄ in water (4.0 mL) and stirred for 10 minutes at 20-22° C. Then HCl (1N, 15 mL) is added and the mixture is stirred for 5 minutes. The solution is diluted with EtOAc (80 mL) and the organic layer is dried (MgSO₄), filtered and concentrated to afford crude product, which is purified by silic gel chromatography with 50% EtOAc/hexane to give (3-chloro-4-hydroxymethyl-phenyl)-acetic acid methyl ester 22 as a wax-like solid: ¹H-NMR (400 MHz, CDCl₃) δ=7.44 (d, J=8.0 Hz, 1H), 7.30 (d, J=1.2 Hz, 1H), 7.20 (dd, J=1.2 Hz, J=8.0 Hz, 1H), 4.76 (s, 2H), 3.70 (s, 3H), 3.61 (s, 2H).

Step E: To a solution of (3-chloro-4-hydroxymethyl-phenyl)-acetic acid methyl ester 22 (0.5 g, 2.33 mmol) in dry DMF (5 mL) is added lithium chloride (108.6 mg, 2.56 mmol) and s-collidine (310.2 mg, 2.56 mmol). The mixture is cooled to 0° C. and MeSO₂Cl (2.56 mmol) is added slowly. The mixture is stirred for two hours at 0° C., then poured on ice-water and extracted with EtOAc. The organic layer is washed with water, dried (MgSO₄), filtered and concentrated to afford (3-chloro-4-chloromethyl-phenyl)-acetic acid methyl ester 23: ¹H-NMR (400 MHz, CDCl₃) δ=7.42 (m, 1H), 7.34 (m, 1H), 7.19 (m, 1H), 4.68 (s, 2H), 3.71 (s, 3H), 3.61 (s, 2H). MS calculated for C₁₀H₁₁Cl₂O₂ (M+1)⁺ 233.0, found 233.00.

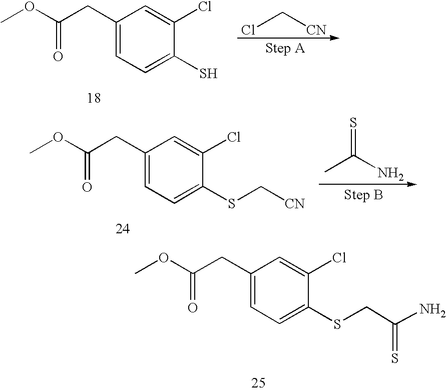

Intermediate 25. (3-Chloro-4-thiocarbamoyl-methylsulfanyl-phenyl)-acetic Acid Methyl Ester.

Step A: (3-Chloro-4-mercapto-phenyl)-acetic acid methyl ester 18 (1.04 g, 4.8 mmol) is dissolved in dry acetonitrile. Cesium carbonate (3.16 g, 9.7 mmol, 2 equiv.) is added, followed by chloroacetonitrile (0.45 mL, 7.13 mmol, 1.5 equiv.). The mixture is stirred under nitrogen for 18 hours. The resulting red suspension is filtered, the solids are washed with more acetonitrile, and the resulting clear red solution is concentrated to yield 24 as an orange oil (1.26 g, quantitative). ¹H-NMR (400 MHz, DMSO-d₆) δ=7.50 (d, J=8.0 Hz, 1H), 7.49 (s, 1H), 7.35 (dd, J=1.6, 8.0 Hz, 1H), 4.35 (s, 2H), 3.75 (s, 2H), 3.63 (s, 3H). MS calculated for C₁₁H₁₁ClNO₂S (M+H⁺) 256.0, found 256.3.

Step B: (3-Chloro-4-cyanomethylsulfanyl-phenyl)-acetic acid methyl ester 24 (1.12 g, 4.38 mmol), thioacetamide (1.52 g, 20.2 mmol, 4.6 equiv.), and a hydrogen chloride solution in dioxane (4.0 M, 5 mL, 20 mmol, 4.6 equiv.) are dissolved in 2 mL dimethylacetamide and 3 mL dioxane. The mixture is heated to 95° C. for 48 hours. The reaction mixture is then cooled to room temperature, diluted with ethyl acetate and washed with water, saturated NaHCO₃, saturated NH₄Cl, and brine. The organic layer is dried (Na₂SO₄), filtered and concentrated to afford a red oil. Silica gel chromatography (20% to 60% ethyl acetate in hexanes) yielded (3-chloro-4-thiocarbamoylmethylsulfanyl-phenyl)-acetic acid methyl ester 25 as a brown syrup. ¹H-NMR (400 MHz, DMSO-d₆) δ=9.80 (s, 1H), 9.39 (s, 1H) 7.32 (d, J=1.6 Hz, 1H), 7.36 (d, J=8.4 Hz, 1H), 7.20 (dd, J=1.6, 8.4 Hz, 1H), 4.10 (s, 2H), 3.67 (s, 3H), 3.59 (s, 3H). MS calculated for C₁₁H₁₃ClNO₂S₂ (M+H⁺) 290.0, found 290.2.

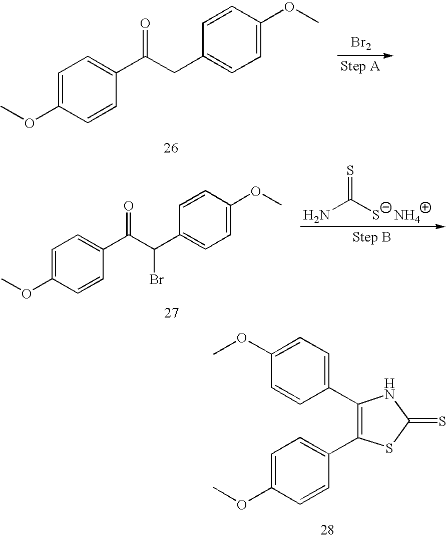

Intermediate 28. 4,5-Bis-(4-methoxy-phenyl)-3H-thiazole-2-thione.

Step A: To a solution of desoxyanisoin 26 (10 g, 39.0 mmol) in anhydrous CHCl₃ (200 mL) is added bromine (2.4 mL, 46.8 mmol) dropwise. After the addition is complete (indicated by a colour change to red), the solvent is evaporated, the remainder is triturated with ether and the precipitated product is filtered to give 27 as a white solid: 1H-NMR (400 MHz, CD₃OD) δ=7.98 (d, J=9.0 Hz, 2H), 7.35 (d, J=8.7 Hz, 2H), 6.93 (d, J=9.0 Hz, 2H), 6.89 (d, J=8.7 Hz, 2H), 5.72 (s, 1H), 3.83 (s, 3H), 3.75 (s, 3H). MS calculated for C₁₆H₁₅O₃ (M−Br⁺) 255.1, found 255.4.

Step B: 2-Bromo-1,2-bis-(4-methoxy-phenyl)-ethanone 27 (3.0 g, 8.9 mmol) and ammonium dithiocarbamate (1.5 g, 13.4 mmol) are dissolved in EtOH (50 mL) and heated to 60° C. for 3 hours. The solvent is then partially removed, the precipitate filtered and recrystallized from EtOH to yield 28 as a white solid: ¹H-NMR (400 MHz, CD₃OD) δ=7.26 (d, J=8.8 Hz, 2H), 7.11 (d, J=8.8 Hz, 2H), 6.92 (d, J=8.8 Hz, 2H), 6.84 (d, J=8.8 Hz, 2H), 3.81 (s, 3H), 3.77 (s, 3H). MS calculated for C₁₇H₁₆NO₂S₂ (M+H⁺) 330.1, found 330.2.

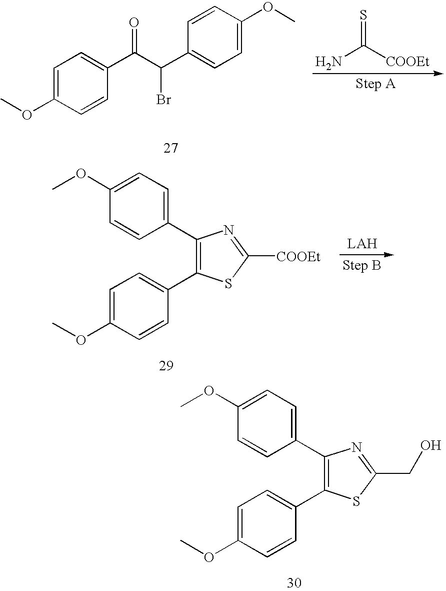

Intermediate 30. [4,5-Bis-(4-methoxy-phenyl)-thiazol-2-yl]-methanol.

Step A: A solution of 2-bromo-1,2-bis-(4-methoxy-phenyl)-ethanone 27 (3.0 g, 9.0 mmol) and ethyl thiooxamate (1.2 g, 9.0 mmol) in anhydrous EtOH (20 mL) is heated to reflux for 12 hours. The solvent is removed in vacuo and the remainder is purified by chromatography (silica, DCM/MeOH gradient) to afford 29 as a colorless semi-solid: ¹H-NMR (400 MHz, CD₃OD) δ=7.39 (d, J=8.9 Hz, 2H), 7.27 (d, J=8.8 Hz, 2H), 6.92 (d, J=8.9 Hz, 2H), 6.87 (d, J=8.8 Hz, 2H), 4.45 (q, J=7.1, 2H), 3.81 (s, 3H), 3.79 (s, 3H), 1.42 (t, J=7.1 Hz, 3H). MS calculated for C₂₀H₂₀NO₄S (M+H⁺) 370.1, found 370.4.

Step B: 4,5-Bis-(4-methoxy-phenyl)-thiazole-2-carboxylic acid ethyl ester 29 (1.0 g, 2.7 mmol) is dissolved in dry THF (20 mL) and cooled to 0° C. A solution of 1 M lithium aluminium hydride in THF (4 mL, 4.1 mmol) is added dropwise via cannula and the mixture is stirred at 0° C. for 1 hour. Sodium sulfate decahydrate (1.3 g, 4.1 mmol) is added slowly and the mixture is stirred an additional 1 h at room temperature. The suspension is then filtered over celite, dried (MgSO₄) and concentrated. The concentrate is purified by chromatography (silica, DCM/MeOH gradient) to yield 30 as a yellow oil: ¹H-NMR (400 MHz, CD₃OD) δ=7.34 (d, J=8.8 Hz, 2H), 7.22 (d, J=8.8 Hz, 2H), 6.89 (d, J=8.8 Hz, 2H), 6.84 (d, J=8.8 Hz, 2H), 3.96 (s, 2H), 3.80 (s, 3H), 3.78 (s, 3H). MS calculated for C₁₈H₈NO₃S (M+H⁺) 328.1, found 328.4.

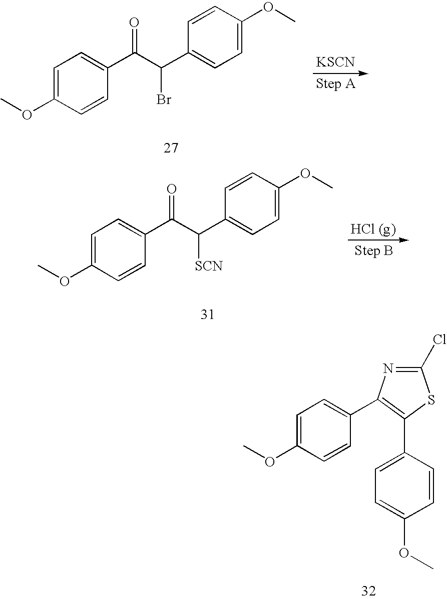

Intermediate 32: 2-Chloro-4,5-bis-(4-methoxy-phenyl)-thiazole.

Step A: 2-Bromo-1,2-bis-(4-methoxy-phenyl)-ethanone 27 (500 mg, 1.49 mmol) and potassium rhodanide (145 mg, 1.49 mmol) are heated to reflux in acetone (20 mL) for 8 hours. The mixture is cooled, diluted with water (50 mL), extracted with EtOAc (3×50 mL) and washed with brine (30 mL). The organic layer is dried (MgSO₄), filtered and concentrated to give crude 1,2-Bis-(4-methoxy-phenyl)-2-thiocyanato-ethanone 31, which is used without further purification in Step B.

Step B: The crude 1,2-bis-(4-methoxy-phenyl)-2-thiocyanato-ethanone 31 (440 mg, 1.40 mmol) is dissolved in EtOAc (100 mL), then HCl gas is bubbled through the solution for two hours. The mixture is neutralized with aqueous NaOH to pH 6, then extracted with EtOAc (3×50 mL) and washed sequentially with water (30 mL) and brine (30 mL). The organic layer is dried (MgSO₄), filtered, concentrated and purified on silica (EtOAc/Hexane gradient) to afford the title compound 32 as a white solid: ¹H-NMR (400 MHz, CDCl₃) δ=7.42 (d, J=9.0 Hz, 2H), 7.25 (d, J=9.3 Hz, 2H), 6.87 (d, J=8.8 Hz, 2H), 6.80 (d, J=9.3 Hz, 2H), 3.83 (s, 3H), 3.80 (s, 3H). MS calculated for C₁₇H₁₅ClNO₂S (M+H⁺) 332.0, found 332.3.

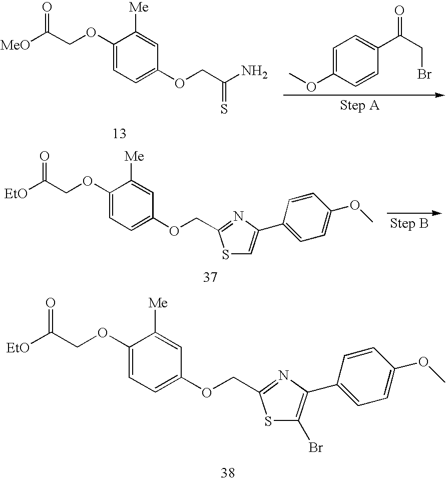

Intermediate 38: {4-[5-Bromo-4-(4-methoxy-phenyl)-thiazol-2-ylmethoxy]-2-methyl-phenoxy}-acetic Acid Ethyl Ester.

Step A: Intermediate 13 (200 mg, 0.743 mmol), and 2-bromo-4′-methoxyacetophenone (186 mg, 0.817 mmol) are heated at reflux in EtOH (4 mL) for 2 hours. The mixture is cooled, filtered, and washed with methanol to provide {4-[4-(4-methoxy-phenyl)-thiazol-2-ylmethoxy]-2-methyl-phenoxy}-acetic acid ethyl ester 37 as a white solid: ¹H-NMR (400 MHz, CDCl₃) δ=7.77 (d, J=6.8 Hz, 1H), 7.75 (d, J=6.8 Hz, 1H), 7.32 (s, 1H), 6.93 (d, J=8.8 Hz, 1H), 6.91 (d, J=8.8 Hz, 1H), 6.83 (d, J=3.0 Hz, 1H), 6.73 (dd, J=3.0, 8.9 Hz, 1H), 6.65 (d, J=8.8 Hz, 1H), 5.29 (s, 2H), 4.51 (s, 2H), 4.18 (q, J=7.2 Hz, 2H), 3.79 (s, 3H), 2.22 (s, 3H), 1.20 (t, J=7.2 Hz, 3H). MS calculated for C₂₂H₂₄NO₅S (M+H⁺) 414.1, found 414.4.

Step B: Intermediate 37 (184.6 mg, 0.45 mmol) is dissolved in dichloromethane (2 mL), then bromine (39 μL, 0.76 mmol) in acetic acid (100 μL) is added and the mixture is stirred at room temperature for 1 hour. The mixture is diluted with saturated NaHCO₃, extracted with dichloromethane and washed with brine (10 mL). The organic layer is dried (MgSO₄), filtered and concentrated to give intermediate 38 as a white glassy substance: ¹H-NMR (400 MHz, CDCl₃) δ=7.80 (d, J=6.8 Hz, 1H), 7.79 (d, J=6.8 Hz, 1H), 6.92 (d, J=8.8 Hz, 1H), 6.90 (d, J=8.8 Hz, 1H), 6.79 (d, J=3.0 Hz, 1H), 6.67 (dd, J=3.0, 8.9 Hz, 1H), 6.59 (d, J=8.8 Hz, 1H), 5.20 (s, 2H), 4.51 (s, 2H), 4.18 (q, J=7.2 Hz, 2H), 3.79 (s, 3H), 2.22 (s, 3H), 1.20 (t, J=7.2 Hz, 3H). MS calculated for C₂₂H₂₃BrNO₅S (M+H⁺) 492.0, found 492.2.

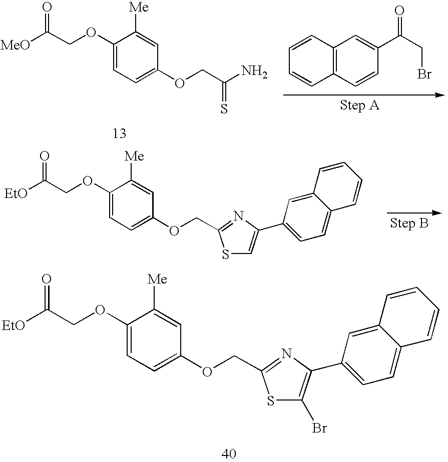

Intermediate 40: [4-(5-Bromo-4-naphthalen-2-yl-thiazol-2-ylmethoxy)-2-methyl-phenoxy]-acetic Acid Ethyl Ester.

Following the procedure of Intermediate 38, except substituting 2-Bromo-1-naphthalen-2-yl-ethanone for 2-bromo-4′-methoxyacetophenone in Step A, the title compound is purified on reverse phase HPLC (H₂O/MeCN gradient) to afford a white solid: ¹H-NMR (400 MHz, CDCl₃) δ=8.42 (s, 1H), 8.03 (dd, J=1.6, 8.4 Hz, 1H), 7.92 (d, J=8.8 Hz, 2H), 7.88 (m, 1H), 7.52 (m, 2H), 6.89 (d, J=3.2 Hz, 1H), 6.77 (dd, J=3.2, 8.8 Hz, 1H), 6.68 (d, J=8.8 Hz, 1H), 5.33 (s, 2H) 4.60 (s, 2H), 4.26 (q, J=7.2 Hz, 2H) 2.30 (s, 3H), 1.28 (t, J=7.2 Hz, 3H). MS calculated for C₂₅H₂₃BrNO₄S (M+H⁺) 512.1, found 511.5.

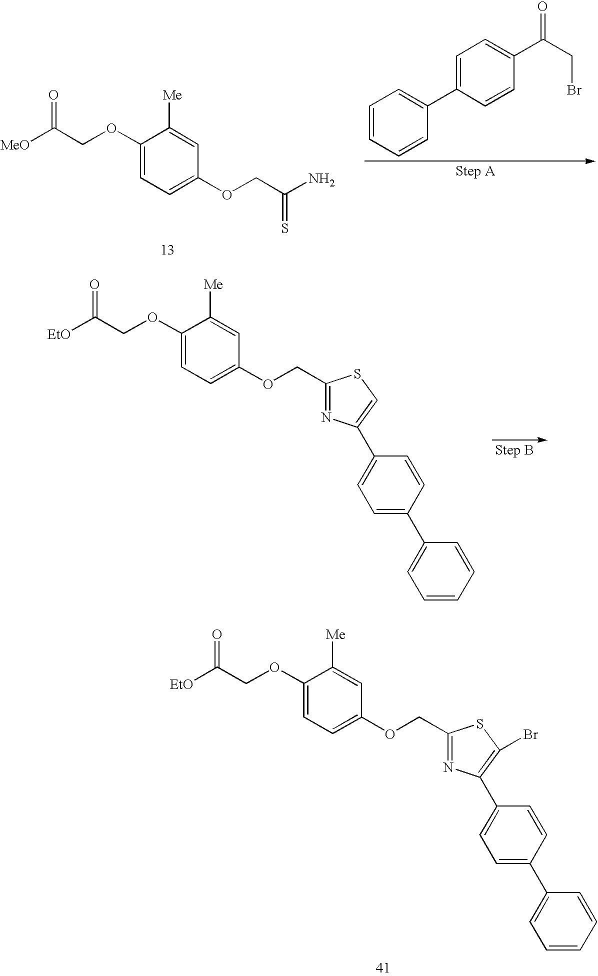

Intermediate 41: [4-(4-Biphenyl-4-yl-5-bromo-thiazol-2-ylmethoxy)-2-methyl-phenoxy]-acetic Acid Ethyl Ester.

Following the procedure of Intermediate 38, except substituting 1-biphenyl-4-yl-2-bromo-ethanone for 2-bromo-4′-methoxyacetophenone in Step A, the title compound is purified on reverse phase HPLC (H₂O/MeCN gradient) to afford a white solid: ¹H-NMR (400 MHz, CDCl₃) δ=7.99 (d, J=8.4 Hz, 2H), 7.66 (d, J=8.4 Hz, 2H), 7.61 (d, J=7.6 Hz, 2H), 7.43 (t, J=7.2 Hz, 2H), 7.34 (t, J=7.2 Hz, 1H), 6.84 (d, J=2.4 Hz, 1H), 6.72 (dd, J=2.8, 8.8 Hz, 1H), 6.64 (d, J=8.8 Hz, 1H), 5.26 (s, 2H) 4.56 (s, 2H), 4.22 (q, J=7.2 Hz, 2H), 2.27 (s, 3H), 1.24 (t, J=7.2 Hz, 3H). MS calculated for C₂₇H₂₅BrNO₄S (M+H⁺) 538.1, found 538.0.

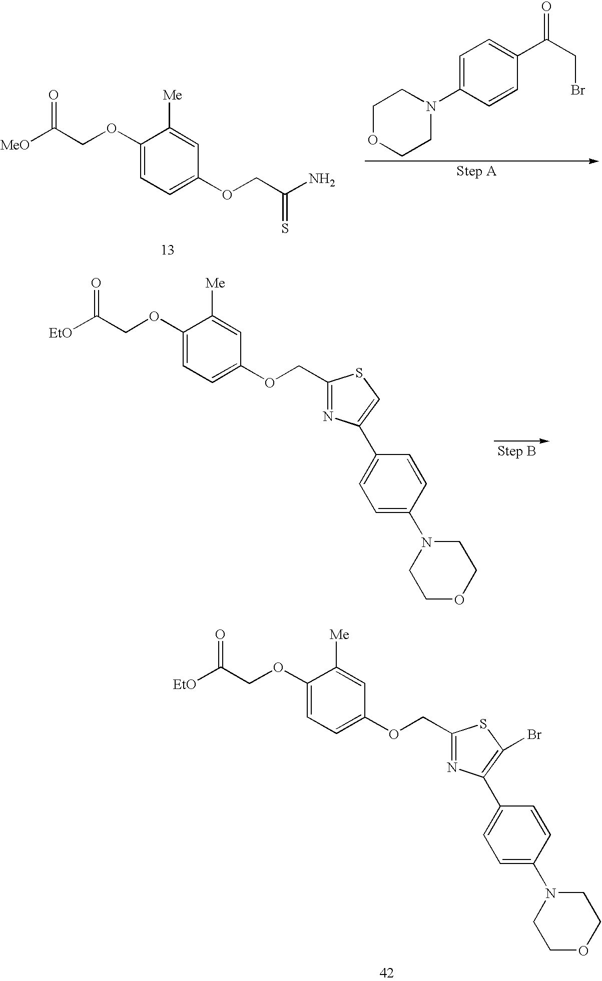

Intermediate 42: {4-[5-Bromo-4-(4-morpholin-4-yl-phenyl)-thiazol-2-ylmethoxy]-2-methyl-phenoxy}-acetic Acid Ethyl Ester.

Following the procedure of Intermediate 38, except substituting 2-Bromo-1-(4-morpholin-4-yl-phenyl)-ethanone for 2-bromo-4′-methoxyacetophenone in Step A, the title compound is prepared as a yellow solid: ¹H-NMR (400 MHz, CDCl₃) δ=7.99 (d, J=9.2 Hz, 2H), 7.33 (d, J=8.8 Hz, 2H), 6.85 (d, J=2.8 Hz, 1H), 6.74 (dd, J=2.8, 8.8 Hz, 1H), 6.66 (d, J=8.8 Hz, 1H), 5.27 (s, 2H) 4.59 (s, 2H), 4.25 (q, J=7.2 Hz, 2H), 4.04 (m, 4H), 3.45 (m, 4H), 2.29 (s, 3H), 1.28 (t, J=7.2 Hz, 3H). MS calculated for C₂₅H₂₈BrN₂O₅S (M+H⁺) 547.1, found 547.3.

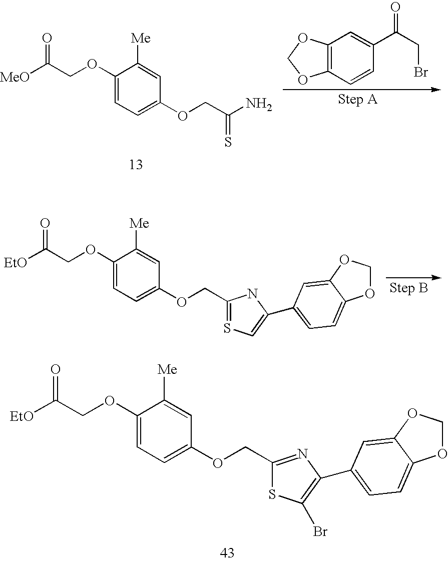

Intermediate 43: [4-(4-Benzo[1,3]dioxol-5-yl-5-bromo-thiazol-2-ylmethoxy)-2-methyl-phenoxy]-acetic Acid Ethyl Ester.

Following the procedure of Intermediate 38, except substituting 1-Benzo[1,3]dioxol-5-yl-2-bromo-ethanone for 2-bromo-4′-methoxyacetophenone in Step A, the title compound is prepared as a yellow solid: ¹H-NMR (400 MHz, CDCl₃) δ 7.43 (dd, J=1.6, 8.0 Hz, 1H), 7.41 (d, J=1.6 Hz, 1H), 6.89 (d, J=8.0 Hz, 1H), 6.85 (d, J=2.8 Hz, 1H), 6.74 (dd, J=2.8, 8.8 Hz, 1H), 6.66 (d, J=8.8 Hz, 1H), 6.02 (s, 2H), 5.27 (s, 2H) 4.59 (s, 2H), 4.25 (q, J=7.2 Hz, 2H), 2.27 (s, 3H), 1.29 (t, J=7.2 Hz, 3H). MS calculated for C₂₂H₂₁BrNO₆S (M+H⁺) 506.0, found 506.2.

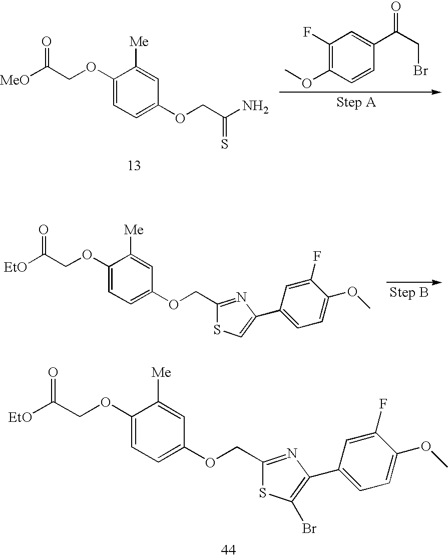

Intermediate 44: {4-[5-Bromo-4-(3-fluoro-4-methoxy-phenyl)-thiazol-2-ylmethoxy]-2-methyl-phenoxy}-acetic Acid Ethyl Ester.

Following the procedure of Intermediate 38, except substituting 2-bromo-1-(3-fluoro-4-methoxy-phenyl)-ethanone for 2-bromo-4′-methoxyacetophenone in Step A, the title compound is prepared as a white solid: ¹H-NMR (400 MHz, CDCl₃) δ=7.70 (m, 2H), 7.07 (t, J=8.4 Hz, 1H), 6.83 (d, J=2.4 Hz, 1H), 6.71 (dd, J=2.4, 8.8 Hz, 1H), 6.64 (d, J=8.8 Hz, 1H), 5.24 (s, 2H), 4.56 (s, 2H), 4.23 (q, J=7.2 Hz, 2H), 3.92 (s, 3H), 2.26 (s, 3H), 1.27 (t, J=7.2 Hz, 3H). MS calculated for C₂₂H₂₂BrFNO₅S (M+H⁺) 510.0, found 510.3.

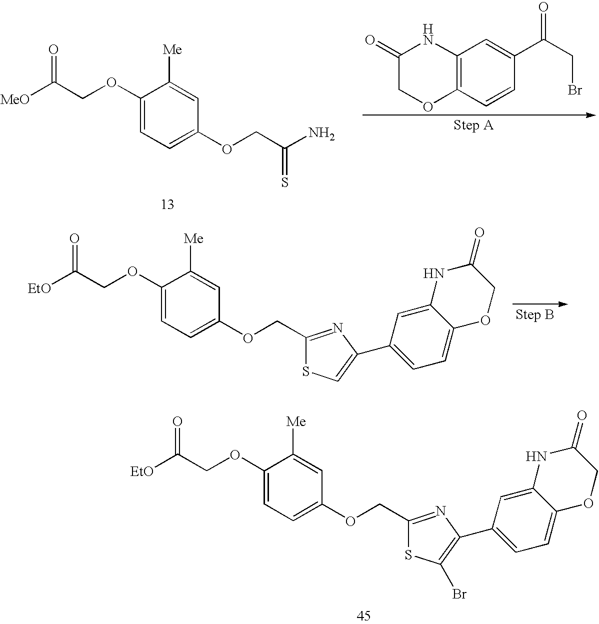

Intermediate 45; {4-[5-Bromo-4-(3-oxo-3,4-dihydro-2H-benzo[1,4]oxazin-6-yl)-thiazol-2-ylmethoxy]-2-methyl-phenoxy}-acetic Acid Ethyl Ester.

Following the procedure of Intermediate 38, except substituting 6-(2-bromo-acetyl)-4H-benzo[1,4]oxazin-3-one for 2-bromo-4′-methoxyacetophenone in Step A, the title compound is prepared as a yellow solid: ¹H-NMR (400 MHz, CDCl₃) δ=7.76 (s, 1H), 7.65 (dd, J=2.0, 8.4 Hz, 1H), 7.39 (d, J=2.0 Hz 1H), 7.10 (d, J=8.4 Hz, 1H), 6.89 (d, J=2.8 Hz, 1H), 6.78 (dd, J=2.8, 8.8 Hz, 1H), 6.71 (d, J=8.8 Hz, 1H), 5.31 (s, 2H), 4.71 (s, 2H), 4.64 (s, 2H), 4.31 (q, J=7.2 Hz, 2H), 2.33 (s, 3H), 1.33 (t, J=7.2 Hz, 3H). MS calculated for C₂₃H₂₂BrN₂O₆S (M+H⁺) 533.0, found 533.0.

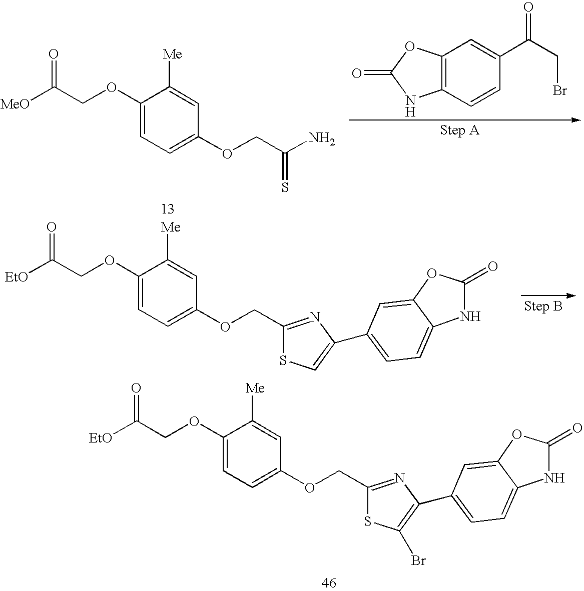

Intermediate 46: {4-[5-Bromo-4-(2-oxo-2,3-dihydro-benzooxazol-6-yl)-thiazol-2-ylmethoxy]-2-methyl-phenoxy}-acetic Acid Ethyl Ester.

Following the procedure of Intermediate 38, except substituting 6-(2-bromo-acetyl)-3H-benzooxazol-2-one for 2-bromo-4′-methoxyacetophenone in Step A, the title compound is prepared as a yellow solid: ¹H-NMR (400 MHz, CDCl₃) δ=8.07 (s, 1H), 7.82 (s, 1H), 7.78 (dd, J=1.6, 8.4 Hz, 1H), 7.12 (d, J=8.0 Hz, 1H), 6.86 (d, J=2.8 Hz, 1H), 6.74 (dd, J=2.8, 8.8 Hz, 1H), 6.67 (d, J=8.8 Hz, 1H), 5.28 (s, 2H), 4.60 (s, 2H), 4.26 (q, J=7.2 Hz, 2H), 2.29 (s, 3H), 1.29 (t, J=7.2 Hz, 3H). MS calculated for C₂₂H₂₀BrN₂O₆S (M+H⁺) 519.0, found 519.0.

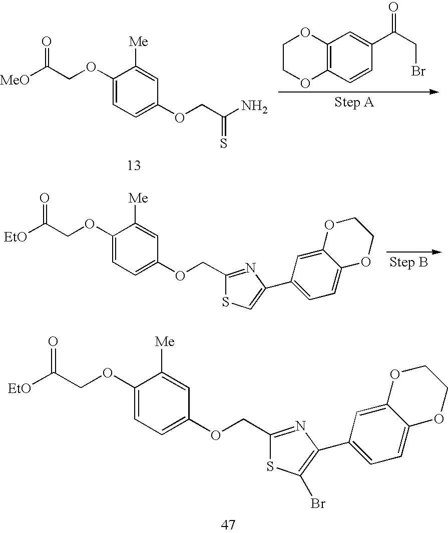

Intermediate 47: {4-[5-Bromo-4-(2,3-dihydro-benzo[1,4]dioxin-6-yl)-thiazol-2-ylmethoxy]-2-methyl-phenoxy}-acetic Acid Ethyl Ester.

Following the procedure of Intermediate 38, except substituting 2-bromo-1-(2,3-dihydro-benzo[1,4]dioxin-6-yl)-ethanone for 2-bromo-4′-methoxyacetophenone in Step A, the title compound is used without purification in the next step. MS calculated for C₂₃H₂₃BrNO₆S (M+H⁺) 520.0, found 520.0.

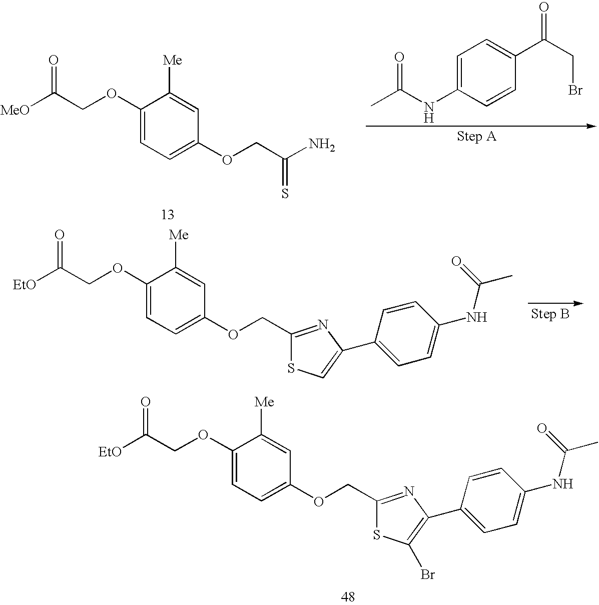

Intermediate 48: {4-[4-(4-Acetylamino-phenyl)-5-bromo-thiazol-2-ylmethoxy]-2-methyl-phenoxy}-acetic Acid Ethyl Ester.

Following the procedure of Intermediate 38, except substituting N-[4-(2-Bromo-acetyl)-phenyl]-acetamide for 2-bromo-4′-methoxyacetophenone in Step A, the title compound is prepared as a yellow solid: ¹H-NMR (400 MHz, CDCl₃) δ=7.91 (d, J=8.4 Hz, 2H), 7.60 (d, J=8.4 Hz, 2H), 6.86 (d, J=2.8 Hz, 1H), 6.74 (dd, J=2.8, 8.8 Hz, 1H), 6.67 (d, J=8.8 Hz, 1H), 5.35 (s, 2H), 4.59 (s, 2H), 4.26 (q, J=7.2 Hz, 2H), 2.29 (s, 3H), 2.21 (s, 3H), 1.28 (t, J=7.2 Hz, 3H). MS calculated for C₂₃H₂₄BrN₂O₅S (M+H⁺) 519.0, found 519.1.

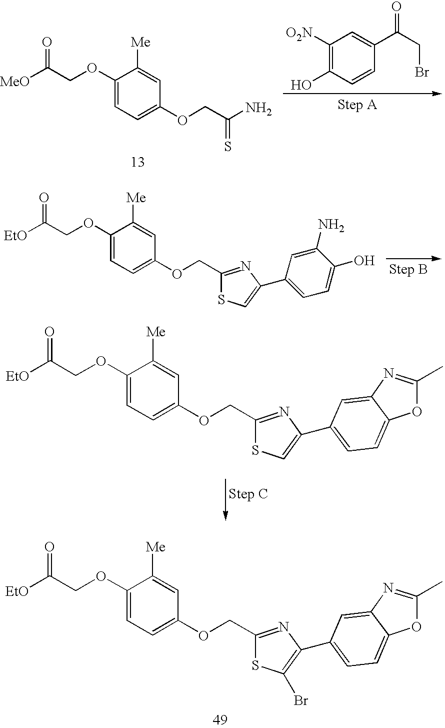

Intermediate 49: {4-[5-Bromo-4-(2-methyl-benzooxazol-5-yl)-thiazol-2-ylmethoxy]-2-methyl-phenoxy}-acetic Acid Ethyl Ester.

Step A: Intermediate 13 (2.1 g, 7.79 mmol), and 2-Bromo-1-(4-hydroxy-3-nitro-phenyl)-ethanone (2.0 g, 7.79 mmol) are heated to reflux in EtOH (40 mL) for 4 hours. Tin(II)chloride (4.4 g, 23 mmol) is added and the mixture is heated to reflux for an additional 2 hours. Then the mixture is extracted with ethyl acetate, washed with saturated sodium bicarbonate solution, and filtered through celite. The organic layer is dried (MgSO₄), filtered, concentrated and purified on reverse phase HPLC (H₂O/MeCN gradient) to afford {4-[4-(3-Amino-4-hydroxy-phenyl)-thiazol-2-ylmethoxy]-2-methyl-phenoxy}-acetic acid ethyl ester: MS calculated for C₂₁H₂₃N₂O₅S (M+H⁺) 415.1, found 415.4.

Step B: {4-[4-(3-Amino-4-hydroxy-phenyl)-thiazol-2-ylmethoxy]-2-methyl-phenoxy}-acetic acid ethyl ester (245 mg, 0.59 mmol) is dissolved in toluene (20 mL). Acetic anhydride (59 μL, 0.62 mmol) is added and the mixture is heated to reflux for 2 hours. Then p-toluene sulfonic acid (169 mg, 0.88 mmol) is added and the mixture is heated to reflux for another 2 h using a Dean-Stark trap to remove water. The mixture is diluted with saturated NaHCO₃, extracted with EtOAc and washed with brine (10 mL). The organic layer is dried (MgSO₄), filtered and concentrated to yield {2-methyl-4-[4-(2-methyl-benzooxazol-5-yl)-thiazol-2-ylmethoxy]-phenoxy}-acetic acid ethyl ester as a pale yellow solid, which is used without further purification in Step C.

Step C: Crude {2-Methyl-4-[4-(2-methyl-benzooxazol-5-yl)-thiazol-2-ylmethoxy]-phenoxy}-acetic acid ethyl ester (208 mg, 0.47 mmol) is dissolved in dichloromethane (10 mL) and pyridine (2 drops), then bromine (27 μL, 0.52 mmol) is added and the mixture is stirred at room temperature for 1 hour. The mixture is diluted with saturated NaHCO₃, extracted into dichloromethane and washed with brine (10 mL). The organic layer is dried (MgSO₄), filtered and concentrated to give Intermediate 49 as a white powder: MS calculated for C₂₃H₂₂BrN₂O₅S (M+H⁺) 517.0, found 517.0.

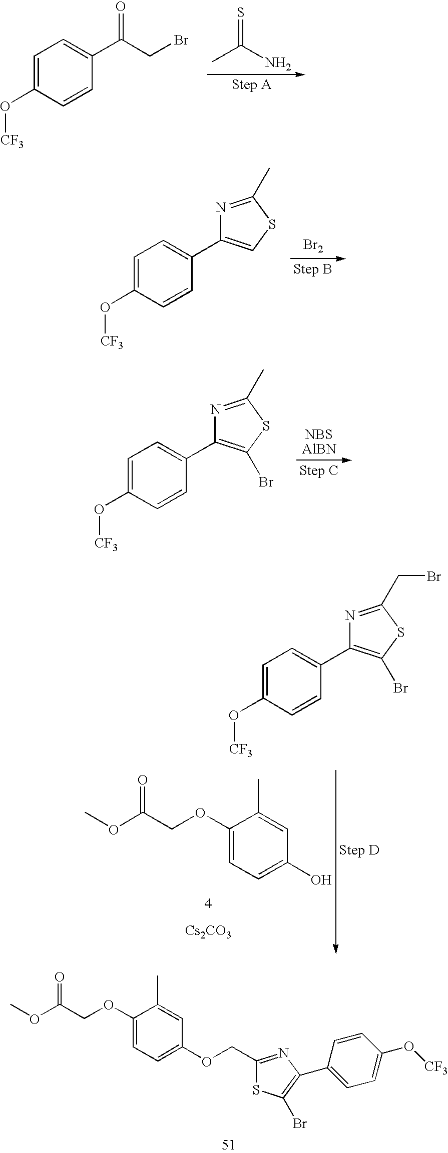

Intermediate 50: {4-[5-Bromo-4-(4-trifluoromethoxy-phenyl)-thiazol-2-ylmethoxy]-2-methyl-phenoxy}-acetic Acid Methyl Ester.

Step A: 2-Bromo-1-(4-trifluoromethoxy-phenyl)-ethanone (500 mg, 1.76 mmol) and thioacetamide (146 mg, 1.94 mmol) is dissolved in ethanol and heated to reflux for 2 hours. The solvent is removed in vacuo to afford crude 2-methyl-4-(4-trifluoromethoxy-phenyl)-thiazole, which is used without further purification in Step B.

Step B: 2-Methyl-4-(4-trifluoromethoxy-phenyl)-thiazole (1.76 mmol) is dissolved in dichloromethane (5 mL) containing acetic acid (1 mL). Bromine (0.20 mL, 3.9 mmol) is added and the mixture is heated at 40° C. for 2 hours. The mixture is diluted with saturated NaHCO₃, extracted into dichloromethane and washed with brine (10 mL). The organic layer is dried (MgSO₄), filtered and concentrated to give 5-bromo-2-methyl-4-(4-trifluoromethoxy-phenyl)-thiazole 50 as a yellow oil. ¹H-NMR (400 MHz, CDCl₃) δ=7.95 (d, J=8.4 Hz, 2H), 7.68 (d, J=8.8 Hz, 2H), 2.71 (s, 3H). MS calculated for C₁₁H₈BrF₃NOS (M+H⁺) 337.9, found 337.9.

Step C: 5-Bromo-2-methyl-4-(4-trifluoromethoxy-phenyl)-thiazole 50 (548 mg, 1.62 mmol) and N-bromosuccinimide (317 mg, 1.78 mmol) are dissolved in carbon tetrachloride (40 mL) and heated to 50° C. Azo-bis-isobutyronitrile (20 mg) is predissolved in carbon tetrachloride (10 mL) and added dropwise to the mixture, then the mixture is heated at 50° C. for 96 hours. The mixture is diluted with saturated NaHCO₃, extracted into dichloromethane and washed with brine (10 mL). The organic layer is dried (MgSO₄), filtered and concentrated to give crude 5-Bromo-2-bromomethyl-4-(4-trifluoromethoxy-phenyl)-thiazole which is used without further purification in Step D.

Step D: 5-Bromo-2-bromomethyl-4-(4-trifluoromethoxy-phenyl)-thiazole (1.62 mmol), Intermediate 4 (222 mg, 1.13 mmol), and cesium carbonate (736 mg, 2.26 mmol) are slurried in acetonitrile at room temperature for 1 hour. The mixture is filtered, the solvent evaporated, and the remainder purified by flash chromatography using a mixture of hexane and ethyl acetate (5:1) to afford 51 as a white solid: ¹H-NMR (400 MHz, CDCl₃) δ=7.97 (d, J=8.8 Hz, 2H), 7.30 (d, J=8.4 Hz, 2H), 6.86 (d, J=2.8 Hz, 1H), 6.74 (dd, J=2.8, 8.8 Hz, 1H), 6.66 (d, J=8.8 Hz, 1H), 5.27 (s, 2H), 4.61 (s, 2H), 3.80 (s, 3H), 2.29 (s, 3H). MS calculated for C₂₁H₁₈BrF₃NO₅S (M+H⁺) 532.0, found 532.0.

Intermediate 52: {4-[5-Bromo-4-(4-trifluoromethyl-phenyl)-thiazol-2-ylmethoxy]-2-methyl-phenoxy}-acetic Acid Methyl Ester.

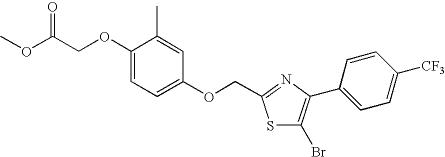

Following the procedure of Intermediate 51, except substituting 2-bromo-1-(4-trifluoromethylphenyl)-ethanone for 2-bromo-1-(4-trifluoromethoxy-phenyl)-ethanone in Step A, the title compound is prepared as a white solid: ¹H-NMR (400 MHz, CDCl₃) δ=8.08 (d, J=8.4 Hz, 2H), 7.71 (d, J=8.4 Hz, 2H), 6.86 (d, J=2.8 Hz, 1H), 6.75 (dd, J=2.8, 8.8 Hz, 1H), 6.66 (d, J=8.8 Hz, 1H), 5.28 (s, 2H), 4.61 (s, 2H), 3.80 (s, 3H), 2.29 (s, 3H). MS calculated for C₂₁H₁₈BrF₃NO₄S (M+H⁺) 516.0, found 516.3.

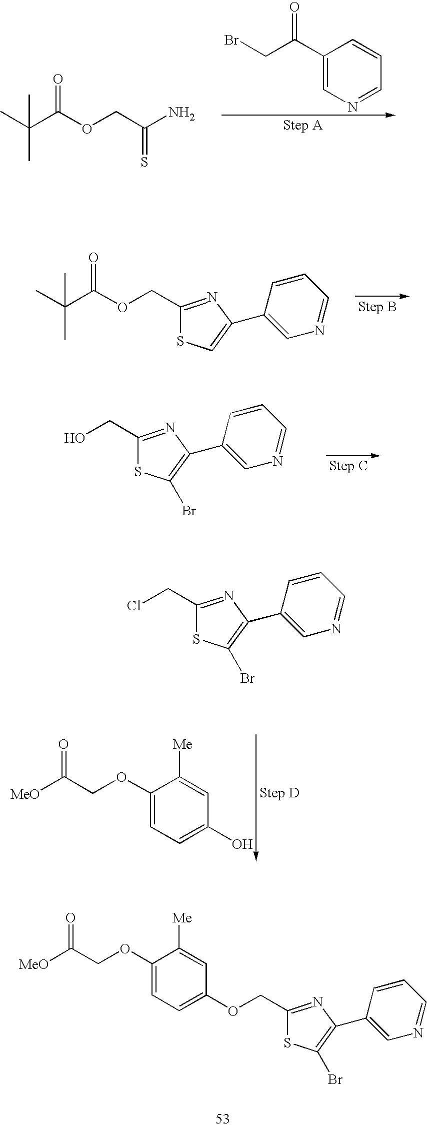

Intermediate 53: [4-(5-Bromo-4-pyridin-3-yl-thiazol-2-ylmethoxy)-2-methyl-phenoxy]-acetic Acid Methyl Ester.

Step A: 2-Bromo-1-pyridin-3-yl-ethanone (200 mg, 0.71 mmol) and 2-amino-2-thioxoethyl pivalate (131 mg, 0.75 mmol) are dissolved in ethanol and heated to reflux for 1 hour. The mixture is diluted with saturated NaHCO₃, extracted into dichloromethane and washed with brine (10 mL). The organic layer is dried (MgSO₄), filtered and concentrated to give 2,2-dimethyl-propionic acid 4-pyridin-3-yl-thiazol-2-ylmethyl ester, which is used without further purification in Step B.

Step B: Crude 2,2-Dimethyl-propionic acid 4-pyridin-3-yl-thiazol-2-ylmethyl ester (0.71 mmol) is dissolved in dichloromethane (10 mL) containing pyridine (2 drops), then bromine (47 μL, 0.93 mmol) is added and the mixture is stirred for 16 h at room temperature. The mixture is diluted with saturated NaHCO₃, extracted into dichloromethane and washed with brine (10 mL). The organic layer is dried (MgSO₄), filtered and concentrated. The residue is immediately dissolved in tetrahydrofuran (5 mL), then lithium hydroxide (1.0 N, 2 mL) is added and the mixture is stirred for 1 hour. The mixture is diluted with saturated NaHCO₃, extracted into ethyl acetate (2×) and washed with brine (10 mL). The organic layer is dried (MgSO₄), filtered and concentrated to give (5-Bromo-4-pyridin-3-yl-thiazol-2-yl)-methanol, which is used without further purification in Step C.

Step C: Crude (5-bromo-4-pyridin-3-yl-thiazol-2-yl)-methanol (0.71 mmol) is dissolved in dry tetrahydrofuran, then thionyl chloride (0.30 mL, 4.1 mmol) is added and the mixture is stirred for 1 hour. The mixture is diluted with saturated NaHCO₃, extracted into dichloromethane and washed with brine (10 mL). The organic layer is dried (MgSO₄), filtered and concentrated to give 3-(5-Bromo-2-chloromethyl-thiazol-4-yl)-pyridine, which is used without further purification in Step D.

Step D: Crude 3-(5-bromo-2-chloromethyl-thiazol-4-yl)-pyridine (0.71 mmol), Intermediate 4 (139 mg, 0.71 mmol) and cesium carbonate (464 mg, 1.42 mmol) are suspended in dry acetonitrile and stirred for 2 hours. Then the mixture is filtered, the solvent evaporated, and the remainder purified on reverse phase HPLC (H₂O/MeCN gradient) to afford Intermediate 53 as the major component of a mixture of compounds (by ¹H nmr). This mixture is used directly in the next step without further purification.

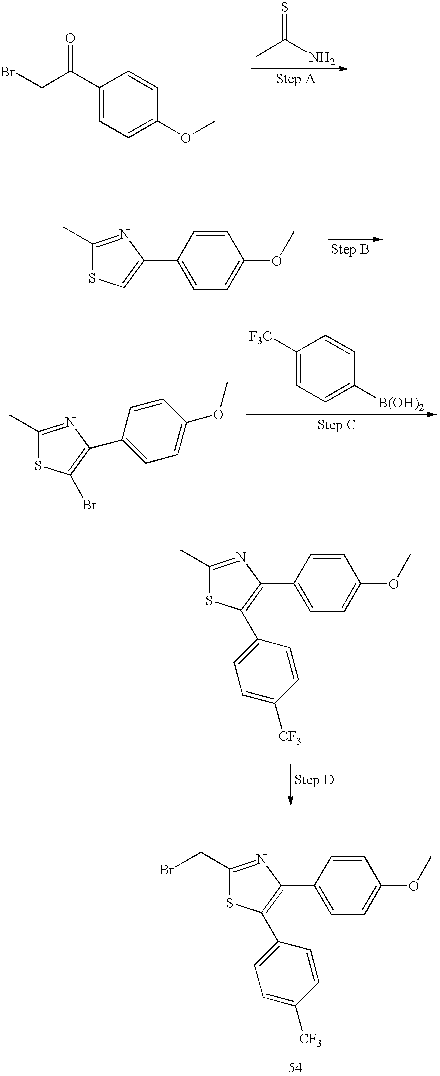

Intermediate 54: 2-Bromomethyl-4-(4-methoxy-phenyl)-5-(4-trifluoromethyl-phenyl)-thiazole.

Step A: 2-Bromo-1-(4-methoxy-phenyl)-ethanone (25.0 g, 109 mmol) and thioacetamide (9.0 g, 120 mmol) are dissolved in ethanol (60 mL) and heated to reflux for 2 hours. The solvent is removed in vacuo to afford crude 4-(4-Methoxy-phenyl)-2-methyl-thiazole, which is used without further purification in Step B.

Step B: 4-(4-Methoxy-phenyl)-2-methyl-thiazole (109 mmol) is dissolved in dichloromethane (300 mL). Bromine (6.20 mL, 120 mmol) is added and the mixture is heated at 40° C. for 3 hours. The mixture is diluted with saturated NaHCO₃, extracted into dichloromethane and washed with brine (50 mL). The organic layer is dried (MgSO₄), filtered and concentrated to give 5-bromo-4-(4-methoxy-phenyl)-2-methyl-thiazole. MS calculated for C₁₁H₁₁BrNOS (M+H⁺) 284.0, found 284.1.

Step C: 5-Bromo-4-(4-methoxy-phenyl)-2-methyl-thiazole (4 g, 14.1 mmol), 4-trifluoromethylphenylboronic acid (3.2 g, 16.9 mmol) and sodium carbonate (4.5 g, 42.3 mmol) are dissolved in H₂O (12.6 mL), ethanol (9.3 mL) and 1,2-dimethoxyethane (37.8 mL) and the mixture is degassed by bubbling Argon through the solution for 10 minutes. Pd(PPh₃)₄ (490 mg, 0.42 mmol) is added and the mixture is heated at 170° C. by microwave in a sealed tube for 10 minutes. The mixture is diluted with water (50 mL), extracted into EtOAc (200 mL) and washed with brine (50 mL). The organic layer is dried (MgSO₄), filtered, concentrated and purified on a column of silica gel using a mixture of hexane and ethyl acetate to afford 4-(4-Methoxy-phenyl)-2-methyl-5-(4-trifluoromethyl-phenyl)-thiazole: ¹H-NMR (400 MHz, CDCl₃) δ=7.54 (d, J=8.0 Hz, 2H), 7.42 (d, J=8.0 Hz, 2H), 7.40 (d, J=8.8 Hz, 2H), 6.84 (d, J=8.8 Hz, 2H), 3.81 (s, 3H), 2.77 (s, 3H). MS calculated for C₁₈H₁₅F₃NOS (M+H⁺) 350.1, found 350.0.

Step D: 4-(4-Methoxy-phenyl)-2-methyl-5-(4-trifluoromethyl-phenyl)-thiazole (3.66 g, 10.5 mmol) and N-bromosuccinimide (2.05 g, 11.5 mmol), are dissolved in carbon tetrachloride (60 mL) and heated to 50° C. Azo-bis-isobutyronitrile (172 mg) is predissolved in carbon tetrachloride (10 mL) and added dropwise to the mixture, then the mixture is heated at 60° C. for 16 hours. The mixture is diluted with saturated NaHCO₃, extracted into dichloromethane and washed with brine (50 mL). The organic layer is dried (MgSO₄), filtered, concentrated and purified by flash chromatography using a mixture of hexane and ethyl acetate to afford Intermediate 54. MS calculated for C₁₈H₁₄BrF₃NOS (M+H⁺) 428.0, found 428.0.

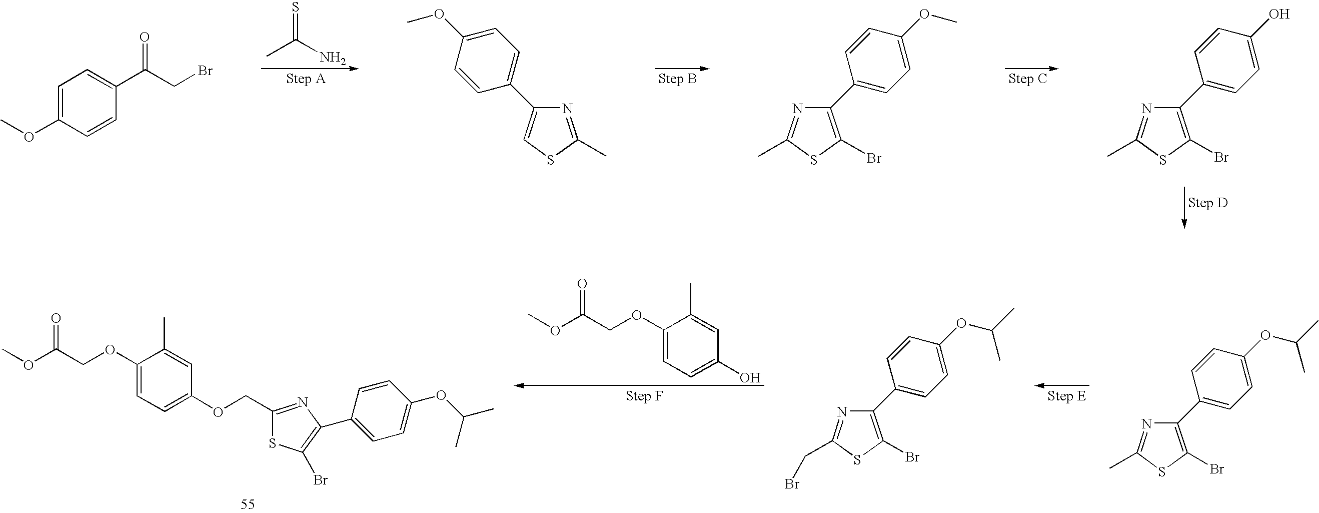

Intermediate 55: {4-[5-Bromo-4-(4-isopropoxy-phenyl)-thiazol-2-ylmethoxy]-2-methyl-phenoxy}-acetic Acid Methyl Ester.

Step A: Thioacetamide (9.0 g, 120 mmol) and 2-bromo-1-(4-methoxy-phenyl)-ethanone (25 g, 109 mmol) are dissolved in ethanol (60 mL) and heated to reflux for 2 hours. The ethanol is removed under vacuum and the crude 4-(4-methoxy-phenyl)-2-methyl-thiazole is used in Step B without further purification.

Step B: 4-(4-Methoxy-phenyl)-2-methyl-thiazole (109 mmol) is dissolved in dichloromethane (300 mL). Bromine (6.2 ml, 120 mmol) is added and the mixture is heated to reflux for 3 hours. The mixture is quenched with saturated NaHCO₃(aq), extracted into dichloromethane, washed with saturated NaHCO₃(aq), dried over magnesium sulfate, filtered and evaporated to give 5-bromo-4-(4-methoxy-phenyl)-2-methyl-thiazole as a pale beige powder: ¹H-NMR (400 MHz, CDCl₃) δ=7.85 (d, J=8.8 Hz, 2H), 6.96 (d, J=8.8 Hz, 2H), 3.85 (s, 3H), 2.69 (s, 3H). MS calculated for C₁₁H₁₁BrNOS (M+H⁺) 284.0, found 284.1.

Step C: 5-Bromo-4-(4-methoxy-phenyl)-2-methyl-thiazole (15.0 g, 52.8 mmol) is dissolved in dichloromethane (200 mL). Boron tribromide (15 mL, 158.3 mmol) is added and the mixture is stirred at room temperature for 1 hour. The mixture is quenched with saturated NaHCO₃(aq), extracted into dichloromethane, washed with saturated NaHCO—₃(aq), dried over magnesium sulfate, filtered and evaporated to yield crude 4-(5-Bromo-2-methyl-thiazol-4-yl)-phenol (15.4 g), which is used without purification in Step D: ¹H-NMR (400 MHz, CDCl₃) δ=7.79 (d, J=8.4 Hz, 2H), 6.88 (d, J=8.4 Hz, 2H), 3.16 (s, 1H), 2.70 (s, 3H). MS calculated for C₁₀H₉BrNOS (M+H⁺) 270.0, found 270.2.

Step D: 4-(5-Bromo-2-methyl-thiazol-4-yl)-phenol (38.3 mmol) is dissolved in acetone (100 mL). K₂CO₃ (10.6 g, 76.6 mmol) is added, followed by 2-iodopropane (7.7 mL, 76.6 mmol) and the resulting mixture is heated to reflux for 18 hours. The solvent is evaporated in vacuo and the residue is purified by flash chromatography using a mixture of hexane and ethyl acetate to afford 5-bromo-4-(4-isopropoxy-phenyl)-2-methyl-thiazole. MS calculated for C₁₃H₁₅BrNOS (M+H⁺) 312.0, found 312.0.

Step E: 5-Bromo-4-(4-isopropoxy-phenyl)-2-methyl-thiazole (3.4 g, 10.89 mmol) is dissolved in carbon tetrachloride (100 mL). N-bromosuccinimide (2.52 g, 14.16 mmol) is added and the mixture is heated to 50° C., then AIBN (179 mg, 1.09 mmol) is added. The mixture is heated to 70° C. for 5 hours. Additional bromine (0.5 g) and AIBN (60 mg) is added and stirring is continued at 70° C. for another 12 hours. The mixture is then cooled, quenched with water, extracted into dichloromethane, dried over MgSO₄, filtered and evaporated to give crude 5-bromo-2-bromomethyl-4-(4-isopropoxy-phenyl)-thiazole, which is used directly in Step F.

Step F: 5-Bromo-2-bromomethyl-4-(4-isopropoxy-phenyl)-thiazole (10.89 mmol) and Intermediate 4 (2.13 g, 10.89 mmol) are dissolved in acetonitrile (100 mL). Cesium carbonate (7.1 g, 21.78 mmol) is added and the mixture is stirred at room temperature for 2 hours. The mixture is filtered, evaporated, and purified by flash chromatography using a mixture of hexane and ethyl acetate to afford Intermediate 55: ¹H-NMR (400 MHz, CDCl₃) δ=7.92 (d, J=8.4 Hz, 2H), 7.03 (d, J=8.4 Hz, 2H), 6.93 (d, J=2.8 Hz, 1H), 6.80 (dd, J=2.8, 8.8 Hz, 1H), 6.73 (d, J=8.8 Hz, 1H), 5.33 (s, 2H), 4.67 (s, 2H), 3.86 (s, 3H), 2.36 (s, 3H), 2.12 (s, 1H, 1.44 (s, 6H). MS calculated for C₂₃H₂₅BrNO₅S (M+H⁺) 506.1, found 506.1.

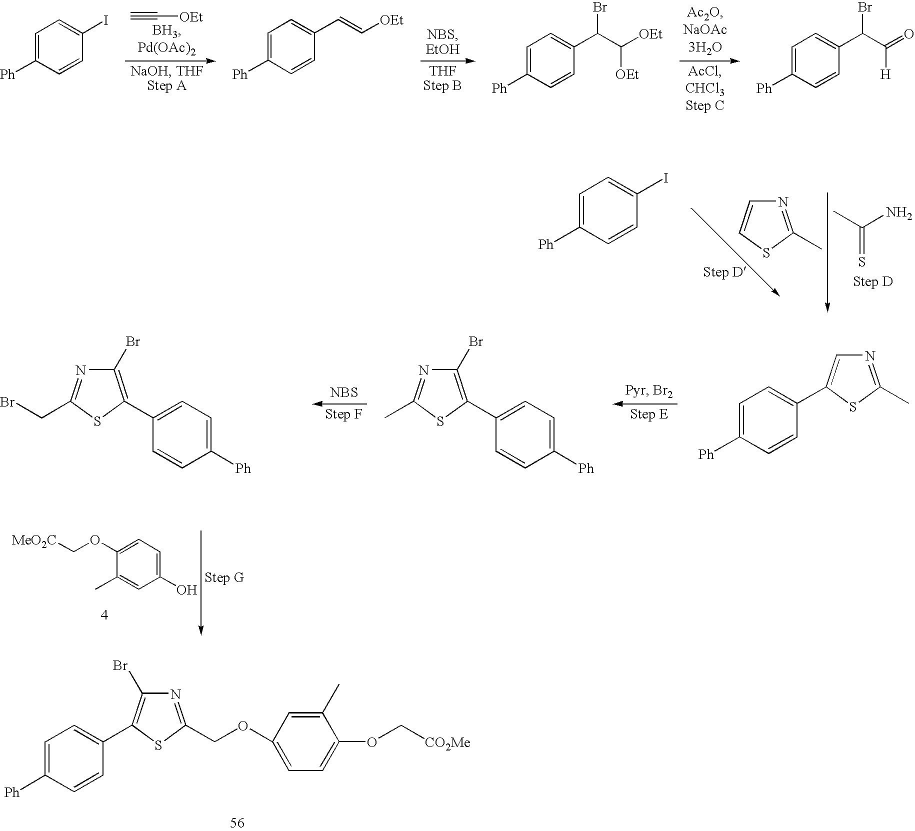

Intermediate 56: [4-(5-biphenyl-4-yl-4-bromo-thiazol-2-ylmethoxy)-2-methyl-phenoxy]-acetic Acid Methyl Ester.

Step A: To a solution of ethyl ethynyl ether (6.0 g, 85.6 mmol) in THF (100 mL) at 0° C. is added borane-tetrahydrofuran complex (1.0 mol in THF, 28.53 mL, 28.53 mmol). The mixture is warmed to room temperature and stirred for 2 hours. The resulting solution is added to a mixture of 4-iodobiphenyl (20.0 g, 71.33 mmol), triphenylphosphine (598 mg, 2.28 mmol), palladium(II)acetate (128 mg, 0.571 mmol) and sodium hydroxide (8.5 g, 214.0 mmol) in THF (200 mL). The mixture is heated to reflux for 15 h, then cooled, diluted with EtOAc (1000 mL), washed with saturated Na₂CO₃, brine and water. The organic layer is dried (MgSO₄), filtered and concentrated to give crude product, which is purified by silica gel chromatography (ether/hexane, gradient) to give 4-(2-ethoxy-vinyl)-biphenyl as a white solid: ¹H-NMR (400 MHz, CDCl₃) δ=7.52-7.19 (m, 9H), 6.97 (d, J=12.8 Hz, 1H), 5.81 (d, J=12.8 Hz, 1H), 3.87 (m, 2H), 1.29 (t, 3H). MS calculated for C₁₆H₁₇O (M+H⁺) 225.1, found 225.1.

Step B: 4-(2-Ethoxy-vinyl)-biphenyl (7.60 g, 33.88 mmol) is dissolved in a mixture of EtOH/THF (120/30 mL), then NBS (6.03 g, 33.88 mmol) is added. The mixture is stirred at room temperature for 2 h, then concentrated and purified by silica gel chromatography (EtOAc/hexane, gradient) to give 4-(1-bromo-2,2-diethoxy-ethyl)-biphenyl as a white solid: ¹H-NMR (400 MHz, CDCl₃) δ=7.33-7.63 (m, 9H), 4.98 (d, J=6.4 Hz, 1H), 4.88 (d, J=6.4 Hz, 1H), 3.81 (m, 1H), 3.64 (m, 2H), 3.45 9(m, 1H), 1.29 (t, J=7.2 Hz, 3H), 1.07 (t, J=7.2 Hz, 3H). MS calculated for C₁₈H₂₁BrO₂ (M⁺) 349.3, found 270.1 (M−Br)⁺.

Step C: 4-(1-Bromo-2,2-diethoxy-ethyl)-biphenyl (750 mg, 2.15 mmol) is dissolved in chloroform (3 mL), then Ac₂O (220 mg, 2.15 mmol), NaOAc.3H₂O (175.4 mg, 1.29 mmol) and AcCl (118 mg, 1.51 mmol) are added successively and the mixture is stirred at 55° C. for 5 hours. The mixture is diluted with CH₂Cl₂ (50 mL) and washed with saturated NaHCO₃ and brine. The organic layer is dried (MgSO₄), filtered and concentrated to give crude biphenyl-4-yl-bromo-acetaldehyde 50 as a thick oil, which is used in the next step without further purification. MS calculated for C₁₄H₁₁BrO (M⁺) 275.2, found 195.1 (M−Br)⁺.

Step D: The aldehyde 50 (0.57 g, 2.07 mmol) is dissolved in EtOH (8 mL), then thioacetamide (156 mg, 2.07 mmol) is added and the mixture is stirred at 90° C. for 15 hours. The solution is diluted with EtOAc (50 mL) and washed with saturated NaHCO₃ (30 mL) and brine (10 mL). The organic layer is dried (MgSO₄), filtered and concentrated to give crude product, which is purified by silic gel chromatography with EtOAc/hexane (gradient) to give 5-biphenyl-4-yl-2-methyl-thiazole as a white solid: ¹H-NMR (400 MHz, CDCl₃) δ=7.78 (s, 1H), 7.51-7.56 (m, 5H), 4.28-7.41 (m, 4H), 2.69 (s, 3H). MS calculated for C₁₆H₁₄NS (M+H⁺) 252.1, found 252.0.

Step D′: An alternative one step coupling reaction to prepare 5-biphenyl-4-yl-2-methyl-thiazole.

4-Iodobiphenyl (40.0 g, 171.6 mmol) is dissolved in DMF (800 mL), then 2-methylthiazole (8.50 g, 85.5 mmol), triphenylphosphine (3.6 g, 13.73 mmol), cesium carbonate (55.9 g, 171.6 mmol), palladium(II)acetate (3.01 g, 13.7 mmol) are added and the mixture is stirred at 140° C. for 24 hours. The reaction mixture is subsequently filtered through Celite 545 and washed with sat. K₂CO₃ and EtOAc. The filtrate is diluted with EtOAc and washed with saturated NaHCO₃, brine and water. The organic layer is dried (MgSO₄), filtered and concentrated to give crude product, which is purified by silic gel chromatography (ether/hexane, gradient) to give 5-biphenyl-4-yl-2-methyl-thiazole.

Step E: 5-Biphenyl-4-yl-2-methyl-thiazole (1.0 g, 3.98 mmol) is dissolved in chloroform (100 mL), then bromine (245 μL, 4.77 mmol) is added and the mixture is stirred at room temperature for 15 hours. Pyridine (354.1 μL, 4.38 mmol) is added and the solution is stirred for 4 h at room temperature. The solution is diluted with CH₂Cl₂ (100 mL) and washed with saturated NaHCO₃ (50 mL) and brine (30 mL). The organic layer is dried (MgSO₄), filtered and concentrated to give crude product, which is purified by silic gel chromatography with ether/hexane (gradient) to give 5-biphenyl-4-yl-4-bromo-2-methyl-thiazole as a white solid: ¹H-NMR (400 MHz, CDCl₃) δ=7.55-7.64 (m, 5H), 4.29-7.42 (m, 4H), 2.68 (s, 3H). MS calculated for C₁₆H₁₃BrNS (M+H⁺) 330.0, found 330.0.

Step F: N-Bromosuccinimide (504 mg, 2.83 mmol) is added to a solution of 5-biphenyl-4-yl-4-bromo-2-methyl-thiazole (850 mg, 2.57 mmol) in carbon tetrachloride (50 mL). The above solution is stirred at 75° C. for 18 hours. The solution is diluted with CH₂Cl₂ (50 mL) and washed with saturated NaHCO₃ (50 mL) and brine (30 mL). The organic layer is dried (MgSO₄), filtered and concentrated to give crude product, which is purified by silic gel chromatography with hexane/ether (gradient) to give 5-biphenyl-4-yl-4-bromo-2-bromomethyl-thiazole as a white solid: ¹H-NMR (400 MHz, CDCl₃) δ=7.55-7.66 (m, 5H), 4.30-7.45 (m, 4H), 4.65 (s, 2H). MS calculated for C₁₆H₁₂Br₂NS (M+H⁺) 410.1, found 410.9.

Step G: Intermediate 4 (169 mg, 0.86 mmol) and Cs₂CO₃ (308 mg, 0.94 mmol) are added to a solution of 5-biphenyl-4-yl-4-bromo-2-bromomethyl-thiazole (336 mg, 0.82 mmol) in MeCN (30 mL). The mixture is stirred for 3 h at room temperature. After the mixture is filtered, the organic solution is concentrated and purified by silic gel chromatography with hexane/ether (gradient) to give [4-(5-biphenyl-4-yl-4-bromo-thiazol-2-ylmethoxy)-2-methyl-phenoxy]-acetic acid methyl ester (56) as a white solid. ¹H-NMR (400 MHz, CDCl₃) δ=7.60-7.72 (m, 5H), 7.35-7.47 (m, 4H), 6.85 (d, J=2.8 Hz, 1H), 6.74(dd, J=2.8 Hz, J=8.8 Hz, 1H), 6.65 (d, J=8.8 Hz, 1H), 5.28 (s, 2H), 4.60 (s, 2H), 3.78 (s, 3H), 2.27 (s, 3H). MS calculated for C₂₆H₂₃BrNO₄S (M+H⁺) 525.0, found 525.0.

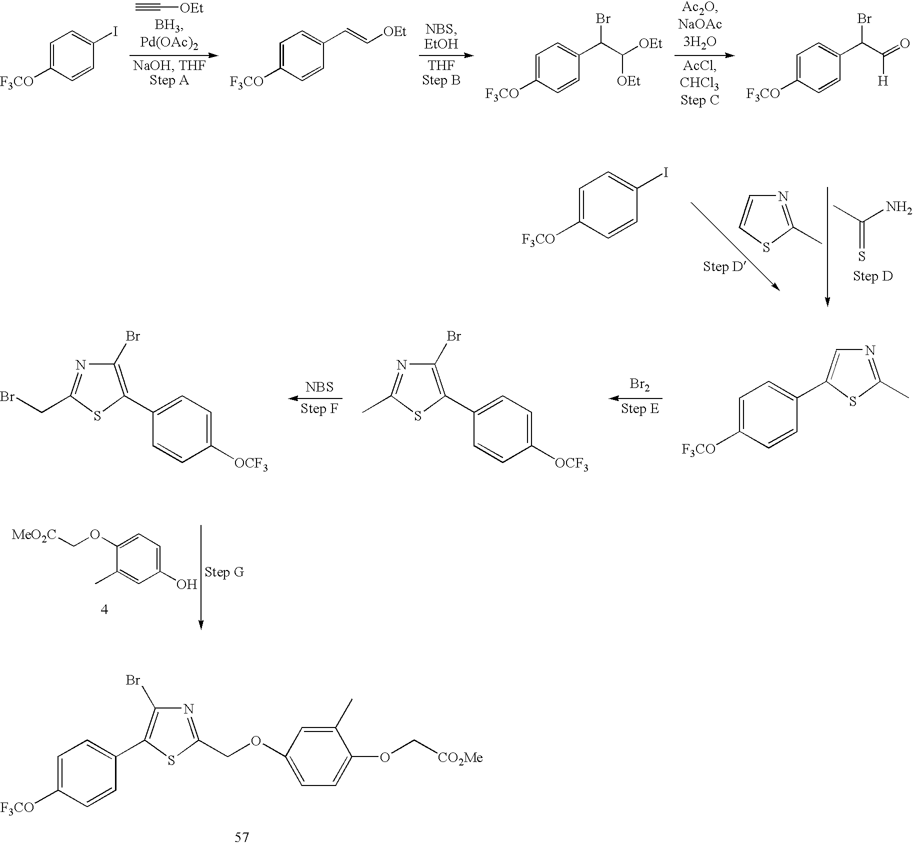

Intermediate 57: {4-[4-bromo-5-(4-trifluoromethoxy-phenyl)-thiazol-2-ylmethoxy]-2-methyl-phenoxy}-acetic Acid Methyl Ester.

Step A: Following the procedure of intermediate 56, except substituting 1-iodo-4-trifluoromethoxy-benzene for 4-iodobiphenyl in step A, 1-(2-ethoxy-vinyl)-4-trifluoromethoxy-benzene is prepared as a white solid: ¹H-NMR (400 MHz, CDCl₃) δ=7.38 (d, J=8.8 Hz, 2H), 7.26 (d, J=8.4 Hz, 2H), 7.13 (d, J=12.8 Hz, 1H), 5.98 (d, J=13.2 Hz, 1H), 4.08 (q, J=7.0 Hz, 2H), 1.50 (t, J=7.0 Hz, 3H). MS calculated for C₁₁H₁₂F₃O₂ (M+H⁺) 233.1, found 233.1.

Step B: Following the procedure of intermediate 56, except substituting 1-(2-ethoxy-vinyl)-4-trifluoromethoxy-benzene for 4-(2-ethoxy-vinyl)-biphenyl in step B, 1-(1-bromo-2,2-diethoxy-ethyl)-4-trifluoromethoxy-benzene is prepared as a white solid: 1H-NMR (400 MHz, CDCl₃) δ=7.52 (d, J=8.8 Hz, 2H), 7.21 (d, J=8.4 Hz, 2H), 4.95 (d, J=6.4 Hz, 1H), 4.83 (d, J=6.0 Hz, 1H), 3.84-3.77 (m, 1H), 3.71-3.61 (m, 2H), 3.50-3.43 (m, 1H), 1.29 (t, J=7.0 Hz, 3H), 1.09 (t, J=7.0 Hz, 3H). MS calculated for C₁₃H₁₆BrF₃O₃ (M⁺) 356.0, found 277.0 (M−Br)⁺.

Step C: Following the procedure of intermediate 56, except substituting 1-(1-bromo-2,2-diethoxy-ethyl)-4-trifluoromethoxy-benzene for 4-(1-bromo-2,2-diethoxy-ethyl)-biphenyl in step C, bromo-(4-trifluoromethoxy-phenyl)-acetaldehyde is prepared as a white solid without purification. MS calculated for C₉H₆BrF₃O₂ (M⁺) 283.1, found 203.1 (M−Br)⁺.

Step D: Following the procedure of intermediate 56, except substituting bromo-(4-trifluoromethoxy-phenyl)-acetaldehyde for biphenyl-4-yl-bromo-acetaldehyde in step D, 2-methyl-5-(4-trifluoromethoxy-phenyl)-thiazole is prepared as a white solid: ¹H-NMR (400 MHz, CDCl₃) δ=7.71 (s, 1H), 7.46 (m, 2H), 7.18 (m, 2H), 2.68 (s, 3H). MS calculated for C₁₁H₉F₃NOS (M+H⁺) 260.0, found 260.0.

Step D′: An alternative one step coupling reaction to prepare 2-methyl-5-(4-trifluoromethoxy-phenyl)-thiazole.

Following the procedure of intermediate 56, except substituting 1-iodo-4-trifluoromethoxy-benzene for 4-iodobiphenyl in step D′. 2-methyl-5-(4-trifluoromethoxy-phenyl)-thiazole is obtained.

Step E: Following the procedure of intermediate 56, except substituting D-methyl-5-(4-trifluoromethoxy-phenyl)-thiazole for 5-biphenyl-4-yl-2-methyl-thiazole and without adding pyridine in step E, 4-bromo-2-methyl-5-(4-trifluoromethoxy-phenyl)-thiazole is prepared as a colorless oil: ¹H-NMR (400 MHz, CDCl₃) δ=7.56 (m, 2H), 7.21 (m, 2H), 2.67 (s, 3H). MS calculated for C₁₁H₈BrF₃NOS (M+H⁺) 337.9, found 337.9.

Step F: Following the procedure of intermediate 56, except substituting 4-bromo-2-methyl-5-(4-trifluoromethoxy-phenyl)-thiazole for 5-biphenyl-4-yl-4-bromo-2-methyl-thiazole in step F, 4-bromo-2-bromomethyl-5-(4-trifluoromethoxy-phenyl)-thiazole is prepared as a yellow oil: ¹H-NMR (400 MHz, CDCl₃) δ=7.59 (m, 2H), 7.23 (m, 2H), 4.63 (s, 2H). MS calculated for C₁₁H₇Br₂F₃NOS (M+2H)⁺ 416.9, found 416.8.

Step G: Following the procedure of intermediate 56, except substituting 4-bromo-2-bromomethyl-5-(4-trifluoromethoxy-phenyl)-thiazole for 5-bromo-2-bromomethyl-4-(4-methoxy-phenyl)-oxazole in step G, {4-[4-bromo-5-(4-trifluoromethoxy-phenyl)-thiazol-2-ylmethoxy]-2-methyl-phenoxy}-acetic acid methyl ester (57) is prepared as a white solid. ¹H-NMR (400 MHz, CDCl₃) δ=7.66 (m, 2H), 7.28 (m, 2H), 6.85 (d, J=2.8 Hz, 1H), 6.74(dd, J=3.2 Hz, J=8.8 Hz, 1H), 6.67 (d, J=8.8 Hz, 1H), 5.28 (s, 2H), 4.61 (s, 2H), 3.80 (s, 3H), 2.29 (s, 3H). MS calculated for C₂₁H₁₈BrF₃NO₅S (M+H⁺) 532.0, found 532.0.

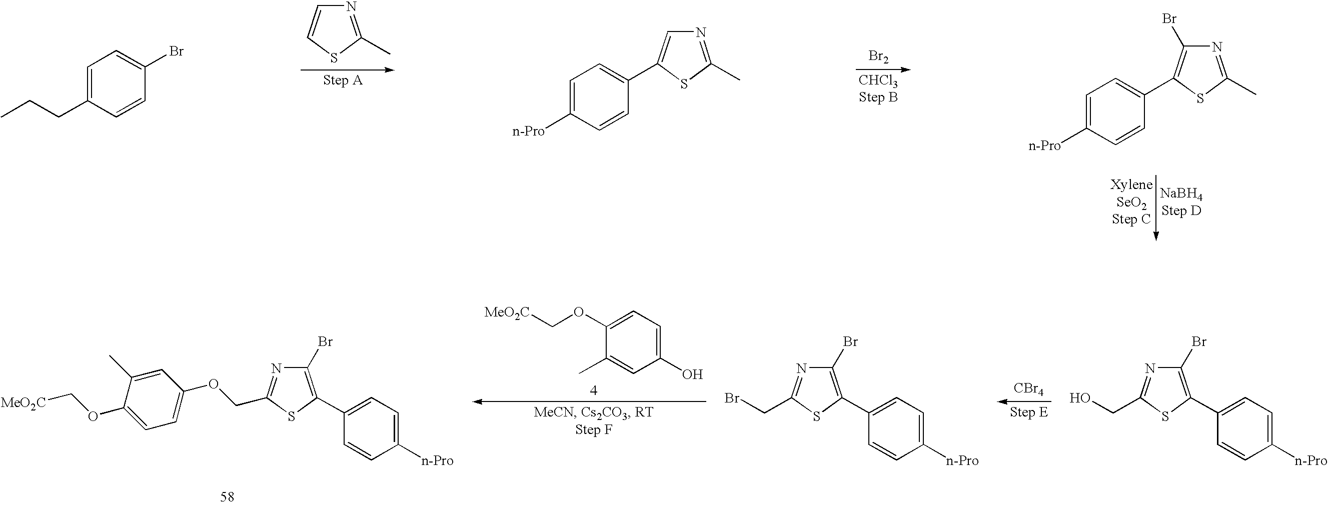

Intermediate 58: {4-[4-bromo-5-(4-propyl-phenyl)-thiazol-2-ylmethoxy]-2-methyl-phenoxy}-acetic Acid Methyl Ester.

Step A: 1-Bromo-4-propyl-benzene (50.0 g, 251.1 mmol) is dissolved in DMF (800 mL), then 2-methylthiazole (12.45 g, 125.6 mmol), triphenylphosphine (3.2 g, 12.56 mmol), cesium carbonate (81.2 g, 251.14 mmol), palladium(II)acetate (4.5 g, 20.09) are added and the mixture is stirred at 140° C. for 24 hours. The reaction mixture is filtered through Celite 545, washed with sat. K₂CO₃ and EtOAc. The solution is diluted with EtOAc and washed with saturated NaHCO₃, brine and water. The organic layer is dried (MgSO₄), filtered and concentrated to give crude product, which is purified by silic gel chromatography with ether/hexane (gradient) to give 2-methyl-5-(4-propyl-phenyl)-thiazole as an oil: ¹H-NMR (400 MHz, CDCl₃) δ=7.83 (s, 1H), 7.49 (d, J=8.4 Hz, 2H), 7.26 (d, J=8.4 Hz, 2H), 2.80 (s, 3H), 2.66 (t, J=7.6 Hz, 2H), 1.71 (m, 2H), 1.02 (t, J=7.2 Hz, 3H). MS calculated for C₁₃H₁₆NS (M+H⁺) 218.1, found 218.1.

Step B: 2-Methyl-5-(4-propyl-phenyl)-thiazole (2.0 g, 9.20 mmol) is dissolved in chloroform (25 mL), then bromine (0.52 mL, 10.12 mmol) is added and the mixture is stirred at room temperature for 2 hours. The solution is diluted with CH₂Cl₂ and washed with saturated NaHCO₃ and brine (100 mL). The organic layer is dried (MgSO₄), filtered and concentrated to give crude product, which is purified by silic gel chromatography with ether/hexane (gradient) to give 4-bromo-2-methyl-5-(4-propyl-phenyl)-thiazole as an oil: ¹H-NMR (400 MHz, CDCl₃) δ=7.52 (d, J=8.0 Hz, 2H), 7.23 (d, J=8.0 Hz, 2H), 2.71 (s, 3H), 2.62 (t, J=7.4 Hz, 2H), 1.67 (m, 2H), 0.99 (t, J=7.4 Hz, 3H). MS calculated for C₁₃H₁₄BrNS (M+H⁺) 296.0, found 296.0.

Step C and D: Selenium dioxide (4.5 g, 40.51 mmol) is added to a solution of 4-bromo-2-methyl-5-(4-propyl-phenyl)-thiazole (6.0 g, 20.25 mmol) in xylene (150 mL). The mixture is stirred at 150° C. for 30 hours. After 15 h an additional 1.2 g of SeO₂ is added to the reaction mixture. Then the solution is diluted with EtOAc and washed with saturated Na₂CO₃ and brine. The organic layer is dried (MgSO₄), filtered and concentrated to give 4-bromo-5-(4-propyl-phenyl)-thiazole-2-carbaldehyde as a crude product, which is used for next reaction.

NaBH₄ (604 mg, 16.0 mmol) is added to a solution of crude 4-bromo-5-(4-propyl-phenyl)-thiazole-2-carbaldehyde in MeOH (100 mL) and the mixture is stirred for 10 min. The solution is concentrated, diluted with EtOAc, washed with saturated Na₂CO₃ and brine. The organic layer is dried (MgSO₄), filtered and concentrated to give a crude mixture, which is purified by silic gel chromatography with hexane/EtOAc (gradient) to give [4-bromo-5-(4-propyl-phenyl)-thiazol-2-yl]-methanol as a white solid: ¹H-NMR (400 MHz, CDCl₃) δ=7.37 (d, J=8.0 Hz, 2H), 7.09 (d, J=8.0 Hz, 2H), 4.79 (s, 2H), 2.47 (t, J=8.0 Hz, 2H), 2.17 (s, bro. 1H), 1.51 (m, 2H), 0.81 (t, J=7.4 Hz, 3H). MS calculated for C₁₃H₁₅BrNOS (M+H⁺) 312.0, found 312.0.

Step E: P(Ph)₃ (2.22 g, 8.46 mmol) is added to the solution of [4-bromo-5-(4-propyl-phenyl)-thiazol-2-yl]-methanol in CH₂Cl₂ (40 mL) and stirred for 10 min at 0° C. Then CBr₄ (2.81 g, 8.46 mmol) dissolved in CH₂Cl₂ (20 mL) is added to the reaction mixture. The mixture is warmed to room temperature and stirred overnight. The solution is concentrated to give a crude mixture, which is purified by silic gel chromatography with hexane/ether (gradient) to give 4-bromo-2-bromomethyl-5-(4-propyl-phenyl)-thiazole as a colorless oil: ¹H-NMR (400 MHz, CDCl₃) δ=7.37 (d, J=8.0 Hz, 2H), 7.08 (d, J=8.0 Hz, 2H), 4.52 (s, 2H), 2.46 (t, J=8.0 Hz, 2H), 1.49 (m, 2H), 0.80 (t, J=8.0 Hz, 3H). MS calculated for C₁₃H₁₄Br₂NO (M+H⁺) 373.9, found 373.9.

Step F: A mixture of 4-bromo-2-bromomethyl-5-(4-propyl-phenyl)-thiazole (910 mg, 2.43 mmol), (4-hydroxy-2-methyl-phenoxy)-acetic acid methyl ester (4) (524 mg, 2.67 mmol) and Cs₂CO₃ (911 mg, 2.79 mmol) in MeCN (15 mL) is stirred at room temperature for 4 hours. The mixture is filtered, then concentrated to give crude product, which is purified by silic gel chromatography with EtOAc/hexane (gradient) to give {4-[4-bromo-5-(4-propyl-phenyl)-thiazol-2-ylmethoxy]-2-methyl-phenoxy}-acetic acid methyl ester (58) as a white solid: ¹H-NMR (400 MHz, CDCl₃) δ=7.54 (d, J=8.4 Hz, 2H), 7.24 (d, J=8.4 Hz, 2H), 6.85 (d, J=2.8 Hz, 1H), 6.74 (m, 1H), 6.65 (m, 1H), 5.27 (s, 2H), 4.61 (s, 2H), 3.80 (s, 3H), 2.65 (t, J=8.0 Hz, 3H), 2.29 (s, 3H), 1.67 (m, 2H), 0.97 (t, J=8.0 Hz, 3H). MS calculated for C₂₃H₂₅BrNO₄S (M+H⁺) 490.1, found 490.1.

Intermediate 59: 2-Isopropoxy-5-pyridineboronic Acid.

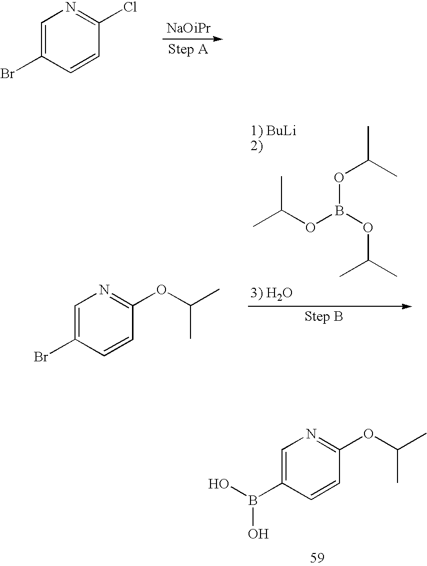

Step A: NaH (5.2 g, 130 mmol) is suspended in isopropanol (50 mL). The mixture is stirred for 30 min at 60° C. After the gas evolution ceased, 2-chloro-5-bromopyridine (10.0 g, 52 mmol) dissolved in isopropanol (100 mL) is added and the mixture is heated to reflux for 24 hours. The solvent is removed in vacuo, and the remainder is taken up in H₂O and extracted with EtOAc. The organic layer is separated and dried over MgSO₄, filtered and concentrated to afford 2-isopropoxy-5-bromo-pyridine as a light brown oil: ¹H-NMR (400 MHz, CDCl₃) δ=8.10 (d, J=2.5 Hz, 1H), 7.54 (dd, J=2.5 Hz, J=8.8 Hz, 1H), 6.52 (d, J=8.8 Hz, 1H), 5.17 (m, 1H), 1.26 (d, J=6.2 Hz, 6H). MS calculated for C₈H₁₁BrNO (M+H⁺) 216.0, found 215.9.

Step B: 2-Isopropoxy-5-bromo-pyridine (0.65 g, 3 mmol) is dissolved in dry ether (10 mL) and cooled to −78° C. under argon. Butyl lithium (1.6 M in hexane, 2.81 mL, 4.5 mmol) is added dropwise and the mixture is stirred at −78° C. for 2 hours. Then triisopropyl borate (1.72 mL, 7.5 mmol) is added quickly and the mixture is stirred for another 2 h at −78° C. The mixture is allowed to warm to room temperature, quenched with H₂O (20 mL) and stirred overnight at room temperature. The ether is removed in vacuo, the aqueous layer is adjusted to pH 10 (with 2 M NaOH) and washed with ether. Then the aqueous layer is adjusted to pH 3 (with 48% aq. HBr) and extracted with EtOAc three times. The organic layer is separated and dried over MgSO₄, filtered and concentrated to afford 2-isopropoxy-5-pyridineboronic acid 59 as a colorless glass: MS calculated for C₈H₁₃BNO₃ (M+H⁺) 182.1, found 182.1.

Intermediate 60. 2-Isopropoxy-5-pyrimidineboronic Acid.

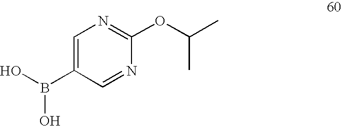

Following the procedure of Intermediate 59, except substituting 2-chloro-5-bromopyrimidine for 2-chloro-5-bromopyridine in Step A, the title compound is prepared as a white solid: MS calculated for C₇H₁₂BN₂O₃ (M+H⁺) 183.1, found 183.1.

Intermediate 61: 2-Morpholino-5-pyrimidineboronic Acid.

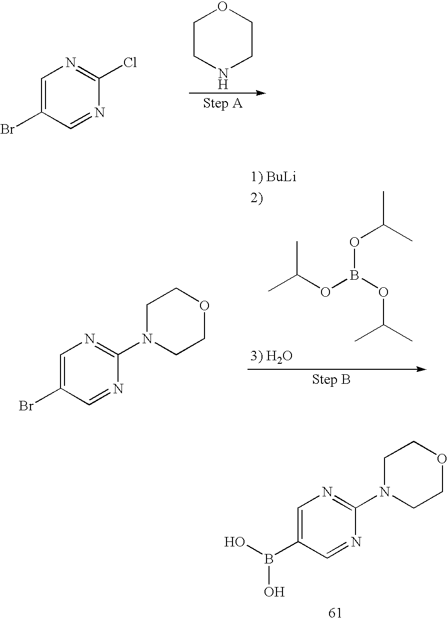

Step A: Morpholine (5.4 ml, 62.4 mmol) is dissolved in MeCN (250 mL). K₂CO₃ (8.6 g, 62.4 mmol) is added and the mixture is stirred at room temperature for 1 hour. Then 2-chloro-5-bromo-pyrimidine (10.0 g, 52 mmol) is added and the mixture is heated to reflux for 5 hours. The solvent is partially removed in vacuo and the remainder is taken up in H₂O and extracted with EtOAc. The organic layer is separated and dried over MgSO₄, filtered and concentrated to afford 2-isopropoxy-5-bromo-pyrimidine as a light brown oil: ¹H-NMR (400 MHz, CDCl₃) δ=8.24 (s, 2H), 3.69 (m, 8H). MS calculated for C₈H₁₁BrN₃O (M+H⁺) 244.0, found 243.9.

Step B: Following the procedure of Intermediate 59 Step B, except substituting 2-isopropoxy-5-bromo-pyrimidine for 2-isopropoxy-5-bromo-pyridine, the title compound is prepared as a white solid: MS calculated for C₉H₁₃BN₃O₃ (M+H⁺) 210.1, found 210.1.

Intermediate 66: (4-Hydroxy-2-propyl-phenoxy)-acetic Acid Methyl Ester.

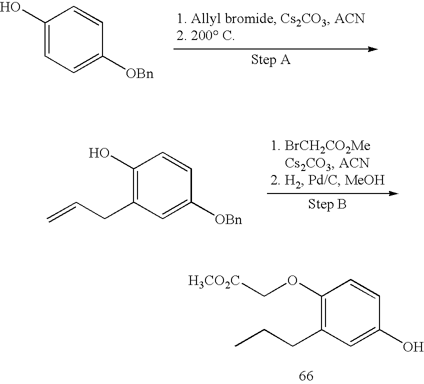

Step A: 4-Benzyloxy-phenol (5.0 g, 25 mmol) is dissolved in acetonitrile (70 mL). Powdered cesium carbonate (10.50 g, 32.2 mmol) is added with stirring, followed by allyl bromide (2.25 mL, 26.6 mmol). The mixture is vigorously stirred overnight. Filtration through a plug of Celite 545, washing the solids with more acetonitrile, drying the solution over Na₂SO₄ and concentration yielded the allyl ether as a white solid. The ether (1.83 g, 7.62 mmol) is heated under nitrogen in a sealed vial to 200° C. After about 4.5 h, the mixture is cooled to yield a light-brown oil: ¹H-NMR (400 MHz, CDCl₃) δ=7.42 (m, 2H), 7.38 (m, 2H), 7.32 (m, 1H), 6.78 (s, 1H), 6.75 (s, 2H), 6.00 (dddd, 1H), 5.18 (m, 1H), 5.14 (m, 1H), 5.00 (s, 1H), 4.62 (br. s, 1H), 3.38 (d, J=6.0 Hz, 2H).

Step B: 2-Allyl-4-benzyloxy-phenol (0.30 g, 1.25 mmol) is dissolved in dry acetonitrile (3 mL). Powdered cesium carbonate (0.68 g, 2.1 mmol) is added with vigorous stirring, followed by methyl bromoacetate (0.15 mL, 1.6 mmol). The suspension is stirred at room temperature overnight. Dilution with 1 N aqueous HCl, extraction with ethyl acetate, drying over MgSO₄ and concentration yields an oil. A portion of this product (0.075 g, 0.24 mmol) is dissolved in methanol (5 mL) and ethyl acetate (30 mL). Palladium black on carbon (5%, 10 mg, 2 mol %) is added. The mixture is degassed and stirred vigorously under 1 atm of hydrogen overnight. Filtration and concentration yields the phenol 66: ¹H-NMR (400 MHz, CDCl₃) δ=6.66 (d, J=2.8 Hz, 1H), 6.61 (d, J=8.4 Hz, 1H), 6.57 (dd, J 2.8, 8.4 Hz, 1H), 4.58 (s, 2H), 4.48 (s, 1H), 3.79 (s, 3H), 2.60 (t, J=7.6 Hz, 2H), 1.61 (m, 2H), 0.95 (t, J=7.4 Hz, 3H). MS calculated for C₁₂H₁₇O₄ (M+H⁺) 225.1, found 225.1.

Intermediate 67: (2-Acetyl-4-hydroxy-phenoxy)-acetic Acid Methyl Ester.

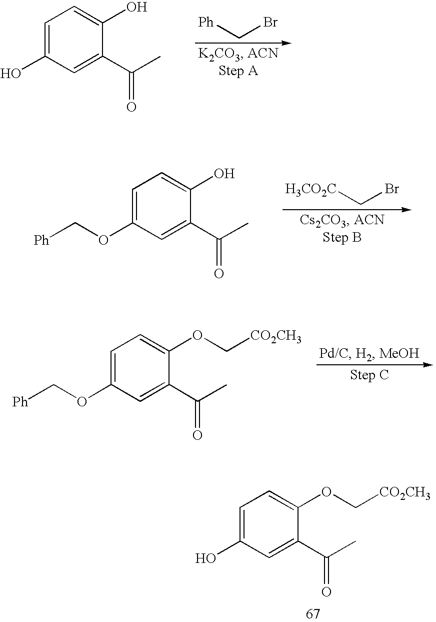

Step A: 1-(2,5-Dihydroxy-phenyl)-ethanone (5.0 g, 33 mmol) is dissolved in 30 mL acetonitrile. Powdered potassium carbonate (7.10 g, 51.4 mmol) is added with stirring, followed by dropwise addition of benzyl bromide (4.0 mL, 33.4 mmol). The resulting suspension is stirred at room temperature under nitrogen overnight, then filtered through a plug of Celite 545 and concentrated to yield a light-brown oil. Silica gel chromatography (hexane to 30% ethyl acetate in hexane) yielded the pure benzyl ether as a near-colorless oil: ¹H-NMR (400 MHz, DMSO-d₆) δ=11.50 (s, 1H), 7.35 (m, 6H), 7.25 (dd, J=3.1, 9.0 Hz, 1H), 6.92 (d, J=9.0 Hz, 1H), 5.10 (s, 2H), 2.63 (s, 3H); MS calculated for C₁₅H₁₅O₃ (M+H⁺) 243.2, found 243.1.

Step B: 1-(5-Benzyloxy-2-hydroxy-phenyl)-ethanone (7.27 g, 29.4 mmol) is dissolved in acetonitrile (100 mL). Powdered cesium carbonate (14.36 g, 44.1 mmol) is added with stirring, followed by methyl bromoacetate (3.5 mL, 38 mmol). The resulting suspension is stirred at 80° C. under nitrogen for 3 h. It is filtered through a plug of Celite 545 and concentrated to yield (2-acetyl-4-benzyloxy-phenoxy)-acetic acid methyl ester as a near-colorless oil that slowly solidifies: ¹H-NMR (400 MHz, CDCl₃) δ=7.36 (m, 6H), 7.25 (dd, J=3.2, 9.0 Hz, 1H), 6.92 (d, J=9.0 Hz, 1H), 5.04 (s, 2H), 4.69 (s, 2H), 3.80 (s, 3H), 2.71 (s, 3H); MS calculated for C₁₈H₁₉O₅ (M+H⁺) 315.2, found 315.1.

Step C: (2-acetyl-4-benzyloxy-phenoxy)-acetic acid methyl ester (9.17 g, 29.2 mmol) is dissolved in methanol (50 mL) and ethyl acetate (50 mL). Palladium black on carbon (5%, 1.53 g, 2.4 mol %) is added. The mixture is degassed and stirred vigorously under 1 atm of hydrogen overnight. Filtration and concentration yields the phenol 67: ¹H-NMR (400 MHz, CDCl₃) δ=7.32 (d, J=3.2 Hz, 1H), 6.97 (dd, J=3.2, 8.9 Hz, 1H), 6.74 (d, J=8.9 Hz, 1H), 4.68 (s, 2H), 3.81 (s, 3H), 2.71 (s, 3H). MS calculated for C₁₁H₁₃O₅ (M+H⁺) 225.1, found 225.1.

Intermediate 68: (2-Bromo-4-hydroxy-phenoxy)-acetic Acid Methyl Ester.

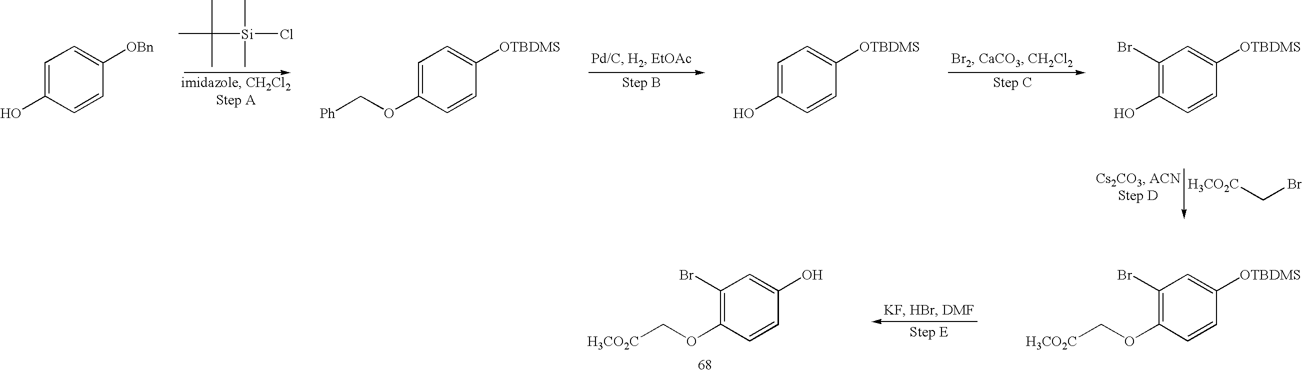

Step A: 4-Benzyloxy-phenol (5.01 g, 25.0 mmol) is suspended in 65 mL dichloromethane. Solid imidazole (4.05 g, 26.9 mmol) is added and the stirring is continued until the mixture turned homogenous. tert-Butyl-chloro-dimethyl-silane (2.49 g, 36.6 mmol) is added in portions, and a white precipitate started to form. The suspension is stirred at room temperature overnight. It is then filtered and concentrated to yield (4-Benzyloxy-phenoxy)-tert-butyl-dimethyl-silane as a white powder: ¹H-NMR (400 MHz, CDCl₃)=7.43 (m, 2H), 7.38 (m, 2H), 7.32 (m, 1H), 6.85 (d, J=9.0 Hz, 2H), 6.76 (d, J=9.0 Hz, 2H), 5.00 (s, 2H), 0.98 (s, 9H), 0.17 (s, 6H); MS calculated for C₁₉H₂₇O₂Si (M+H⁺) 315.2, found 315.1.

Step B: (4-Benzyloxy-phenoxy)-tert-butyl-dimethyl-silane (7.73 g, 24.6 mmol) is dissolved in methanol (10 mL) and ethyl acetate (80 mL). Palladium black on charcoal (5%, 0.6 g, 1 mol %) is added and the mixture is vigorously stirred under hydrogen at room temperature for 48 hours. Filtration and concentration yielded the 4-(tert-butyl-dimethyl-silanyloxy)-phenol: ¹H-NMR (400 MHz, CDCl₃) δ=6.70 (s, 4H), 3.90 (br. s, 1H), 0.97 (s, 9H), 0.16 (s, 6H). MS calculated for C₁₂H₂₁O₂Si (M+H⁺) 225.1, found 225.0.

Step C: 4-(tert-Butyl-dimethyl-silanyloxy)-phenol (4.66 g, 20.8 mmol) is dissolved in dichloromethane (100 mL). Powdered calcium carbonate (4.61 g, 46.7 mmol) is suspended into the solution and the mixture is stirred vigorously at 0° C. Bromine (1.10 mL, 21.4 mmol) is added dropwise with vigorous stirring. After 1.5 h at 0° C., the mixture is warmed up to room temperature, treated with anhydrous MgSO₄, filtered and concentrated to yield 2-bromo-4-(tert-butyl-dimethyl-silanyloxy)-phenol as an oil that slowly solidified: ¹H-NMR (400 MHz, CDCl₃) δ=6.96 (d, J=2.8 Hz, 1H), 6.88 (d, J=8.8 Hz, 1H), 6.71 (dd, J=2.8, 8.8 Hz, 1H), 5.14 (br. s, 1H), 0.97 (s, 9H), 0.17 (s, 6H). MS calculated for C₁₂H₂₀BrO₂Si (M+H⁺) 303.1, found 303.0.

Step D: 2-Bromo-4-(tert-butyl-dimethyl-silanyloxy)-phenol (5.19 g, 17 mmol) is dissolved in acetonitrile (100 mL). Cesium carbonate (14.10 g, 43 mmol) is added, followed by methyl bromoacetate (1.60 mL, 17.4 mmol); the mixture is stirred overnight at room temperature. Filtration and concentration yields [2-bromo-4-(tert-butyl-dimethyl-silanyloxy)-phenoxy]-acetic acid methyl ester as an oil: MS calculated for C₁₅H₂₄BrO₄Si (M+H⁺) 375.2, found 375.1.

Step E: [2-bromo-4-(tert-butyl-dimethyl-silanyloxy)-phenoxy]-acetic acid methyl ester (6.19 g, 16.5 mmol) is dissolved in dimethylformamide (80 mL). Powdered potassium fluoride (2.10 g, 36 mmol) is added, followed by aqueous concentrated hydrogen bromide solution (48%, 1.0 mL, 5.9 mmol). The mixture is stirred overnight at room temperature. Dilution with water, extraction with dichloromethane (4×100 mL), followed by drying over Na₂SO₄; filtration and concentration yields an oil; drying overnight at low pressure yields (2-Bromo-4-hydroxy-phenoxy)-acetic acid methyl ester 68 as a solid: MS calculated for C₉H₁₀BrO₄ (M+H⁺) 261.0, found 260.9.

Intermediate 69: (4-Hydroxy-3-methyl-phenoxy)-acetic Acid Methyl Ester.

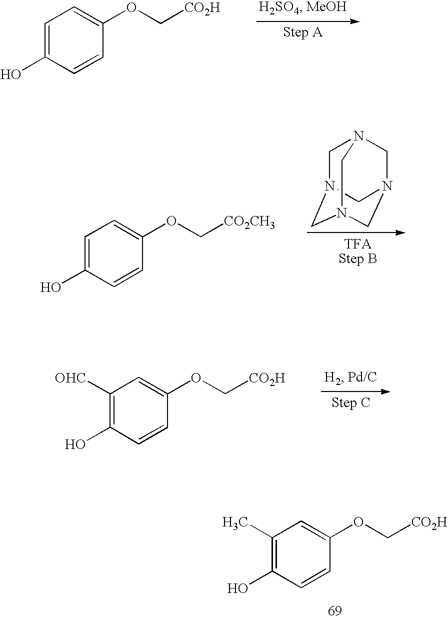

Step A: (4-Hydroxy-phenoxy)-acetic acid (14.96 g, 89 mmol) is suspended in methanol (35 mL). Concentrated sulfuric acid (0.25 mL, cat.) is added and the mixture is refluxed overnight. Cooling to room temperature and concentrating to dryness yields (4-hydroxy-phenoxy)-acetic acid methyl ester as a solid (16 g, quantitative). ¹H-NMR (400 MHz, CDCl₃) δ=6.78 (m, 4H), 4.81 (s, 1H), 4.58 (s, 2H), 3.80 (s, 3H).

Step B: (4-Hydroxy-phenoxy)-acetic acid methyl ester (4.25 g, 23.3 mmol) is dissolved in trifluoroacetic acid (25 mL). Hexamethylene-tetramine (5.11 g, 36.5 mmol) is added. The resulting homogenous mixture is stirred at 70° C. for 3 hours. Cooling to room temperature and concentrating to dryness yields a paste. Silica gel chromatography (10% to 60% ethyl acetate in hexanes) yields (3-formyl-4-hydroxy-phenoxy)-acetic acid methyl ester: ¹H-NMR (400 MHz, CDCl₃) δ=10.70 (s, 1H), 9.84 (s, 1H), 7.20 (dd, J=3.2, 9.2 Hz, 1H), 7.03 (d, J=3.2 Hz, 1H), 6.95 (d, J=8.8 Hz, 1H), 4.64 (s, 2H), 3.82 (s, 3H); MS calculated for C₉H₁₀BrO₄ (M+H⁺) 261.0, found 260.9.

Step C: (3-Formyl-4-hydroxy-phenoxy)-acetic acid methyl ester (0.26 g, 1.24 mmol) is dissolved in methanol (15 mL). Palladium black on charcoal (10 mg, 0.4 mol %) is added and the mixture is stirred overnight under hydrogen (1 atm). Reversed-phase HPLC purification yields (4-hydroxy-3-methyl-phenoxy)-acetic acid methyl ester 69 as an oil: ¹H-NMR (400 MHz, CDCl₃) δ=6.8 (m, 3H), 4.93 (s, 1H), 4.69 (s, 2H), 3.93 (s, 3H), 2.35 (s, 3H).

Intermediate 70: 3-(4-Hydroxy-2-methyl-phenyl)-propionic Acid Methyl Ester

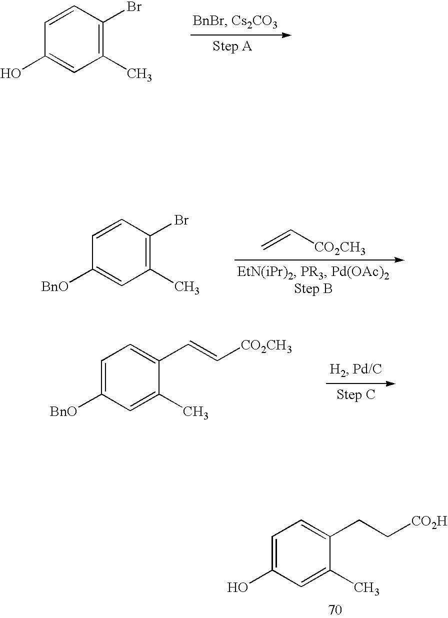

Step A: 4-Bromo-3-methyl-phenol (13.71 g, 73.3 mmol) is dissolved in acetonitrile (100 mL). Cesium carbonate (30.46 g, 93.5 mmol) and benzyl bromide (10 mL, 84.2 mmol) are added and the mixture is stirred overnight at room temperature. Filtration and concentration to dryness yields 4-benzyloxy-1-bromo-2-methyl-benzene as a solid (23.5 g, quantitative). ¹H-NMR (400 MHz, CDCl₃) δ=7.4 (m, 6H), 6.87 (d, J=3.2 Hz, 1H), 6.68 (dd, J=3.2, 8.8 Hz, 1H), 5.03 (s, 2H), 2.36 (s, 3H); no mass spectrum could be obtained.

Step B: 4-Benzyloxy-1-bromo-2-methyl-benzene (9.29 g, 33.5 mmol) is dissolved in propionitrile (80 mL). Ethyldiisopropylamine (12 mL, 72.6 mmol) and methyl acrylate (12 mL, 133 mmol) are added. The mixture is degassed with argon, and tri-ortho-tolylphosphine (4.11 g, 20.1 mmol) and palladium acetate (1.53 g, 6.8 mmol) is added. The mixture is heated to 100° C. overnight. Cooling to room temperature and concentrating to dryness yields a paste. The residue is taken up in ethyl acetate and washed with water and brine, dried over MgSO₄ and concentrated. Silica gel chromatography (0% to 60% ethyl acetate in hexanes) yields 3-(4-benzyloxy-2-methyl-phenyl)-acrylic acid methyl ester: ¹H-NMR (400 MHz, CDCl₃) δ=7.92 (d, J=15.6 Hz, 1H), 7.52 (d, J=9.6 Hz, 1H), 7.4 (m, 5H), 7.68 (m, 2H), 6.26 (d, J=15.6 Hz, 1H), 5.08 (s, 2H), 3.80 (s, 3H), 2.42 (s, 3H); MS calculated for C₁₈H₁₉O₃ (M+H⁺) 283.1, found 283.1.

Step C: 3-(4-Benzyloxy-2-methyl-phenyl)-acrylic acid methyl ester (4.83 g, 17 mmol) is dissolved in methanol (85 mL) and ethyl acetate (25 mL). Palladium black on charcoal (5%, 0.51 g, 1.4 mol %) is added and the mixture is stirred under hydrogen for 36 hours. Concentration yields 3-(4-Hydroxy-2-methyl-phenyl)-propionic acid methyl ester 70 as an oil: ¹H-NMR (400 MHz, CDCl₃) δ=6.98 (d, J=8.4 Hz, 1H), 6.64 (d, J=2.8 Hz, 1H), 6.60 (dd, J=2.8, 8.4 Hz, 1H), 3.68 (s, 3H), 2.87 (t, J=7.6 Hz, 2H), 2.55 (t, J=7.6 Hz, 2H), 2.26 (s, 3H).

Intermediate 71: 2-(4-Hydroxy-phenoxy)-2-methyl-propionic Acid Methyl Ester.

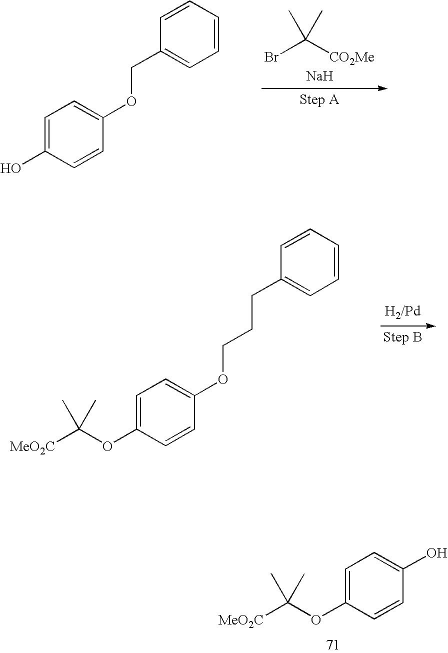

Step A: 4-(Benzyloxy)phenol (5.0 g, 25 mmol) is dissolved in DMF (40 mL). To the solution is added NaH (60% dispersion, 1.1 g, 27.5 mmol) in portions while the temperature is kept at room temperature. After stirring the suspension for 30 min at room temperature methyl-□-bromoisobutyrate (9.05 g, 50 mmol) is added dropwise. The mixture is stirred at 50° C. for 3 h, then concentrated. The remainder is diluted with water (2001 mL) and extracted with EtOAc (3×150 mL). The organic layer is separated and dried over MgSO₄, filtered and concentrated. The crude product is purified by flash chromatography (silica, Hex/EtOAc gradient) to afford 2-(4-benzyloxy-phenoxy)-2-methyl-propionic acid methyl ester as a clear oil: ¹H-NMR (400 MHz, CDCl₃) δ=7.44-7.33 (m, 5H), 6.85 (m, 4H), 5.01 (s, 2H), 3.78 (s, 3H), 1.55 (s, 6H). MS calculated for C₁₈H₂₁O₄ (M+H⁺) 301.1, found 301.4.

Step B: 2-(4-benzyloxy-phenoxy)-2-methyl-propionic acid methyl ester (0.5 g, 1.7 mmol) is dissolved in EtOH (15 mL). After addition of a catalytic amount of Palladium(0) on charcoal the mixture is subjected to 1 atm hydrogen and stirred for 5 h at room temperature. Then the mixture is filtered through celite, the solvent is removed and the remainder dried on high vacuum to yield 2-(4-hydroxy-phenoxy)-2-methyl-propionic acid methyl ester 71 as a brownish oil: ¹H-NMR (400 MHz, CDCl₃) δ=6.76 (d, J=9.0 Hz, 2H), 6.69 (d, J=9.0 Hz, 2H), 3.78 (s, 3H), 1.53 (s, 6H). MS calculated for C₁₁H₁₅O₄ (M+H⁺) 211.1, found 211.3.

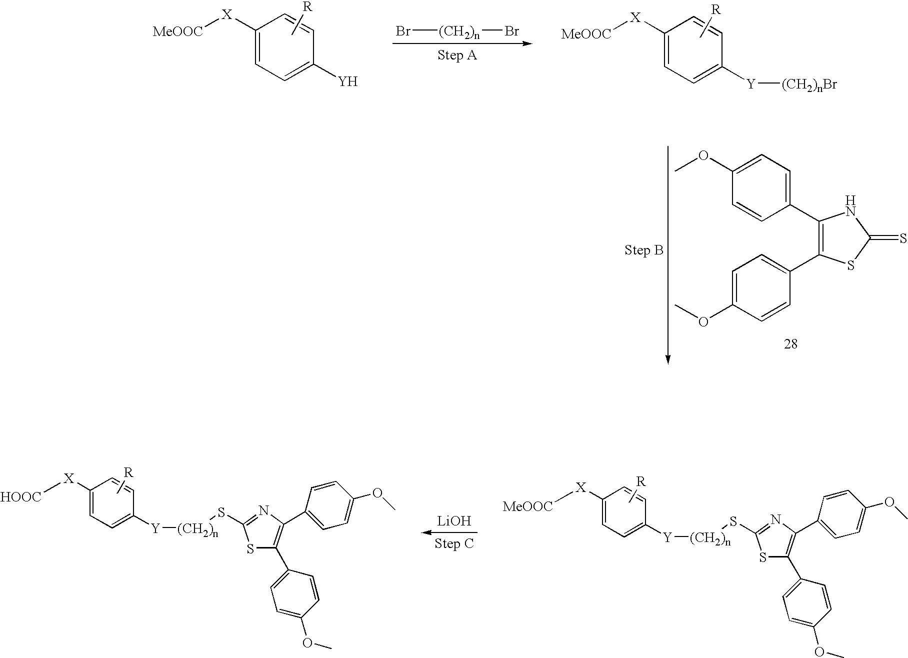

### Example A1

### (4-{2-[4,5-Bis-(4-methoxy-phenyl)-thiazol-2-ylsulfanyl]-ethoxy}-2-methyl-phenoxy)-acetic acid

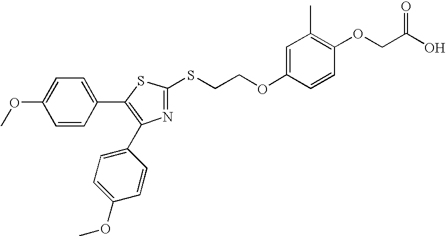

Step A: Intermediate 4 (0.5 g, 2.8 mmol), 1,2-dibromoethane (2.4 mL, 27.7 mmol) and Cs₂CO₃ (4.5 g, 13.9 mmol) are suspended in dry acetone. The mixture is heated to reflux overnight. The reaction mixture is cooled to room temperature, filtered and the solvent is removed in vacuo. The remainder is purified by chromatography (silica, DCM/MeOH gradient) to afford [4-(2-Bromo-ethoxy)-2-methyl-phenoxy]-acetic acid methyl ester as a white solid: MS calculated for C₁₁H₁₄BrO₄ (M+H⁺) 303.0, found 303.2.

Step B: [4-(2-Bromo-ethoxy)-2-methyl-phenoxy]-acetic acid methyl ester (91 mg, 0.30 mmol) is added dropwise to a solution of NaOMe (23 mg, 0.33 mmol) and intermediate 28 in EtOH (5 mL). After stirring at room temperature for 24 h the solvent is removed to afford crude (4-{2-[4,5-Bis-(4-methoxy-phenyl)-thiazol-2-ylsulfanyl]-ethoxy}-2-methyl-phenoxy)-acetic acid methyl ester.

Step C: The crude (4-{2-[4,5-Bis-(4-methoxy-phenyl)-thiazol-2-ylsulfanyl]-ethoxy}-2-methyl-phenoxy)-acetic acid methyl ester is dissolved in THF (3 mL), a solution of 1 M LiOH in H₂O (0.6 mL) is added and the mixture is stirred overnight at room temperature. The mixture is acidified with 1 M HCl, EtOAc (10 mL) is added and the organic layer washed with H₂O (3×5 mL). The organic layer is dried (MgSO₄), filtered, concentrated and purified on reverse phase HPLC (H₂O/MeCN gradient) to afford the title compound A1 as a white solid: ¹H-NMR (400 MHz, CD₃OD) δ=7.34 (d, J=8.9 Hz, 2H), 7.19 (d, J=8.8 Hz, 2H), 6.88 (d, J=8.9 Hz, 2H), 6.83 (d, J=8.8 Hz, 2H), 6.73-6.64 (m, 3H), 4.56 (s, 2H), 4.29 (t, J=6.4 Hz), 3.79 (s, 3H), 3.78 (s, 3H), 3.57 (t, J=6.4 Hz, 2H), 2.18 (s, 3H). MS calculated for C₂₈H₂₈NO₆S₂ (M+H⁺) 538.1, found 538.4.

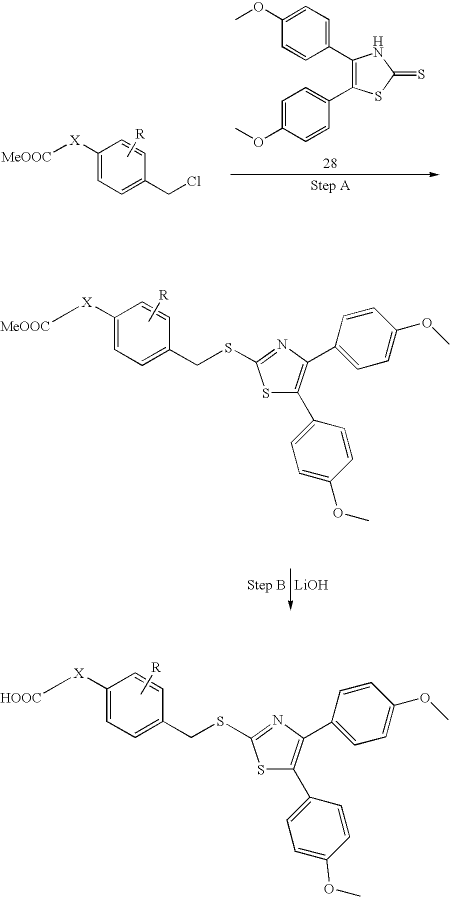

### Example B1

### {4-[4,5-Bis-(4-methoxy-phenyl)-thiazol-2-ylsulfanylmethyl]-2-methyl-phenoxy}-acetic acid

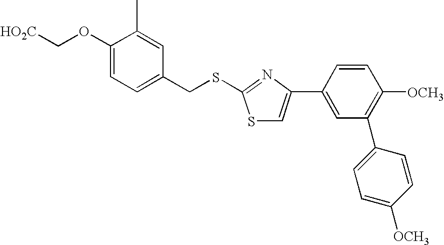

Step A: NaOEt (23 mg, 0.33 mmol) is dissolved in absolute EtOH (5 mL). 4,5-Bis-(4-methoxy-phenyl)-3H-thiazole-2-thione 28 (73 mg, 0.30 mmol) and (4-chloromethyl-2-methyl-phenoxy)-acetic acid ethyl ester (intermediate 11) (100 mg, 0.30 mmol) is added successively. The reaction is stirred for 12 h at room temperature to afford the crude product.

Step B: 2 N LiOH (3.0 mL) is added into the reaction mixture from step A and it is stirred for 3 h at 60° C. The reaction is cooled to room temperature and acidified to PH 2-3 by 2 N HCl. Then it is extracted with CH₂Cl₂. The organic layer is separated, dried (MgSO₄) and concentrated. The product is recrystallized in ethyl acetate and hexane to afford the title compound B1 as a slightly yellow solid: ¹H-NMR (400 MHz, CDCl₃) δ=7.44 (d, J=8.8 Hz, 2H), 7.24-7.18 (m, 4H), 6.86-6.80 (m, 4H), 6.66 (d, J=8.4 Hz, 1H), 5.30 (s, 2H), 4.67 (s, 2H), 3.81 (s, 3H), 3.80 (s, 3H), 2.27 (s, 3H). MS calculated for C₂₇H₂₆NO₅S₂ (M+H⁺) 508.12, found 508.10.

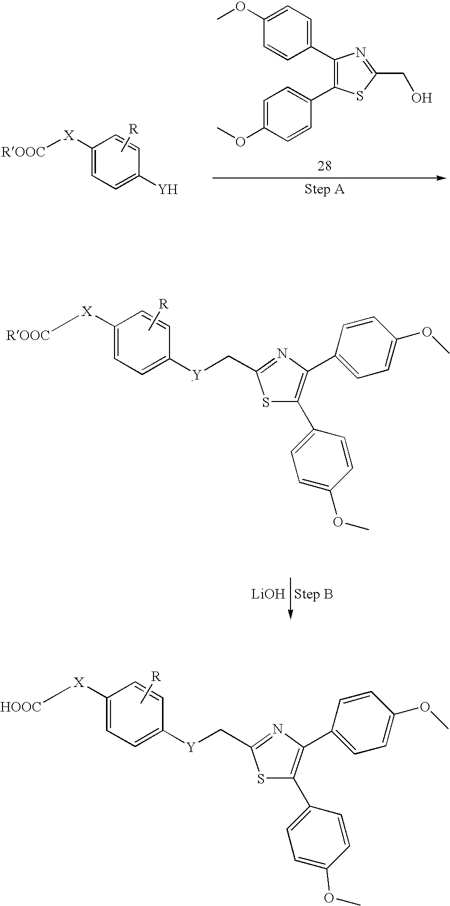

### Example C1

### {4-[4,5-Bis-(4-methoxy-phenyl)-thiazol-2-ylmethoxy]-2-methyl-phenoxy}-acetic acid

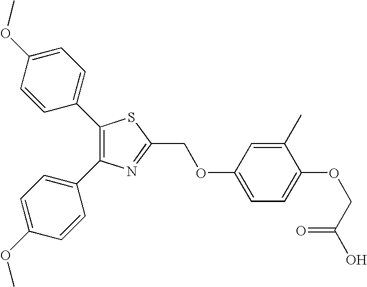

Step A: Intermediate 30 (25 mg, 0.08 mmol), intermediate 4 (18 mg, 0.09 mmol) and triphenylphosphine (30 mg, 0.11 mmol) are dissolved in dry DCM (1 mL) and cooled to 0° C. After the slow addition of diethyl azodicarboxylate (24 □L, 0.15 mmol) the solution is stirred at room temperature overnight. The solvent is removed to afford crude {4-[4,5-Bis-(4-methoxy-phenyl)-thiazol-2-ylmethoxy]-2-methyl-phenoxy}-acetic acid methyl ester which is used without further purification in step B.

Step B: The crude {4-[4,5-Bis-(4-methoxy-phenyl)-thiazol-2-ylmethoxy]-2-methyl-phenoxy}-acetic acid methyl ester is dissolved in THF (1 mL), a solution of 1 M LiOH in H₂O (0.2 mL) is added and the mixture is stirred overnight at room temperature. The mixture is acidified with 1 M HCl (0.25 mL), EtOAc (10 mL) is added and the organic layer washed with H₂O (3×5 mL). The organic layer is dried (MgSO₄), filtered, concentrated and purified on reverse phase HPLC (H₂O/MeCN gradient) to afford the title compound C1 as a colorless glass: ¹H-NMR (400 MHz, CD₃OD) δ=7.36 (d, J=8.9 Hz, 2H), 7.22 (d, J=8.8 Hz, 2H), 6.91-6.75 (m, 7H), 5.30 (s, 2H), 4.62 (s, 2H), 3.79 (s, 3H), 3.78 (s, 3H), 2.25 (s, 3H). MS calculated for C₂₇H₂₆NO₆S (M+H) 492.1, found 492.4.

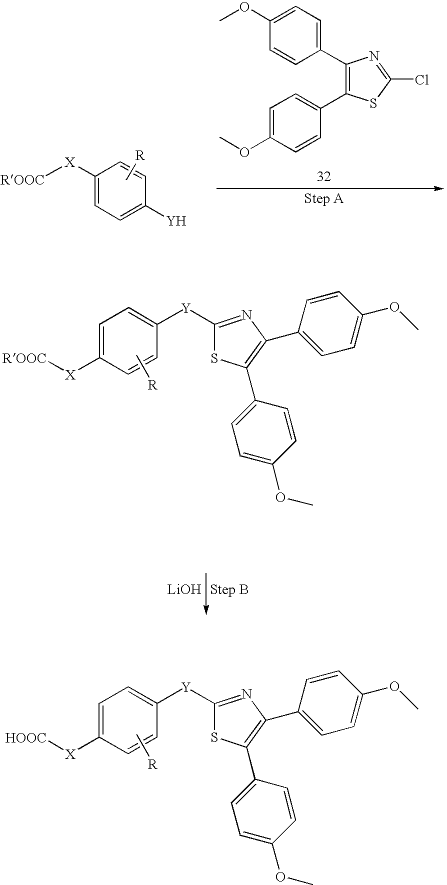

### Example D1

### {4-[4,5-Bis-(4-methoxy-phenyl)-thiazol-2-ylsulfanyl]-2-methyl-phenoxy}-acetic acid

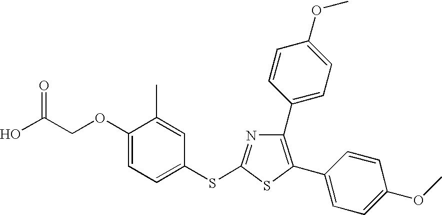

Step A: Intermediate 10 (28 mg, 0.131 mmol), intermediate 32 (40 mg, 0.131 mmol), and NaOEt (18 mg, 0.262 mmol) are dissolved in EtOH (1 mL) and heated to reflux for 6 hours. The mixture is acidified with aqueous 1 N HCl (1 mL) and extracted with EtOAc (2×4 mL). The organic layer is dried (MgSO₄), filtered, and concentrated to provide crude {4-[4,5-Bis-(4-methoxy-phenyl)-thiazol-2-ylsulfanyl]-2-methyl-phenoxy}-acetic acid ethyl ester.

Step B: {4-[4,5-Bis-(4-methoxy-phenyl)-thiazol-2-ylsulfanyl]-2-methyl-phenoxy}-acetic acid ethyl ester is then dissolved in THF (1 mL) and treated with 1 N LiOH (200 μL) and stirred at room temperature for 2 hours. The mixture is acidified with aqueous HCl (1 N, 300 μL), extracted with EtOAc (2×4 mL), dried (MgSO₄), filtered, concentrated, and purified on reverse phase HPLC (H₂O/MeCN gradient) to afford the title compound D1 as a white solid: ¹H-NMR (400 MHz, CDCl₃) δ=7.45 (s, 1H), 7.42 (d, J=8.4 Hz, 1H), 7.33 (d, 8.8 Hz, 2H), 7.06 (d, J=8.8 Hz, 2H), 6.70 (m, 5H), 4.65 (s, 2H), 3.71 (s, 3H), 3.69 (s, 3H), 2.22 (s, 3H). MS calculated for C₂₆H₂₄NO₅S₂ (M+H⁺) 494.1, found 494.4.

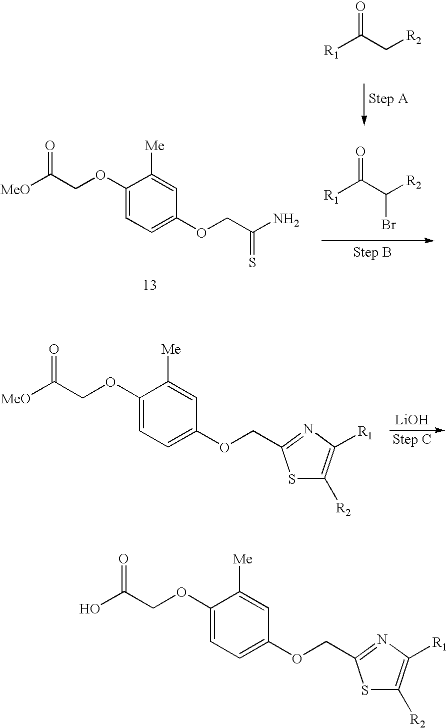

### Example E1

### {4-[4-(4-Bromo-phenyl)-5-phenyl-thiazol-2-ylmethoxy]-2-methyl-phenoxy}-acetic acid

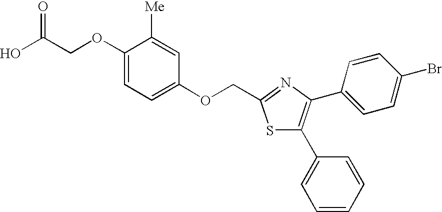

Step A: 1-(4-Bromo-phenyl)-2-phenyl-ethanone (0.24 g, 0.87 mmol) is dissolved in glacial acetic acid (3 mL). Bromine (50 CL, 0.97 mmol) is added and the mixture is stirred for 30 minutes at room temperature. Dilution with water (40 mL) yields a white solid. Filtration, washing with water, and drying yields 2-bromo-1-(4-bromo-phenyl)-2-phenyl-ethanone as a white powder: 0.30 g. ¹H-NMR (400 MHz, CDCl₃) δ=7.85 (d, J=6.8 Hz, 2H), 7.59 (d, J=6.8 Hz, 2H) 7.50 (dd, J=2.0, 8.4 Hz, 2H), 7.36 (m, 3H), 6.30 (s, 1H). MS calculated for C₁₁H₁₁Br₂O (M+H⁺) 354.9, found 355.1.

Step B: (2-Methyl-4-thiocarbamoylmethoxy-phenoxy)-acetic acid methyl ester (Intermediate 13) (46 mg, 0.17 mmol) and 2-bromo-1-(4-bromo-phenyl)-2-phenyl-ethanone are suspended in ethanol and heated to 75° C. for 18 hours. Cooling to room temperature, concentration, and purification by chromatography (silica, 10% to 40% ethyl acetate in hexane gradient) affords {4-[4-(4-bromo-phenyl)-5-phenyl-thiazol-2-ylmethoxy]-2-methyl-phenoxy}-acetic acid ethyl ester as a white solid: ¹H-NMR (400 MHz, CDCl₃) δ=7.28 (m, 4H), 7.21 (m, 5H), 6.77 (d, J=2.8 Hz, 1H), 6.66 (dd, J=2.8, 8.8 Hz, 1H), 6.55 (d, J=8.8 Hz, 1H), 5.23 (s, 2H), 4.47 (s, 2H), 4.13 (q, J=7.2 Hz, 2H), 2.17 (s, 3H), 1.17 (q, J=7.2 Hz, 3H).

Step C: {4-[4-(4-Bromo-phenyl)-5-phenyl-thiazol-2-ylmethoxy]-2-methyl-phenoxy}-acetic acid ethyl ester (55.2 mg, 0.11 mmol) is dissolved in dioxane. Lithium hydroxide monohydrate (13.0 mg, 0.31 mmol) dissolved in water (0.5 mL) is added. After 40 minutes the mixture became homogenous. Concentration to a syrup, dilution with ethyl acetate, washing with 10% citric acid solution, water, saturated aqueous ammonium chloride, and brine, drying over Na₂SO₄ and concentration yields the title compound {4-[4-(4-bromo-phenyl)-5-phenyl-thiazol-2-ylmethoxy]-2-methyl-phenoxy}-acetic acid E1 as a white solid: Reversed phase HPLC purification yields the pure acid: ¹H-NMR (400 MHz, DMSO-d₆) δ=12.87 (s, 1H), 7.53 (m, 2H), 7.41-7.34 (m, 7H), 6.95 (d, J=2.9 Hz, 1H), 6.85 (dd, J=2.9, 8.9 Hz, 1H), 6.77 (d, J=8.9 Hz, 1H), 5.39 (s, 2H), 4.61 (s, 2H), 2.18 (s, 3H). MS calculated for C₂₅H₂₁BrNO₄S (M+H⁺) 512.0, found 512.3.

### Example E2

### [4-(4,5-Diphenyl-thiazol-2-ylmethoxy)-2-methyl-phenoxy]-acetic acid

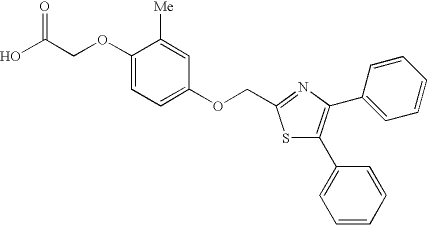

Step A: For the title compound, the intermediate bromide is purchased and used directly in Step B.

Step B: Intermediate 13 (20 mg, 0.076 mmol), and desyl bromide (23 mg, 0.084 mmol) are dissolved in EtOH (2 mL) and heated to reflux for 2 hours. The solvent is removed by evaporation to afford crude [4-(4,5-diphenyl-thiazol-2-ylmethoxy)-2-methyl-phenoxy]-acetic acid methyl ester which is used without further purification in Step C.

Step C: The crude [4-(4,5-diphenyl-thiazol-2-ylmethoxy)-2-methyl-phenoxy]-acetic acid methyl ester is dissolved in THF (1 mL), a solution of 1 M LiOH in H₂O (0.2 mL) is added and the mixture is stirred for 1 h at room temperature. The mixture is acidified with 1 M HCl (0.25 mL), EtOAc (10 mL) is added and the organic layer washed with brine (5 mL). The organic layer is dried (MgSO₄), filtered, concentrated and purified on reverse phase HPLC (H₂O/MeCN gradient) to afford the title compound E2 as a white solid: ¹H-NMR (400 MHz, CDCl₃) δ=7.40 (m, 2H), 7.23 (m, 8H), 6.82 (d, J=2.9 Hz, 1H), 6.69 (dd, J=3.0, 8.9 Hz, 1H), 6.61 (d, J=8.9 Hz, 1H), 5.26 (s, 2H), 4.53 (s, 2H), 2.20 (s, 3H). MS calculated for C₂₅H₂₂NO₄S (M+H⁺) 432.1, found 432.4.

### Example E3

### {4-[4-(4-Bromo-phenyl)-5-phenyl-thiazol-2-ylmethoxy]-2-methyl-phenoxy}-acetic acid

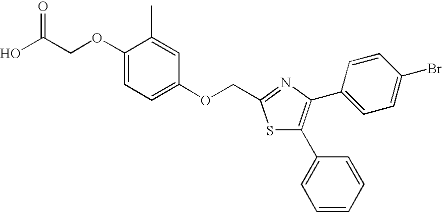

Step A: 1-(4-Bromo-phenyl)-2-phenyl-ethanone (275 mg, 1.00 mmol) is dissolved in DCM (2 mL). Pyridinium tribromide (352 mg, 1.1 mmol) is added and the mixture is stirred at room temperature for 2 hours. Then the mixture is diluted with DCM (1 mL) and washed with H₂O (2 mL). The organic layer is concentrated in vacuo to afford crude 1-(4-bromo-phenyl)-2-bromo-2-phenyl-ethanone as a yellow solid, and is used in Step B without further purification.

Step B: A mixture of 1-(4-bromo-phenyl)-2-bromo-2-phenyl-ethanone (43 mg, 0.12 mmol) and (2-Methyl-4-thiocarbamoylmethoxy-phenoxy)-acetic acid methyl ester (Intermediate 13, 32 mg, 0.12 mmol) in EtOH (1 mL) is heated at 180° C. for 5 min in a microwave apparatus. The resulting solution is used directly in the next step.

Step C: THF (2 mL) and 1 N LiOH (0.5 mL) are added to the solution derived from step B. The mixture is stirred overnight at room temperature, then acidified with 1 N HCl (1 mL). The reaction mixture is extracted with EtOAc (3 mL), the organic layer is separated and concentrated in vacuo. The remainder is taken up in DMSO (1 mL) and purified on reverse phase HPLC (H₂O/MeCN gradient) to afford the title compound E59 as a white solid: ¹H-NMR (600 MHz, (CD₃)₂SO) δ=7.54-7.31 (m, 9H), 6.96 (d, J=3.0 Hz, 1H), 6.87 (dd, J=3.0 Hz, J=8.9 Hz, 1H), 6.79 (d, J=8.9 Hz, 1H), 5.40 (s, 2H), 4.63 (s, 2H), 2.19 (s, 3H). MS calculated for C₂₅H₂₁BrNO₄S (M+H⁺) 510.0, found 510.3.

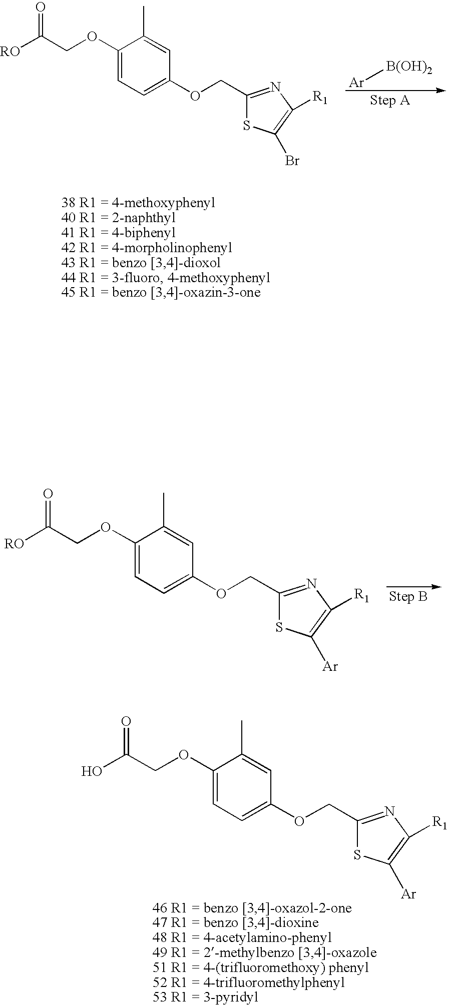

### Example F1

### {4-[4-(4-Methoxy-phenyl)-5-(4-trifluoromethoxy-phenyl)-thiazol-2-ylmethoxy]-2-methyl-phenoxy}-acetic acid

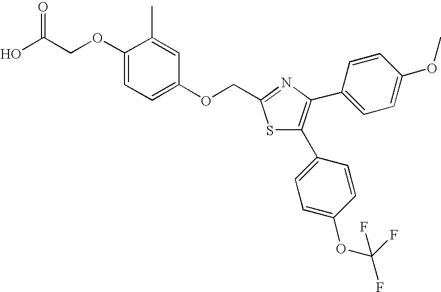

Step A: Intermediate 38 (21 mg, 0.042 mmol), 4-trifluoromethoxyphenyl-boronic acid (10.3 mg, 0.050 mmol) and sodium carbonate (13 mg, 0.126 mmol) are dissolved in water (120 μL), ethanol (90 μL) and 1,2-dimethoxyethane (360 μL) and the mixture is degassed with bubbling Argon for 2 minutes. Pd(PPh₃)₄ (10 mol %) is added and the mixture is subjected to microwave (180° C.) for 5 min in a sealed tube. The mixture is diluted with saturated water (5 mL), extracted into EtOAc (10 mL) and washed with brine (5 mL). The organic layer is dried (MgSO₄), filtered and concentrated to give crude {4-[4-(4-Methoxy-phenyl)-5-(4-trifluoromethoxy-phenyl)-thiazol-2-ylmethoxy]-2-methyl-phenoxy}-acetic acid methyl ester, which is used without further purification in Step B.

Step B: The {4-[4-(4-Methoxy-phenyl)-5-(4-trifluoromethoxy-phenyl)-thiazol-2-ylmethoxy]-2-methyl-phenoxy}-acetic acid methyl ester is dissolved in THF (1 mL), a solution of 1 M LiOH in H₂O (0.2 mL) is added and the mixture is stirred for 1 h at room temperature. The mixture is acidified with 1 M HCl (0.25 mL), EtOAc (10 mL) is added and the organic layer washed with brine (5 mL). The organic layer is dried (MgSO₄), filtered, concentrated and purified on reverse phase HPLC (H₂O/MeCN gradient) to afford the title compound F1 as a white solid: ¹H-NMR (400 MHz, CDCl₃) δ=7.32 (d, J=8.8 Hz, 2H), 7.28 (d, J=8.7 Hz, 2H), 7.07 (d, J=8.1 Hz, 2H), 6.82 (d, J=2.9 Hz, 1H), 6.76 (d, J=8.8 Hz, 2H), 6.71 (dd, J=2.8, 8.8 Hz, 1H), 6.63 (d, J=8.9 Hz, 1H), 5.25 (s, 2H), 4.56 (s, 2H), 3.75 (s, 3H), 2.21 (s, 3H). MS calculated for C₂₇H₂₃F₃NO₆S (M+H⁺) 546.1, found 546.3.

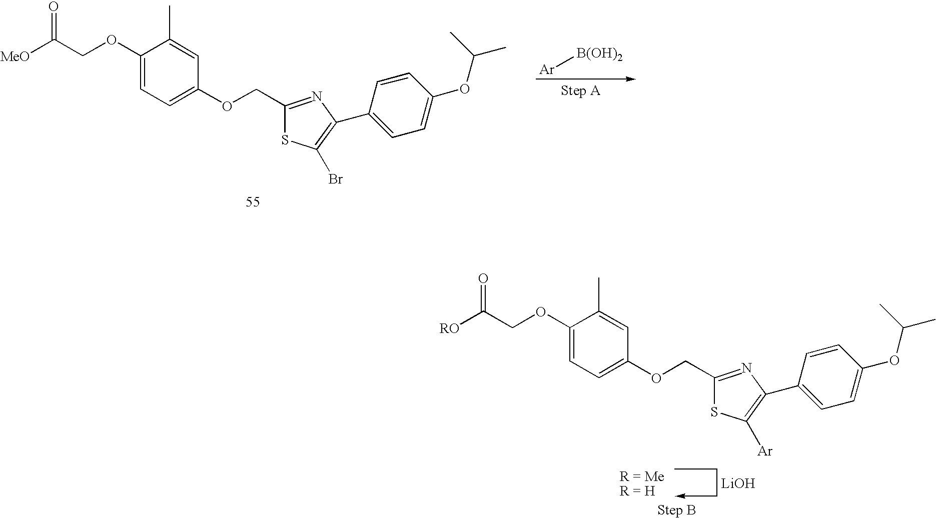

### Example G1

### {4-[4-(4-Isopropoxy-phenyl)-5-(4-trifluoromethoxy-phenyl)-thiazol-2-ylmethoxy]-2-methyl-phenoxy}-acetic acid

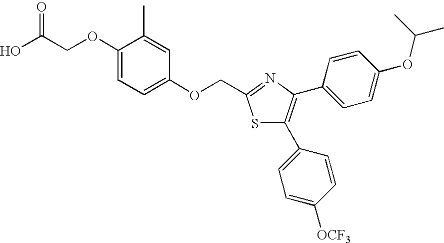

Step A: Intermediate 55 (21 mg, 0.041 mmol), 4-trifluoromethoxyphenyl-boronic acid (10.3 mg, 0.050 mmol) and sodium carbonate (13 mg, 0.126 mmol) are dissolved in water (120 μL), ethanol (90 μL) and 1,2-dimethoxyethane (360 μL). The mixture is degassed with argon for 2 min. Pd(PPh₃)₄ (10 mol %) is added and the mixture is subjected to microwave (170° C.) for 5 min. The mixture is diluted with water (5 mL), extracted into EtOAc (10 mL) and washed with brine (5 mL). The organic layer is dried (MgSO₄), filtered and concentrated to give crude {4-[4-(4-Isopropoxy-phenyl)-5-(4-trifluoromethoxy-phenyl)-thiazol-2-ylmethoxy]-2-methyl-phenoxy}-acetic acid methyl ester, which is used without further purification in Step B.

Step B: The crude {4-[4-(4-Isopropoxy-phenyl)-5-(4-trifluoromethoxy-phenyl)-thiazol-2-ylmethoxy]-2-methyl-phenoxy}-acetic acid methyl ester is dissolved in THF (1 mL), a solution of 1 M LiOH in H₂O (0.2 mL) is added and the mixture is stirred for 1 h at room temperature. The mixture is acidified with 1 M HCl (0.25 mL), EtOAc (10 mL) is added and the organic layer washed with brine (5 mL). The organic layer is dried (MgSO₄), filtered, concentrated and purified on reverse phase HPLC (H₂O/MeCN gradient) to afford the title compound G1 as a white solid: ¹H-NMR (400 MHz, MeOD) δ=7.34 (d, J=8.8 Hz, 2H), 7.26 (d, J=8.8 Hz, 2H), 7.17 (d, J=8.4 Hz, 2H), 6.83 (d, J=2.8 Hz, 1H), 6.77 (d, J=8.4 Hz, 2H), 6.75 (dd, J=2.8, 8.8 Hz, 1H), 6.68 (d, J=8.8 Hz, 1H), 5.25 (s, 2H), 4.53 (m, 3H), 2.17 (s, 3H), 1.23 (s, 3H), 1.21 (s, 3H). MS calculated for C₂₉H₂₇F₃NO₆S (M+H⁺) 574.1, found 574.1.

### Example G2

### {4-[4-(4-Isopropoxy-phenyl)-5-(4-trifluoromethyl-phenyl)-thiazol-2-ylmethoxy]-2-methyl-phenoxy}-acetic acid

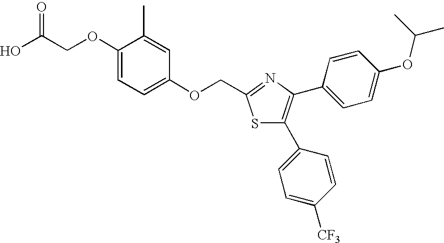

Step A: Intermediate 55 (21 mg, 0.04 mmol), 4-trifluoromethylphenyl-boronic acid (9.5 mg, 0.05 mmol) and sodium carbonate (13 mg, 0.13 mmol) are dissolved in water (120 μL), ethanol (90 μL) and 1,2-dimethoxyethane (360 μL). The mixture is degassed with Argon for 2 minutes. Pd(PPh₃)₄ (10 mol %) is added and the mixture is subjected to microwave (170° C.) for 5 min. The mixture is diluted with water (5 mL), extracted into EtOAc (10 mL) and washed with brine (5 mL). The organic layer is dried (MgSO₄), filtered and concentrated to give crude {4-[4-(4-Isopropoxy-phenyl)-5-(4-trifluoromethyl-phenyl)-thiazol-2-ylmethoxy]-2-methyl-phenoxy}-acetic acid methyl ester, which is used without further purification in Step B.

Step B: The crude {4-[4-(4-Isopropoxy-phenyl)-5-(4-trifluoromethyl-phenyl)-thiazol-2-ylmethoxy]-2-methyl-phenoxy}-acetic acid methyl ester is dissolved in THF (1 mL), a solution of 1 M LiOH in H₂O (0.2 mL) is added and the mixture is stirred for 1 h at room temperature. The mixture is acidified with 1 M HCl (0.25 mL), EtOAc (10 mL) is added and the organic layer washed with brine (5 mL). The organic layer is dried (MgSO₄), filtered, concentrated and purified on reverse phase HPLC (H₂O/MeCN gradient) to afford the title compound G2 is prepared as a white solid: ¹H-NMR (400 MHz, CDCl₃) δ 7.70 (d, J=8.0 Hz, 2H), 7.58 (d, J=8.0 Hz, 2H), 7.40 (d, J=8.8 Hz, 2H), 6.98 (d, J=2.8 Hz, 1H), 6.92 (d, J=8.8 Hz, 2H), 6.90 (dd, J=2.8, 8.8 Hz, 1H), 6.83 (d, J=8.8 Hz, 1H), 5.41 (s, 2H), 4.67 (m, 3H), 2.32 (s, 3H), 1.37 (s, 3H), 1.36 (s, 3H). MS calculated for C₂₉H₂₇F₃NO₅S (M+H⁺) 558.1, found 558.2.

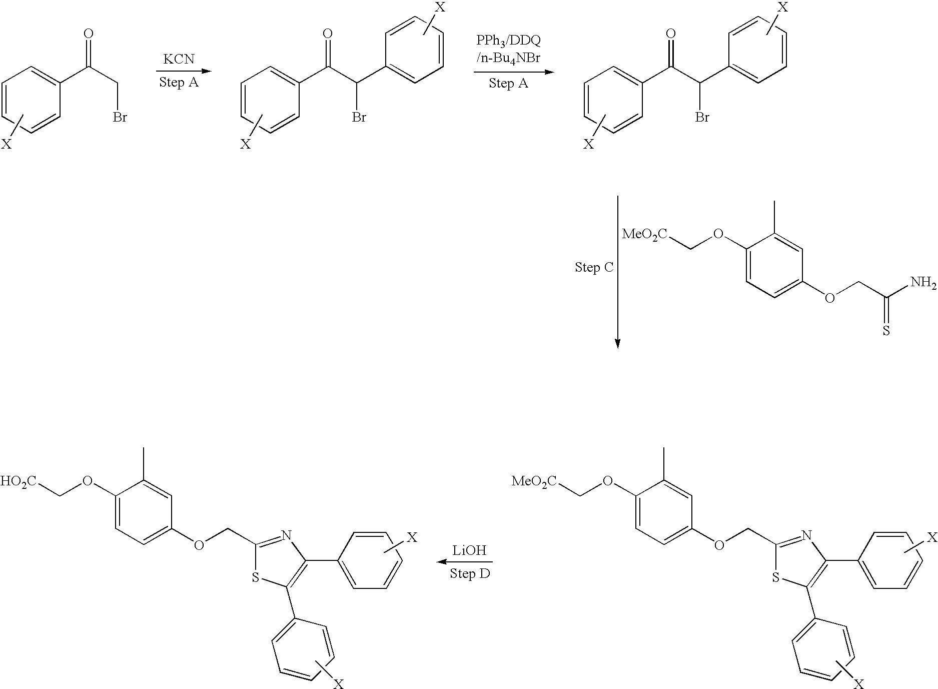

### Example H1

### {4-[4,5-Bis-(4-chloro-phenyl)-thiazol-2-ylmethoxy]-2-methyl-phenoxy}-acetic acid

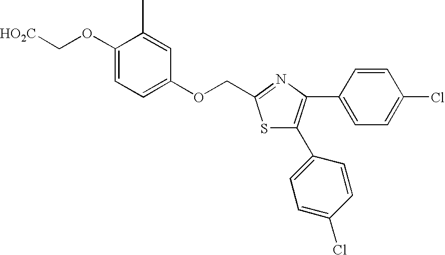

Step A: To a solution of 4-chloro-benzaldehyde (0.57 g, 2.04 mmol) in EtOH (8 mL) is added KCN (18.0 mg, 0.27 mmol) dissolved in water (4 mL). The mixture is heated to reflux for 7 hours, then cooled to room temperature and extracted with ethyl acetate (100 mL). The organic layer is dried (MgSO₄), filtered, and concentrated. The residue is purified by flash chromatography with 40% ether/hexane to give a solid: NMR (400 MHz, CDCl₃) δ=7.83 (m, 2H), 7.39 (m, 2H), 7.31 (m, 2H), 7.25 (m, 2H), 5.88 (s, 1H), 4.50 (bro. 1H). MS calculated for C₁₄H₉OCl₂ (M−OH⁻) 263.0, found 262.9.

Step B: To a solution of 2,3-dichloro-5,6-dicyanobenzoquinone (242 mg, 1.07 mmol) and triphenylphosphine (280 mg, 1.07 mmol) in dry CH₂Cl₂ (5 mL) is subsequently added tetrabutylammonium bromide (344 mg (1.07 mmol) and 1,2-Bis-(4-chloro-phenyl)-2-hydroxy-ethanone (200 mg, 0.71 mmol). The suspension is kept stirring for 1.5 h at room temperature. The resulting brown solution is concentrated in vacuo and purified by flash chromatography (hexane/ethyl acetate 5:1) to afford 2-bromo-1,2-bis-(4-chloro-phenyl)-ethanone as a colorless oil: ¹H-NMR (400 MHz, CDCl₃) δ=7.86 (d, J=8.8 Hz, 2H), 7.40-7.35 (m, 4H), 7.29 (d, J=8.4 Hz, 2H), 6.17 (s, 1H). MS calculated for C₁₄H₉OCl₂ (M−Br⁻) 263.0, found 262.9.

Step C: A mixture of 2-bromo-1,2-bis-(4-chloro-phenyl)-ethanone (44.0 mg, 0.13 mmol), (2-methyl-4-thiocarbamoylmethoxy-phenoxy)-acetic acid methyl ester 13 (34.0 mg, 0.13 mmol) and EtOH (1 mL) is subjected to microwave (180° C.) for 5 min. The resulting solution is used directly in the next step.

Step D: The crude {4-[4,5-bis-(4-chloro-phenyl)-thiazol-2-ylmethoxy]-2-methyl-phenoxy}-acetic acid methyl ester from step C is dissolved in THF (1 mL) and H₂O (0.5 mL). LiOH.H₂O (53.7 mg, 0.64 mmol) is added. The mixture is stirred for 2 h at room temperature, then acidified with 1 N HCl. EtOAc (20 mL) is added and the product is extracted. The organic layer is dried (MgSO₄), filtered, concentrated and purified on reverse phase HPLC (H₂O/MeCN gradient) to afford the title compound H1 as a white solid: ¹H-NMR (400 MHz, CD₃OD) δ=7.45 (d, J=8.4 Hz, 2H), 7.39 (d, J=8.4 Hz, 2H), 7.34-7.30 (m, 4H), 6.91 (d, J=2.8 Hz, 1H), 6.85-6.76 (m, 2H), 5.34 (s, 2H), 4.62 (s, 2H), 2.26 (s, 3H). MS calculated for C₂₅H₂₀Cl₂NO₄S (M+H⁺) 500.04, found 501.00.

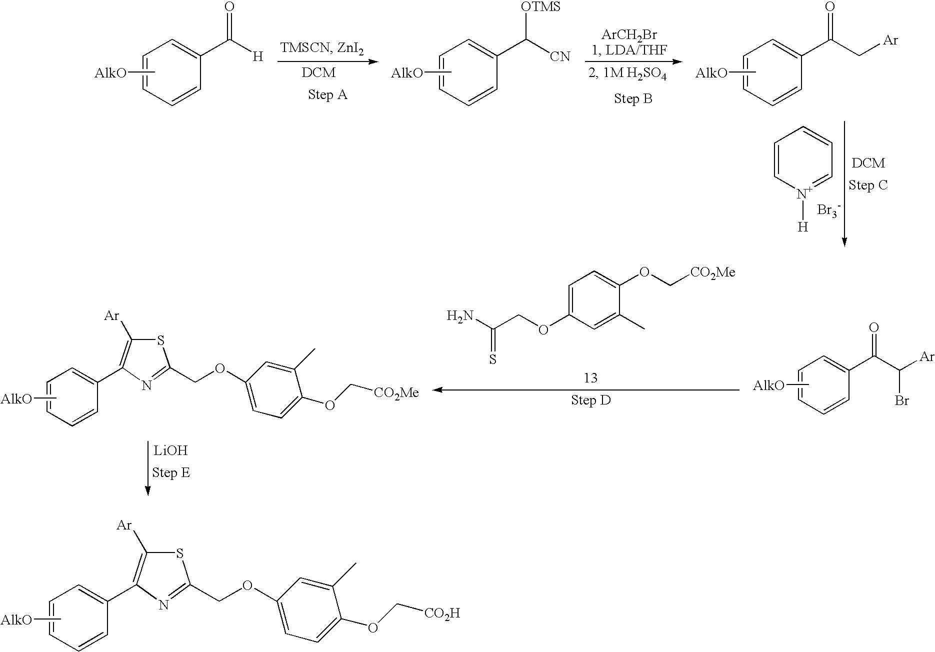

### Example J1

### {4-[4-(4-Isopropoxy-phenyl)-5-(4-trifluoromethoxy-phenyl)-thiazol-2-ylmethoxy]-2-methyl-phenoxy}-acetic acid

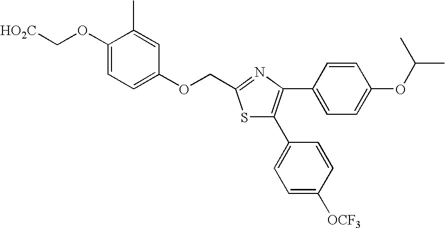

Step A: 4-Isopropoxy-benzaldehyde (5.0 g, 30.45 mmol) and trimethylsilyl cyanide (3.02 g, 30.45 mmol) are dissolved in dry DCM (50 mL). The solution is cooled to 0° C., then zinc iodide (42.76 mg, 1.13 mmol) is added. The reaction mixture is then warmed to room temperature and kept stirring over night. The mixture is concentrated, redissolved in ether and filtered through activated charcoal. The filtrate is dried (MgSO₄) and concentrated to give (4-isopropoxy-phenyl)-trimethylsilanyloxy-acetonitrile as a colorless oil: ¹H-NMR (400 MHz, CDCl₃) δ=7.16 (d, J=8.8 Hz, 1H), 6.70 (d, J=8.8 Hz, 1H), 5.22 (s, 1H), 4.38-4.32 (m, 1H), 1.13 (d, J=4.0 Hz, 6H), 0.00 (s, 9H). MS calculated for C₁₄H₂₁NO₂Si, (M⁺) 263.1, found 237.1 (M−CN).

Step B: (4-Isopropoxy-phenyl)-trimethylsilanyloxy-acetonitrile (1.0 g, 3.78 mmol) is dissolved in dry THF (8 mL). The solution is added dropwise into a solution of LDA (2 M in THF, 1.89 mL) in THF (4 mL) at −78° C. The reaction mixture is stirred for 0.5 h followed by addition a solution of 4-(trifluoromethoxy)benzyl bromide (0.97 g, 3.78 mmol) in THF (2 mL). The reaction mixture is allowed to warm to room temperature and kept stirring for 18 hours. The reaction mixture is poured into H₂O (10 mL) and extracted with EtOAc three times. The organic layers are combined and washed by brine, dried (MgSO₄) and concentrated. The residue is redissolved in MeOH (10 mL), then H₂SO₄ (1 M, 4 mL) is added. After stirring at room temperature over night, the reaction mixture is adjusted to pH 10 by adding 1 N NaOH, then extracted with EtOAc three times. The organic layers are combined and washed with H₂O and brine, dried and concentrated to give crude 1-(4-isopropoxy-phenyl)-2-(4-trifluoromethoxy-phenyl)-ethanone, which is used directly in the next step without purification. MS calculated for C₁₈H₈F₃O₃, (M+H⁺) 339.1, found 339.4.

Step C: The crude 1-(4-isopropoxy-phenyl)-2-(4-trifluoromethoxy-phenyl)-ethanone (100 mg) is dissolved in DCM (2 mL). Pyridinium tribromide (94.5 mg, 0.30 mmol) is added. The reaction mixture is stirred for 2 h at room temperature. The solvent is removed to give crude 2-bromo-1-(4-isopropoxy-phenyl)-2-(4-trifluoromethoxy-phenyl)-ethanone, which is used directly in the next step without purification. MS calculated for C₁₈H₁₇BrF₃O₃, (M+H⁺) 417.0, found 417.3.

Step D: Crude 2-bromo-1-(4-isopropoxy-phenyl)-2-(4-trifluoromethoxy-phenyl)-ethanone is dissolved in EtOH (1.0 mL) in a 5 mL microwave reaction vial. (2-methyl-4-thiocarbamoylmethoxy-phenoxy)-acetic acid methyl ester (13) (79.6 mg, 0.30 mmol) is added and the vial is sealed. Crude {-4-[4-(4-isopropoxy-phenyl)-5-(4-trifluoromethoxy-phenyl)-thiazol-2-ylmethoxy]-2-methyl-phenoxy}-acetic acid methyl ester is obtained after subjection to microwave (5 min at 180° C.) and is used directly in the next step without purification. MS calculated for C₃₀H₂₉F₃NO₆S (M+H⁺) 588.2, found 588.1.

Step E: THF (0.8 mL), H₂O (0.5 mL) and LiOH.H₂O (62 mg, 1.48 mmol) are added to the reaction mixture of step D. The reaction mixture is stirred for 1 h at room temperature, then it is purified on reverse phase HPLC (H₂O/MeCN gradient) to afford the title compound J1 as a white solid: ¹H-NMR (400 MHz, CD₃OD) δ=7.34 (d, J=8.8 Hz, 2H), 7.26 (d, J=8.8 Hz, 2H), 7.17 (d, J=8.4 Hz, 2H), 6.82 (d, J=2.8 Hz, 1H), 6.78-6.67 (m, 4H), 5.24 (s, 2H), 4.53 (s, 2H), 4.51 (m, 1H), 2.16 (s, 3H), 1.22 (s, 3H), 1.20 (s, 3H). MS calculated for C₂₉H₂₇F₃NO₆S (M+H⁺) 574.1, found 574.2.

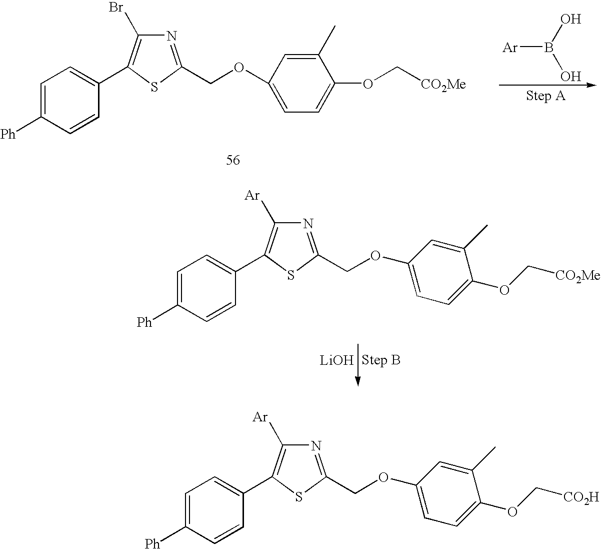

### Example K1

### [4-(5-Biphenyl-4-yl-4-pyridin-3-yl-thiazol-2-ylmethoxy)-2-methyl-phenoxy]-acetic acid

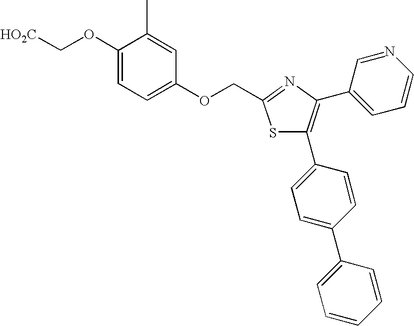

Step A: A mixture of [4-(5-biphenyl-4-yl-4-bromo-thiazol-2-ylmethoxy)-2-methyl-phenoxy]-acetic acid methyl ester (56) (10.0 mg, 0.019 mmol), 3-pyridineboronic acid (3.7 mg, 0.023 mmol), tetrakis(triphenylphosphine)palladium (2.2 mg, 0.0019 mmol), potassium carbonate (10.5 mg, 76.0 mmol), 1,4-dioxane (1 mL), EtOH (0.3 mL) and H₂O (0.2 mL) in a sealed vial is heated to 120° C. and stirred at this temperature overnight. The reaction mixture is cooled to room temperature and used in the next step without further purification. MS calculated for C₃₀H₂₅N₂O₄S (M+H⁺) 523.2, found 523.2.

Step B: LiOH.H₂O (4.0 mg, 0.095 mmol) is added to the reaction mixture from step hours. The mixture is stirred at room temperature for 2 h, then filtered. The filtrate is purified on reverse phase HPLC (H₂O/MeCN gradient) to afford the title compound K1 as a white solid: ¹H-NMR (400 MHz, CD₃OD) δ=8.77 (bs, 1H), 8.55 (bs, 1H), 8.33 (d, J=8.4 Hz, 1H), 7.70 (t, J=6.4 Hz, 1H), 7.64-7.42 (m, 4H), 7.41-7.35 (m, 4H), 7.30-7.26 (m, 1H), 6.84 (d, J=2.8 Hz, 1H), 6.78-6.68 (m, 2H), 5.31 (s, 2H), 4.54 (s, 2H), 2.17 (s, 3H). MS calculated for C₃₀H₂₅N₂O₄S (M+H⁺) 509.2, found 509.1.

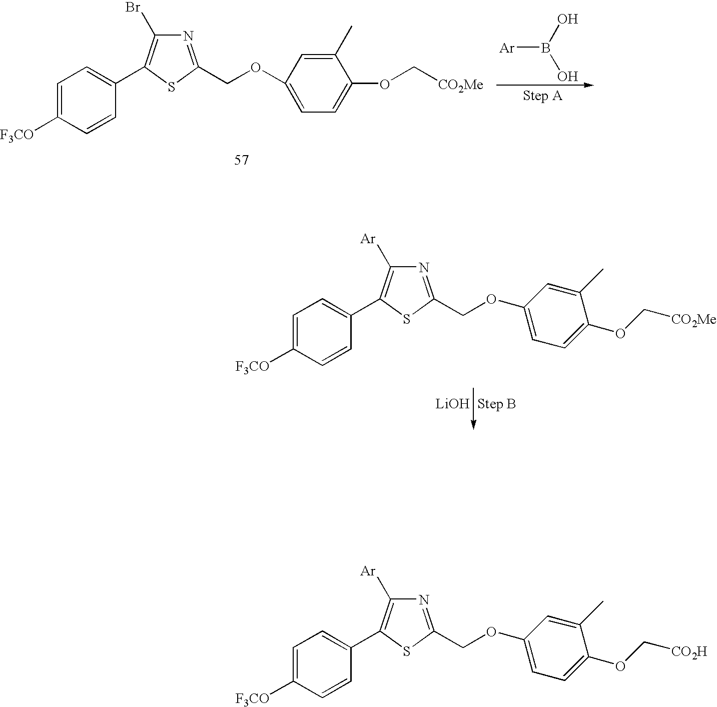

### Example L1

### {4-[4-(6-Methoxy-pyridin-3-yl)-5-(4-trifluoromethoxy-phenyl)-thiazol-2-ylmethoxy]-2-methyl-phenoxy}-acetic acid

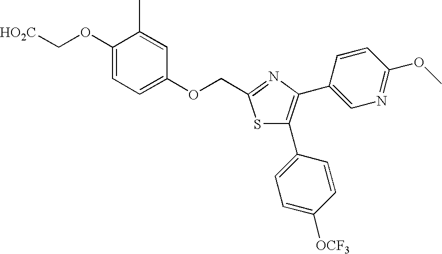

Step A: A mixture of {4-[4-bromo-5-(4-trifluoromethoxy-phenyl)-thiazol-2-ylmethoxy]-2-methyl-phenoxy}-acetic acid methyl ester (400 mg, 0.75 mmol), 2-methoxy-5-pyridineboronic acid (229.7 mg, 1.50 mmol), tetrakis triphenylphosphine)palladium (86.9 mg, 0.075 mmol), potassium carbonate (1.0 N, 3.0 mL, 3.0 mmol), 1,4-dioxane (10.0 mL) and EtOH (6.0 mL) in a sealed vial is heated to 120° C. overnight. The reaction mixture is cooled to room temperature and used in the next step without further purification.

Step B: LiOH.H₂O (158 mg, 3.75 mmol) is added to the reaction mixture from step hours. The mixture is stirred at room temperature for 2 h, then filtered. The filtrate is purified on reverse phase HPLC (H₂O/MeCN gradient) to afford the title compound L1 as a white solid: ¹H-NMR (400 MHz, CD₃OD) δ=8.13 (d, J=2.0 Hz, 1H), 7.72 (dd, J=2.4 Hz, J=8.4 Hz, 2H), 7.39 (d, J=8.8 Hz, 2H), 7.24 (d, J=8.8 Hz, 2H), 6.84 (d, J=2.8 Hz, 1H), 6.77-6.68 (m, 3H), 5.27 (s, 2H), 4.54 (s, 2H), 3.83 (s, 3H), 2.17 (s, 3H). MS calculated for C₂₆H₂₂F₃N₂O₆S (M+H⁺) 547.1, found 547.1.

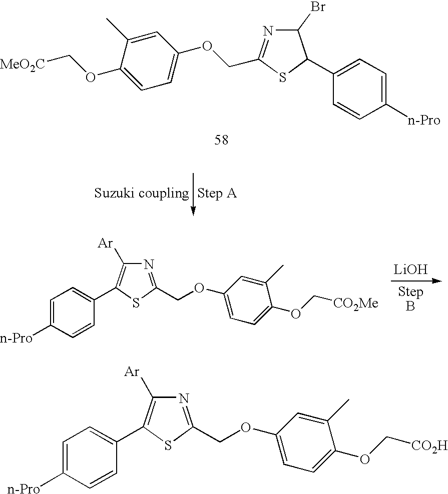

### Example M1

### {4-[4-(6-Methoxy-pyridin-3-yl)-5-(4-propyl-phenyl)-thiazol-2-ylmethoxy]-2-methyl-phenoxy}-acetic acid

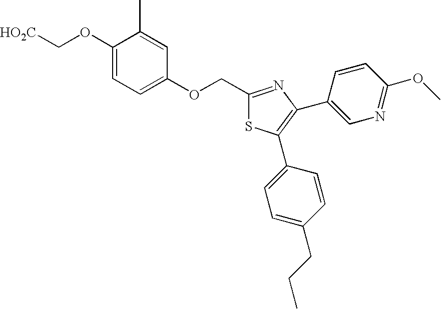

Step A: A mixture of {4-[4-Bromo-5-(4-propyl-phenyl)-thiazol-2-ylmethoxy]-2-methyl-phenoxy}-acetic acid methyl ester 58 (20 mg, 0.041 mmol), 2-methoxy-5-pyridineboronic acid (12.5 mg, 0.082 mmol), tetrakis triphenylphosphine)palladium (4.7 mg, 0.0041 mmol), potassium carbonate (1.0 N, 0.16 mL, 0.16 mmol), 1,4-dioxane (0.6 mL) and EtOH (0.3 mL) in a sealed vial is subjected to microwave (5 min at 170° C.). Crude {4-[4-(6-Methoxy-pyridin-3-yl)-5-(4-trifluoromethoxy-phenyl)-thiazole-2-ylmethoxy]-2-methyl-phenoxy}-acetic acid methyl ester is obtained and is used directly in the next step without purification. MS calculated for C₂₇H₂₄F₃N₂O₇ (M+H⁺) 519.2, found 519.2.

Step B: LiOH.H₂O (17.0 mg, 0.41 mmol), MeOH (0.4 mL), THF (0.3 mL) and H₂O (0.2 mL) are added to the reaction mixture from step G. The mixture is stirred at room temperature for 2 h, then filtered. The filtrate is purified on reverse phase HPLC (H₂O/MeCN gradient) to afford the title compound M1 as a white solid: ¹H-NMR (400 MHz, CD₃OD) δ=8.12 (s, 1H), 7.71 (dd, J=2.4 Hz, J=8.8 Hz, 1H), 7.17-7.11 (m, 4H), 6.82 (d, J=2.8 Hz, 1H), 6.73-6.66 (m, 3H), 5.24 (s, 2H), 4.53 (s, 2H), 3.82 (s, 3H), 2.51 (t, J=7.6 Hz, 2H), 2.16 (s, 3H), 1.61-1.51 (m, 2H), 0.86 (t, J=7.2 Hz, 3H). MS calculated for C₂₈H₂₉N₂O₅S (M+H⁺) 505.2, found 505.2.

### Example N1

### 2-{4-[4-(4-Methoxy-phenyl)-5-(4-trifluoromethyl-phenyl)-thiazol-2-ylmethoxy]-phenoxy}-propionic acid

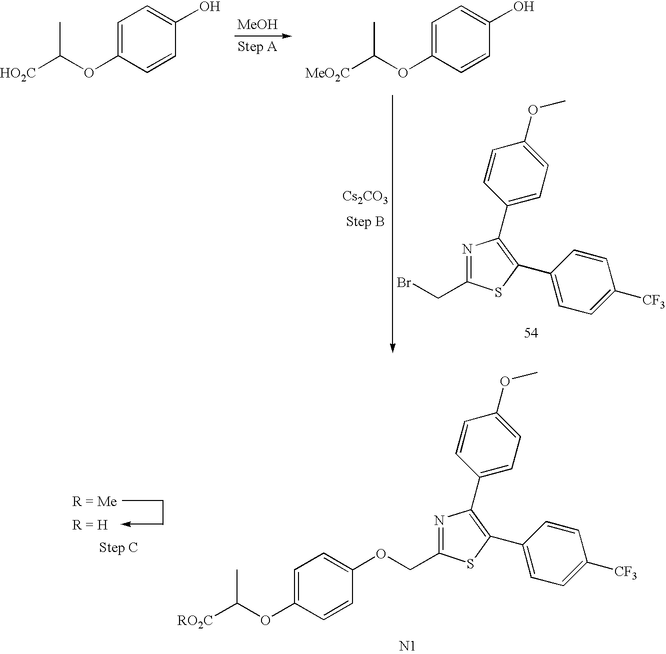

Step A: 2-(4-Hydroxy-phenoxy)-propionic acid (25 mg, 0.14 mmol) is dissolved in MeOH (20 mL). Thienyl chloride (5 □L, 0.06 mmol) is added and the solution is stirred at 60° C. for 2 hours. The solvent is removed in vacuo to afford crude 2-(4-hydroxy-phenoxy)-propionic acid methyl ester as a white solid: ¹H-NMR (400 MHz, CDCl₃) δ=6.77 (d, J=9.2 Hz, 2H), 6.72 (d, J=9.2 Hz, 1H), 4.66 (q, J=6.8 Hz, 1H), 3.75 (s, 3H), 1.59 (d, J=6.8 Hz, 3H). MS calculated for C₁₀H₁₃O₄ (M+H⁺) 197.1, found 197.4.

Step B: 2-(4-hydroxy-phenoxy)-propionic acid methyl ester (27 mg, 0.14 mmol) and Cs₂CO₃ (137 mg, 0.42 mmol) are added to a solution of intermediate 54 (60 mg, 0.14 mmol) in MeCN (5 mL). The mixture is stirred for 3 h at room temperature. After the mixture is filtered, the organic solution is concentrated to afford crude 2-{4-[4-(4-Methoxy-phenyl)-5-(4-trifluoromethyl-phenyl)-thiazol-2-ylmethoxy]-phenoxy}-propionic acid methyl ester, which is used in the next step without further purification.

Step C: THF (2 mL) and 1 N LiOH (0.5 mL) are added to the crude product from step B. The mixture is stirred overnight at room temperature, then acidified with 1 N HCl (1 mL). The reaction mixture is extracted with EtOAc (3 mL), the organic layer is separated and concentrated in vacuo. The remainder is taken up in DMSO (1 mL) and purified on reverse phase HPLC (H₂O/MeCN gradient) to afford the title compound N1 as a white solid: ¹H-NMR (400 MHz, CDCl₃) δ=7.55 (d, J=8.2 Hz, 2H), 7.43 (d, J=8.2 Hz, 2H), 7.39 (d, J=8.8 Hz, 2H), 6.95-6.83 (m, 6H), 5.31 (s, 2H), 4.69 (q, J=6.8 Hz, 1H), 3.81 (s, 3H), 1.60 (d, J=6.8 Hz, 3H). MS calculated for C₂₇H₂₃F₃NO₅S (M+H⁺) 530.1, found 530.4.

By repeating the procedures described in the above examples, using appropriate starting materials, the following compounds of Formula I, as identified in Table 1, are obtained.

TABLE 1

| | | Physical Data |
|---|---|---|
| Compound | Compound | ¹H NMR 400 MHz (DMSO-d₆) |
| Number | Structure | and/or MS (m/z) |
| A2 | 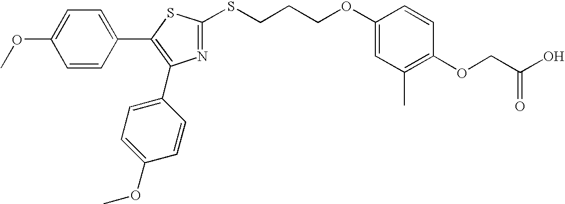 | ¹H-NMR (400MHz, CD₃OD) δ =7.34 (d, J=8.9 Hz, 2H), 7.14 (d, J=8.9 Hz, 2H), 6.87 (d, J=8.9 Hz, 2H), 6.82 (d, J=8.9 Hz, 2H), 6.73-6.63 (m, 3H), 4.55 (s, 2H), 4.06 (t, J=5.9 Hz, 2H), 3.79 (s, 3H), 3.78 (s, 3H), 3.42 (t, J=7.0 Hz, 2H), 2.23 (m, 2H), 2.18 (s, 3H). MS calculated for C₂₉H₃₀NO₆S₂ (M + H⁺) 552.1, found 552.4. |
| A3 | 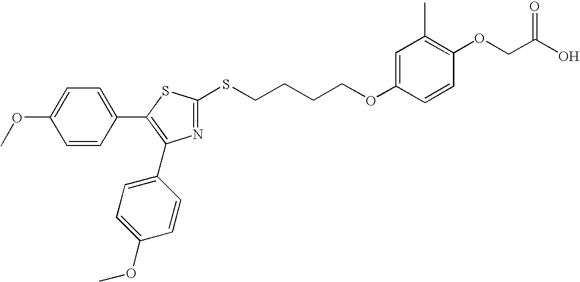 | ¹H-NMR (400MHz, CD₃OD) δ =7.34 (d, J=8.9 Hz, 2H), 7.16 (d, J=8.9 Hz, 2H), 6.87 (d, J=8.9 Hz, 2H), 6.80 (d, J=8.9 Hz, 2H), 6.72-6.61 (m, 3H), 4.56 (s, 2H), 3.95 (t, J=6.0 Hz, 2H), 3.79 (s, 3H), 3.77 (s, 3H), 3.31 (t, J=7.2 Hz, 2H), 2.19 (s, 3H), 2.01-1.89 (m, 4H). MS calculated for C₃₀H₃₂NO₆S₂ (M + H⁺) 566.2, found 566.4. |
| A4 | 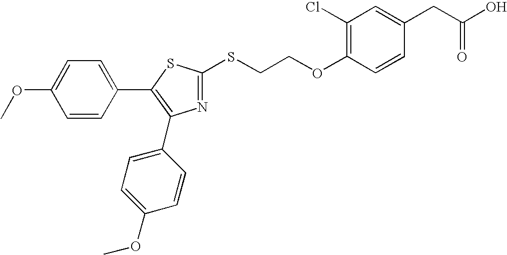 | ¹H-NMR (400MHz, CD₃OD) δ =7.37-6.82 (m, 11H), 4.42 (t, J=6.4 Hz, 2H), 3.79 (s, 3H), 3.78 (s, 3H), 3.66 (t, J=6.4 Hz, 2H), 3.50 (s, 2H). MS calculated for C₂₇H₂₅ClNO₅S₂ (M + H⁺) 542.1, found 542.3. |
| A5 | 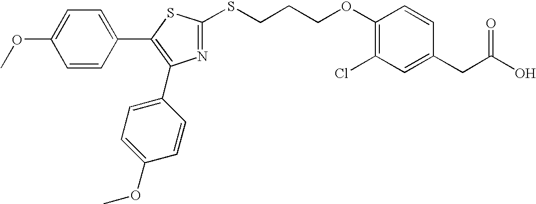 | ¹H-NMR (400MHz, CD₃OD) δ =7.35-6.80 (m, 11H), 4.19 (t, J=5.8 Hz, 2H), 3.79 (s, 3H), 3.77 (s, 3H), 3.55 (s, 2H), 3.47 (t, J=7.0 Hz, 2H), 2.30 (m, 2H). MS calculated for C₂₈H₂₇ClNO₅S₂(M + H⁺) 556.1, found 556.4. |
| A6 | 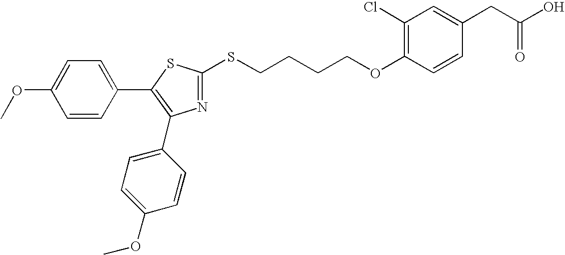 | ¹H-NMR (400MHz, CD₃OD) δ =7.36-6.80 (m, 11H), 4.10 (t, J=5.7 Hz, 2H), 3.80 (s, 3H), 3.78 (s, 3H), 3.51 (s, 2H), 3.36 (t, J=7.1 Hz, 2H), 2.08-1.98 (m, 4H). MS calculated for C₂₉H₂₉ClNO₅S₂(M + H⁺) 570.1, found 570.4. |
| B2 | 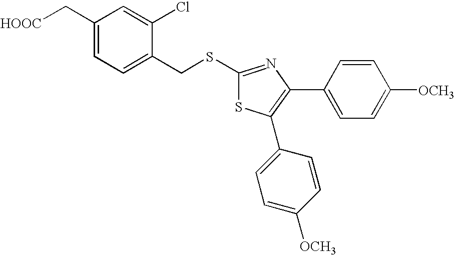 | ¹H-NMR (400MHz, CDCl₃) δ =7.51-7.45 (m, 3H), 7.35 (d, J=1.2 Hz, 1H), 7.22 (s, 1H), 7.20 (s, 1H), 7.13 (dd, J=1.2 Hz, J=8.0 Hz, 1H), 4.57 (s, 2H), 3.802 (s, 3H), 3.809 (s, 3H), 3.62 (s, 2H). MS calculated for C₂₆H₂₃ClNO₄S₂(M + H⁺) 512.0, found 512.00. |
| C2 | 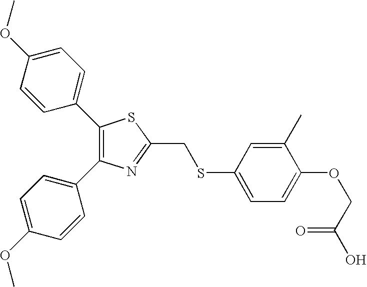 | ¹H-NMR (400MHz, CDCl₃) δ =7.74-6.63 (m, 11H), 4.62 (s, 2H), 4.35 (s, 2H), 3.80 (s, 3H), 3.77 (s, 3H), 2.23 (s, 3H). MS calculated for C₂₇H₂₆NO₅S₂ (M + H⁺) 508.1, found 508.4. |
| C3 | 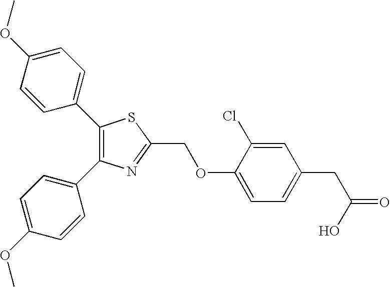 | ¹H-NMR (400MHz, CDCl₃) δ =7.74-6.80 (m, 11H), 5.43 (s, 2H), 3.81 (s, 3H), 3.80 (s, 3H), 3.57 (s, 2H). MS calculated for C₂₆H₂₃ClNO₅S (M + H⁺) 496.1, found 496.3. |
| C4 | 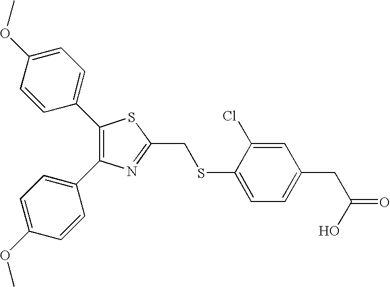 | ¹H-NMR (400MHz, CDCl₃) δ =7.74-6.78 (m, 11H), 4.51 (s, 2H), 3.79 (s, 3H), 3.79 (s, 3H), 3.58 (s, 2H). MS calculated for C₂₆H₂₃ClNO₄S₂ (M + H⁺) 512.1, found 512.3. |
| D2 | 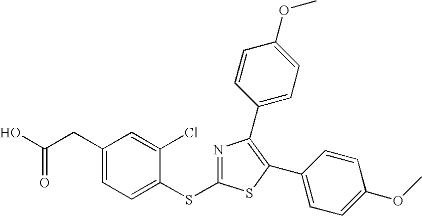 | ¹H-NMR (400Mhz, CDCl₃) δ =7.59 (d, J=8.0 Hz, 1H), 7.44 (s, 1H), 7.37 (d, 8.8 Hz, 2H), 7.21 (d, J=8.4 Hz, 1H), 7.15 (d, J=8.8 Hz, 2H), 6.80 (d, J=8.4 Hz, 4H), 3.77 (s, 6H), 3.64 (s, 2H). MS calculated for C₂₆H₂₄NO₅S₂(M + H⁺) 498.0, found 498.3. |
| E48 | 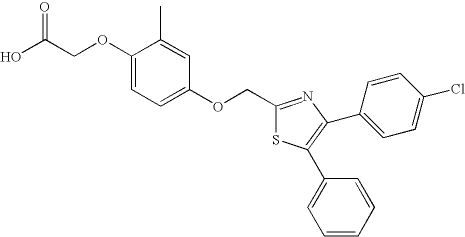 | ¹H-NMR (600 MHz, (CD₃)₂SO) δ =7.46-7.31 (m, 9H), 6.96 (d, J=3.0 Hz, 1H), 6.87 (dd, J=3.0 Hz, J=8.9 Hz, 1H), 6.79 (d, J=8.9 Hz, 1H), 5.40 (s, 2H), 4.63 (s, 2H), 2.19 (s, 3H). MS calculated for C₂₅H₂₁ClNO₄S (M + H⁺) 466.1, found 466.3. |
| E49 | 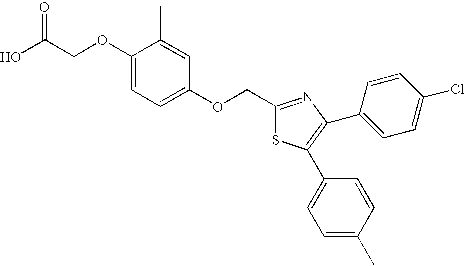 | ¹H-NMR (600 MHz, (CD₃)₂SO) δ =7.46-7.08 (m, 8H), 6.96 (d, J=3.0 Hz, 1H), 6.86 (dd, J=3.0 Hz, J=8.9 Hz, 1H), 6.78 (d, J=8.9 Hz, 1H), 5.39 (s, 2H), 4.63 (s, 2H), 2.69 (m, 3H), 2.19 (s, 3H). MS calculated for C₂₆H₂₃ClNO₄S (M + H⁺) 480.1, found 480.3. |
| E60 | 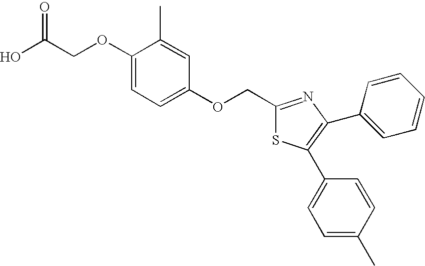 | ¹H-NMR (600 MHz, (CD₃)₂SO) δ =7.46-7.19 (m, 9H), 6.96 (d, J=2.9 Hz, 1H), 6.87 (dd, J=3.0 Hz, J=8.9 Hz, 1H), 6.79 (d, J=8.9 Hz, 1H), 5.39 (s, 2H), 4.63 (s, 2H), 2.32 (s, 3H), 2.19 (s, 3H). MS calculated for C₂₆H₂₄NO₄S (M + H⁺) 446.1, found 446.4. |
| E61 | 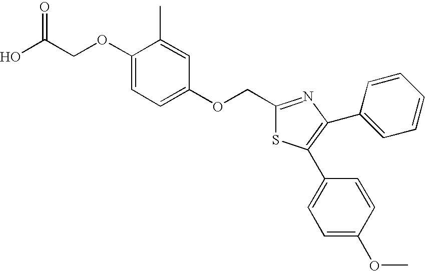 | ¹H-NMR (600 MHz, (CD₃)₂SO) δ =7.47-7.26 (m, 7H), 6.95 (m, 3H), 6.87 (dd, J=3.0 Hz, J=8.9 Hz, 1H), 6.79 (d, J=8.9 Hz, 1H), 5.38 (s, 2H), 4.63 (s, 2H), 3.77 (s, 3H), 2.19 (s, 3H). MS calculated for C₂₆H₂₄NO₅S (M + H⁺) 462.1, found 462.4. |
| E62 | 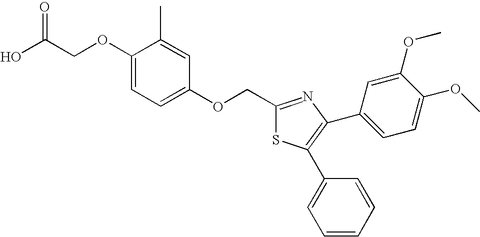 | ¹H-NMR (600 MHz, (CD₃)₂SO) δ =7.50-7.44 (m, 5H), 7.09-6.85 (m, 6H), 5.46 (s, 2H), 4.70 (s, 2H), 3.81 (s, 3H), 3.59 (s, 3H), 2.26 (s, 3H). MS calculated for C₂₇H₂₆NO₆S (M + H⁺) 492.1, found 492.4. |
| E63 | 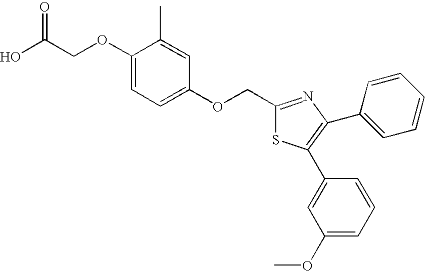 | ¹H-NMR (600 MHz, (CD₃)₂SO) δ =7.45-7.26 (m, 6H), 6.94-6.76 (m, 6H), 5.37 (s, 2H), 4.61 (s, 2H), 3.64 (s, 3H), 2.17 (s, 3H). MS calculated for C₂₆H₂₄NO₅S (M + H⁺) 462.1, found 462.4. |
| E64 | 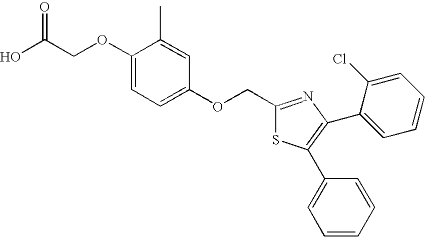 | ¹H-NMR (600 MHz, (CD₃)₂SO) δ =7.54-7.19 (m, 9H), 6.96 (d, J=3.0 Hz, 1H), 6.88 (dd, J=3.0 Hz, J=8.9 Hz, 1H), 6.79 (d, J=8.9 Hz, 1H), 5.39 (s, 2H), 4.64 (s, 2H), 2.19 (s, 3H). MS calculated for C₂₅H₂₁ClNO₄S (M + H⁺) 466.1, found 466.3. |
| E65 | 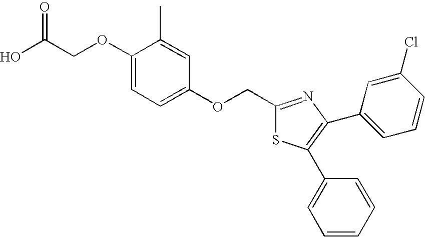 | ¹H-NMR (600 MHz, (CD₃)₂SO) δ =7.46-7.27 (m, 9H), 6.94 (d, J=3.0 Hz, 1H), 6.84 (dd, J=3.0 Hz, J=8.9 Hz, 1H), 6.76 (d, J=8.9 Hz, 1H), 5.38 (s, 2H), 4.61 (s, 2H), 2.17 (s, 3H). MS calculated for C₂₅H₂₁ClNO₄S (M + H⁺) 466.1, found 466.3. |
| E66 | 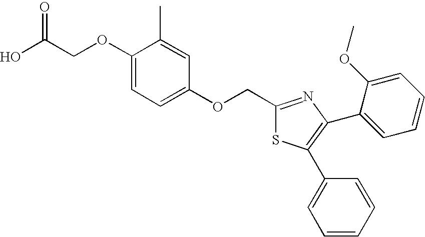 | ¹H-NMR (600 MHz, (CD₃)₂SO) δ =7.39-7.19 (m, 7H), 7.01-6.96 (m, 3H), 6.87 (dd, J=3.0 Hz, J=8.9 Hz, 1H), 6.79 (d, J=8.9 Hz, 1H), 5.36 (s, 2H), 4.63 (s, 2H), 3.39 (s, 3H), 2.20 (s, 3H). MS calculated for C₂₆H₂₄NO₅S (M + H⁺) 462.1, found 462.4. |
| E67 | 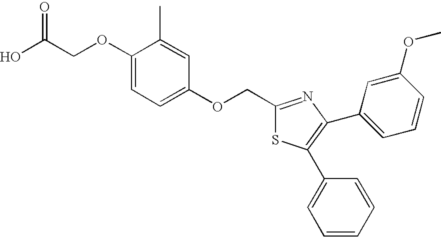 | ¹H-NMR (600 MHz, (CD₃)₂SO) δ =7.41-7.22 (m, 7H), 7.03-6.97 (m, 3H), 6.88 (dd, J=3.0 Hz, J=8.9 Hz, 1H), 6.79 (d, J=8.9 Hz, 1H), 5.40 (s, 2H), 4.63 (s, 2H), 3.62 (s, 3H), 2.19 (s, 3H). MS calculated for C₂₆H₂₄NO₅S (M + H⁺) 462.1, found 462.4. |
| E68 | 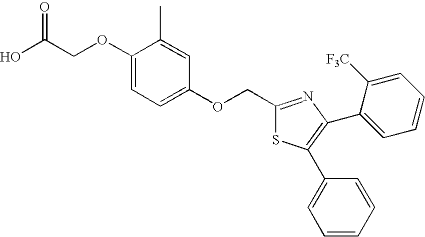 | MS calculated for C₂₆H₂₁F₃NO₄S (M + H⁺) 500.1, found 500.4. |
| E69 | 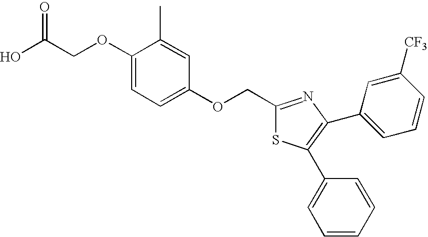 | ¹H-NMR (600 MHz, (CD₃)₂SO) δ =7.75-7.34 (m, 9H), 6.98 (d, J=3 Hz, 1H), 6.88 (dd, J=3.0 Hz, J=8.9 Hz, 1H), 6.79 (d, J=8.9 Hz, 1H), 5.43 (s, 2H), 4.64 (s, 2H), 2.20 (s, 3H). MS calculated for C₂₆H₂₁F₃NO₄S (M + H⁺) 500.1, found 500.3. |
| E70 | 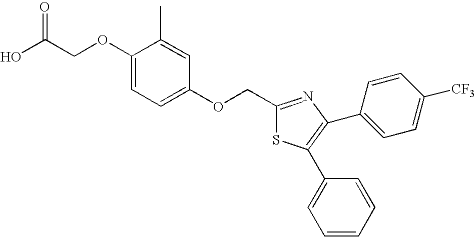 | ¹H-NMR (600 MHz, (CD₃)₂SO) δ =7.71-7.63 (m, 4H), 7.44-7.33 (m, 5H), 6.97 (d, J=3 Hz, 1H), 6.88 (dd, J=3.0 Hz, J=8.9 Hz, 1H), 6.79 (d, J=8.9 Hz, 1H), 5.42 (s, 2H), 4.64 (s, 2H), 2.19 (s, 3H). MS calculated for C₂₆H₂₁F₃NO₄S (M + H⁺) 500.1, found 500.4. |
| E71 | 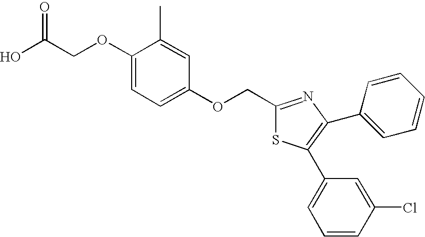 | ¹H-NMR (600 MHz, (CD₃)₂SO) δ =7.46-7.26 (m, 9H), 6.97 (d, J=3.0 Hz, 1H), 6.88 (dd, J=3.0 Hz, J=8.9 Hz, 1H), 6.79 (d, J=8.9 Hz, 1H), 5.41 (s, 2H), 4.64 (s, 2H), 2.19 (s, 3H). MS calculated for C₂₅H₂₁ClNO₄S (M + H⁺) 466.1, found 466.3. |
| E72 | 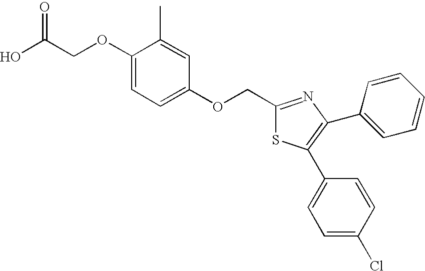 | ¹H-NMR (600 MHz, (CD₃)₂SO) δ =7.47-7.30 (m, 9H), 6.96 (d, J=3.0 Hz, 1H), 6.87 (dd, J=3.0 Hz, J=8.9 Hz, 1H), 6.79 (d, J=8.9 Hz, 1H), 5.40 (s, 2H), 4.64 (s, 2H), 2.19 (s, 3H). MS calculated for C₂₅H₂₁ClNO₄S (M + H⁺) 466.1, found 466.3. |
| E73 | 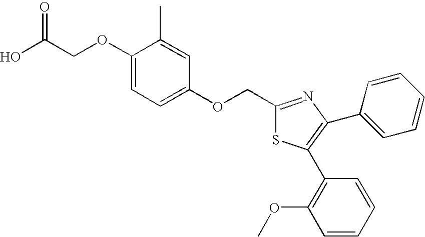 | ¹H-NMR (600 MHz, (CD₃)₂SO) δ =7.43-7.12 (m, 8H), 6.97-6.79 (m, 4H), 5.39 (s, 2H), 4.64 (s, 2H), 3.64 (s, 3H), 2.19 (s, 3H). MS calculated for C₂₆H₂₄NO₅S (M + H⁺) 462.1, found 462.4. |
| E74 | 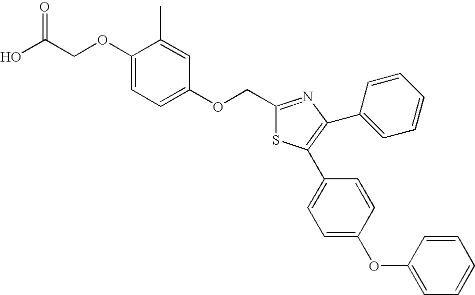 | ¹H-NMR (600 MHz, (CD₃)₂SO) δ =7.45-7.31 (m, 8H), 7.17-6.78 (m, 9H), 5.38 (s, 2H), 4.63 (s, 2H), 2.19 (s, 3H). MS calculated for C₃₁H₂₆NO₅S (M + H⁺) 524.2, found 524.4. |
| E75 | 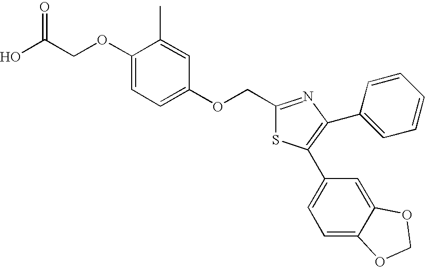 | ¹H-NMR (600 MHz, (CD₃)₂SO) δ =7.48-7.30 (m, 5H), 6.96-6.78 (m, 6H), 6.06 (s, 2H), 5.37 (s, 2H), 4.63 (s, 2H), 2.19 (s, 3H). MS calculated for C₂₆H₂₂NO₆S (M + H⁺) 476.1, found 476.4. |
| E76 | 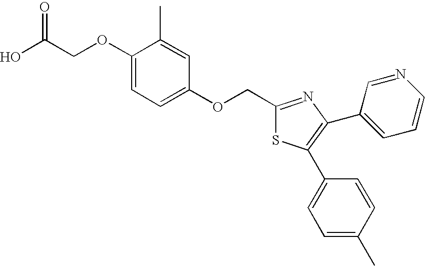 | ¹H-NMR (600 MHz, (CD₃)₂SO) δ =8.64 (d, J=1.7 Hz, 1H), 8.54 (dd, J=1.4 Hz, J=4.8 Hz, 1H), 7.91 (d, J=8.0 Hz), 7.47 (dd, J=5.0 Hz, J=7.9 Hz, 1H), 7.27 (d, J=8.1 Hz, 2H), 7.24 (d, J=8.1 Hz, 2H), 6.97 (d, J=3.0 Hz, 1H), 6.87 (dd, J=3.0 Hz, J=8.9 Hz, 1H), 6.79 (d, J=8.9 Hz, 1H), 5.41 (s, 2H), 4.64 (s, 2H), 2.34 (s, 3H), 2.19 (s, 3H). MS calculated |
| # for C₂₅H₂₃N₂O₄S (M + H⁺) 447.1, found 447.4. | | |
| E87 | 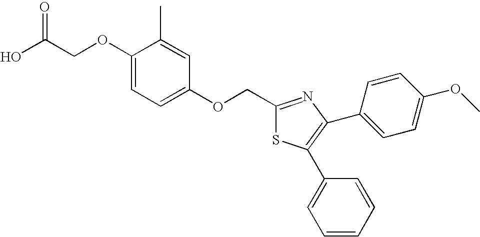 | ¹H-NMR (600 MHz, (CD₃)₂SO) δ =7.40-7.34 (m, 7H), 6.96 (d, J=3.0 Hz, 1H), 6.87 (m, 3H), 6.78 (d, J=8.9 Hz, 1H), 5.38 (s, 2H), 4.63 (s, 2H), 3.74 (s, 3H), 2.19 (s, 3H). MS calculated for C₂₆H₂₄NO₅S (M + H⁺) 462.1, found 462.4. |
| E90 | 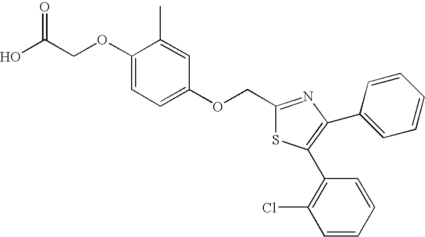 | ¹H-NMR (600 MHz, (CD₃)₂SO) δ =7.62-7.25 (m, 9H), 6.98 (d, J=3.0 Hz, 1H), 6.89 (dd, J=3.0 Hz, J=8.9 Hz, 1H), 6.80 (d, J=8.9 Hz, 1H), 5.44 (s, 2H), 4.64 (s, 2H), 2.20 (s, 3H). MS calculated for C₂₅H₂₁ClNO₄S (M + H⁺) 466.1, found 466.3. |
| E91 | 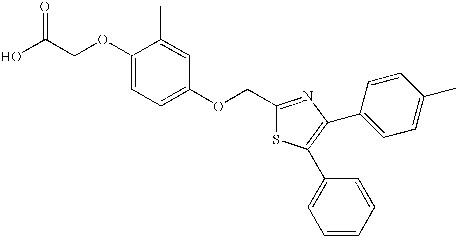 | ¹H-NMR (600 MHz, (CD₃)₂SO) δ =7.40-7.33 (m, 7H), 7.13 (d, J=8.0 Hz, 2H), 6.96 (d, J=3.0 Hz, 1H), 6.87 (dd, J=3.0 Hz, J=8.9 Hz, 1H), 6.79 (d, J=8.9 Hz, 1H), 5.39 (s, 2H), 4.63 (s, 2H), 2.29 (s, 3H), 2.19 (s, 3H). MS calculated for C₂₆H₂₄NO₄S (M + H⁺) 446.1, found 446.4. |
| F2 | 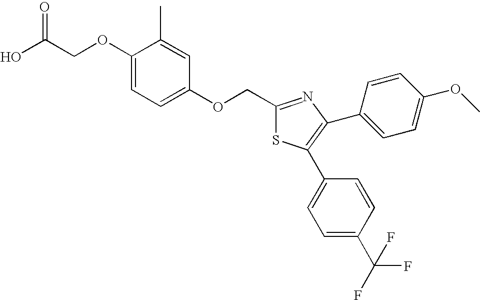 | ¹H-NMR (400 MHz, CDCl₃) δ =7.48 (d, J=8.2 Hz, 2H), 7.36 (d, J=8.2 Hz, 2H), 7.32 (d, J=8.8 Hz, 2H), 6.83 (d, J=3.0 Hz, 1H), 6.77 (d, J=8.8 Hz, 2H), 6.71 (dd, J=3.0, 8.9 Hz, 1H), 6.64 (d, J=8.8 Hz, 1H), 5.26 (s, 2H), 4.56 (s, 2H), 3.74 (s, 3H), 2.21 (s, 3H). MS calculated for C₂₇H₂₃F₃NO₅S (M + H⁺) 530.1, found 530.3. |
| F3 | 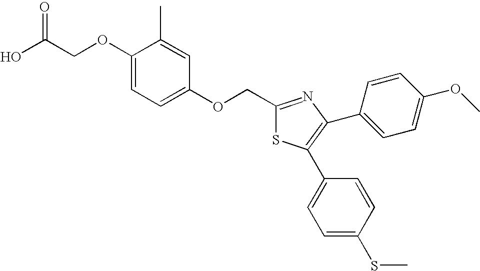 | ¹H-NMR (400 MHz, CDCl₃) δ =7.35 (d, J=8.8 Hz, 2H), 7.15 (d, J=8.3 Hz, 2H), 7.09 (d, J=8.4 Hz, 2H), 6.82 (d, J=2.8 Hz, 1H), 6.76 (d, J=8.8 Hz, 2H), 6.71 (dd, J=3.1, 8.9 Hz, 1H), 6.63 (d, J=8.8 Hz, 1H), 5.26 (s, 2H), 4.55 (s, 2H), 3.74 (s, 3H), 2.41 (s, 3H), 2.21 (s, 3H). MS calculated for C₂₇H₂₆NO₅S₂ (M + H⁺) 508.1, found 508.3. |
| F4 | 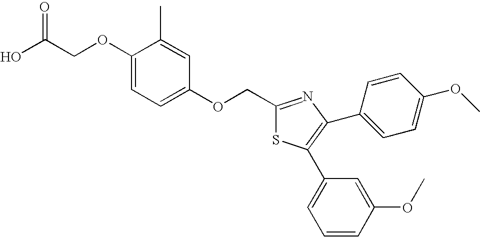 | ¹H-NMR (400 MHz, CDCl₃) δ =7.42 (d, J=8.8 Hz, 2H), 7.22 (t, J=7.5 Hz, 1H), 6.90 (m, 2H), 6.86 (m, 4H), 6.77 (dd, J=3.1, 8.8 Hz, 1H), 6.70 (d, J=8.8 Hz, 1H), 5.35 (s, 2H), 4.63 (s, 2H), 3.81 (s, 3H), 3.70 (s, 3H), 2.28 (s, 3H). MS calculated for C₂₇H₂₆NO₆S (M + H⁺) 492.1, found 492.4. |
| F5 | 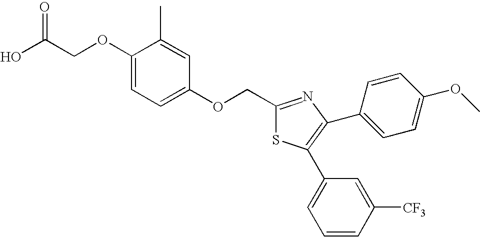 | ¹H-NMR (400 MHz, CDCl₃) δ =7.61 (s, 1H), 7.56 (d, J=7.6 Hz, 1H), 7.47 (d, J=8.0 Hz, 1H), 7.43 (d, J=7.6 Hz, 1H), 7.38 (d, J=8.8 Hz, 2H), 6.90 (d, J=2.8 Hz, 1H), 6.85 (d, J=8.8 Hz, 2H), 6.78 (dd, J=3.2, 8.8 Hz, 1H), 6.71 (d, J=8.8 Hz, 1H), 5.37 (s, 2H), 4.64 (s, 2H), 3.82 (s, 3H), 2.29 (s, 3H). MS calculated for C₂₇H₂₃F₃NO₅S (M + H⁺) |
| # 530.1, found 530.4. | | |
| F6 | 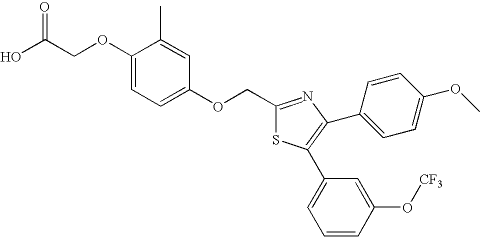 | ¹H-NMR (400 MHz, CDCl₃) δ =7.39 (d, J=8.8 Hz, 2H), 7.34 (t, J=7.2 Hz, 1H), 7.27 (d, J=7.2 Hz, 1H), 7.15 (m, 2H), 6.90 (d, J=2.8 Hz, 1H), 6.85 (d, J=8.8 Hz, 2H), 6.78 (dd, J=2.8, 8.8 Hz, 1H), 6.70 (d, J=8.8 Hz, 1H), 5.35 (s, 2H), 4.64 (s, 2H), 3.82 (s, 3H), 2.29 (s, 3H). MS calculated for C₂₇H₂₃F₃NO₆S (M + H⁺) 546.1, found 546.0. |
| F7 | 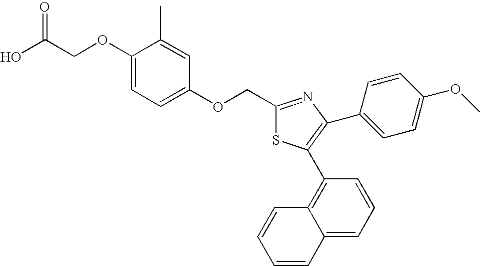 | ¹H-NMR (400 MHz, CDCl₃) δ =7.91 (m, 2H), 7.75 (d, J=8.4 Hz, 1H), 7.48 (m, 3H), 7.40 (m, 1H), 7.33 (d, J=8.9 Hz, 2H), 6.92 (d, J=2.8 Hz, 1H), 6.82 (dd, J=2.8, 8.8 Hz, 1H), 6.73 (d, J=8.8 Hz, 1H), 6.64 (d, J=8.9 Hz, 2H), 5.42 (s, 2H), 4.65 (s, 2H), 3.69 (s, 3H), 2.30 (s, 3H). MS calculated for C₃₀H₂₆NO₅S (M + H⁺) 512.2, found 512.1. |
| F8 | 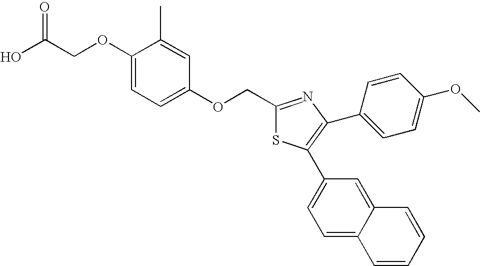 | ¹H-NMR (400 MHz, CDCl₃) δ =7.88 (s, 1H), 7.76 (m, 3H), 7.49 (m, 2H), 7.45 (d, J=8.8 Hz, 2H), 7.34 (dd, J=1.6, 8.4 Hz, 1H), 6.92 (d, J=2.8 Hz, 1H), 6.81 (d, J=8.8 Hz, 2H), 6.80 (dd, J=2.8, 8.8 Hz, 1H), 6.71 (d, J=8.8 Hz, 1H), 5.39 (s, 2H), 4.64 (s, 2H), 3.80 (s, 3H), 2.29 (s, 3H). MS calculated for C₃₀H₂₆NO₅S (M + H⁺) 512.2, found 512.1. |
| F9 | 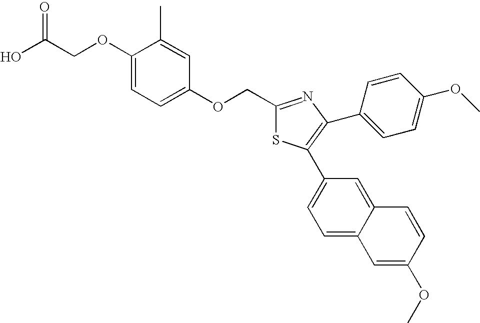 | ¹H-NMR (400 MHz, CDCl₃) δ =7.78 (s, 1H), 7.66 (d, J=9.2 Hz, 1H), 7.63 (d, J=8.8 Hz, 1H), 7.43 (d, J=8.8 Hz, 2H), 7.29 (dd, J=2.0, 8.4 Hz, 1H), 7.16 (dd, J=2.4, 8.8 Hz, 1H), 7.11 (d, J=2.4 Hz, 1H), 6.91 (d, J=2.8 Hz, 1H), 6.81 (d, J=9.2 Hz, 2H), 6.79 (dd, J=2.8, 8.8 Hz, 1H), 6.71 (d, J=8.8 Hz, 1H), 5.39 (s, 2H), 4.64 (s, 2H), 3.95 (s, 3H), 3.80 (s, 3H), 2.29 |
| # (s, 3H). MS calculated for C₃₁H₂₈NO₆S (M + H⁺) 542.2, found 542.4. | | |
| F10 | 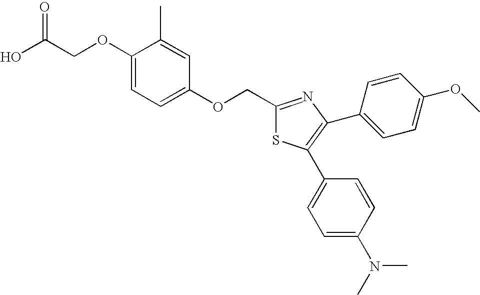 | ¹H-NMR (400 MHz, CDCl₃) δ =7.50 (d, J=8.8 Hz, 2H), 7.45 (d, J=8.8 Hz, 2H), 7.26 (d, J=8.8 Hz, 2H), 7.00 (d, J=2.8 Hz, 1H), 6.96 (d, J=8.8 Hz, 2H), 6.88 (dd, J=2.8, 8.8 Hz, 1H), 6.80 (d, J=8.8 Hz, 1H), 5.46 (s, 2H), 4.73 (s, 2H), 3.93 (s, 3H), 3.22 (s, 6H), 2.39 (s, 3H). MS calculated for C₂₈H₂₉NO₅S (M + H⁺) 505.2, found 505.4. |
| F11 | 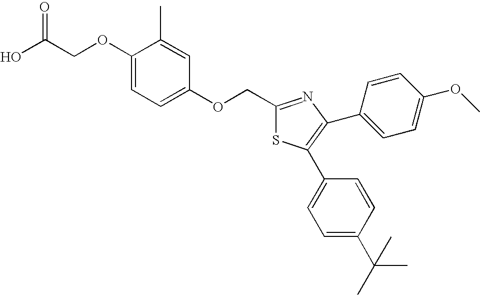 | ¹H-NMR (400 MHz, CDCl₃) δ =7.40 (d, J=8.8 Hz, 2H), 7.34 (d, J=8.4 Hz, 2H), 7.23 (d, J=8.4 Hz, 2H), 6.89 (d, J=2.4 Hz, 1H), 6.86 (d, J=8.8 Hz, 2H), 6.78 (dd, J=2.8, 8.8 Hz, 1H), 6.70 (d, J=8.8 Hz, 1H), 5.44 (s, 2H), 4.63 (s, 2H), 3.83 (s, 3H), 2.28 (s, 3H), 1.31 (s, 9H). MS calculated for C₃₀H₃₂NO₅S (M + H⁺) 518.2, found 518.4. |
| F12 | 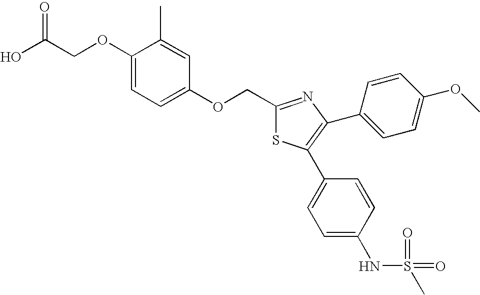 | ¹H-NMR (400 MHz, DMSO-d6) δ =9.98 (s, 1H), 7.40 (d, J=8.8 Hz, 2H), 7.31 (d, J=8.8 Hz, 2H), 7.20 (d, J=8.8 Hz, 2H), 6.95 (d, J=2.8 Hz, 1H), 6.89 (d, J=8.8 Hz, 2H), 6.86 (dd, J=2.8, 8.8 Hz, 1H), 6.78 (d, J=8.8 Hz, 1H), 5.37 (s, 2H), 4.63 (s, 2H), 3.76 (s, 3H), 2.51 (s, 3H), 2.20 (s, 3H). MS calculated for C₂₇H₂₇N₂O₇S₂(M + H⁺) |
| # 555.1, found 555.3. | | |
| F13 | 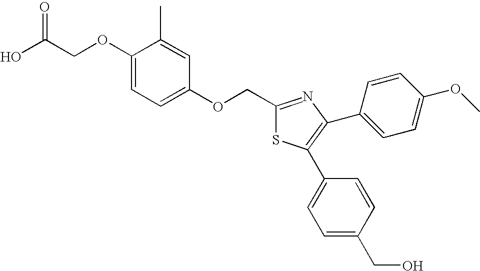 | ¹H-NMR (400 MHz, CDCl₃) δ =7.39 (d, J=8.8 Hz, 2H), 7.30 (s, 4H), 6.88 (d, J=2.8 Hz, 1H), 6.81 (d, J=8.8 Hz, 2H), 6.76 (dd, J=2.8, 9.2 Hz, 1H), 6.68 (d, J=9.2 Hz, 1H), 5.33 (s, 2H), 4.69 (s, 2H), 4.61 (s, 2H), 3.79 (s, 3H), 2.26 (s, 3H). MS calculated for C₂₇H₂₆NO₆S (M + H⁺) 492.1, found 492.4. |
| F14 | 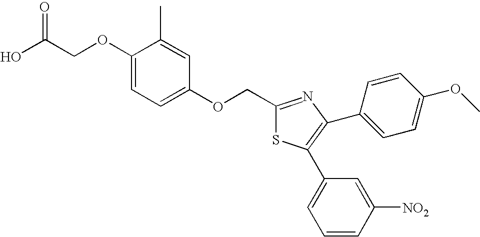 | ¹H-NMR (400 MHz, DMSO-d6) δ =8.21 (d, J=7.2 Hz, 1H), 8.14 (s, 1H), 7.78 (d, J=8.0 Hz, 1H), 7.68 (t, J=8.0 Hz, 1H), 7.39 (d, J=8.8 Hz, 2H), 6.96 (d, J=2.8 Hz, 1H), 6.91 (d, J=8.8 Hz, 2H), 6.85 (dd, J=2.8, 8.8 Hz, 1H), 6.79 (d, J=9.2 Hz, 1H), 5.42 (s, 2H), 4.63 (s, 2H), 3.76 (s, 3H), 2.20 (s, 3H). MS calculated for C₂₆H₂₃N₂O₇S (M + H⁺) |
| # 507.1, found 507.4. | | |
| F15 | 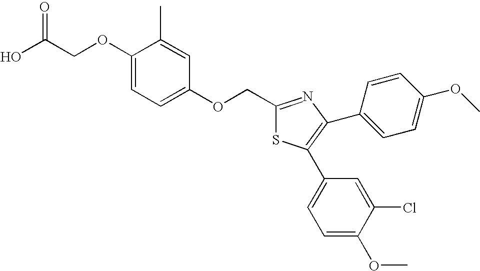 | ¹H-NMR (400 MHz, CDCl₃) δ =7.41 (d, J=8.8 Hz, 2H), 7.36 (d, J=2.4 Hz, 1H), 7.15 (dd, J=2.0, 8.4 Hz, 1H), 6.89 (d, J=2.8 Hz, 1H), 6.84 (m, 3H), 6.76 (dd, J=2.8, 8.8 Hz, 1H), 6.70 (d, J=8.8 Hz, 1H), 5.32 (s, 2H), 4.63 (s, 2H), 3.94 (s, 3H), 3.82 (s, 3H), 2.28 (s, 3H). MS calculated for C₂₇H₂₅ClNO₆S (M + H⁺) 526.1, found 526.3. |
| F16 | 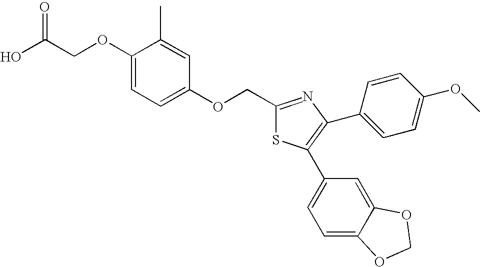 | ¹H-NMR (400 MHz, DMSO-d6) δ =7.40 (d, J=8.8 Hz, 2H), 6.96 (s, 1H), 6.94 (s, 1H), 6.88 (d, J=8.8 Hz, 1H), 6.86 (m, 3H), 6.78 (d, J=9.2 Hz, 1H), 6.10 (s, 2H), 5.37 (s, 2H), 4.63 (s, 2H), 3.76 (s, 3H), 2.19 (s, 3H). MS calculated for C₂₇H₂₄NO₇S (M + H⁺) 506.1, found 506.4. |
| F17 | 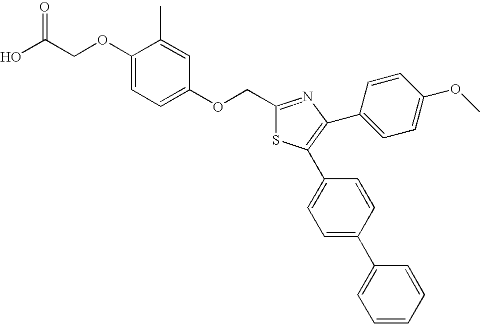 | ¹H-NMR (400 MHz, CDCl₃) δ =7.60 (d, J=7.6 Hz, 2H), 7.55 (d, J=8.4 Hz, 1H), 7.41 (m, 7H), 6.91 (d, J=2.8 Hz, 1H), 6.85 (d, J=8.4 Hz, 2H), 6.79 (dd, J=2.4, 8.8 Hz, 1H), 6.71 (d, J=8.8 Hz, 1H), 5.35 (s, 2H), 4.64 (s, 2H), 3.82 (s, 3H), 2.29 (s, 3H). MS calculated for C₃₂H₂₈NO₅S (M + H⁺) 538.2, found 538.4. |
| F18 | 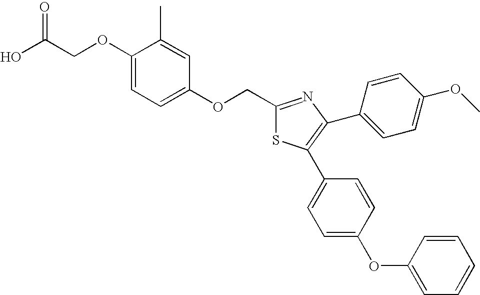 | ¹H-NMR (400 MHz, CDCl₃) δ =7.43 (d, J=8.4 Hz, 2H), 7.36 (t, J=7.6 Hz, 2H), 7.27 (d, J=8.4 Hz, 2H), 7.14 (t, J=7.6 Hz, 1H), 7.04 (d, J=7.6 Hz, 2H), 6.92 (d, J=8.4 Hz, 2H), 6.90 (d, J=2.4 Hz, 1H), 6.84 (d, J=8.8 Hz, 2H), 6.78 (dd, J=2.4, 8.4 Hz, 1H), 6.70 (d, J=8.8 Hz, 1H), 5.34 (s, 2H), 4.63 (s, 2H), 3.82 (s, 3H), 2.29 (s, 3H).NL MS calculated for |
| # C₃₂H₂₈NO₆S (M + H⁺) 554.2, found 554.4. | | |
| F19 | 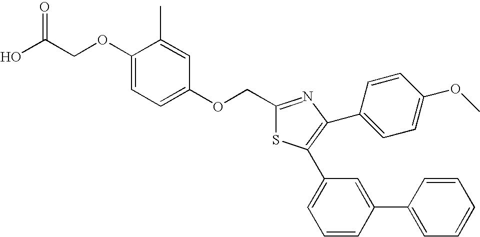 | ¹H-NMR (400 MHz, CDCl₃) δ =7.56 (t, J=1.6 Hz, 1H), 7.53 (dt, J=1.6, 8.0 Hz, 1H), 7.47 (d, J=8.8 Hz, 4H), 7.40 (m, 3H), 7.32 (m, 2H), 6.91 (d, J=3.2 Hz, 1H), 6.86 (d, J=8.8 Hz, 2H), 6.79 (dd, J=2.8, 8.8 Hz, 1H), 6.71 (d, J=8.8 Hz, 1H), 5.36 (s, 2H), 4.63 (s, 2H), 3.82 (s, 3H), 2.29 (s, 3H). MS calculated for C₃₂H₂₈NO₅S (M + H⁺) 538.2, |
| # found 538.1. | | |
| F20 | 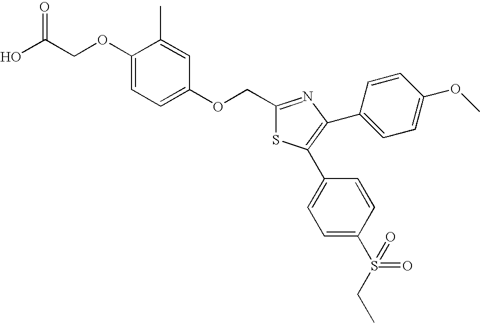 | ¹H-NMR (400 MHz, CDCl₃) δ =7.83 (d, J=8.4 Hz, 2H), 7.52 (d, J=8.8 Hz, 1H), 7.37 (d, J=8.8 Hz, 2H), 6.90 (d, J=2.8 Hz, 1H), 6.85 (d, J=8.8 Hz, 2H), 6.79 (dd, J=2.4, 8.8 Hz, 1H), 6.71 (d, J=8.8 Hz, 1H), 5.37 (s, 2H), 4.64 (s, 2H), 3.85 (s, 3H), 3.13 (q, J=7.2 Hz, 2H), 2.29 (s, 3H), 1.30 (t, J=7.6 Hz, 3H). MS calculated for C₂₈H₂₈NO₇S₂ |
| # (M + H⁺) 554.1, found 554.0. | | |
| F21 | 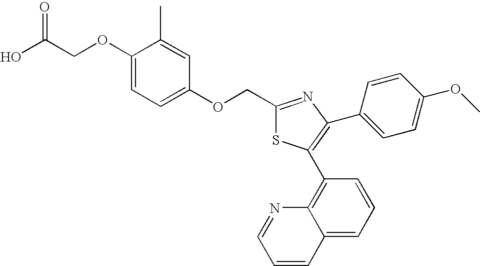 | ¹H-NMR (400 MHz, CDCl₃) δ =9.10 (dd, J=1.6, 4.8 Hz, 1H), 8.55 (dd, J=1.6, 8.4 Hz, 1H), 8.03 (dd, J=1.2, 8.4 Hz, 1H), 7.94 (dd, J=1.6, 7.2 Hz, 1H), 7.75 (d, J=7.2 Hz, 1H), 7.70 (dd, J=4.8, 8.0 Hz, 1H), 7.25 (d, J=8.8 Hz, 2H), 6.90 (d, J=2.4 Hz, 1H), 6.79 (dd, J=2.4, 8.8 Hz, 1H), 6.67 (m, 3H), 5.44 (s, 2H), 4.61 (s, 2H), 3.73 (s, 3H), 2.26 (s, 3H). MS |
| # calculated for C₂₉H₂₅N₂O₅S (M + H⁺) 513.1, found 513.0. | | |
| F22 | 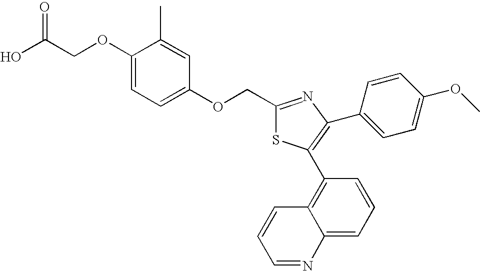 | ¹H-NMR (400 MHz, CDCl₃) δ =9.19 (d, J=4.8 Hz, 1H), 8.57 (d, J=8.4 Hz, 1H), 8.51 (d, J=8.8 Hz, 1H), 8.03 (t, J=7.6 Hz, 1H), 7.91 (d, J=7.2 Hz, 1H), 7.63 (m, 2H), 7.50 (m, 1H), 7.22 (d, J=8.8 Hz, 2H), 6.91 (d, J=2.8 Hz, 1H), 6.80 (dd, J=2.8, 8.8 Hz, 1H), 6.72 (d, J=8.8 Hz, 1H), 6.66 (d, J=8.8 Hz, 2H), 5.42 (s, 2H), 4.65 (s, 2H), 3.70 (s, 3H), 2.29 (s, 3H). |
| # MS calculated for C₂₉H₂₅N₂O₅S (M + H⁺) 513.1, found 513.4. | | |
| F23 | 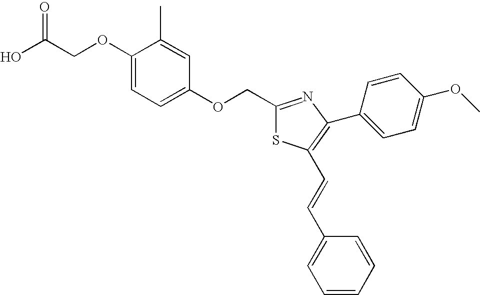 | ¹H-NMR (400 MHz, DMSO-d6) δ =7.54 (d, J=8.8 Hz, 2H), 7.47 (d, J=7.6 Hz, 2H), 7.24 (m, 2H), 7.20 (m, 2H), 7.02 (d, J=8.8 Hz, 2H), 6.96 (d, J=16.0 Hz, 1H), 6.88 (d, J=2.8 Hz, 1H), 6.78 (dd, J=2.8, 8.8 Hz, 1H), 6.71 (d, J=8.8 Hz, 1H), 5.31 (s, 2H), 4.56 (s, 2H), 3.76 (s, 3H), 2.12 (s, 3H). MS calculated for C₂₈H₂₆NO₅S (M + H⁺) 488.1, found 488.4. |
| F24 | 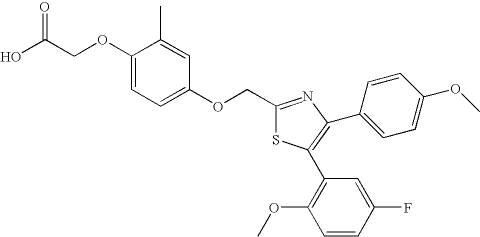 | ¹H-NMR (400 MHz, CDCl₃) δ =7.84 (d, J=8.8 Hz, 0.7H), 7.38 (d, J=8.8 Hz, 1.3H), 6.70-7.02 (m, 8H), 5.36 (s, 1.3H), 5.27 (s, 0.7H), 4.63 (s, 2H), 3.86 (s, 1.1H), 3.80 (s, 1.9H), 3.66 (s, 3H), 2.29 (s, 3H). MS calculated for C₂₇H₂₅FNO₆S (M + H⁺) 510.1, found 510.0. |
| F25 | 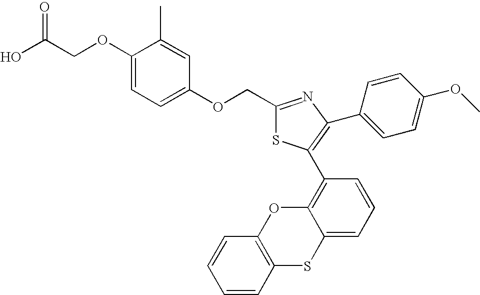 | ¹H-NMR (400 MHz, CDCl₃) δ =7.43 (d, J=8.8 Hz, 2H), 7.12 (dd, J=1.6, 7.6 Hz, 1H), 7.09 (dd, J=2.0, 9.2 Hz, 1H), 7.00 (m, 5H), 6.80 (m, 3H), 6.72 (d, J=8.8 Hz, 1H), 6.63 (dd, J=2.0, 7.6 Hz, 1H), 5.47 (s, 2H), 4.65 (s, 2H), 3.76 (s, 3H), 2.30 (s, 3H). MS calculated for C₃₂H₂₆NO₆S₂(M + H⁺) 584.1, found 584.0. |
| F26 | 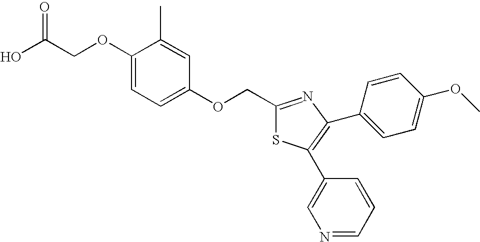 | ¹H-NMR (400 MHz, CDCl₃) δ =8.74 (d, J=2.0 Hz, 1H), 8.66 (dd, J=1.6, 5.2 Hz, 1H), 7.94 (d, J=8.0 Hz, 1H), 7.54 (m, 1H), 7.36 (d, J=8.8 Hz, 2H), 6.88 (m, 3H), 6.77 (dd, J=2.8, 8.4 Hz, 1H), 6.72 (d, J=8.8 Hz, 1H), 5.38 (s, 2H), 4.67 (s, 2H), 3.83 (s, 3H), 2.29 (s, 3H). MS calculated for C₂₅H₂₃N₂O₅S (M + H⁺) 463.1, found 463.0. |
| F27 | 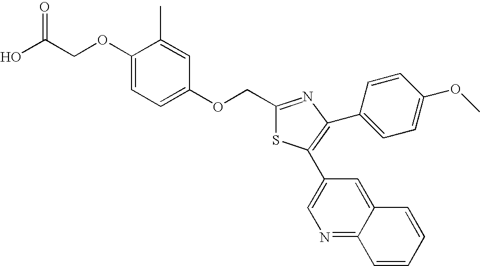 | ¹H-NMR (400 MHz, CDCl₃) δ =8.86 (d, J=2.0 Hz, 1H), 8.24 (s, 1H), 8.20 (d, J=8.4 Hz, 1H), 7.77 (m, 2H), 7.62 (t, J=7.2 Hz, 1H), 7.39 (d, J=8.4 Hz, 2H), 6.85 (d, J=2.8 Hz, 1H), 6.81 (d, J=8.8 Hz, 2H), 6.79 (dd, J=2.8, 8.8 Hz, 1H), 6.70 (d, J=8.8 Hz, 1H), 5.36 (s, 2H), 4.63 (s, 2H), 3.77 (s, 3H), 2.27 (s, 3H). MS calculated for C₂₉H₂₅N₂O₅S |
| # (M + H⁺) 513.1, found 513.0. | | |
| F28 | 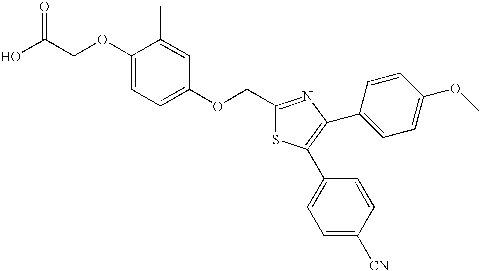 | ¹H-NMR (400 MHz, CDCl₃) δ =7.59 (d, J=8.4 Hz, 2H), 7.42 (d, J=8.4 Hz, 2H), 7.36 (d, J=8.8 Hz, 2H), 6.90 (d, J=2.8 Hz, 1H), 6.86 (d, J=8.8 Hz, 2H), 6.78 (dd, J=3.2, 8.8 Hz, 1H), 6.71 (d, J=8.8 Hz, 1H), 5.36 (s, 2H), 4.65 (s, 2H), 3.83 (s, 3H), 2.29 (s, 3H). MS calculated for C₂₇H₂₃N₂O₅S (M + H⁺) 487.1, found 487.3. |
| F29 | 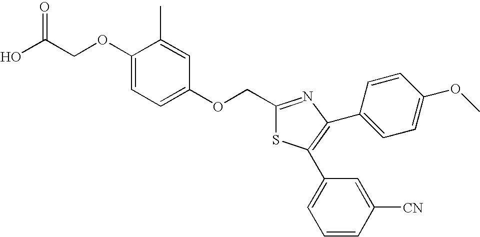 | ¹H-NMR (400 MHz, CDCl₃) δ =7.55 (d, J=1.2 Hz, 1H), 7.51 (dd, J=1.2, 7.6 Hz, 1H), 7.48 (dd, J=1.2, 8.0 Hz, 1H), 7.36 (d, J=7.6 Hz, 1H), 7.29 (d, J=8.8 Hz, 2H), 6.83 (d, J=2.8 Hz, 1H), 6.78 (d, J=8.8 Hz, 2H), 6.71 (dd, J=2.4, 8.8 Hz, 1H), 6.63 (d, J=8.8 Hz, 1H), 5.28 (s, 2H), 4.57 (s, 2H), 3.75 (s, 3H), 2.22 (s, 3H). MS calculated for |
| # C₂₇H₂₃N₂O₅S (M + H⁺) 487.1, found 487.4. | | |
| F30 | 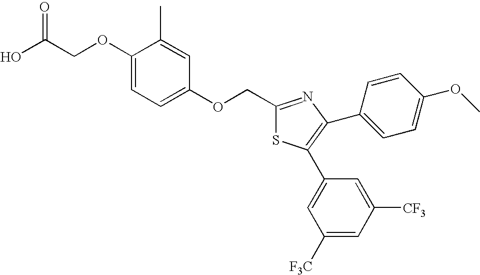 | ¹H-NMR (400 MHz, CDCl₃) δ =7.78 (s, 1H), 7.76 (s, 2H), 7.37 (d, J=8.8 Hz, 2H), 6.90 (d, J=2.8 Hz, 1H), 6.86 (d, J=8.8 Hz, 2H), 6.79 (dd, J=2.8, 8.8 Hz, 1H), 6.71 (d, J=8.8 Hz, 1H), 5.36 (s, 2H), 4.65 (s, 2H), 3.82 (s, 3H), 2.29 (s, 3H). MS calculated for C₂₈H₂₂F₆NO₅S (M + H⁺) 598.1, found 598.3. |
| F31 | 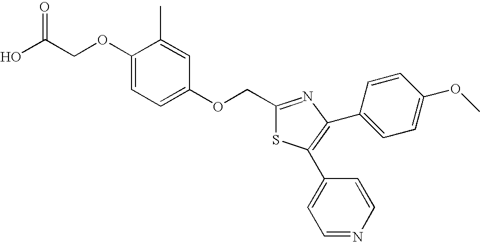 | ¹H-NMR (400 MHz, CDCl₃) δ =8.60 (d, J=5.6 Hz, 2H), 7.62 (d, J=5.6 Hz, 2H), 7.34 (d, J=8.4 Hz, 2H), 6.88 (d, J=8.4 Hz, 2H), 6.83 (d, J=2.8 Hz, 1H), 6.71 (dd, J=2.8, 8.8 Hz, 1H), 6.65 (d, J=8.8 Hz, 1H), 5.30 (s, 2H), 4.59 (s, 2H), 3.80 (s, 3H), 2.23 (s, 3H). MS calculated for C₂₅H₂₃N₂O₅S (M + H⁺) 463.1, found 463.1. |
| F32 | 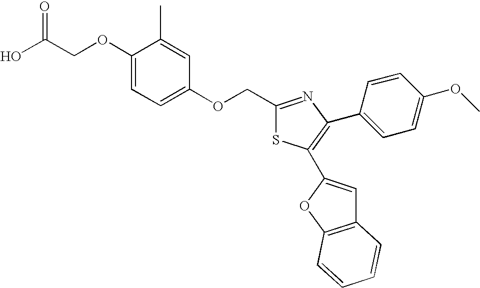 | ¹H-NMR (400 MHz, DMSO-d6) δ =7.48 (d, J=8.4 Hz, 2H), 7.45 (d, J=7.6 Hz, 1H), 7.39 (d, J=8.8 Hz, 1H), 7.18 (t, J=7.2 Hz, 1H), 7.11 (d, J=7.6 Hz, 1H), 6.88 (d, J=8.8 Hz, 2H), 6.83 (d, J=3.2 Hz, 1H), 6.73 (dd, J=3.2, 8.8 Hz, 1H), 6.72 (s, 1H), 6.64 (d, J=8.8 Hz, 1H), 5.28 (s, 2H), 4.48 (s, 2H), 3.71 (s, 3H), 2.10 (s, 3H). MS calculated for C₂₈H₂₄NO₆S |
| # (M + H⁺) 502.1, found 502.3. | | |
| F33 | 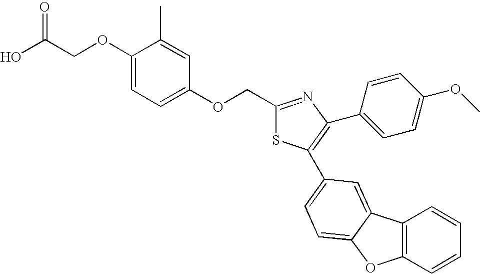 | ¹H-NMR (400 MHz, CDCl₃) δ =7.85 (d, J=8.0 Hz, 1H), 7.82 (dd, J=1.2, 7.6 Hz, 1H), 7.34 (m, 2H), 7.32 (d, J=8.8 Hz, 2H), 7.24 (m, 2H), 7.17 (d, J=7.6 Hz, 1H), 6.83 (d, J=2.8 Hz, 1H), 6.72 (dd, J=2.8, 8.8 Hz, 1H), 6.64 (d, J=8.8 Hz, 2H), 6.62 (d, J=8.8 Hz, 1H), 5.32 (s, 2H), 4.53 (s, 2H), 3.65 (s, 3H), 2.19 (s, 3H). MS calculated for C₃₂H₂₆NO₆S |
| # (M + H⁺) 552.1, found 552.4. | | |
| F34 | 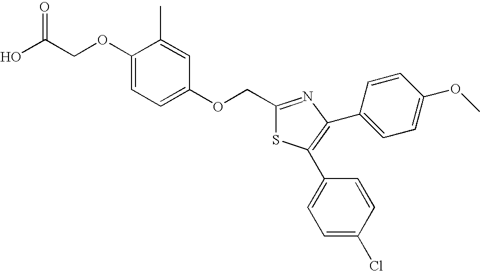 | ¹H-NMR (400 MHz, CDCl₃) δ =7.40 (d, J=8.8 Hz, 2H), 7.29 (d, J=8.4 Hz, 2H), 7.26 (d, J=8.8 Hz, 2H), 6.90 (d, J=2.8 Hz, 1H), 6.84 (d, J=8.8 Hz, 2H), 6.78 (dd, J=3.2, 8.8 Hz, 1H), 6.70 (d, J=8.8 Hz, 1H), 5.33 (s, 2H), 4.63 (s, 2H), 3.82 (s, 3H), 2.29 (s, 3H). MS calculated for C₂₆H₂₃ClNO₅S (M + H⁺) 496.1, found 496.3. |
| F35 | 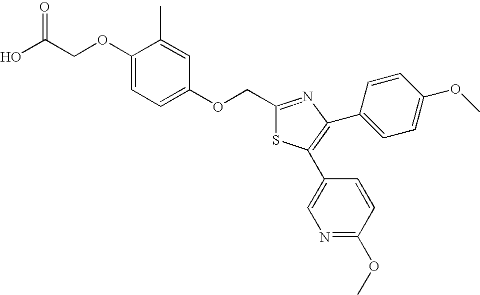 | ¹H-NMR (400 MHz, CDCl₃) δ =8.18 (d, J=2.0 Hz, 1H), 7.47 (dd, J=2.0, 8.8 Hz, 1H), 7.40 (d, J=8.8 Hz, 2H), 6.90 (d, J=2.8 Hz, 1H), 6.84 (d, J=8.8 Hz, 2H), 6.76 (dd, J=2.8, 8.8 Hz, 1H), 6.70 (d, J=8.8 Hz, 1H), 6.69 (d, J=8.4 Hz, 1H), 5.33 (s, 2H), 4.63 (s, 2H), 3.95 (s, 3H), 3.81 (s, 3H), 2.28 (s, 3H). MS calculated for C₂₆H₂₅N₂O₆S |
| # (M + H⁺) 493.1, found 493.1. | | |
| F36 | 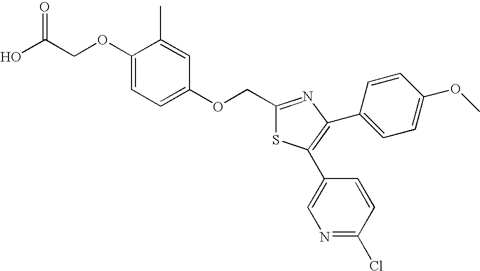 | ¹H-NMR (400 MHz, CDCl₃) δ =8.40 (d, J=2.4 Hz, 1H), 7.57 (dd, J=2.4, 8.4 Hz, 1H), 7.36 (d, J=8.8 Hz, 2H), 7.28 (d, J=8.4 Hz, 1H), 6.90 (d, J=2.8 Hz, 1H), 6.87 (d, J=8.8 Hz, 2H), 6.78 (dd, J=2.8, 8.8 Hz, 1H), 6.71 (d, J=8.8 Hz, 1H), 5.36 (s, 2H), 4.65 (s, 2H), 3.82 (s, 3H), 2.29 (s, 3H). MS calculated for C₂₅H₂₂ClN₂O₅S (M + H⁺) |
| # 497.1, found 497.0. | | |
| F37 | 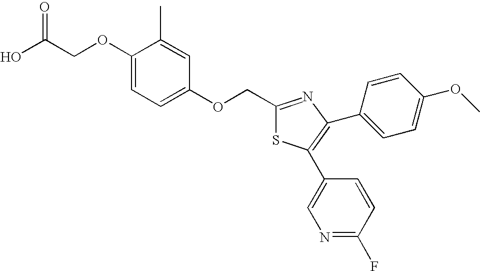 | ¹H-NMR (400 MHz, CDCl₃) δ =8.22 (d, J=2.0 Hz, 1H), 7.69 (dt, J=2.4, 8.4 Hz, 1H), 7.37 (d, J=8.8 Hz, 2H), 6.88 (m, 2H), 6.85 (d, J=8.8 Hz, 2H), 6.77 (dd, J=2.8, 8.8 Hz, 1H), 6.70 (d, J=8.8 Hz, 1H), 5.34 (s, 2H), 4.64 (s, 2H), 3.82 (s, 3H), 2.29 (s, 3H). MS calculated for |
| # C₂₅H₂₂FN₂O₅S (M + H⁺) 481.1, found 481.0. | | |
| F38 | 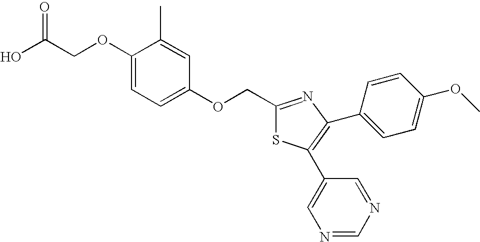 | ¹H-NMR (400 MHz, CDCl₃) δ =9.14 (s, 1H), 8.71 (s, 2H), 7.37 (d, J=8.8 Hz, 2H), 6.87 (m, 3H), 6.78 (dd, J=2.8, 8.8 Hz, 1H), 6.71 (d, J=8.8 Hz, 1H), 5.36 (s, 2H), 4.64 (s, 2H), 3.82 (s, 3H), 2.29 (s, 3H). MS calculated for C₂₄H₂₂N₃O₅S (M + H⁺) 464.1, found 464.1. |
| F39 | 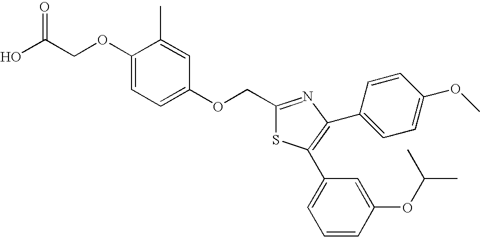 | ¹H-NMR (400 MHz, CDCl₃) δ =7.42 (d, J=8.8 Hz, 2H), 7.20 (t, J=8.0 Hz, 1H), 6.85 (m, 6H), 6.76 (dd, J=2.8, 8.8 Hz, 1H), 6.69 (d, J=9.2 Hz, 1H), 5.35 (s, 2H), 4.63 (s, 2H), 4.39 (m, 1H), 3.80 (s, 3H), 2.28 (s, 3H), 1.25 (s, 3H), 1.24 (s, 3H). MS calculated for C₂₉H₃₀NO₆S (M + H⁺) 520.2, found 520.1. |
| F40 | 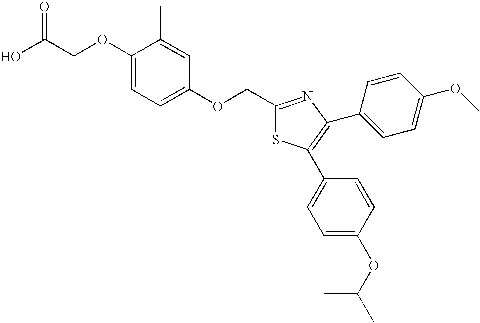 | ¹H-NMR (400 MHz, CDCl₃) δ =7.41 (d, J=8.8 Hz, 2H), 7.21 (d, J=8.8 Hz, 2H), 6.89 (d, J=2.8 Hz, 1H), 6.83 (d, J=9.2 Hz, 2H), 6.81 (d, J=8.8 Hz, 2H), 6.76 (dd, J=2.8, 8.8 Hz, 1H), 6.69 (d, J=9.2 Hz, 1H), 5.35 (s, 2H), 4.62 (s, 2H), 4.55 (m, 1H), 3.81 (s, 3H), 2.28 (s, 3H), 1.35 (s, 3H), 1.34 (s, 3H). MS calculated for C₂₉H₃₀NO₆S (M + H⁺) |
| # 520.2, found 520.1. | | |
| F41 | 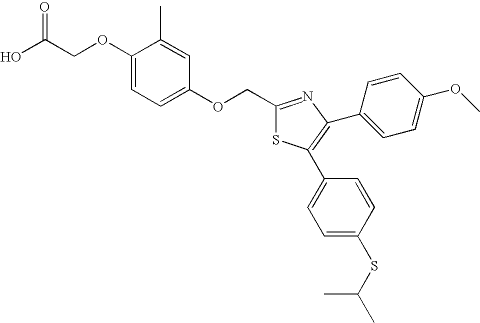 | ¹H-NMR (400 MHz, CDCl₃) δ =7.40 (d, J=8.8 Hz, 2H), 7.28 (d, J=8.4 Hz, 2H), 7.22 (d, J=8.4 Hz, 2H), 6.89 (d, J=2.8 Hz, 1H), 6.84 (d, J=8.8 Hz, 2H), 6.77 (dd, J=2.8, 8.8 Hz, 1H), 6.69 (d, J=8.8 Hz, 1H), 5.35 (s, 2H), 4.63 (s, 2H), 3.81 (s, 3H), 3.42 (m, 1H), 2.28 (s, 3H), 1.33 (s, 3H), 1.31 (s, 3H). MS calculated for C₂₉H₃₀NO₅S₂ |
| # (M + H⁺) 536.2, found 536.1. | | |
| F42 | 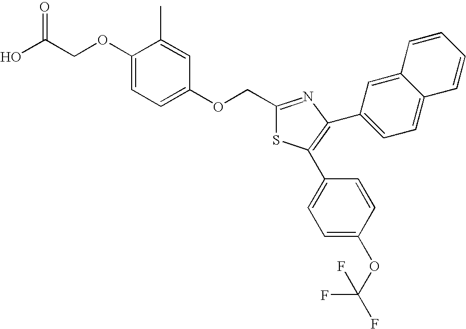 | ¹H-NMR (400 MHz, CDCl₃) δ =8.05 (s, 1H), 7.81 (dd, J=2.4, 6.8 Hz, 1H), 7.75 (d, J=8.4 Hz, 2H), 7.48 (m, 3H), 7.37 (d, J=8.4 Hz, 2H), 7.14 (d, J=8.4 Hz, 2H), 6.92 (d, J=2.8 Hz, 1H), 6.81 (dd, J=2.8, 8.8 Hz, 1H), 6.72 (d, J=8.8 Hz, 1H), 5.38 (s, 2H), 4.64 (s, 2H), 2.29 (s, 3H). MS calculated for C₃₀H₂₃F₃NO₅S (M + H⁺) |
| # 566.2, found 566.1. | | |
| F43 | 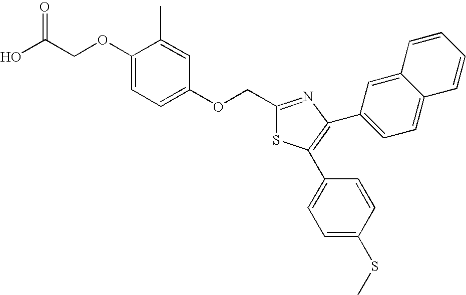 | ¹H-NMR (400 MHz, CDCl₃) δ =8.09 (s, 1H), 7.80 (m, 2H), 7.74 (d, J=8.4 Hz, 1H), 7.51 (dd, J=1.6, 8.4 Hz, 1H), 7.47 (m, 2H), 7.26 (d, J=8.4 Hz, 2H), 7.15 (d, J=8.4 Hz, 2H), 6.92 (d, J=2.8 Hz, 1H), 6.80 (dd, J=2.8, 8.8 Hz, 1H), 6.72 (d, J=8.8 Hz, 1H), 5.38 (s, 2H), 4.64 (s, 2H), 2.47 (s, 3H), 2.29 (s, 3H). MS calculated for C₃₀H₂₆NO₄S₂ |
| # (M + H⁺) 528.1, found 528.1. | | |
| F44 | 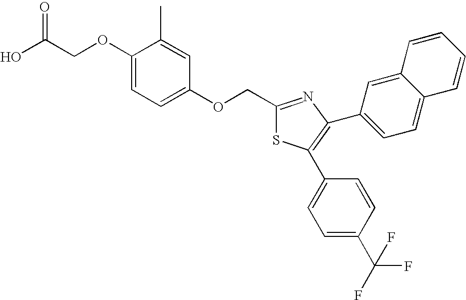 | ¹H-NMR (400 MHz, CDCl₃) δ =8.05 (s, 1H), 7.82 (dd, J=2.0, 7.2 Hz, 1H) 7.77 (d, J=8.4 Hz, 2), 7.55 (d, J=8.4 Hz, 2H), 7.48 (m, 5H), 6.92 (d, J=2.8 Hz, 1H), 6.82 (dd, J=2.8, 8.8 Hz, 1H), 6.72 (d, J=8.8 Hz, 1H), 5.41 (s, 2H), 4.65 (s, 2H), 2.30 (s, 3H). MS calculated for C₃₀H₂₃F₃NO₄S (M + H⁺) 550.1, found 550.1. |
| F45 | 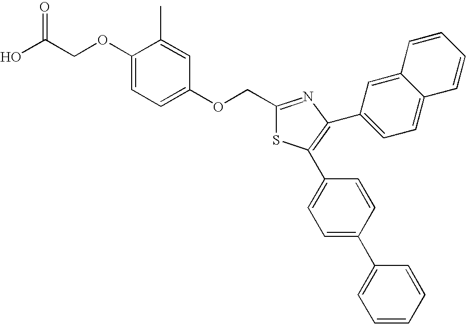 | ¹H-NMR (400 MHz, CDCl₃) δ =8.14 (s, 1H), 7.80 (m, 2H), 7.76 (d, J=8.8 Hz, 1H) 7.56 (m, 5H), 7.46 (m, 6H), 7.37 (d, J=8.4 Hz, 1H), 6.94 (d, J=2.8 Hz, 1H), 6.83 (dd, J=3.2, 8.8 Hz, 1H), 6.74 (d, J=8.8 Hz, 1H), 5.41 (s, 2H), 4.65 (s, 2H), 2.31 (s, 3H). MS calculated for C₃₅H₂₈NO₄S (M + H⁺) 558.2, found 558.1. |
| F46 | 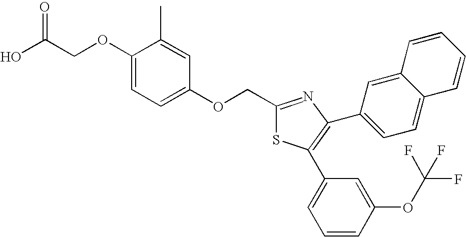 | ¹H-NMR (400 MHz, CDCl₃) δ =8.07 (s, 1H), 7.81 (dd, J=2.0, 8.8 Hz, 1H), 7.75 (m, 2H), 7.47 (m, 3H), 7.31 (m, 2H), 7.23 (s, 1H), 7.17 (d, J=8.0 Hz, 1H), 6.93 (d, J=2.8 Hz, 1H), 6.81 (dd, J=2.8, 8.8 Hz, 1H), 6.73 (d, J=8.8 Hz, 1H), 5.39 (s, 2H), 4.64 (s, 2H), 2.30 (s, 3H). MS calculated for C₃₀H₂₃F₃NO₅S (M + H⁺) 566.1, found 566.1. |
| F47 | 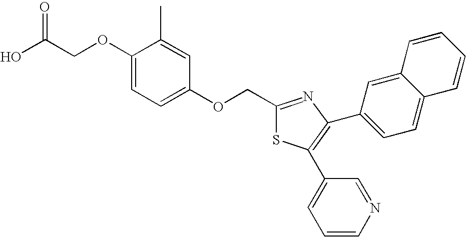 | ¹H-NMR (400 MHz, CDCl₃) δ =8.88 (d, J=1.6 Hz, 1H), 8.72 (d, J=5.6 Hz, 1H), 8.01 (m, 2H), 7.84 (t, J=8.0 Hz, 2H), 7.78 (d, J=7.2 Hz, 1H), 7.54 (m, 3H), 7.43 (dd, J=1.6, 8.4 Hz, 1H), 6.92 (d, J=2.8 Hz, 1H), 6.79 (dd, J=2.8, 8.8 Hz, 1H), 6.74 (d, J=8.8 Hz, 1H), 5.44 (s, 2H), 4.69 (s, 2H), 2.30 (s, 3H). MS calculated for C₂₈H₂₃N₂O₄S |
| # (M + H⁺) 483.1, found 483.1. | | |
| F48 | 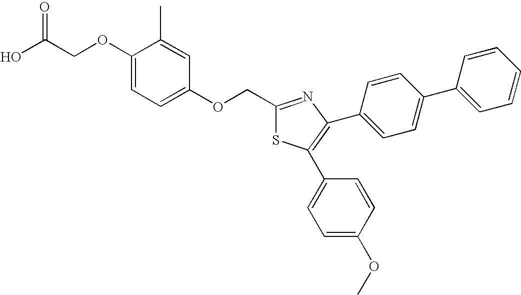 | ¹H-NMR (400 MHz, CDCl₃) δ =7.54 (m, 5H), 7.40 (t, J=7.2 Hz, 2H), 7.31 (t, J=7.2 Hz, 1H), 7.26 (d, J=8.8 Hz, 2H), 6.88 (d, J=2.8 Hz, 1H), 6.83 (d, J=8.8 Hz, 2H), 6.75 (dd, J=2.8, 8.8 Hz, 1H), 6.67 (d, J=8.8 Hz, 1H), 5.31 (s, 2H), 4.60 (s, 2H), 3.79 (s, 3H), 2.26 (s, 3H). MS calculated for C₃₂H₂₈NO₅S (M + H⁺) 538.2, found 538.4. |
| F49 | 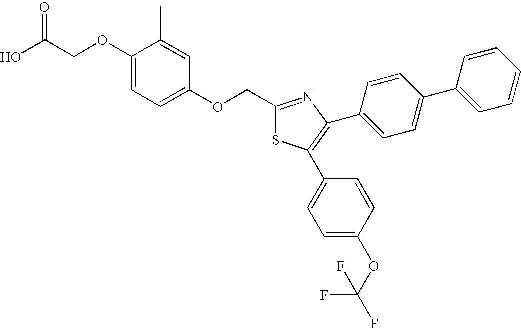 | ¹H-NMR (400 MHz, CDCl₃) δ =7.61 (d, J=7.2 Hz, 2H), 7.56 (s, 4H), 7.44 (t, J=7.2 Hz, 2H), 7.40 (d, J=8.4 Hz, 2H), 7.35 (t, J=7.2 Hz, 1H), 7.18 (d, J=8.0 Hz, 2H), 6.91 (d, J=2.8 Hz, 1H), 6.79 (dd, J=2.8, 8.8 Hz, 1H), 6.71 (d, J=8.8 Hz, 1H), 5.36 (s, 2H), 4.64 (s, 2H), 2.30 (s, 3H). MS calculated for C₃₂H₂₅F₃NO₅S (M + H⁺) |
| # 592.1, found 592.4. | | |
| F50 | 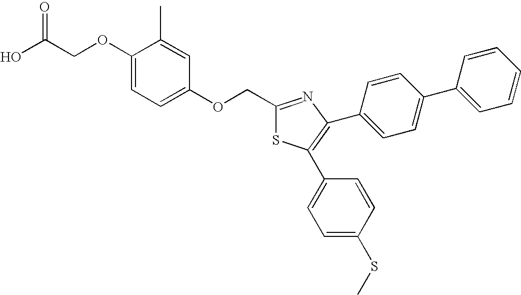 | ¹H-NMR (400 MHz, CDCl₃) δ =7.54 (m, 6H), 7.41 (t, J=7.2 Hz, 2H), 7.32 (t, J=7.2 Hz, 1H), 7.26 (d, J=8.4 Hz, 2H), 7.16 (d, J=8.8 Hz, 2H), 6.89 (d, J=2.8 Hz, 1H), 6.77 (dd, J=2.8, 8.8 Hz, 1H), 6.69 (d, J=8.8 Hz, 1H), 5.33 (s, 2H), 4.61 (s, 2H), 2.47 (s, 3H), 2.27 (s, 3H). MS calculated for C₃₂H₂₈NO₄S₂ (M + H⁺) 554.1, found 554.3. |
| F51 | 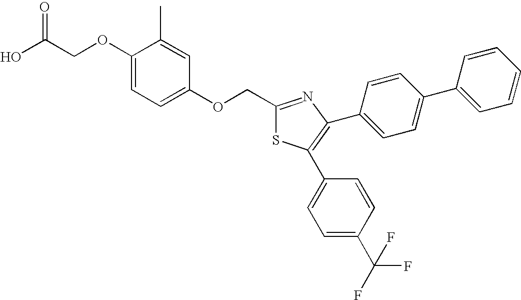 | ¹H-NMR (400 MHz, CDCl₃) δ =7.58 (m, 8H), 7.49 (d, J=8.4 Hz, 2H), 7.44 (t, J=7.6 Hz, 2H), 7.35 (t, J=7.6 Hz, 1H), 6.92 (d, J=2.8 Hz, 1H), 6.81 (dd, J=2.8, 8.8 Hz, 1H), 6.72 (d, J=8.8 Hz, 1H), 5.37 (s, 2H), 4.65 (s, 2H), 2.30 (s, 3H). MS calculated for C₃₂H₂₅F₃NO₄S (M + H⁺) 576.1, found 576.4. |
| F52 | 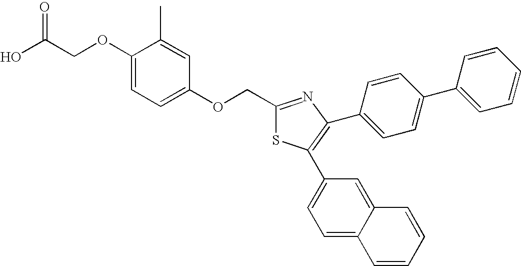 | ¹H-NMR (400 MHz, CDCl₃) δ =7.92 (s, 1H), 7.81 (m, 3H), 7.58 (m, 4H), 7.51 (m, 4H), 7.41 (m, 3H), 7.33 (t, J=7.2 Hz, 1H), 6.94 (d, J=2.8 Hz, 1H), 6.82 (dd, J=2.8, 8.8 Hz, 1H), 6.73 (d, J=8.8 Hz, 1H), 5.40 (s, 2H), 4.65 (s, 2H), 2.30 (s, 3H). MS calculated for C₃₅H₂₈NO₄S (M + H⁺) 558.2, found 558.4. |
| F53 | 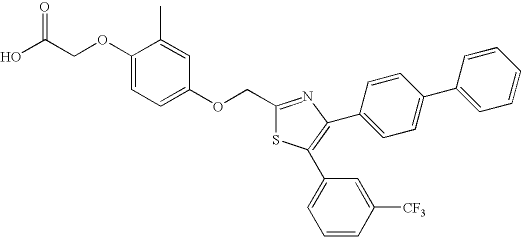 | ¹H-NMR (400 MHz, CDCl₃) δ =7.66 (s, 1H), 7.58 (m, 3H), 7.54 (m, 5H), 7.44 (t, J=7.2 Hz, 3H), 7.35 (t, J=7.2 Hz, 1H), 6.92 (d, J=3.2 Hz, 1H), 6.81 (dd, J=3.2, 8.8 Hz, 1H), 6.72 (d, J=8.8 Hz, 1H), 5.38 (s, 2H), 4.65 (s, 2H), 2.30 (s, 3H). MS calculated for C₃₂H₂₅F₃NO₄S (M + H⁺) 576.1, found 576.3. |
| F54 | 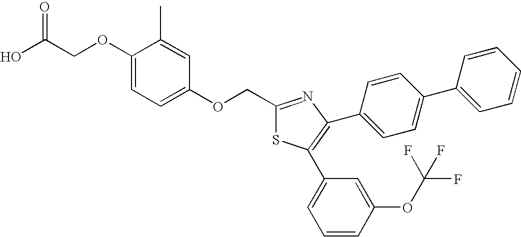 | ¹H-NMR (400 MHz, CDCl₃) δ =7.60 (d, J=7.2 Hz, 2H), 7.56 (s, 4H), 7.44 (t, J=7.2 Hz, 2H), 7.35 (m, 3H), 7.23 (s, 1H), 7.18 (d, J=8.0 Hz, 1H), 6.92 (d, J=2.8 Hz, 1H), 6.80 (dd, J=2.8, 8.8 Hz, 1H), 6.72 (d, J=8.8 Hz, 1H), 5.36 (s, 2H), 4.65 (s, 2H), 2.30 (s, 3H). MS calculated for C₃₂H₂₅F₃NO₅S (M + H⁺) 592.1, found 592.4. |
| F55 | 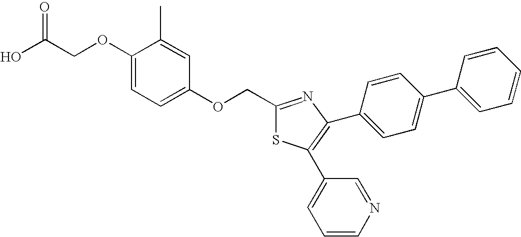 | ¹H-NMR (400 MHz, DMSO-d6) δ =8.59 (m, 2H), 7.85 (dt, J=2.0, 8.0 Hz, 1H), 7.69 (m, 4H), 7.53 (d, J=8.4 Hz, 2H), 7.47 (t, J=7.2 Hz, 2H), 7.37 (t, J=7.6 Hz, 1H), 6.98 (d, J=2.8 Hz, 1H), 6.89 (dd, J=2.8, 8.8 Hz, 1H), 6.80 (d, J=8.8 Hz, 1H), 5.45 (s, 2H), 4.65 (s, 2H), 2.20 (s, 3H). MS calculated for C₃₀H₂₅N₂O₄S (M + H⁺) 509.2, found |
| # 509.4. | | |
| F56 | 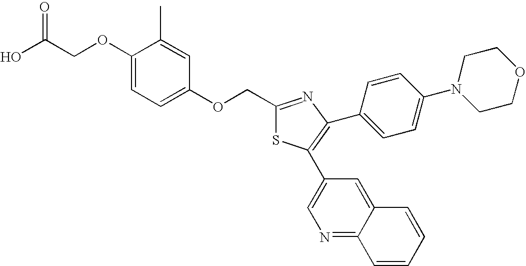 | ¹H-NMR (400 MHz, CDCl₃) δ =8.97 (d, J=1.6 Hz, 1H), 8.47 (d, J=1.6 Hz, 1H), 8.36 (d, J=8.4 Hz, 1H), 7.90 (m, 2H), 7.74 (t, J=7.6 Hz, 1H), 7.39 (d, J=8.8 Hz, 2H), 6.89 (m, 3H), 6.75 (dd, J=2.8, 8.8 Hz, 1H), 6.69 (d, J=8.8 Hz, 1H), 5.37 (s, 2H), 4.63 (s, 2H), 3.87 (m, 4H), 3.21 (m, 4H), 2.27 (s, 3H). MS calculated for C₃₂H₃₀N₃O₅S |
| # (M + H⁺) 568.2, found 568.2. | | |
| F57 | 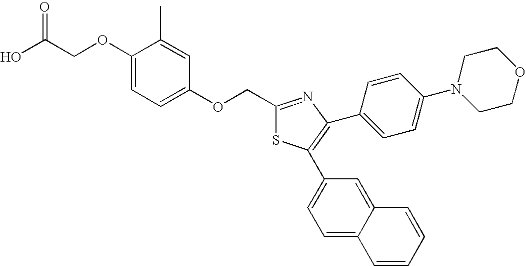 | ¹H-NMR (400 MHz, CDCl₃) δ =7.88 (s, 1H), 7.81 (m, 1H), 7.77 (d, J=8.0 Hz, 2H), 7.52 (m, 5H), 7.32 (dd, J=1.2, 8.4 Hz, 1H), 7.15 (d, J=8.8 Hz, 2H), 6.90 (d, J=2.8 Hz, 1H), 6.75 (dd, J=2.8, 8.8 Hz, 1H), 6.70 (d, J=8.8 Hz, 1H), 5.38 (s, 2H), 4.64 (s, 2H), 3.99 (m, 4H), 3.37 (m, 4H), 2.28 (s, 3H). MS calculated for C₃₃H₃₁N₂O₅S (M + H⁺) |
| # 567.2, found 567.2. | | |
| F58 | 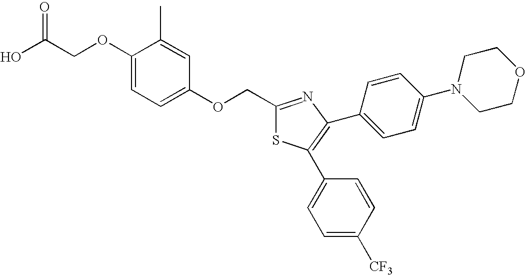 | ¹H-NMR (400 MHz, CDCl₃) δ =7.57 (d, J=8.4 Hz, 2H), 7.45 (d, J=8.0 Hz, 2H), 7.42 (d, J=8.8 Hz, 2H), 6.97 (d, J=8.8 Hz, 2H), 6.89 (d, J=2.8 Hz, 1H), 6.78 (dd, J=2.8, 8.8 Hz, 1H), 6.70 (d, J=8.8 Hz, 1H), 5.34 (s, 2H), 4.63 (s, 2H), 3.92 (m, 4H), 3.27 (m, 4H), 2.28 (s, 3H). MS calculated for C₃₀H₂₈F₃N₂O₅S (M + H⁺) |
| # 585.2, found 585.2. | | |
| F59 | 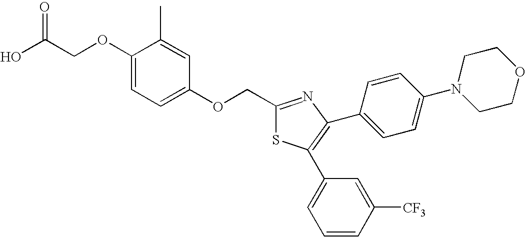 | ¹H-NMR (400 MHz, CDCl₃) δ =7.60 (s, 1H), 7.57 (d, J=8.0 Hz, 1H), 7.49 (d, J=8.0 Hz, 1H), 7.43 (m, 3H), 7.03 (d, J=8.8 Hz, 2H), 6.89 (d, J=2.8 Hz, 1H), 6.77 (dd, J=2.8, 8.8 Hz, 1H), 6.69 (d, J=8.8 Hz, 1H), 5.34 (s, 2H), 4.63 (s, 2H), 3.94 (m, 4H), 3.29 (m, 4H), 2.28 (s, 3H). MS calculated for C₃₀H₂₈F₃N₂O₅S (M + H⁺) |
| # 585.2, found 585.1. | | |
| F60 | 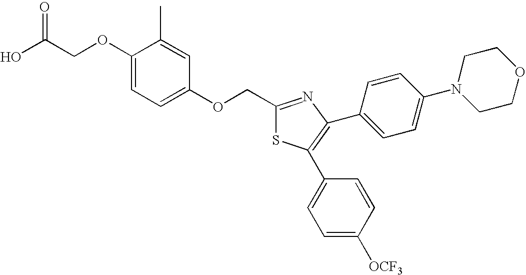 | ¹H-NMR (400 MHz, CDCl₃) δ =7.42 (d, J=8.8 Hz, 2H), 7.35 (d, J=8.4 Hz, 2H), 7.16 (d, J=8.0 Hz, 2H), 6.98 (d, J=8.4 Hz, 2H), 6.89 (d, J=2.8 Hz, 1H), 6.77 (dd, J=2.8, 8.8 Hz, 1H), 6.69 (d, J=8.8 Hz, 1H), 5.33 (s, 2H), 4.63 (s, 2H), 3.93 (m, 4H), 3.27 (m, 4H), 2.28 (s, 3H). MS calculated for C₃₀H₂₈F₃N₂O₆S (M + H⁺) |
| # 601.2, found 601.2. | | |
| F61 | 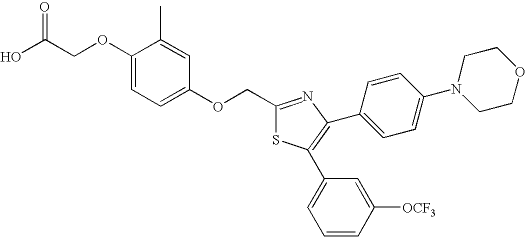 | ¹H-NMR (400 MHz, CDCl₃) δ =7.45 (d, J=8.8 Hz, 2H), 7.36 (t, J=8.4 Hz, 1H), 7.27 (d, J=8.4 Hz, 1H), 7.17 (d, J=8.0 Hz, 2H), 7.05 (d, J=8.8 Hz, 2H), 6.89 (d, J=2.8 Hz, 1H), 6.77 (dd, J=2.8, 8.8 Hz, 1H), 6.69 (d, J=8.8 Hz, 1H), 5.34 (s, 2H), 4.63 (s, 2H), 3.96 (m, 4H), 3.30 (m, 4H), 2.28 (s, 3H). MS calculated for C₃₀H₂₈F₃N₂O₆S |
| # (M + H⁺) 601.2, found 601.2. | | |
| F62 | 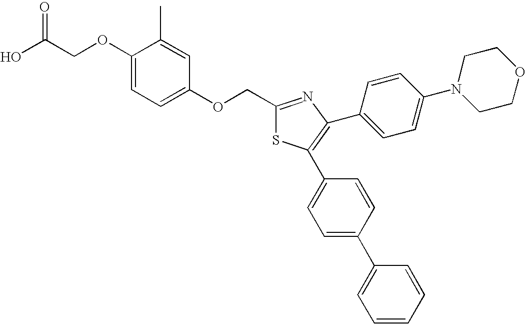 | ¹H-NMR (400 MHz, CDCl₃) δ =7.52 (d, J=8.4 Hz, 2H), 7.47 (t, J=8.4 Hz, 4H), 7.37 (t, J=7.6 Hz, 2H), 7.30 (m, 3H), 7.04 (d, J=8.8 Hz, 2H), 6.81 (d, J=2.8 Hz, 1H), 6.70 (dd, J=2.8, 8.8 Hz, 1H), 6.61 (d, J=8.8 Hz, 1H), 5.27 (s, 2H), 4.55 (s, 2H), 3.90 (m, 4H), 3.26 (m, 4H), 2.20 (s, 3H). MS calculated for C₃₅H₃₃N₂O₅S (M + H⁺) |
| # 593.2, found 593.2. | | |
| F63 | 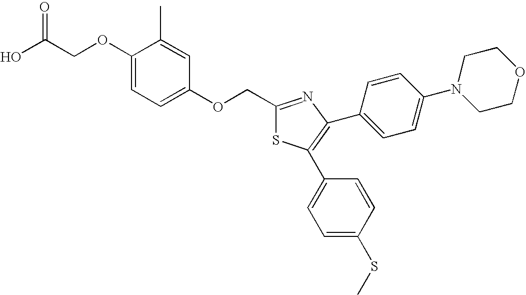 | ¹H-NMR (400 MHz, CDCl₃) δ =7.54 (d, J=8.8 Hz, 2H), 7.21 (m, 6H), 6.88 (d, J=2.8 Hz, 1H), 6.77 (dd, J=2.8, 8.8 Hz, 1H), 6.69 (d, J=8.8 Hz, 1H), 5.33 (s, 2H), 4.63 (s, 2H), 4.03 (m, 4H), 3.40 (m, 4H), 2.49 (s, 3H), 2.28 (s, 3H). MS calculated for C₃₀H₃₁N₂O₅S₂(M + H⁺) 563.2, found 563.2. |
| F64 | 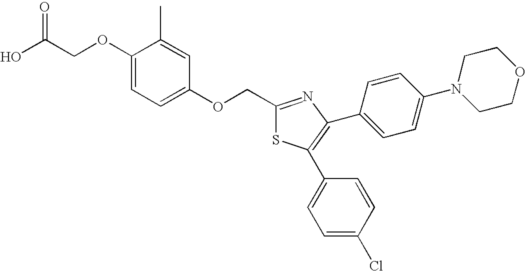 | ¹H-NMR (400 MHz, CDCl₃) δ =7.48 (d, J=8.8 Hz, 2H), 7.31 (d, J=8.8 Hz, 2H), 7.26 (d, J=8.4 Hz, 2H), 7.14 (d, J=8.8 Hz, 2H), 6.88 (d, J=2.8 Hz, 1H), 6.77 (dd, J=2.8, 8.8 Hz, 1H), 6.69 (d, J=8.8 Hz, 1H), 5.33 (s, 2H), 4.63 (s, 2H), 4.00 (m, 4H), 3.36 (m, 4H), 2.28 (s, 3H), 2.28 (s, 3H). MS calculated for C₂₉H₂₈ClN₂O₅S (M + H⁺) |
| # 551.1, found 551.2. | | |
| F65 | 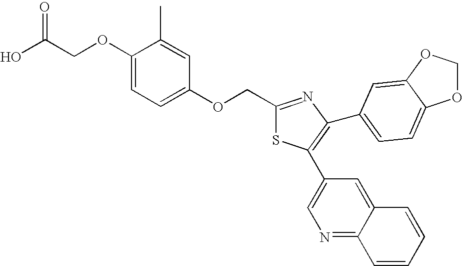 | ¹H-NMR (400 MHz, CDCl₃) δ =8.92 (d, J=1.6 Hz, 1H), 8.36 (s, 1H), 8.29 (d, J=8.8 Hz, 1H), 7.84 (m, 2H), 7.68 (t, J=8.0 Hz, 1H), 6.97 (d, J=1.2 Hz, 1H), 6.91 (m, 2H), 6.77 (dd, J=2.8, 8.8 Hz, 1H), 6.73 (d, J=8.0 Hz, 1H), 6.71 (d, J=8.8 Hz, 1H), 5.97 (s, 2H), 5.37 (s, 2H), 4.65 (s, 2H), 2.28 (s, 3H). MS calculated for C₂₉H₂₃N₂O₆S |
| # (M + H⁺) 527.1, found 527.3. | | |
| F66 | 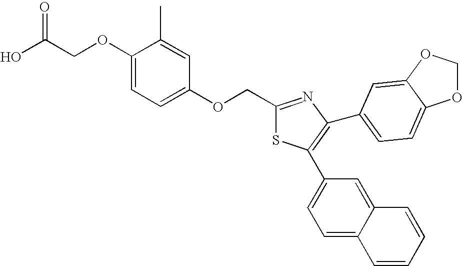 | ¹H-NMR (400 MHz, CDCl₃) δ =7.87 (s, 1H), 7.81 (m, 2H), 7.76 (d, J=8.8 Hz, 1H), 7.51 (m, 2H), 7.33 (dd, J=2.0, 8.8 Hz, 1H), 6.99 (m, 2H), 6.92 (d, J=2.8 Hz, 1H), 6.81 (dd, J=2.8, 8.8 Hz, 1H), 6.72 (d, J=8.0 Hz, 2H), 5.95 (s, 2H), 5.40 (s, 2H), 4.65 (s, 2H), 2.30 (s, 3H). MS calculated for C₃₀H₂₄NO₆S (M + H⁺) 526.1, found 526.3. |
| F67 | 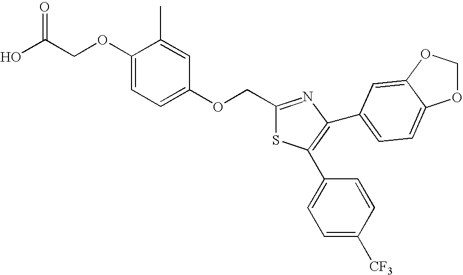 | ¹H-NMR (400 MHz, CDCl₃) δ =7.57 (d, J=8.0 Hz, 2H), 7.44 (d, J=8.4 Hz, 2H), 6.94 (m, 2H), 6.90 (d, J=3.2 Hz, 1H), 6.77 (m, 2H), 6.70 (d, J=8.8 Hz, 1H), 5.98 (s, 2H), 5.33 (s, 2H), 4.64 (s, 2H), 2.29 (s, 3H). MS calculated for C₂₇H₂₁F₃NO₆S (M + H⁺) 544.1, found 544.3. |
| F68 | 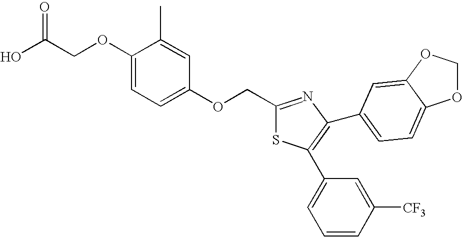 | ¹H-NMR (400 MHz, CDCl₃) δ =7.60 (s, 1H), 7.57 (d, J=8.0 Hz, 1H), 7.49 (d, J=8.0 Hz, 1H), 7.44 (d, J=7.6 Hz, 1H), 6.93 (m, 2H), 6.90 (d, J=3.2 Hz, 1H), 6.78 (dd, J=3.2, 8.8 Hz, 1H), 6.75 (d, J=8.4 Hz, 1H), 6.71 (d, J=8.8 Hz, 1H), 5.97 (s, 2H), 5.35 (s, 2H), 4.65 (s, 2H), 2.29 (s, 3H). MS calculated for C₂₇H₂₁F₃NO₆S (M + H⁺) |
| # 544.1, found 544.3. | | |
| F69 | 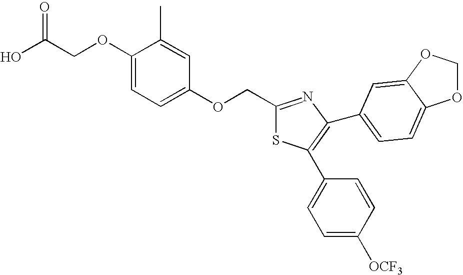 | ¹H-NMR (400 MHz, CDCl₃) δ =7.35 (d, J=8.8 Hz, 2H), 7.17 (d, J=8.4 Hz, 2H), 6.94 (m, 2H), 6.89 (d, J=2.8 Hz, 1H), 6.77 (m, 2H), 6.70 (d, J=8.8 Hz, 1H), 5.97 (s, 2H), 5.33 (s, 2H), 4.64 (s, 2H), 2.29 (s, 3H). MS calculated for C₂₇H₂₁F₃NO₇S (M + H⁺) 560.1, found 560.3. |
| F70 | 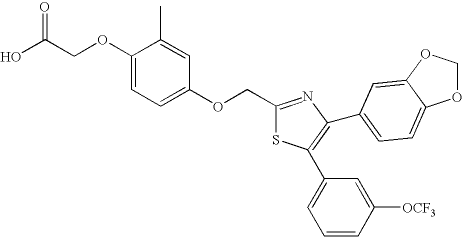 | ¹H-NMR (400 MHz, CDCl₃) δ =7.36 (t, J=8.0 Hz, 1H), 7.28 (d, J=8.0 Hz, 1H), 7.16 (m, 2H), 6.95 (d, J=8.0 Hz, 1H), 6.93 (d, J=1.2 Hz, 1H), 6.90 (d, J=2.0 Hz, 1H), 6.77 (m, 2H), 6.70 (d, J=8.8 Hz, 1H), 5.97 (s, 2H), 5.33 (s, 2H), 4.64 (s, 2H), 2.29 (s, 3H). MS calculated for C₂₇H₂₁F₃NO₇S (M + H⁺) 560.1, found 560.3. |
| F71 | 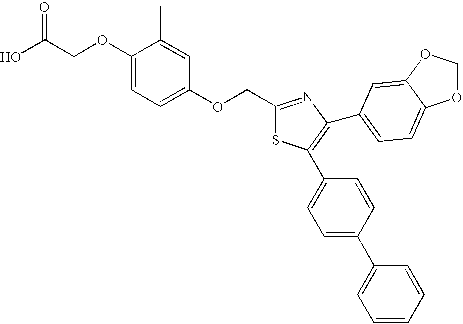 | ¹H-NMR (400 MHz, CDCl₃) δ =7.58 (d, J=8.0 Hz, 2H), 7.54 (d, J=8.0 Hz, 2H), 7.44 (t, J=7.6 Hz, 2H), 7.37 (m, 3H), 7.02 (dd, J=1.6, 8.0 Hz, 1H), 6.98 (d, J=1.6 Hz, 1H), 6.90 (d, J=2.4 Hz, 1H), 6.79 (dd, J=2.8, 8.8 Hz, 1H), 6.76 (d, J=8.0 Hz, 1H), 6.71 (d, J=8.8 Hz, 1H), 5.96 (s, 2H), 5.36 (s, 2H), 4.63 (s, 2H), 2.28 (s, 3H). MS calculated for |
| # C₃₂H₂₆NO₆S (M + H⁺) 552.1, found 552.4. | | |
| F72 | 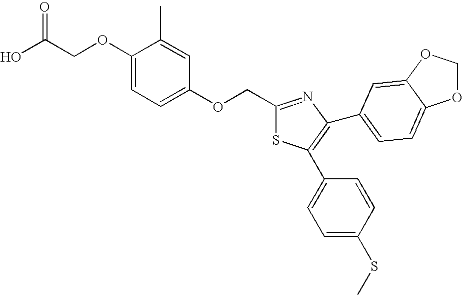 | ¹H-NMR (400 MHz, CDCl₃) δ =7.25 (d, J=8.4 Hz, 2H), 7.18 (d, J=8.4 Hz, 2H), 6.69 (dd, J=1.6, 8.0 Hz, 1H), 6.97 (d, J=1.6 Hz, 1H), 6.90 (d, J=3.2 Hz, 1H), 6.78 (dd, J=2.8, 8.8 Hz, 1H), 6.75 (d, J=8.4 Hz, 1H), 6.70 (d, J=8.8 Hz, 1H), 5.96 (s, 2H), 5.33 (s, 2H), 4.63 (s, 2H), 2.49 (s, 3H), 2.29 (s, 3H). MS calculated for C₂₇H₂₄NO₆S₂ |
| # (M + H⁺) 522.1, found 522.3. | | |
| F73 | 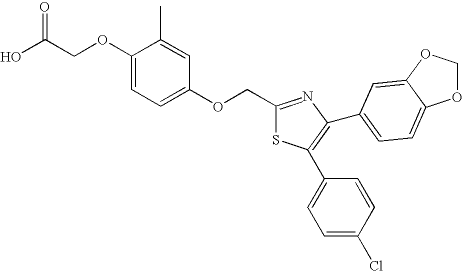 | ¹H-NMR (400 MHz, CDCl₃) δ =7.30 (d, J=8.4 Hz, 2H), 7.26 (d, J=8.8 Hz, 2H), 6.95 (dd, J=1.6, 8.0 Hz, 1H), 6.93 (d, J=1.6 Hz, 1H), 6.89 (d, J=2.8 Hz, 1H), 6.76 (m, 2H), 6.70 (d, J=8.8 Hz, 1H), 5.97 (s, 2H), 5.33 (s, 2H), 4.64 (s, 2H), 2.29 (s, 3H). MS calculated for C₂₆H₂₁ClNO₆S (M + H⁺) 510.1, found 510.2. |
| F74 | 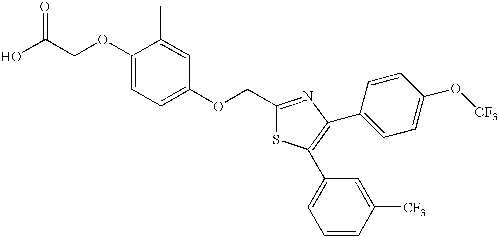 | ¹H-NMR (400 MHz, CDCl₃) δ =7.59 (d, J=8.8 Hz, 2H), 7.48 (m, 4H), 7.16 (d, J=8.4 Hz, 2H), 6.91 (d, J=2.8 Hz, 1H), 6.79 (dd, J=2.8, 8.8 Hz, 1H), 6.71 (d, J=8.8 Hz, 1H), 5.35 (s, 2H), 4.65 (s, 2H), 2.29 (s, 3H). MS calculated for C₂₇H₂₀F₆NO₅S (M + H⁺) 584.1, found 584.3. |
| F75 | 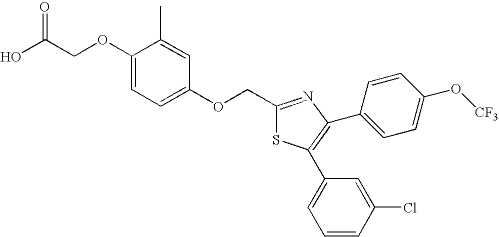 | ¹H-NMR (400 MHz, CDCl₃) δ =7.52 (d, J=8.8 Hz, 2H), 7.32 (d, J=8.8 Hz, 2H), 7.26 (t, J=8.0 Hz, 1H), 7.17 (t, J=8.4 Hz, 2H), 6.90 (d, J=2.8 Hz, 1H), 6.79 (dd, J=2.8, 8.8 Hz, 1H), 6.71 (d, J=8.8 Hz, 1H), 5.34 (s, 2H), 4.65 (s, 2H), 2.29 (s, 3H). MS calculated for C₂₆H₂₀ClF₃NO₅S (M + H⁺) 550.1, found 550.3. |
| F76 | 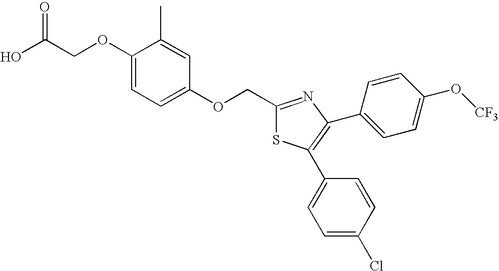 | ¹H-NMR (400 MHz, CDCl₃) δ =7.49 (d, J=8.8 Hz, 2H), 7.30 (d, J=8.4 Hz, 2H), 7.23 (d, J=8.4 Hz, 2H), 7.13 (d, J=8.0 Hz, 2H), 6.87 (d, J=2.8 Hz, 1H), 6.76 (dd, J=2.8, 8.8 Hz, 1H), 6.68 (d, J=8.8 Hz, 1H), 5.30 (s, 2H), 4.62 (s, 2H), 2.26 (s, 3H). MS calculated for C₂₆H₂₀ClF₃NO₅S (M + H⁺) 550.1, found 550.3. |
| F77 | 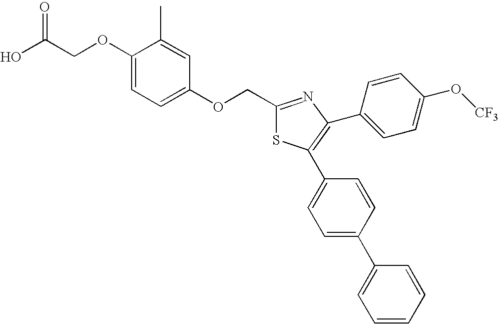 | ¹H-NMR (400 MHz, CDCl₃) δ =7.57 (m, 6H), 7.43 (t, J=7.6 Hz, 2H), 7.36 (t, J=7.6 Hz, 3H), 7.13 (d, J=8.8 Hz, 2H), 6.89 (d, J=2.8 Hz, 1H), 6.75 (dd, J=2.8, 8.8 Hz, 1H), 6.68 (d, J=8.8 Hz, 1H), 5.33 (s, 2H), 4.62 (s, 2H), 2.27 (s, 3H). MS calculated for C₃₂H₂₅F₃NO₅S (M + H⁺) 592.1, found 592.4. |
| F78 | 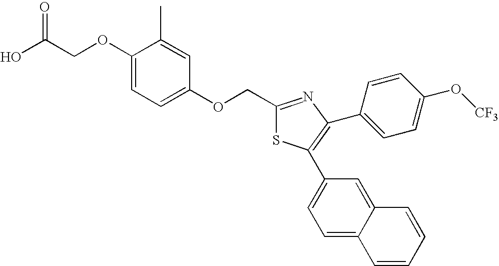 | ¹H-NMR (400 MHz, CDCl₃) δ =7.87 (s, 1H), 7.83 (m, 1H), 7.78 (d, J=8.8 Hz, 2H), 7.56 (d, J=8.8 Hz, 2H), 7.51 (t, J=8.0 Hz, 2H), 7.34 (dd, J=1.6, 8.4 Hz, 1H), 7.11 (d, J=8.4 Hz, 2H), 6.92 (d, J=2.8 Hz, 1H), 6.81 (dd, J=2.8, 8.8 Hz, 1H), 6.72 (d, J=8.8 Hz, 1H), 5.37 (s, 2H), 4.65 (s, 2H), 2.30 (s, 3H). MS calculated for C₃₀H₂₃F₃NO₅S |
| # (M + H⁺) 566.1, found 566.3. | | |
| F79 | 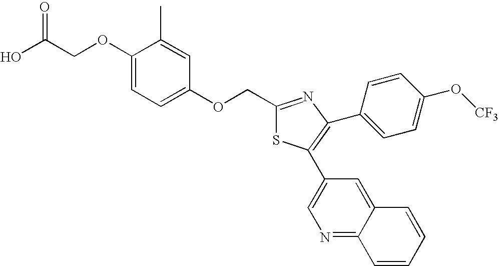 | ¹H-NMR (400 MHz, CDCl₃) δ =8.90 (d, J=2.0 Hz, 1H), 8.23 (m, 2H), 7.80 (m, 2H), 7.65 (t, J=7.2 Hz, 1H), 7.53 (d, J=8.8 Hz, 2H), 7.15 (d, J=8.0 Hz, 2H), 6.91 (d, J=3.2 Hz, 1H), 6.79 (dd, J=2.8, 8.8 Hz, 1H), 6.73 (d, J=8.8 Hz, 1H), 5.39 (s, 2H), 4.66 (s, 2H), 2.30 (s, 3H). MS calculated for C₂₉H₂₂F₃N₂O₅S (M + H⁺) |
| # 567.1, found 567.3. | | |
| F80 | 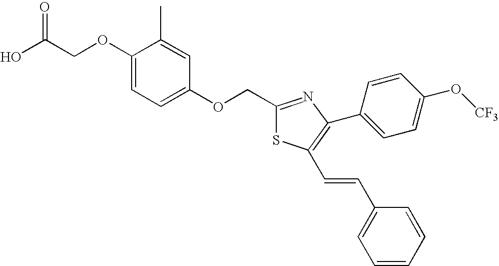 | ¹H-NMR (400 MHz, DMSO-d6) δ =8.10 (s, 1H), 7.66 (d, J=8.8 Hz, 2H), 7.40 (d, J=7.2 Hz, 2H), 7.32 (d, J=7.2 Hz, 2H), 7.17 (m, 4H), 6.92 (d, J=16.4 Hz, 1H), 6.78 (d, J=2.8 Hz, 1H), 6.69 (dd, J=2.8, 8.8 Hz, 1H), 6.62 (d, J=8.8 Hz, 1H), 5.22 (s, 2H), 4.46 (s, 2H), 2.07 (s, 3H). MS calculated for C₂₈H₂₃F₃NO₅S (M + H⁺) 542.1, found 542.3. |
| F81 | 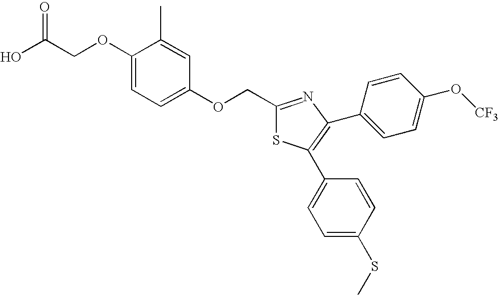 | ¹H-NMR (400 MHz, CDCl₃) δ =7.54 (d, J=8.8 Hz, 2H), 7.23 (d, J=8.8 Hz, 2H), 7.18 (d, J=8.8 Hz, 2H), 7.14 (d, J=8.4 Hz, 2H), 6.90 (d, J=2.8 Hz, 1H), 6.78 (dd, J=2.8, 8.8 Hz, 1H), 6.71 (d, J=8.8 Hz, 1H), 5.34 (s, 2H), 4.64 (s, 2H), 2.50 (s, 3H), 2.29 (s, 3H). MS calculated for C₂₇H₂₃F₃NO₅S₂(M + H⁺) 562.1, found 562.3. |
| F82 | 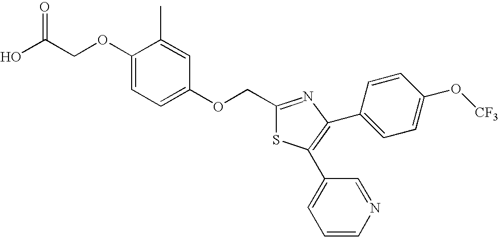 | ¹H-NMR (400 MHz, DMSO-d6) δ =8.58 (dd, J=1.6, 8.8 Hz, 1H), 8.54 (d, J=2.0 Hz, 1H), 8.24 (s, 1H), 7.80 (dt, J=2.0, 8.0 Hz, 1H), 7.55 (d, J=8.8 Hz, 2H), 7.44 (dd, J=5.2, 8.0 Hz, 1H), 7.29 (d, J=8.0 Hz, 2H), 6.93 (d, J=2.8 Hz, 1H), 6.85 (dd, J=3.2, 8.8 Hz, 1H), 6.76 (d, J=8.8 Hz, 1H), 5.40 (s, 2H), 4.60 (s, 2H), 2.20 (s, 3H). MS calculated for |
| # C₂₅H₂₀F₃N₂O₅S (M + H⁺) 517.1, found 517.3. | | |
| F83 | 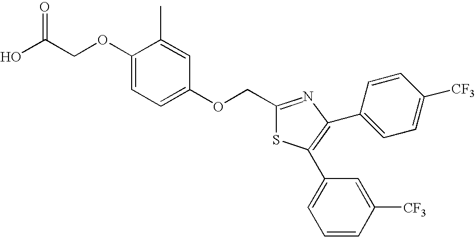 | ¹H-NMR (400 MHz, CDCl₃) δ =7.61 (d, J=2.8 Hz, 2H), 7.57 (m, 4H), 7.47 (s, 1H), 7.46 (d, J=3.2 Hz, 1H), 6.91 (d, J=2.8 Hz, 1H), 6.80 (dd, J=2.8, 8.8 Hz, 1H), 6.71 (d, J=8.8 Hz, 1H), 5.36 (s, 2H), 4.65 (s, 2H), 2.30 (s, 3H). MS calculated for C₂₇H₂₀F₆NO₄S (M + H⁺) 568.1, found 567.9. |
| F84 | 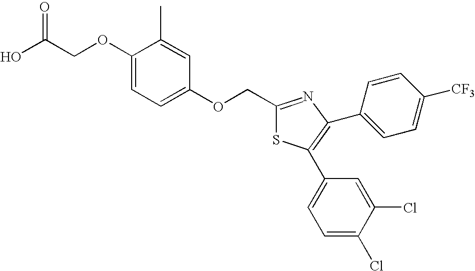 | ¹H-NMR (400 MHz, CDCl₃) δ =7.56 (m, 4H), 7.42 (d, J=2.0 Hz, 1H), 7.36 (d, J=8.4 Hz, 1H), 7.07 (dd, J=2.0, 8.4 Hz, 1H), 6.86 (d, J=2.8 Hz, 1H), 6.75 (dd, J=2.8, 8.8 Hz, 1H), 6.68 (d, J=8.8 Hz, 1H), 5.30 (s, 2H), 4.61 (s, 2H), 2.26 (s, 3H). MS calculated for C₂₆H₁₉Cl₂F₃NO₄S (M + H⁺) 568.0, found 567.9. |
| F85 | 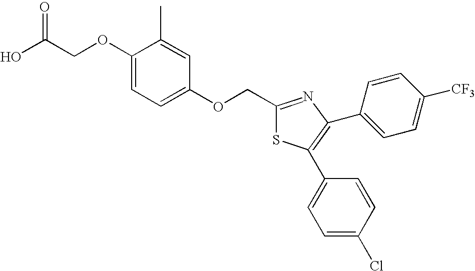 | ¹H-NMR (400 MHz, CDCl₃) δ =7.61 (d, J=8.0 Hz, 2H), 7.56 (d, J=8.4 Hz, 2H), 7.33 (d, J=8.4 Hz, 2H), 7.26 (d, J=8.4 Hz, 2H), 6.91 (d, J=3.2 Hz, 1H), 6.79 (dd, J=3.2, 8.8 Hz, 1H), 6.71 (d, J=8.8 Hz, 1H), 5.35 (s, 2H), 4.65 (s, 2H), 2.30 (s, 3H). MS calculated for C₂₆H₂₀ClF₃NO₄S (M + H⁺) 534.1, found 534.0. |
| F86 | 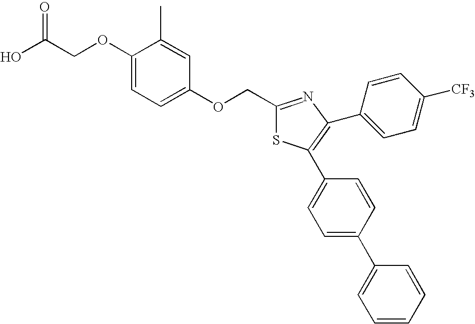 | ¹H-NMR (400 MHz, CDCl₃) δ =7.67 (d, J=8.4 Hz, 2H), 7.58 (m, 6H), 7.45 (t, J=7.2 Hz, 2H), 7.39 (m, 3H), 6.92 (d, J=2.8 Hz, 1H), 6.81 (dd, J=2.8, 8.8 Hz, 1H), 6.72 (d, J=8.8 Hz, 1H), 5.36 (s, 2H), 4.65 (s, 2H), 2.30 (s, 3H). MS calculated for C₃₂H₂₅F₃NO₄S (M + H⁺) 576.1, found 576.0. |
| F87 | 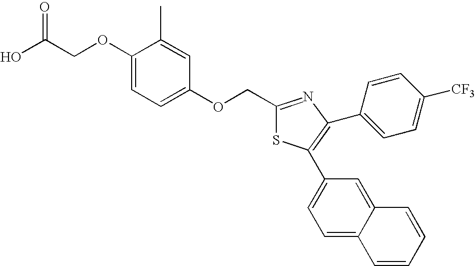 | ¹H-NMR (400 MHz, CDCl₃) δ =7.88 (s, 1H), 7.85 (m, 1H), 7.79 (d, J=8.8 Hz, 2H), 7.65 (d, J=8.0 Hz, 2H), 7.52 (m, 4H), 7.32 (dd, J=1.2, 8.4 Hz, 1H), 6.93 (d, J=2.8 Hz, 1H), 6.82 (dd, J=2.8, 8.8 Hz, 1H), 6.72 (d, J=8.8 Hz, 1H), 5.38 (s, 2H), 4.65 (s, 2H), 2.30 (s, 3H). MS calculated for C₃₀H₂₃F₃NO₄S (M + H⁺) 550.1, found 550.0. |
| F88 | 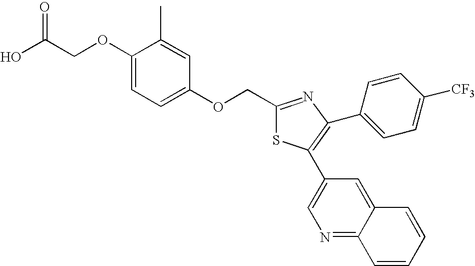 | ¹H-NMR (400 MHz, CDCl₃) δ =8.89 (d, J=2.0 Hz, 1H), 8.25 (s, 1H), 8.22 (d, J=8.0 Hz, 1H), 7.82 (m, 2H), 7.66 (d, J=7.2 Hz, 1H), 7.62 (d, J=8.0 Hz, 2H), 7.56 (d, J=8.4 Hz, 2H), 6.91 (d, J=2.8 Hz, 1H), 6.80 (dd, J=2.8, 8.8 Hz, 1H), 6.73 (d, J=8.8 Hz, 1H), 5.40 (s, 2H), 4.66 (s, 2H), 2.30 (s, 3H). MS calculated for C₂₉H₂₂F₃N₂O₄S |
| # (M + H⁺) 551.1, found 551.0. | | |
| F89 | 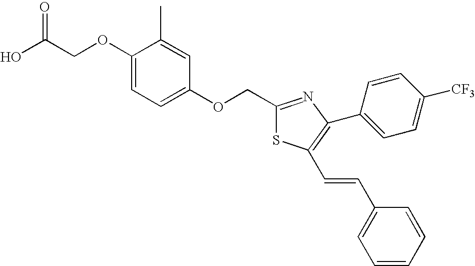 | ¹H-NMR (400 MHz, DMSO-d6) δ =8.30 (t, J=2.0 Hz, 1H), 7.91 (d, J=8.4 Hz, 2H), 7.87 (d, J=8.0 Hz, 2H), 7.58 (d, J=8.0 Hz, 2H), 7.37 (m, 3H), 7.30 (d, J=7.2 Hz, 1H), 7.13 (d, J=15.6 Hz, 1H), 6.95 (d, J=2.8 Hz, 1H), 6.86 (dd, J=2.8, 8.8 Hz, 1H), 6.78 (d, J=8.8 Hz, 1H), 5.40 (s, 2H), 4,62 (s, 2H), 2.20 (s, 3H). MS calculated for C₂₈H₂₃F₃NO₄S |
| # (M + H⁺) 526.1, found 526.0. | | |
| F90 | 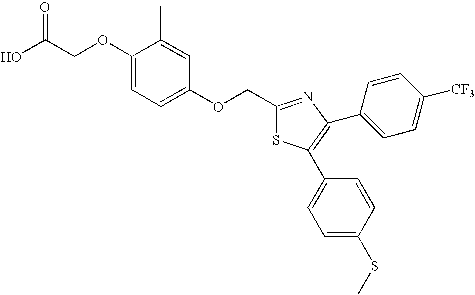 | ¹H-NMR (400 MHz, CDCl₃) δ =7.63 (d, J=8.4 Hz, 2H), 7.55 (d, J=8.0 Hz, 2H), 7.21 (m, 4H), 6.90 (d, J=2.8 Hz, 1H), 6.79 (dd, J=2.8, 8.8 Hz, 1H), 6.71 (d, J=8.8 Hz, 1H), 5.34 (s, 2H), 4.64 (s, 2H), 2.50 (s, 3H), 2.29 (s, 3H). MS calculated for C₂₇H₂₃F₃NO₄S₂(M + H⁺) 546.1, found 546.0. |
| F91 | 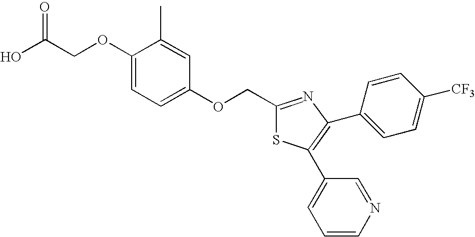 | ¹H-NMR (400 MHz, CDCl₃) δ =8.36 (d, J=2.4 Hz, 1H), 8.31 (dd, J=1.2, 8.8 Hz, 1H), 7.40 (d, J=8.0 Hz, 1H), 7.26 (s, 4H), 7.08 (dd, J=4.8, 8.0 Hz, 1H), 6.57 (d, J=2.8 Hz, 1H), 6.58 (dd, J=2.8, 8.8 Hz, 1H), 6.40 (d, J=8.8 Hz, 1H), 5.05 (s, 2H), 4.33 (s, 2H), 1.97 (s, 3H). MS calculated for C₂₅H₁₉F₃N₂O₄S (M + H⁺) 501.1, |
| # found 501.0. | | |
| F92 | 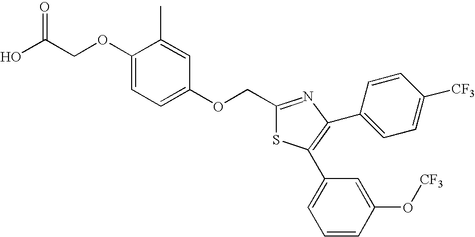 | ¹H-NMR (400 MHz, CDCl₃) δ =7.60 (d, J=8.8 Hz, 2H), 7.56 (d, J=8.4 Hz, 2H), 7.40 (t, J=8.0 Hz, 1H), 7.26 (d, J=8.4 Hz, 1H), 7.21 (d, J=8.4 Hz, 1H), 7.15 (s, 1H), 6.91 (d, J=3.2 Hz, 1H), 6.79 (dd, J=3.2, 8.8 Hz, 1H), 6.71 (d, J=8.8 Hz, 1H), 5.35 (s, 2H), 4.65 (s, 2H), 2.30 (s, 3H). MS calculated for C₂₇H₂₀F₆NO₅S (M + H⁺) |
| # 584.1, found 584.0. | | |
| F93 | 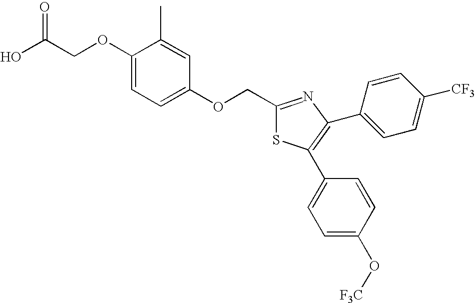 | ¹H-NMR (400 MHz, CDCl₃) δ =7.60 (d, J=8.8 Hz, 2H), 7.56 (d, J=8.8 Hz, 2H), 7.35 (d, J=8.8 Hz, 2H), 7.20 (d, J=8.8 Hz, 2H), 6.91 (d, J=3.2 Hz, 1H), 6.79 (dd, J=3.2, 8.8 Hz, 1H), 6.71 (d, J=8.8 Hz, 1H), 5.35 (s, 2H), 4.65 (s, 2H), 2.30 (s, 3H). MS calculated for C₂₇H₂₀F₆NO₅S (M + H⁺) 584.1, found 584.0. |
| F94 | 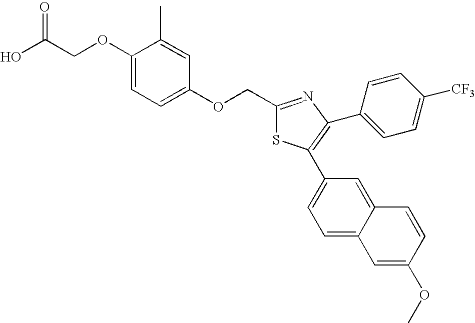 | ¹H-NMR (400 MHz, CDCl₃) δ =7.79 (s, 1H), 7.69 (d, J=2.4 Hz, 1H), 7.65 (d, J=8.4 Hz, 3H), 7.51 (d, J=8.0 Hz, 2H), 7.29 (dd, J=1.6, 8.4 Hz, 1H), 7.18 (dd, J=2.4, 8.8 Hz, 1H), 7.13 (d, J=2.4 Hz, 1H), 6.92 (d, J=3.2 Hz, 1H), 6.82 (dd, J=3.2, 8.8 Hz, 1H), 6.72 (d, J=8.8 Hz, 1H), 5.37 (s, 2H), 4.65 (s, 2H), 3.94 (s, 3H), 2.30 (s, 3H). MS calculated for |
| # C₃₁H₂₅F₃NO₅S (M + H⁺) 580.1, found 580.0. | | |
| F95 | 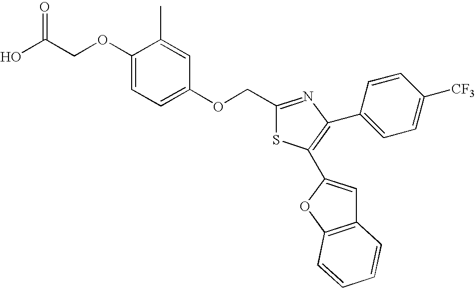 | ¹H-NMR (400 MHz, DMSO-d6) δ =8.33 (s, 1H), 7.91 (d, J=8.0 Hz, 2H), 7.85 (d, J=8.4 Hz, 2H), 7.65 (d, J=8.0 Hz, 1H), 7.58 (d, J=8.4 Hz, 1H), 7.37 (t, J=6.8 Hz, 1H), 7.29 (t, J=7.2 Hz, 1H), 7.06 (s, 1H), 7.01 (d, J=3.2 Hz, 1H), 6.91 (dd, J=3.2, 8.8 Hz, 1H), 6.81 (d, J=8.8 Hz, 1H), 5.49 (s, 2H), 4.66 (s, 2H), 2.23 (s, 3H). MS calculated for |
| # C₂₈H₂₁F₃NO₅S (M + H⁺) 540.1, found 540.0. | | |
| F96 | 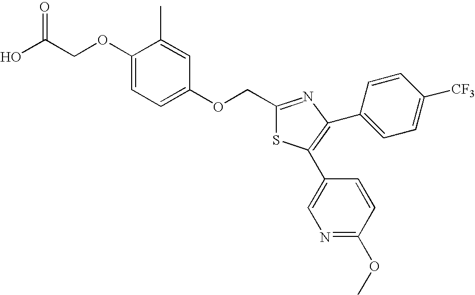 | ¹H-NMR (400 MHz, CDCl₃) δ =8.17 (d, J=2.0 Hz, 1H), 7.62 (d, J=8.4 Hz, 2H), 7.57 (d, J=8.4 Hz, 2H), 7.47 (dd, J=2.8, 8.4 Hz, 1H), 6.91 (d, J=2.8 Hz, 1H), 6.80 (dd, J=2.8, 8.8 Hz, 1H), 6.73 (t, J=8.8 Hz, 2H), 5.34 (s, 2H), 4.65 (s, 2H), 3.96 (s, 3H), 2.30 (s, 3H). MS calculated for C₂₆H₂₁F₃N₂O₅S (M + H⁺) 531.1, found 531.0. |
| F97 | 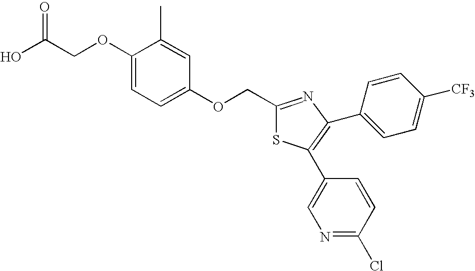 | ¹H-NMR (400 MHz, CDCl₃) δ =8.35 (d, J=2.4 Hz, 1H), 7.56 (m, 4H), 7.53 (dd, J=2.4, 8.4 Hz, 1H), 7.29 (d, J=8.0 Hz, 1H), 6.87 (d, J=2.8 Hz, 1H), 6.76 (dd, J=2.8, 8.8 Hz, 1H), 6.69 (d, J=8.8 Hz, 1H), 5.32 (s, 2H), 4.62 (s, 2H), 2.26 (s, 3H). MS calculated for C₂₅H₁₉ClF₃N₂O₄S (M + H⁺) 535.1, found 535.0. |
| F98 | 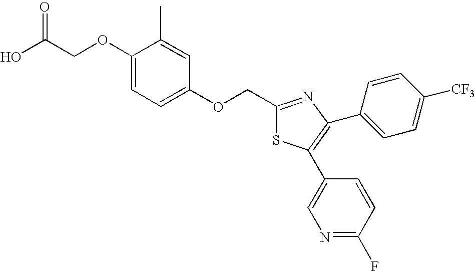 | ¹H-NMR (400 MHz, CDCl₃) δ =8.23 (d, J=2.4 Hz, 1H), 7.70 (dt, J=2.8, 8.4 Hz, 1H), 7.58 (m, 4H), 7.53 (dd, J=2.4, 8.4 Hz, 1H), 6.95 (dd, J=2.0, 8.4 Hz, 1H), 6.90 (d, J=2.8 Hz, 1H), 6.79 (dd, J=2.8, 8.8 Hz, 1H), 6.71 (d, J=8.8 Hz, 1H), 5.36 (s, 2H), 4.65 (s, 2H), 2.29 (s, 3H). MS calculated for C₂₅H₁₉F₄N₂O₄S (M + H⁺) |
| # 519.1, found 519.0. | | |
| F99 | 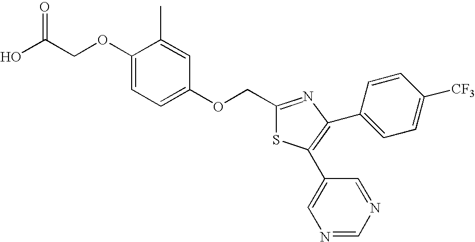 | ¹H-NMR (400 MHz, DMSO-d6) δ =9.21 (s, 1H), 8.82 (s, 2H), 7.74 (d, J=8.4 Hz, 2H), 7.67 (d, J=8.0 Hz, 2H), 6.97 (d, J=2.8 Hz, 1H), 6.88 (dd, J=2.8, 8.8 Hz, 1H), 6.79 (d, J=8.8 Hz, 1H), 5.47 (s, 2H), 4.63 (s, 2H), 2.20 (s, 3H). MS calculated for C₂₄H₁₉F₃N₃O₄S (M + H⁺) 502.1, found 502.0. |
| F100 | 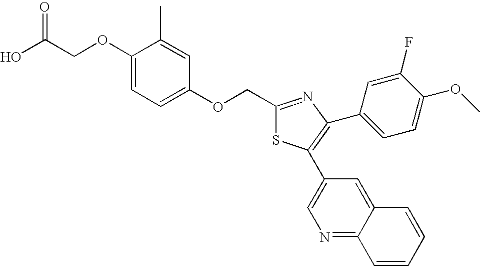 | ¹H-NMR (400 MHz, CDCl₃) δ =9.06 (d, J=1.6 Hz, 1H), 8.56 (s, 1H), 8.46 (d, J=8.8 Hz, 1H), 7.80 (t, J=7.2 Hz, 1H), 7.95 (d, J=8.0 Hz, 1H), 7.82 (t, J=7.6 Hz, 1H), 7.28 (m, 1H), 7.12 (d, J=8.4 Hz, 1H), 6.89 (m, 2H), 6.76 (dd, J=2.8, 8.8 Hz, 1H), 6.70 (d, J=8.8 Hz, 1H), 5.39 (s, 2H), 4.66 (s, 2H), 3.89 (s, 3H), 2.28 (s, 3H). MS calculated for |
| # C₂₉H₂₄FN₂O₅S (M + H⁺) 531.1, found 531.3. | | |
| F101 | 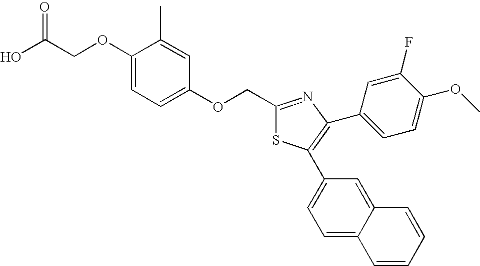 | ¹H-NMR (400 MHz, CDCl₃) δ =7.88 (s, 1H), 7.80 (m, 3H), 7.51 (m, 2H), 7.34 (dd, J=2.0, 8.8 Hz, 1H), 7.30 (dd, J=2.0, 12.4 Hz, 1H), 7.21 (d, J=8.4 Hz, 1H), 6.92 (d, J=2.8 Hz, 1H), 6.84 (t, J=8.4 Hz, 1H), 6.80 (dd, J=2.8, 8.8 Hz, 1H), 6.71 (d, J=8.8 Hz, 1H), 5.37 (s, 2H), 4.65 (s, 2H), 3.87 (s, 3H), 2.29 (s, 3H). MS calculated for C₃₀H₂₅FNO₅S |
| # (M + H⁺) 530.1, found 530.3. | | |
| F102 | 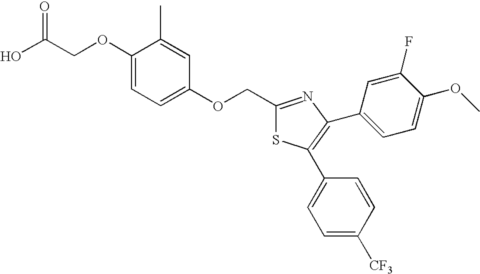 | ¹H-NMR (400 MHz, CDCl₃) δ =7.59 (d, J=8.0 Hz, 2H), 7.44 (d, J=8.4 Hz, 2H), 7.24 (dd, J=1.6, 12 Hz, 1H), 7.14 (d, J=8.8 Hz, 1H), 6.89 (m, 2H), 6.78 (dd, J=2.8, 8.8 Hz, 1H), 6.71 (d, J=8.8 Hz, 1H), 5.36 (s, 2H), 4.65 (s, 2H), 3.90 (s, 3H), 2.29 (s, 3H). MS calculated for C₂₇H₂₂F₄NO₅S (M + H⁺) 548.1, found 548.4. |
| F103 | 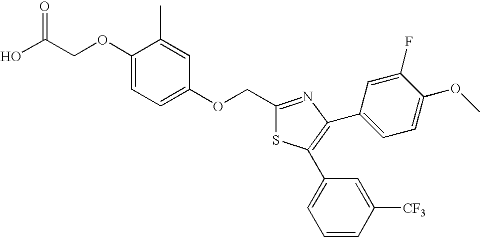 | ¹H-NMR (400 MHz, CDCl₃) δ =7.59 (m, 2H), 7.47 (m, 2H), 7.22 (dd, J=2.0, 12.4 Hz, 1H), 7.16 (d, J=8.8 Hz, 1H), 7.90 (d, J=2.8 Hz, 1H), 6.87 (d, J=8.8 Hz, 1H), 6.79 (dd, J=2.8, 8.8 Hz, 1H), 6.71 (d, J=8.8 Hz, 1H), 5.36 (s, 2H), 4.65 (s, 2H), 3.90 (s, 3H), 2.29 (s, 3H). MS calculated for C₂₇H₂₂F₄NO₅S (M + H⁺) 548.1, found 548.4. |
| F104 | 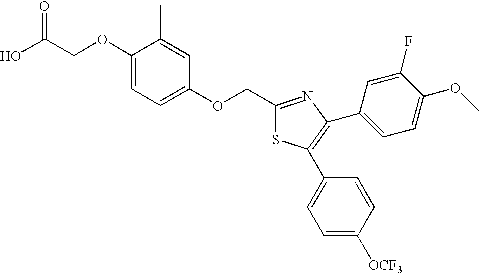 | ¹H-NMR (400 MHz, CDCl₃) δ =7.35 (d, J=8.4 Hz, 2H), 7.21 (m, 3H), 6.89 (m, 2H), 6.78 (dd, J=2.8, 8.8 Hz, 1H), 6.70 (d, J=8.8 Hz, 1H), 5.34 (s, 2H), 4.65 (s, 2H), 3.90 (s, 3H), 2.29 (s, 3H). MS calculated for C₂₇H₂₂F₄NO₆S (M + H⁺) 564.1, found 564.3. |
| F105 | 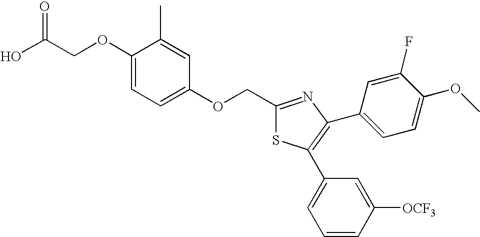 | ¹H-NMR (400 MHz, CDCl₃) δ =7.53 (t, J=7.6 Hz, 1H), 7.41 (m, 2H), 7.35 (m, 3H), 7.04 (m, 2H), 6.93 (dd, J=2.8, 8.8 Hz, 1H), 6.86 (d, J=8.8 Hz, 1H), 5.49 (s, 2H), 4.80 (s, 2H), 4.05 (s, 3H), 2.44 (s, 3H). MS calculated for C₂₇H₂₂F₄NO₆S (M + H⁺) 564.1, found 564.3. |
| F106 | 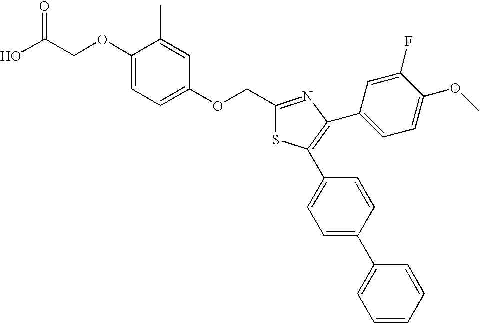 | ¹H-NMR (400 MHz, CDCl₃) δ =7.40 (d, J=8.0 Hz, 2H), 7.36 (d, J=8.0 Hz, 2H), 7.25 (m, 2H), 7.18 (m, 3H), 7.10 (dd, J=1.2, 12.4 Hz, 1H), 7.06 (m, 1H), 6.70 (d, J=2.4 Hz, 1H), 6.67 (d, J=8.8 Hz, 1H), 6.59 (dd, J=2.8, 8.8 Hz, 1H), 6.50 (d, J=8.8 Hz, 1H), 5.14 (s, 2H), 4.44 (s, 2H), 3.69 (s, 3H), 2.09 (s, 3H). MS calculated for C₃₂H₂₇FNO₅S |
| # (M + H⁺) 556.2, found 556.2. | | |
| F107 | 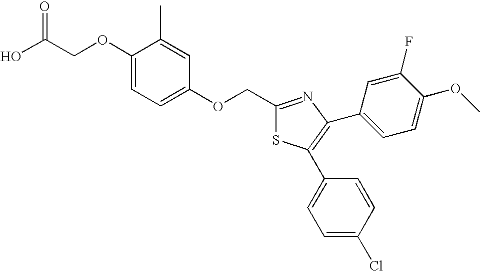 | ¹H-NMR (400 MHz, CDCl₃) δ =7.26 (d, J=8.8 Hz, 2H), 7.20 (m, 3H), 7.11 (d, J=8.4 Hz, 1H), 6.83 (m, 2H), 6.72 (dd, J=2.8, 8.8 Hz, 1H), 6.65 (d, J=8.8 Hz, 1H), 5.28 (s, 2H), 4.58 (s, 2H), 3.83 (s, 3H), 2.29 (s, 3H). MS calculated for C₂₆H₂₂ClFNO₅S (M + H⁺) 514.1, found 514.2. |
| F108 | 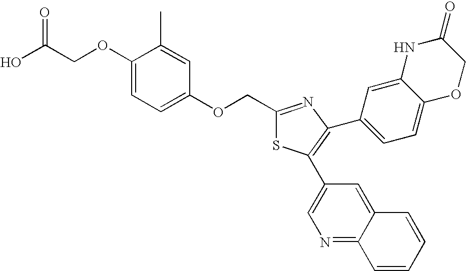 | ¹H-NMR (400 MHz, DMSO-d6) δ =10.61 (s, 1H), 8.60 (d, J=2.0 Hz, 1H), 8.41 (s, 1H), 7.91 (d, J=8.8 Hz, 2H), 7.70 (t, J=7.6 Hz, 1H), 7.55 (t, J=7.2 Hz, 1H), 7.04 (d, J=1.6 Hz, 1H), 6.83 (m, 2H), 6.76 (m, 2H), 6.68 (d, J=8.8 Hz, 1H), 5.33 (s, 2H), 4.52 (s, 2H), 4.48 (s, 2H), 2.09 (s, 3H). MS calculated for C₃₀H₂₄N₃O₆S (M + H⁺) 554.1, |
| # found 554.1. | | |
| F109 | 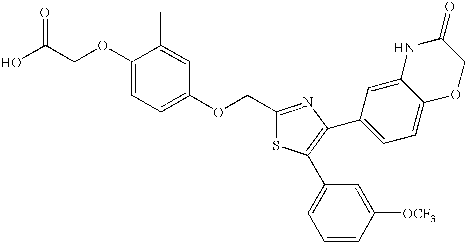 | ¹H-NMR (400 MHz, DMSO-d6) δ =10.66 (s, 1H), 7.45 (t, J=8.0 Hz, 1H), 7.31 (d, J=8.0 Hz, 1H), 7.27 (d, J=8.8 Hz, 1H), 7.17 (s, 1H), 7.05 (d, J=2.4 Hz, 1H), 6.85 (d, J=2.8 Hz, 1H), 6.77 (m, 3H), 6.68 (d, J=8.8 Hz, 1H), 5.29 (s, 2H), 4.53 (s, 2H), 4.48 (s, 2H), 2.11 (s, 3H). MS calculated for C₂₈H₂₂F₃N₂O₇S (M + H⁺) 587.1, found 587.1. |
| F110 | 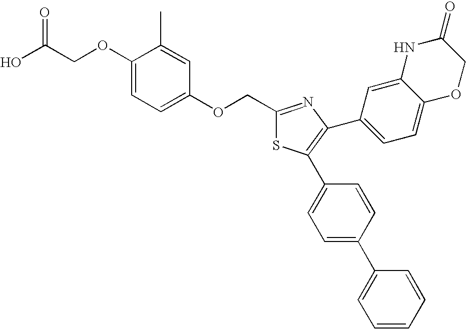 | ¹H-NMR (400 MHz, DMSO-d6) δ =10.73 (s, 1H), 7.66 (d, J=8.0 Hz, 4H), 7.44 (m, 4H), 7.35 (t, J=7.6 Hz, 1H), 7.21 (d, J=1.6 Hz, 1H), 6.93 (m, 2H), 6.75 (d, J=8.8 Hz, 1H), 5.35 (s, 2H), 4.59 (s, 2H), 4.55 (s, 2H), 2.21 (s, 3H). MS calculated for C₃₃H₂₇N₂O₆S (M + H⁺) 579.2, found 579.1. |
| F111 | 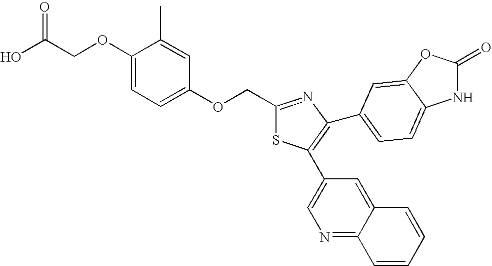 | ¹H-NMR (400 MHz, DMSO-d6) δ =11.60 (s, 1H), 8.75 (d, J=2.4 Hz, 1H), 8.44 (s, 1H), 7.96 (d, J=7.2 Hz, 1H), 7.81 (t, J=7.2 Hz, 1H), 6.97 (d, J=8.4 Hz, 1H), 6.93 (d, J=2.8 Hz, 1H), 6.86 (dd, J=2.8, 8.4 Hz, 1H), 6.76 (d, J=8.4 Hz, 1H), 5.42 (s, 2H), 4.59 (s, 2H), 2.27 (s, 3H). MS calculated for C₂₉H₂₂N₃O₆S (M + H⁺) 540.1, found 540.1. |
| F112 | 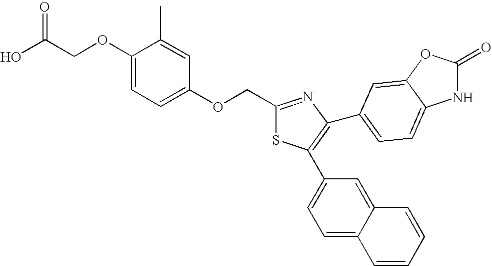 | ¹H-NMR (400 MHz, DMSO-d6) δ =11.55 (s, 1H), 7.93 (s, 1H), 7.84 (m, 3H), 7.50 (m, 2H), 7.32 (dd, J=2.0, 8.8 Hz, 1H), 7.29 (s, 1H), 7.22 (d, J=9.6 Hz, 1H), 6.91 (d, J=8.4 Hz, 1H), 6.89 (d, J=2.4 Hz, 1H), 6.82 (dd, J=2.8, 8.8 Hz, 1H), 6.72 (d, J=8.8 Hz, 1H), 5.35 (s, 2H), 4.56 (s, 2H), 2.21 (s, 3H). MS calculated for C₃₀H₂₃N₂O₆S (M + H⁺) |
| # 539.1, found 539.1. | | |
| F113 | 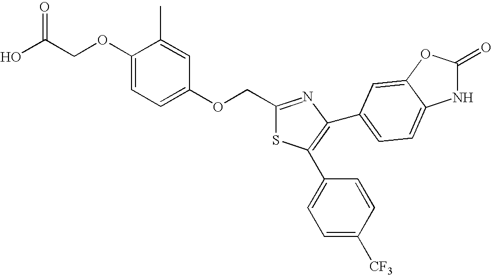 | ¹H-NMR (400 MHz, DMSO-d6) δ =11.62 (s, 1H), 7.67 (d, J=8.4 Hz, 2H), 7.54 (d, J=7.6 Hz, 2H), 7.31 (s, 1H), 7.20 (d, J=7.6 Hz, 1H), 6.99 (d, J=8.0 Hz, 1H), 6.92 (s, 1H), 6.84 (d, J=8.0 Hz, 1H), 6.75 (d, J=8.4 Hz, 1H), 5.38 (s, 2H), 4.59 (s, 2H), 2.25 (s, 3H). MS calculated for C₂₇H₂₀F₃N₂O₆S (M + H⁺) 557.1, found 557.3. |
| F114 | 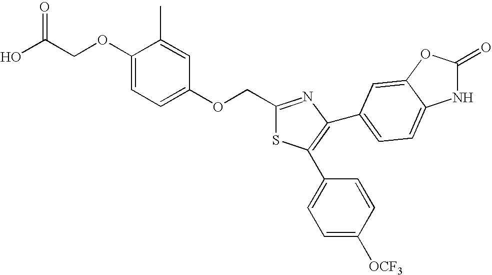 | ¹H-NMR (400 MHz, DMSO-d6) δ =11.64 (s, 1H), 8.13 (m, 2H), 7.49 (m, 2H), 7.33 (br s, 3H), 7.26 (d, J=8.4 Hz, 1H), 7.03 (dd, J=4.0, 8.0 Hz, 1H), 6.95 (s, 1H), 6.88 (m, 1H), 6.79 (dd, J=3.2, 8.8 Hz, 1H), 5.41 (s, 2H), 4.63 (s, 2H), 2.30 (s, 3H). MS calculated for C₂₇H₂₀F₃N₂O₇S (M + H⁺) 573.1, found 573.2. |
| F115 | 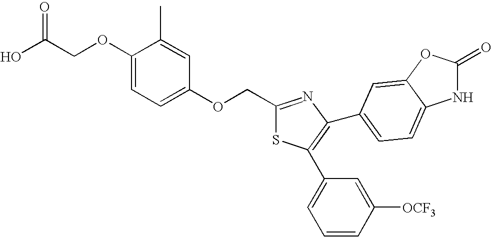 | ¹H-NMR (400 MHz, DMSO-d6) δ =11.35 (s, 1H), 7.88 (m, 2H), 7.24 (t, J=8.0 Hz, 1H), 7.11 (d, J=7.6 Hz, 1H), 7.03 (d, J=9.6 Hz, 2H), 6.95 (d, J=8.4 Hz, 2H), 6.72 (d, J=8.0 Hz, 1H), 6.65 (s, 1H), 6.58 (dd, J=2.4, 8.8 Hz, 1H), 6.49 (d, J=8.8 Hz, 1H), 5.10 (s, 2H), 4.32 (s, 2H), 1.97 (s, 3H). MS calculated for C₂₇H₂₀F₃N₂O₇S (M + H⁺) |
| # 573.1, found 573.2. | | |
| F116 | 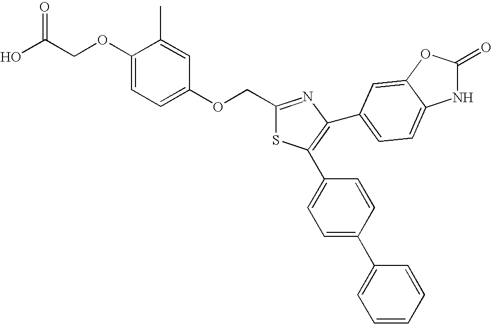 | ¹H-NMR (400 MHz, DMSO-d6) δ =11.68 (s, 1H), 8.23 (m, 2H), 7.67 (d, J=8.0 Hz, 4H), 7.44 (m, 4H), 7.37 (d, J=6.8 Hz, 1H), 7.33 (s, 1H), 7.27 (d, J=8.4 Hz, 1H), 7.01 (d, J=8.0 Hz, 1H), 6.93 (s, 1H), 6.85 (dd, J=2.4, 8.8 Hz, 1H), 6.76 (d, J=8.8 Hz, 1H), 5.37 (s, 2H), 4.60 (s, 2H), 2.21 (s, 3H). MS calculated for C₃₂H₂₅N₂O₆S (M + H⁺) 565.1, |
| # found 565.1. | | |
| F117 | 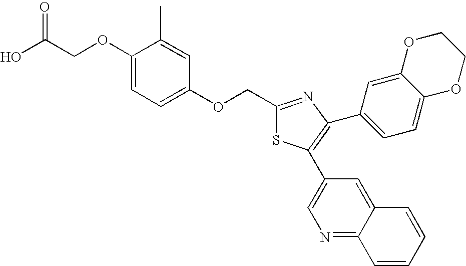 | ¹H-NMR (400 MHz, CDCl₃) δ =8.87 (s, 1H), 8.27 (s, 1H), 8.19 (d, J=8.0 Hz, 1H), 7.79 (m, 2H), 7.63 (t, J=7.6 Hz, 1H), 7.05 (s, 1H), 6.89 (s, 2H), 6.78 (m, 2H), 6.71 (d, J=8.8 Hz, 1H), 5.37 (s, 2H), 4.64 (s, 2H), 4.23 (m, 4H), 2.28 (s, 3H). MS calculated for C₃₀H₂₅N₂O₆S (M + H⁺) 541.1, found 541.1. |
| F118 | 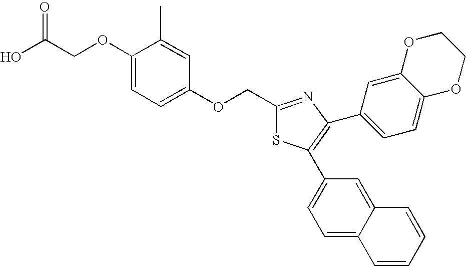 | ¹H-NMR (400 MHz, CDCl₃) δ =7.88 (s, 1H), 7.80 (m, 2H), 7.75 (d, J=8.8 Hz, 1H), 7.49 (m, 2H), 7.36 (d, J=8.4 Hz, 1H), 7.08 (d, J=1.2 Hz, 1H), 6.94 (m, 2H), 6.80 (dd, J=2.8, 8.8 Hz, 1H), 6.73 (t, J=8.8 Hz, 2H), 5.37 (s, 2H), 4.64 (s, 2H), 4.23 (m, 4H), 2.01 (s, 3H). MS calculated for C₃₁H₂₆NO₆S (M + H⁺) 540.1, found 540.2. |
| F119 | 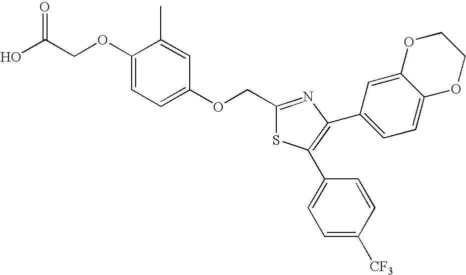 | ¹H-NMR (400 MHz, CDCl₃) δ =7.56 (d, J=8.4 Hz, 2H), 7.45 (d, J=8.8 Hz, 2H), 7.03 (d, J=2.0 Hz, 1H), 6.89 (m, 2H), 6.77 (m, 2H), 6.70 (d, J=8.8 Hz, 1H), 5.32 (s, 2H), 4.63 (s, 2H), 4.26 (m, 4H), 2.28 (s, 3H). MS calculated for C₂₈H₂₃F₃NO₆S (M + H⁺) 558.1, found 558.1. |
| F120 | 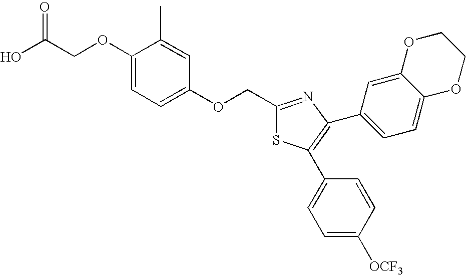 | ¹H-NMR (400 MHz, CDCl₃) δ =7.37 (d, J=8.4 Hz, 2H), 7.16 (d, J=8.4 Hz, 2H), 7.03 (d, J=2.0 Hz, 1H), 6.90 (m, 2H), 6.77 (d, J=8.4 Hz, 2H), 6.70 (d, J=8.8 Hz, 1H), 5.31 (s, 2H), 4.63 (s, 2H), 4.26 (m, 4H), 2.28 (s, 3H). MS calculated for C₂₈H₂₃F₃NO₇S (M + H⁺) 574.1, found 574.1. |
| F121 | 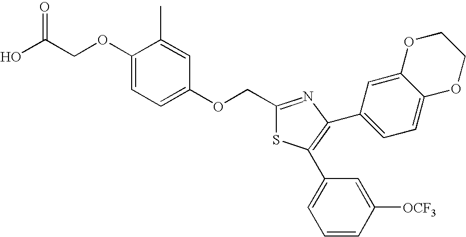 | ¹H-NMR (400 MHz, CDCl₃) δ =7.35 (t, J=7.6 Hz, 1H), 7.28 (d, J=8.0 Hz, 1H), 7.15 (d, J=8.4 Hz, 2H), 7.01 (d, J=2.0 Hz, 1H), 6.92 (dd, J=2.0, 8.4 Hz, 1H), 6.89 (d, J=2.8 Hz, 1H), 6.79 (d, J=8.4 Hz, 1H), 6.77 (dd, J=2.8, 8.8 Hz, 1H), 6.70 (d, J=8.8 Hz, 1H), 5.32 (s, 2H), 4.63 (s, 2H), 4.25 (m, 4H), 2.28 (s, 3H). MS calculated for C₂₈H₂₃F₃NO₇S |
| # (M + H⁺) 574.1, found 574.1. | | |
| F122 | 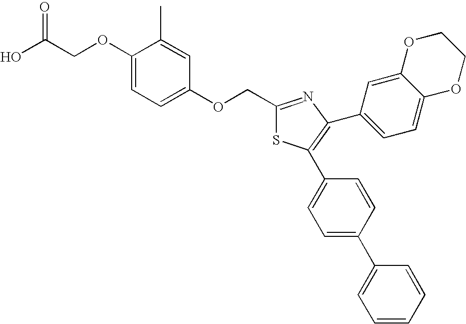 | ¹H-NMR (400 MHz, CDCl₃) δ =7.60 (d, J=7.2 Hz, 2H), 7.56 (d, J=8.4 Hz, 2H), 7.42 (m, 5H), 7.08 (d, J=2.4 Hz, 1H), 6.97 (dd, J=2.0, 8.4 Hz, 1H), 6.91 (d, J=8.8 Hz, 1H), 6.79 (d, J=8.4 Hz, 2H), 6.71 (d, J=8.8 Hz, 1H), 5.35 (s, 2H), 4.64 (s, 2H), 4.25 (m, 4H), 2.29 (s, 3H). MS calculated for C₃₃H₂₈NO₆S (M + H⁺) 566.2, found 566.2. |
| F123 | 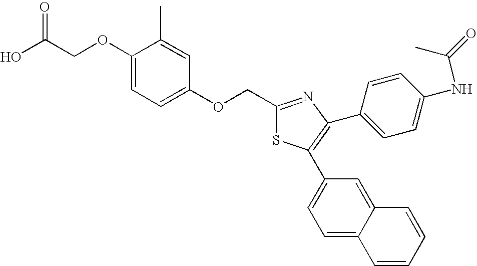 | ¹H-NMR (400 MHz, CDCl₃) δ =8.05 (s, 1H), 8.00 (m, 1H), 7.94 (m, 2H), 7.66 (m, 4H), 7.59 (m, 2H), 7.50 (d, J=9.2 Hz, 1H), 7.10 (s, 1H), 6.99 (d, J=8.4 Hz, 1H), 6.90 (d, J=8.4 Hz, 1H), 5.56 (s, 2H), 4.82 (s, 2H), 2.48 (s, 3H), 2.37 (s, 3H). MS calculated for C₃₁H₂₇N₂O₅S (M + H⁺) 539.2, found 539.2. |
| F124 | 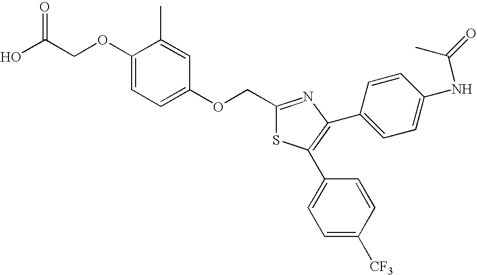 | ¹H-NMR (400 MHz, CDCl₃) δ =7.43 (d, J=8.8 Hz, 2H), 7.37 (d, J=8.4 Hz, 2H), 7.31 (m, 3H), 7.13 (d, J=8.8 Hz, 2H), 6.86 (d, J=2.4 Hz, 1H), 6.76 (dd, J=2.4, 8.8 Hz, 1H), 6.67 (d, J=8.8 Hz, 1H), 5.34 (s, 2H), 4.61 (s, 2H), 2.25 (s, 3H), 2.19 (s, 3H). MS calculated for C₂₈H₂₄F₃N₂O₅S (M + H⁺) 557.1, found 557.1. |
| F125 | 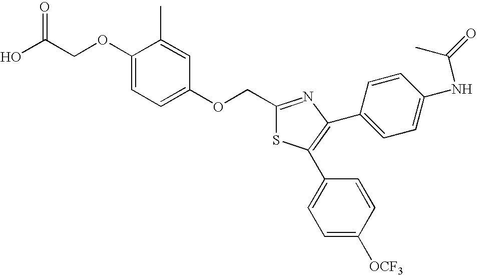 | MS calculated for C₂₈H₂₄F₃N₂O₆S (M + H⁺) 573.1, found 573.1. |
| F126 | 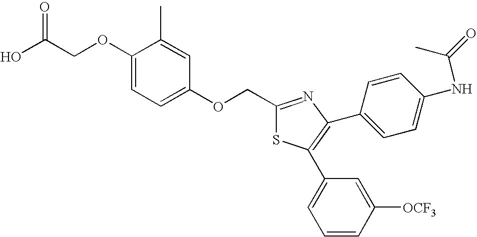 | ¹H-NMR (400 MHz, CDCl₃) δ =7.45 (m, 4H), 7.35 (t, J=8.8 Hz, 1H), 7.22 (s, 1H), 7.18 (m, 2H), 6.90 (d, J=3.2 Hz, 1H), 6.79 (dd, J=2.8, 8.8 Hz, 1H), 6.71 (d, J=8.8 Hz, 1H), 5.34 (s, 2H), 4.64 (s, 2H), 2.29 (s, 3H), 2.19 (s, 3H). MS calculated for C₂₈H₂₄F₃N₂O₆S (M + H⁺) 573.1, found 573.1. |
| F127 | 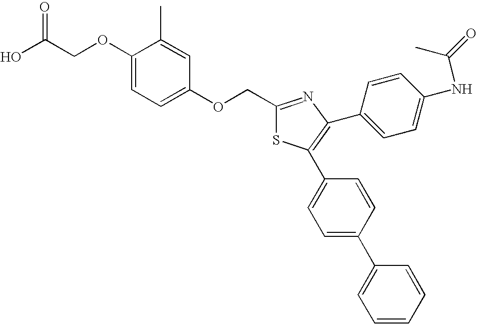 | ¹H-NMR (400 MHz, CDCl₃) δ =7.62 (d, J=7.6 Hz, 2H), 7.57 (d, J=8.4 Hz, 2H), 7.50 (m, 6H), 7.40 (m, 3H) 7.34 (s, 1H), 6.94 (d, J=2.8 Hz, 1H), 6.83 (dd, J=2.8, 8.8 Hz, 1H), 6.74 (d, J=8.8 Hz, 1H), 5.40 (s, 2H), 4.67 (s, 2H), 2.32 (s, 3H), 2.23 (s, 3H). MS calculated for C₃₃H₂₉N₂O₅S (M + H⁺) 565.2, found 565.1. |
| F128 | 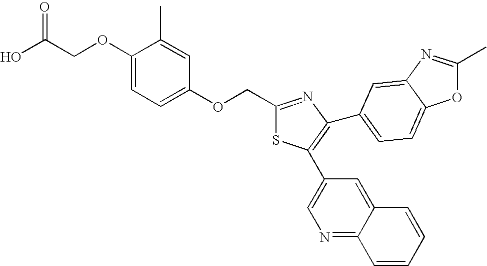 | ¹H-NMR (400 MHz, CDCl₃) δ =8.76 (s, 1H), 8.19 (s, 1H), 8.13 (m, 1H), 7.75 (d, J=8.0 Hz, 2H), 7.70 (s, 1H), 7.59 (t, J=8.0 Hz, 1H), 7.35 (s, 2H), 6.91 (s, 1H), 6.87 (d, J=8.4 Hz, 1H), 6.77 (d, J=8.8 Hz, 1H), 5.40 (s, 2H), 4.69 (s, 2H), 2.64 (s, 3H), 2.31 (s, 3H). MS calculated for C₃₀H₂₄N₃O₅S (M + H⁺) 538.1, found 538.0. |
| F129 | 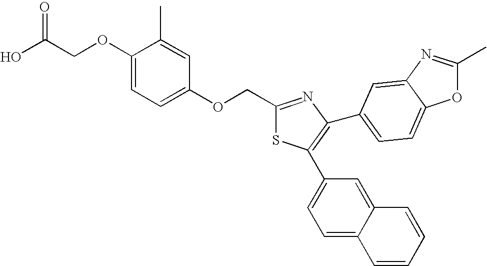 | ¹H-NMR (400 MHz, CDCl₃) δ =7.86 (s, 1H), 7.79 (m, 2H), 7.73 (m, 2H), 7.48 (m, 2H), 7.39 (dd, J=1.6, 8.4 Hz, 1H), 7.31 (d, J=8.0 Hz, 1H), 7.30 (dd, J=1.6, 6.8 Hz, 1H), 6.92 (d, J=2.8 Hz, 1H), 6.85 (dd, J=2.8, 8.8 Hz, 1H), 6.76 (d, J=8.8 Hz, 1H), 5.39 (s, 2H), 4.67 (s, 2H), 2.65 (s, 3H), 2.30 (s, 3H). MS calculated for C₃₁H₂₅N₂O₅S |
| # (M + H⁺) 537.1, found 537.0. | | |
| F130 | 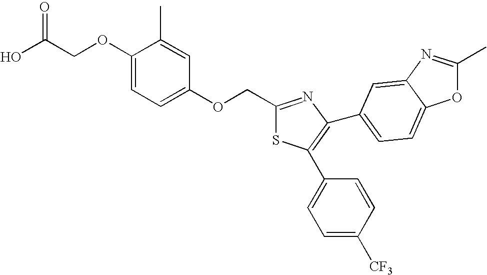 | ¹H-NMR (400 MHz, CDCl₃) δ =7.79 (s, 1H), 7.62 (d, J=8.0 Hz, 2H), 7.47 (m, 4H), 6.90 (d, J=2.4 Hz, 1H), 6.90 (dd, J=2.8, 8.8 Hz, 1H), 6.82 (d, J=8.4 Hz, 1H), 5.44 (s, 2H), 4.74 (s, 2H), 2.75 (s, 3H), 2.37 (s, 3H). MS calculated for C₂₈H₂₂F₃N₂O₅S (M + H⁺) 555.1, found 555.0. |
| F131 | 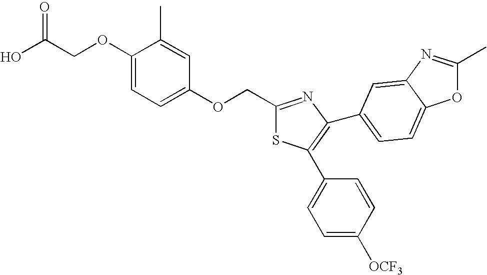 | MS calculated for C₂₈H₂₂F₃N₂O₆S (M + H⁺) 571.1, found 571.0. |
| F132 | 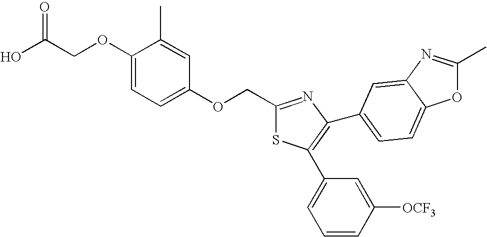 | ¹H-NMR (400 MHz, CDCl₃) δ =7.71 (s, 1H), 7.36 (m, 2H), 7.32 (d, J=8.0 Hz, 1H), 7.25 (m, 1H), 7.15 (m, 2H), 6.90 (d, J=2.8 Hz, 1H), 6.82 (dd, J=2.8, 8.8 Hz, 1H), 6.74 (d, J=8.4 Hz, 1H), 5.35 (s, 2H), 4.67 (s, 2H), 2.66 (s, 3H), 2.30 (s, 3H). MS calculated for C₂₈H₂₂F₃N₂O₆S (M + H⁺) 571.1, found 571.0. |
| F133 | 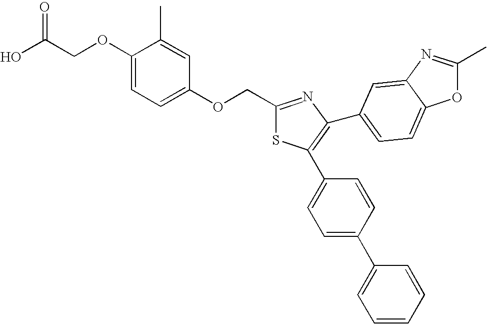 | ¹H-NMR (400 MHz, CDCl₃) δ =7.75 (d, J=1.2 Hz, 1H), 7.58 (d, J=7.2 Hz, 2H), 7.45 (m, 6H), 6.90 (m, 4H), 6.85 (dd, J=2.8, 8.8 Hz, 1H), 6.76 (d, J=8.8 Hz, 1H), 5.37 (s, 2H), 4.67 (s, 2H), 2.65 (s, 3H), 2.30 (s, 3H). MS calculated for C₃₃H₂₇N₂O₅S (M + H⁺) 563.2, found 563.0. |
| F134 | 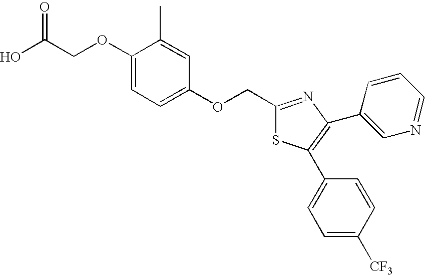 | ¹H-NMR (400 MHz, CDCl₃) δ =8.86 (s, 1H), 8.77 (d, J=4.4 Hz, 1H), 7.97 (d, J=8.4 Hz, 1H), 7.68 (d, J=8.0 Hz, 2H), 7.55 (m, 1H), 7.47 (d, J=8.0 Hz, 2H), 6.89 (dd, J=2.4, 8.8 Hz, 1H), 6.80(d, J=2.4 Hz, 1H), 6.74 (d, J=8.8 Hz, 1H), 5.35 (s, 2H), 4.72 (s, 2H), 2.25 (s, 3H). MS calculated for C₂₅H₂₀F₃N₂O₄S (M + H⁺) 501.1, found |
| # 501.1. | | |
| F135 | 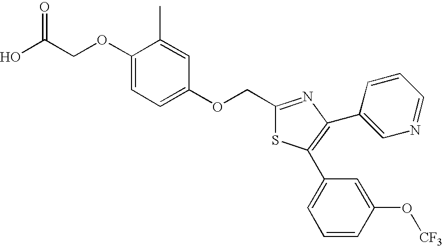 | ¹H-NMR (400 MHz, CDCl₃) δ =8.83 (s, 1H), 8.59 (d, J=4.4 Hz, 1H), 7.83 (d, J=8.0 Hz, 1H), 7.34 (m, 2H), 7.23 (d, J=8.0 Hz, 1H), 7.19 (d, J=8.4 Hz, 1H), 7.12 (s, 1H), 6.83 (d, J=2.8 Hz, 1H), 6.75 (dd, J=2.8, 8.8 Hz, 1H), 6.68 (d, J=8.8 Hz, 1H), 5.30 (s, 2H), 4.61 (s, 2H), 2.25 (s, 3H). MS calculated for C₂₅H₂₀F₃N₂O₅S (M + H⁺) |
| # 517.1, found 517.1. | | |
| H2 | 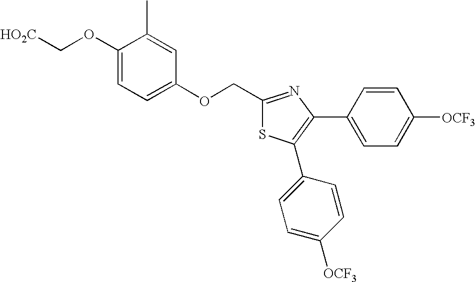 | ¹H-NMR (400 MHz, CD₃OD) δ =7.57 (d, J=8.4 Hz, 2H), 7.45 (d, J=8.4 Hz, 2H), 7.31 (d, J=8.4 Hz, 2H), 7.25 (d, J=8.4 Hz, 2H), 6.92 (d, J=2.8 Hz, 1H), 6.85-6.76 (m, 2H), 5.35 (s, 2H), 4.62 (s, 2H), 2.26 (s, 3H). MS calculated for C₂₇H₂₀F₆NO₆S (M + H⁺) 600.08, found 600.00. |
| H3 | 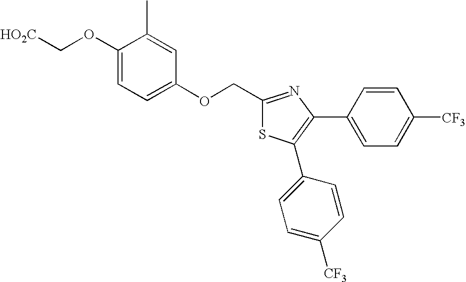 | ¹H-NMR (400 MHz, CD₃OD) δ =7.71 (d, J=8.4 Hz, 2H), 7.65 (s, 4H), 7.56 (d, J=8.4 Hz, 2H), 6.94 (d, J=2.8 Hz, 1H), 6.87-6.77 (m, 2H), 5.39 (s, 2H), 4.63 (s, 2H), 2.27 (s, 3H). MS calculated for C₂₇H₂₀F₆NO₄S (M + H⁺) 568.09, found 568.00. |
| H4 | 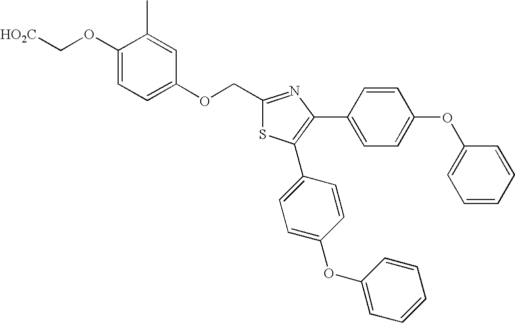 | ¹H-NMR (400 MHz, CD₃OD) δ =7.46 (d, J=6.8 Hz, 2H), 7.40-7.34 (m, 4H), 7.33 (d, J=8.8 Hz, 2H), 7.17-7.12 (m, 2H), 7.04-7.00 (m, 4H), 6.96-6.91 (m, 5H), 6.85-6.76 (m, 2H), 5.33 (s, 2H), 4.62 (s, 2H), 2.26 (s, 3H). MS calculated for C₃₇H₃₀NO₆S (M + H⁺) 616.17, found 616.00. |
| H5 | 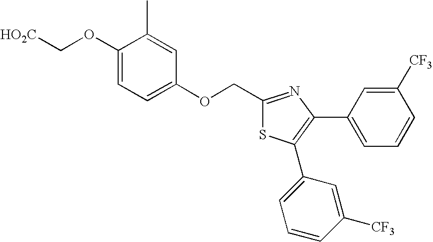 | ¹H-NMR (400 MHz, CD₃OD) δ =7.73-7.69 (m, 3H), 7.64-7.58 (m, 4H), 7.54-7.50 (m, 1H), 6.94 (d, J=2.8 Hz, 1H), 6.87-6.77 (m, 2H), 5.39 (s, 2H), 4.62 (s, 2H), 2.26 (s, 3H). MS calculated for C₂₇H₂₀F₆NO₄S (M + H⁺) 568.09, found 568.00. |
| H6 | 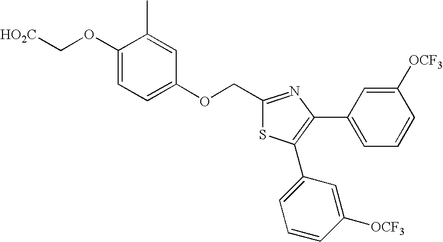 | ¹H-NMR (400 MHz, CD₃OD) δ =7.53-7.37 (m, 4H), 7.32-7.30 (m, 2H), 7.25 (d, J=8.0 Hz, 2H), 7.19 (s, 1H), 6.93 (d, J=2.8 Hz, 1H), 6.86-6.77 (m, 2H), 5.38 (s, 2H), 4.63 (s, 2H), 2.26 (s, 3H). MS calculated for C₂₇H₂₀F₆NO₆S (M + H⁺) 600.08, found 600.00. |
| H7 | 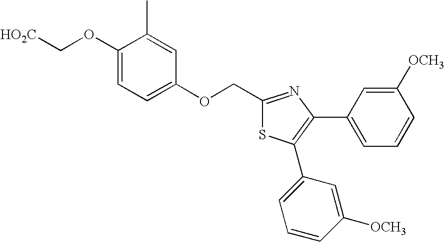 | ¹H-NMR (400 MHz, CD₃OD) δ =7.25 (t, J=8.0 Hz, 2H), 7.18 (t, J=8.0 Hz, 2H), 6.97-6.79 (m, 8H), 6.73-6.71 (m, 1H), 5.33 (s, 2H), 4.57 (s, 2H), 3.62 (s, 3H), 3.58 (s, 3H), 2.12 (s, 3H). MS calculated for C₂₇H₂₆NO₆S (M + H⁺) 492.14, found 492.00. |
| J2 | 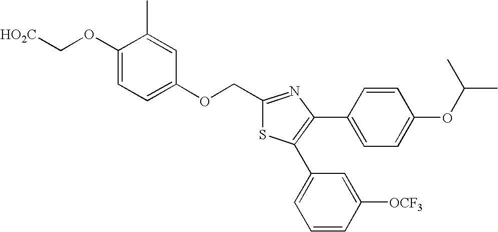 | ¹H-NMR (400 MHz, CD₃OD) δ =7.47 (t, J=8.0 Hz, 1H), 7.39-7.34 (m, 3H), 7.26-7.24 (m, 1H), 7.17 (s, 1H), 6.94 (d, J=3.2 Hz, 1H), 6.89-6.78 (m, 4H), 5.35 (s, 2H), 4.64 (s, 2H), 4.62 (m, 1H), 2.27 (s, 3H), 1.33 (s, 3H), 1.31 (s, 3H). MS calculated for C₂₉H₂₇F₃NO₆S (M + H⁺) 574.1, found 574.2. |
| J3 | 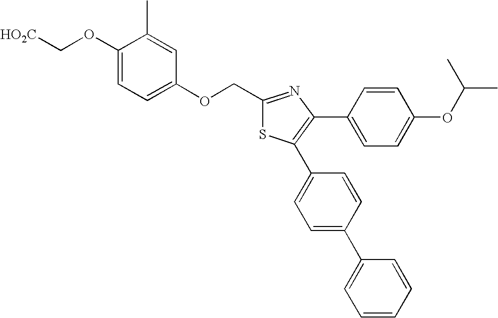 | ¹H-NMR (400 MHz, CD₃OD) δ =7.61-7.57 (m, 4H), 7.40-7.35 (m, 6H), 7.29 (t, J=6.8 Hz, 1H), 6.88 (d, J=2.8 Hz, 1H), 6.80-6.71 (m, 4H), 5.28 (s, 2H), 4.56 (s, 2H), 4.53 (m, 1H), 2.17 (s, 3H), 1.22 (s, 3H), 1.21 (s, 3H). MS calculated for C₂₉H₂₇F₃NO₆S (M + H⁺) 566.2, found 566.2. |
| J4 | 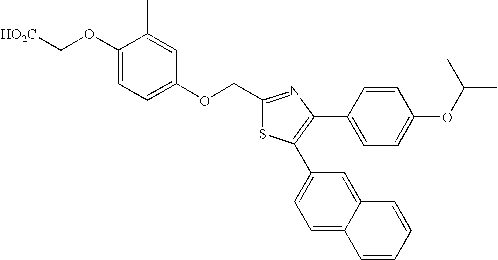 | ¹H-NMR (400 MHz, CD₃OD) δ =7.79-7.68 (m, 4H), 7.42-7.37 (m, 2H), 7.30-7.23 (m, 3H), 6.84 (d, J=2.8 Hz, 1H), 6.77-6.67 (m, 4H), 5.26 (s, 2H), 4.53 (s, 2H), 4.48 (m, 1H), 2.17 (s, 3H), 1.20 (s, 3H), 1.18 (s, 3H). MS calculated for C₂₉H₂₇F₃NO₆S (M + H⁺) 540.2, found 540.2. |
| J5 | 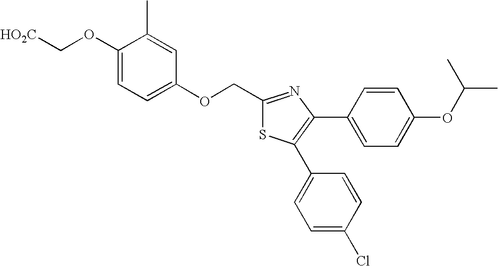 | ¹H-NMR (400 MHz, CD₃OD) δ =7.29-7.22 (m, 6H), 6.83 (d, J=2.4 Hz, 1H), 6.77-6.68 (m, 4H), 5.25 (s, 2H), 4.54 (s, 2H), 4.51 (m, 1H), 2.16 (s, 3H), 1.22 (s, 3H), 1.20 (s, 3H). MS calculated for C₂₈H₂₇ClNO₅S (M + H⁺) 524.1, found 524.1. |
| J6 | 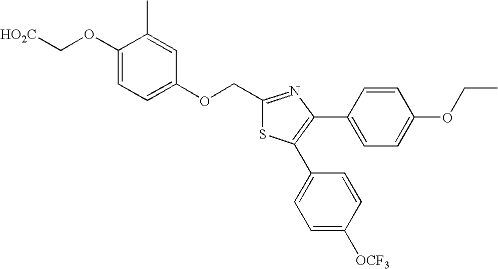 | ¹H-NMR (400 MHz, CD₃OD) δ =7.36-7.16 (m, 6H), 6.85 (d, J=2.8 Hz, 1H), 6.79-6.68 (m, 4H), 5.26 (s, 2H), 4.54 (s, 2H), 3.97-3.91 (m, 2H), 2.16 (s, 3H), 1.28 (t, J=7.2 Hz, 3H). MS calculated for C₂₈H₂₅F₃NO₆S (M + H⁺) 560.1, found 560.2. |
| J7 | 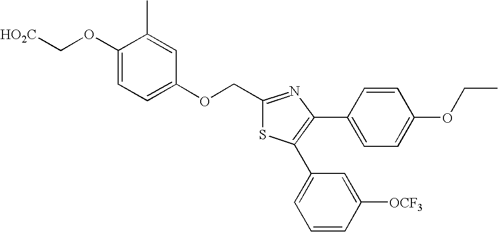 | ¹H-NMR (400 MHz, CD₃OD) δ =7.36-7.00 (m, 6H), 6.80 (d, J=2.8 Hz, 1H), 6.76-6.64 (m, 4H), 5.22 (s, 2H), 4.49 (s, 2H), 3.95-3.89 (m, 2H), 2.14 (s, 3H), 1.26 (t, J=7.2 Hz, 3H). MS calculated for C₂₈H₂₅F₃NO₆S (M + H⁺) 560.1, found 560.2. |
| J8 | 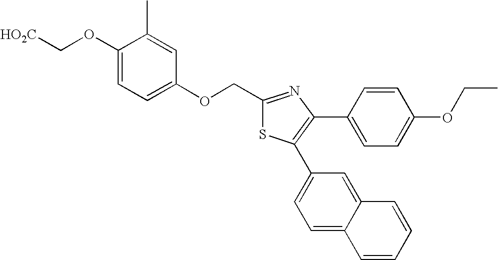 | ¹H-NMR (400 MHz, CD₃OD) δ =7.80 (s, 1H), 7.77-7.69 (m, 3H), 7.43-7.40 (m, 2H), 7.31 (d, J=8.8 Hz, 2H), 7.26 (dd, J=1.8 Hz, J=8.4 Hz, 1H), 6.85 (d, J=2.8 Hz, 1H), 6.78-6.68 (m, 4H), 5.27 (s, 2H), 4.53 (s, 2H), 3.95-3.89 (m, 2H), 2.18 (s, 3H), 1.26 (t, J=7.0 Hz, 3H). MS calculated for C₃₁H₂₈NO₅S (M + H⁺) 526.2, found 526.2. |
| J9 | 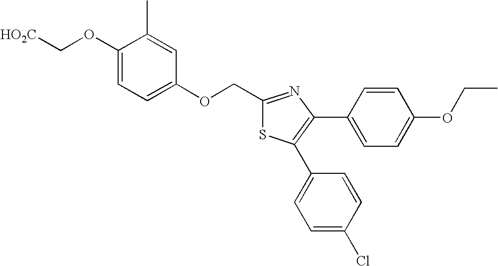 | ¹H-NMR (400 MHz, CD₃OD) δ =7.27-7.19 (m, 6H), 6.82 (d, J=2.8 Hz, 1H), 6.78-6.67 (m, 4H), 5.23 (s, 2H), 4.53 (s, 2H), 3.96-3.91 (m, 2H), 2.16 (s, 3H), 1.29 (t, J=7.0 Hz, 3H). MS calculated for C₂₇H₂₅ClNO₅S (M + H⁺) 510.1, found 510.1. |
| J10 | 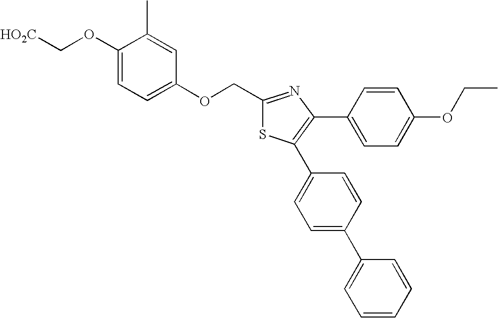 | ¹H-NMR (400 MHz, CD₃OD) δ =7.54-7.49 (m, 4H), 7.34-7.23 (m, 7H), 6.82 (d, J=2.8 Hz, 1H), 6.76-6.65 (m, 4H), 5.22 (s, 2H), 4.50 (s, 2H), 3.92-3.87 (m, 2H), 2.12 (s, 3H), 1.23 (t, J=7.0 Hz, 3H). MS calculated for C₃₃H₃₀NO₅S (M + H⁺) 552.2, found 552.2. |
| J11 | 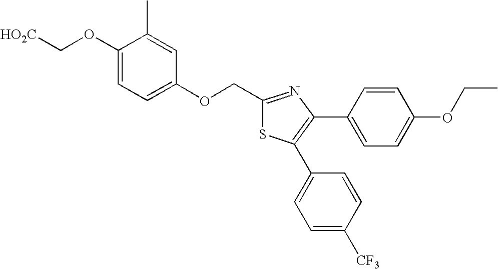 | ¹H-NMR (400 MHz, CD₃OD) δ =7.55 (d, J=8.4 Hz, 2H), 7.43 (d, J=8.0 Hz, 2H), 7.27 (d, J=8.8 Hz, 2H), 6.83 (d, J=2.8 Hz, 1H), 6.80-6.67 (m, 4H), 5.25 (s, 2H), 4.53 (s, 2H), 3.96-3.91 (m, 2H), 2.16 (s, 3H), 1.29 (t, J=7.0 Hz, 3H). MS calculated for C₂₈H₂₅F₃NO₅S (M + H⁺) 544.1, found 544.1. |
| J12 | 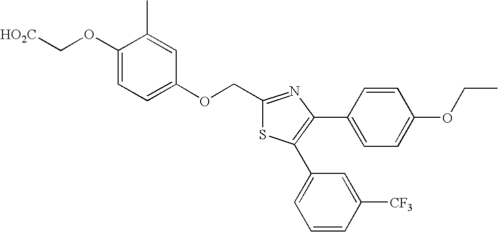 | ¹H-NMR (400 MHz, CD₃OD) δ =7.54-7.42 (m, 4H), 7.25 (d, J=8.8 Hz, 2H), 6.83 (d, J=2.8 Hz, 1H), 6.79-6.67 (m, 4H), 5.25 (s, 2H), 4.53 (s, 2H), 3.96-3.91 (m, 2H), 2.16 (s, 3H), 1.28 (t, J=7.0 Hz, 3H). MS calculated for C₂₈H₂₅F₃NO₅S (M + H⁺) 544.1, found 544.1. |
| J13 | 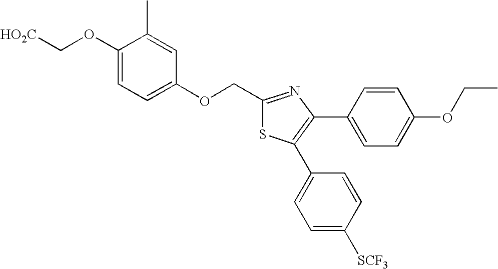 | ¹H-NMR (400 MHz, CD₃OD) δ =7.60 (d, J=8.0 Hz, 2H), 7.39 (d, J=8.4 Hz, 2H), 7.30 (d, J=8.8 Hz, 2H), 6.85 (d, J=2.8 Hz, 1H), 6.80-6.69 (m, 4H), 5.27 (s, 2H), 4.54 (s, 2H), 3.98-3.93 (m, 2H), 2.17 (s, 3H), 1.29 (t, J=7.0 Hz, 3H). MS calculated for C₂₈H₂₅F₃NO₅S₂ (M + H⁺) 576.1, found 576.1. |
| J14 | 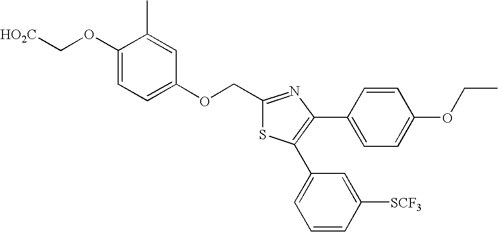 | ¹H-NMR (400 MHz, CD₃OD) δ =7.58-7.45 (m, 4H), 7.27 (d, J=8.8 Hz, 2H), 6.85 (d, J=2.8 Hz, 1H), 6.81-6.68 (m, 4H), 5.25 (s, 2H), 4.53 (s, 2H), 3.96-3.91 (m, 2H), 2.16 (s, 3H), 1.29 (t, J=7.0 Hz, 3H). MS calculated for C₂₈H₂₅F₃NO₅S₂ (M + H⁺) 576.1, found 576.1. |
| J15 | 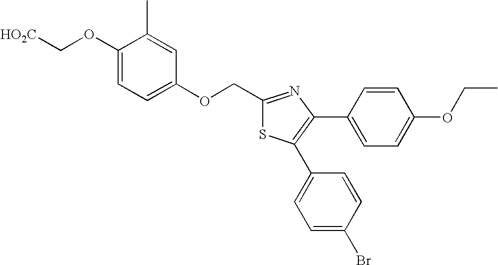 | ¹H-NMR (400 MHz, CD₃OD) δ =7.45 (d, J=8.4 Hz, 2H), 7.30 (d, J=8.8 Hz, 2H), 7.19 (d, J=8.8 Hz, 2H), 6.85 (d, J=2.8 Hz, 1H), 6.80-6.69 (m, 4H), 5.26 (s, 2H), 4.54 (s, 2H), 3.97-3.92 (m, 2H), 2.16 (s, 3H), 1.28 (t, J=7.0 Hz, 3H). MS calculated for C₂₇H₂₅BrNO₅S (M + H⁺) 554.1, found 554.1. |
| J16 | 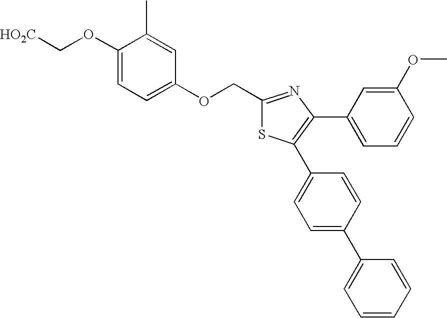 | ¹H-NMR (400 MHz, CD₃OD) δ =7.56-7.51 (m, 5H), 7.37-7.26 (m, 6H), 7.14 (t, J=7.6 Hz, 2H), 6.85 (d, J=2.8 Hz, 1H), 6.81-6.68 (m, 3H), 5.27 (s, 2H), 4.54 (s, 2H), 3.59 (s, 3H), 2.18 (s, 3H). MS calculated for C₃₂H₂₈NO₅S (M + H⁺) 538.2, found 538.2. |
| J17 | 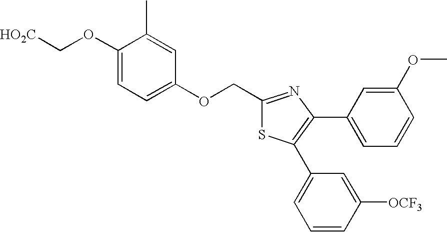 | ¹H-NMR (400 MHz, CD₃OD) δ =7.38 (t, J=8.0 Hz, 1H), 7.29-7.27 (m, 1H), 7.18-7.12 (m, 2H), 7.07 (s, 1H), 7.92-6.79 (m, 4H), 6.74-6.67 (m, 2H), 5.26 (s, 2H), 4.53 (s, 2H), 3.59 (s, 3H), 2.17 (s, 3H). MS calculated for C₂₇H₂₃F₃NO₆S (M + H⁺) 546.1, found 546.1. |
| J18 | 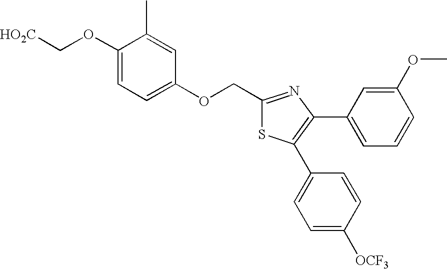 | ¹H-NMR (400 MHz, CD₃OD) δ =7.34 (d, J=8.8 Hz, 2H), 7.18-7.11 (m, 3H), 6.93-6.88 (m, 2H), 6.82 (d, J=2.8 Hz, 1H), 6.80-6.66 (m, 3H), 5.25 (s, 2H), 4.52 (s, 2H), 3.57 (s, 3H), 2.16 (s, 3H). MS calculated for C₂₇H₂₃F₃NO₆S (M + H⁺) 546.1, found 546.1. |
| J19 | 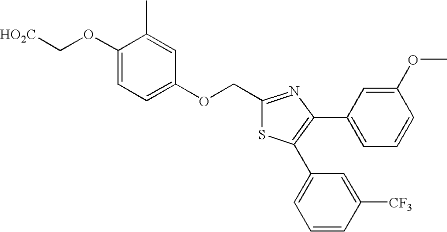 | ¹H-NMR (400 MHz, CD₃OD) δ =7.55 (t, J=8.0 Hz, 2H), 7.44-7.38 (m, 2H), 7.16-7.11 (m, 1H), 6.91-6.67 (m, 6H), 5.27 (s, 2H), 4.53 (s, 2H), 3.59 (s, 3H), 2.16 (s, 3H). MS calculated for C₂₇H₂₃F₃NO₅S (M + H⁺) 530.1, found 530.1. |
| J20 | 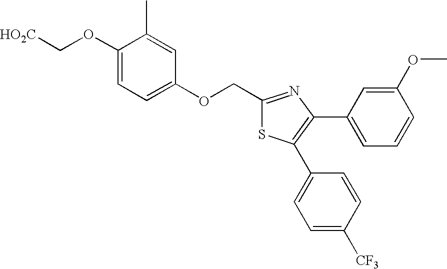 | ¹H-NMR (400 MHz, CD₃OD) δ =7.56-7.45 (m, 4H), 7.12 (t, J=8.4 Hz, 1H), 6.89-6.66 (m, 6H), 5.26 (s, 2H), 4.52 (s, 2H), 3.57 (s, 3H), 2.15 (s, 3H). MS calculated for C₂₇H₂₃F₃NO₅S (M + H⁺) 530.1, found 530.1. |
| J21 | 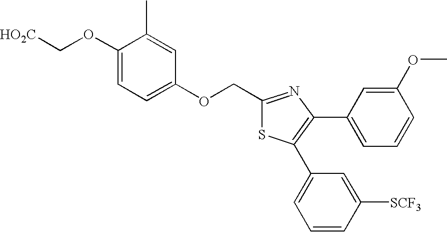 | ¹H-NMR (400 MHz, CD₃OD) δ =7.59 (t, J=8.0 Hz, 1H), 7.51 (s, 1H), 7.48-7.41 (m, 2H), 7.13 (t, J=8.0 Hz, 1H), 6.91-6.87 (m, 2H), 6.84 (d, J=2.8 Hz, 1H), 6.81-6.67 (m, 3H), 5.27 (s, 2H), 4.52 (s, 2H), 3.58 (s, 3H), 2.17 (s, 3H). MS calculated for C₂₇H₂₃F₃NO₅S₂(M + H⁺) 562.1, found 562.0. |
| J22 | 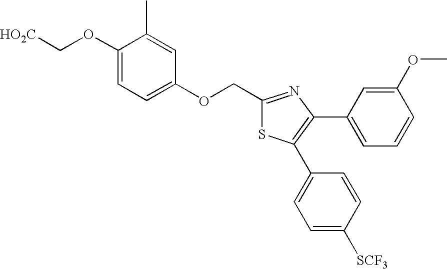 | ¹H-NMR (400 MHz, CD₃OD) δ =7.59 (t, J=8.0 Hz, 2H), 7.38 (t, J=8.4 Hz, 2H), 7.14 (t, J=8.0 Hz, 1H), 6.95-6.87 (m, 2H), 6.83 (d, J=3.2 Hz, 1H), 6.81-6.67 (m, 3H), 5.27 (s, 2H), 4.53 (s, 2H), 3.56 (s, 3H), 2.17 (s, 3H). MS calculated for C₂₇H₂₃F₃NO₅S₂ (M + H⁺) 562.1, found 562.0. |
| J23 | 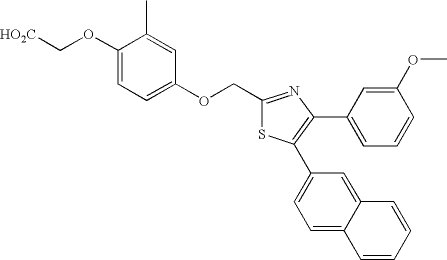 | ¹H-NMR (400 MHz, CD₃OD) δ =7.80 (s, 1H), 7.77-7.68 (m, 4H), 7.42-7.38 (m, 2H), 7.26 (dd, J=1.6 Hz, J=8.4 Hz, 1H), 7.08 (t, J=8.0 Hz, 1H), 6.95-6.93 (m, 2H), 6.84 (d, J=2.8 Hz, 1H), 6.78-6.68 (m, 2H), 5.27 (s, 2H), 4.53 (s, 2H), 3.50 (s, 3H), 2.17 (s, 3H). MS calculated for C₃₀H₂₆NO₅S (M + H⁺) 512.1, found 512.1. |
| J24 | 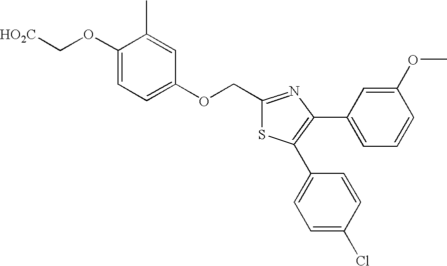 | ¹H-NMR (400 MHz, CD₃OD) δ =7.33-7.25 (m, 4H), 7.15 (t, J=8.0 Hz, 1H), 6.94-6.92 (m, 2H), 6.86 (d, J=2.8 Hz, 1H), 6.82-6.69 (m, 3H), 5.28 (s, 2H), 4.55 (s, 2H), 3.61 (s, 3H), 2.17 (s, 3H). MS calculated for C₂₆H₂₃ClNO₅S (M + H⁺) 496.1, found 496.0. |
| J25 | 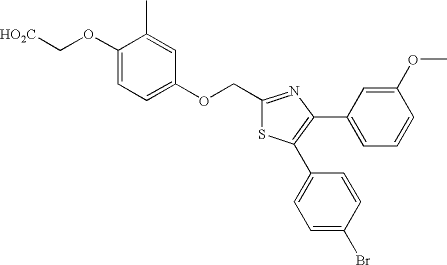 | ¹H-NMR (400 MHz, CD₃OD) δ =7.48 (d, J=8.4 Hz, 2H), 7.21 (d, J=8.4 Hz, 2H), 7.16 (t, J=8.2 Hz, 1H), 6.94-6.92 (m, 2H), 6.87 (d, J=3.2 Hz, 1H), 6.83-6.70 (m, 3H), 5.29 (s, 2H), 4.55 (s, 2H), 3.62 (s, 3H), 2.17 (s, 3H). MS calculated for C₂₆H₂₃BrNO₅S (M + H⁺) 540.0, found 540.1. |
| J26 | 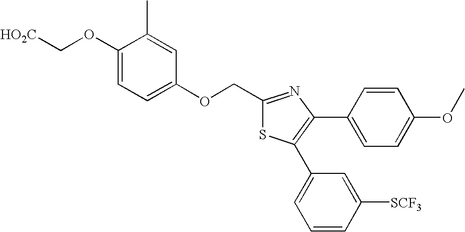 | ¹H-NMR (400 MHz, CD₃OD) δ =7.57 (d, J=8.0 Hz, 1H), 7.50 (s, 1H), 7.47-7.37 (m, 2H), 7.27 (d, J=8.8 Hz, 2H), 6.83 (d, J=2.8 Hz, 1H), 6.79 (d, J=8.8 Hz, 2H), 6.76-6.67 (m, 2H), 5.25 (s, 2H), 4.53 (s, 2H), 3.70 (s, 3H), 2.17 (s, 3H). MS calculated for C₂₇H₂₃F₃NO₅S₂ (M + H⁺) 562.1, found 562.0. |
| J27 | 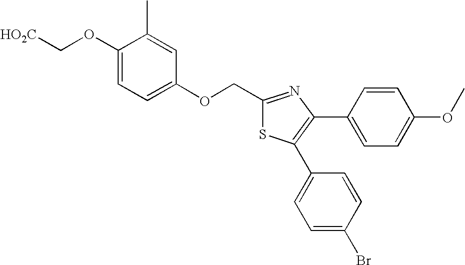 | ¹H-NMR (400 MHz, CD₃OD) δ =7.48 (d, J=8.4 Hz, 2H), 7.33 (d, J=8.8 Hz, 2H), 7.21 (d, J=8.4 Hz, 2H), 6.87 (d, J=2.4 Hz, 1H), 6.84 (d, J=8.8 Hz, 2H), 6.80-6.70 (m, 2H), 5.28 (s, 2H), 4.56 (s, 2H), 3.71 (s, 3H), 2.16 (s, 3H). MS calculated for C₂₆H₂₃BrNO₅S (M + H⁺) 540.0, found 540.0. |
| J21 | 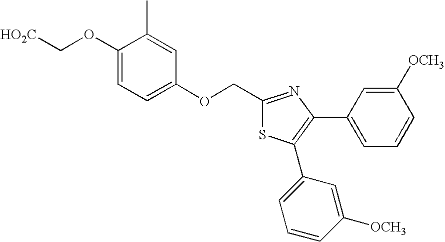 | ¹H-NMR (400 MHz, CD₃OD) δ =7.25 (t, J=8.0 Hz, 2H), 7.18 (t, J=8.0 Hz, 2H), 6.97-6.79 (m, 8H), 6.73-6.71 (m, 1H), 5.33 (s, 2H), 4.57 (s, 2H), 3.62 (s, 3H), 3.58 (s, 3H), 2.12 (s, 3H). MS calculated for C₂₇H₂₆NO₆S (M + H⁺) 492.14, found 492.00. |
| K2 | 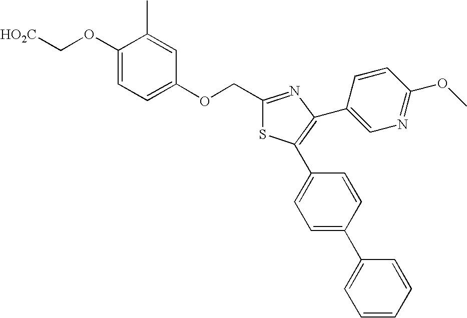 | ¹H-NMR (400 MHz, CD₃OD) δ =8.16 (d, J=2.0 Hz, 1H), 7.75 (dd, J=2.4 Hz, J=8.8 Hz, 1H), 7.57-7.54 (m, 4H), 7.37-7.30 (m, 4H), 7.28-7.24 (m, 1H), 6.84 (d, J=2.8 Hz, 1H), 6.77-6.68 (m, 3H), 5.27 (s, 2H), 4.53 (s, 2H), 3.81 (s, 3H), 2.17 (s, 3H). MS calculated for C₃₁H₂₇N₂O₅S (M + H⁺) 539.2, found 539.1. |
| K3 | 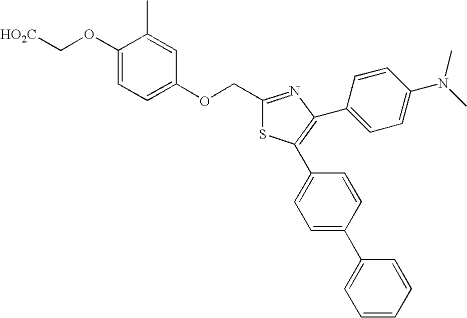 | ¹H-NMR (400 MHz, CD₃OD) δ =7.58-7.55 (m, 4H), 7.41-7.34 (m, 4H), 7.29-7.26 (m, 1H), 6.91 (d, J=8.4 Hz, 2H), 6.86 (d, J=2.8 Hz, 1H), 6.79-6.69 (m, 2H), 5.27 (s, 2H), 4.55 (s, 2H), 2.96 (s, 6H), 2.17 (s, 3H). MS calculated for C₃₃H₃₁N₂O₄S (M + H⁺) 551.2, found 551.2. |
| K4 | 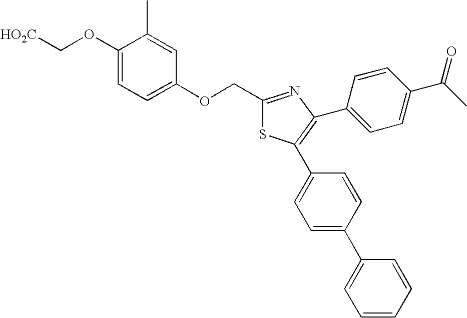 | ¹H-NMR (400 MHz, CD₃OD) δ =7.88 (d, J=8.4 Hz, 4H), 7.58-7.55 (m, 6H), 7.37-7.32 (m, 4H), 7.28-7.25 (m, 1H), 6.85 (d, J=2.8 Hz, 1H), 6.78-6.68 (m, 2H), 5.29 (s, 2H), 4.53 (s, 2H), 2.50 (s, 3H), 2.18 (s, 3H). MS calculated for C₃₃H₂₈NO₅S (M + H⁺) 550.2, found 550.2. |
| K5 | 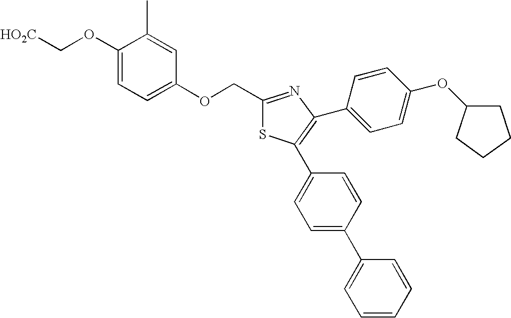 | ¹H-NMR (400 MHz, CD₃OD) δ =7.59-7.55 (m, 4H), 7.37-7.33 (m, 6H), 7.29-7.26 (m, 1H), 6.86 (d, J=2.8 Hz, 1H), 6.78-6.68 (m, 2H), 5.27 (s, 2H), 4.55 (s, 2H), 4.72 9m, 1H), 2.50 (s, 3H), 2.18 (s, 3H), 1.90-1.52 (m, 8H). MS calculated for C₃₆H₃₄NO₅S (M + H⁺) 592.2, found 592.2. |
| K6 | 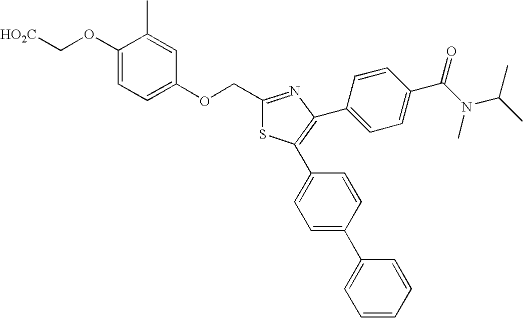 | ¹H-NMR (400 MHz, CD₃OD) δ =7.54-7.51 (m, 6H), 7.36-7.31 (m, 4H), 7.28-7.24 (m, 3H), 6.84 (d, J=2.8 Hz, 1H), 6.77-6.67 (m, 2H), 5.27 (s, 2H), 4.53 (s, 2H), 3.92-3.86 (m, 1H), 2.83-2.74 (m, 3H), 2.17 (s, 3H), 1.14-1.07 (m, 6H). MS calculated for C₃₆H₃₅N₂O₅S (M + H⁺) 607.2, found 607.3. |
| K7 | 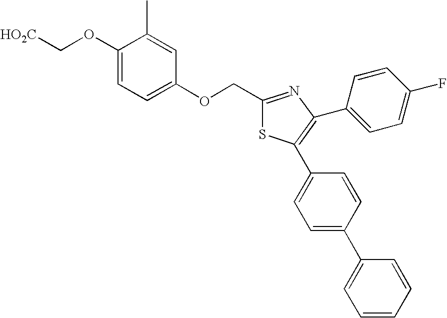 | ¹H-NMR (400 MHz, CD₃OD) δ =7.59-7.56 (m, 5H), 7.38-7.25 (m, 7H), 7.10 (t, J=8.8 Hz, 1H), 6.85 (d, J=2.8 Hz, 1H), 6.78-6.69 (m, 2H), 5.27 (s, 2H), 4.54 (s, 2H), 2.18 (s, 3H). MS calculated for C₃₁H₂₅FNO₄S (M + H⁺) 526.1, found 526.1. |
| K8 | 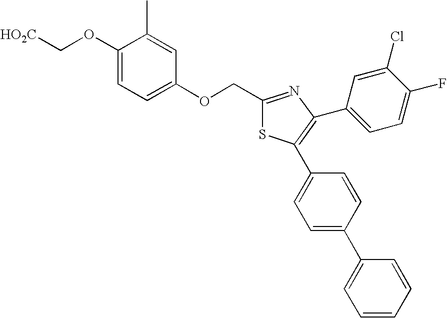 | ¹H-NMR (400 MHz, CD₃OD) δ =7.62 (d, J=8.4 Hz, 4H), 7.49-7.30 (m, 7H), 7.07-7.02 (m, 2H), 6.89 (d, J=2.8 Hz, 1H), 6.82-6.71 (m, 2H), 5.30 (s, 2H), 4.56 (s, 2H), 2.17 (s, 3H). MS calculated for C₃₁H₂₄CIFNO₄S (M + H⁺) 560.1, found 560.1. |
| K9 | 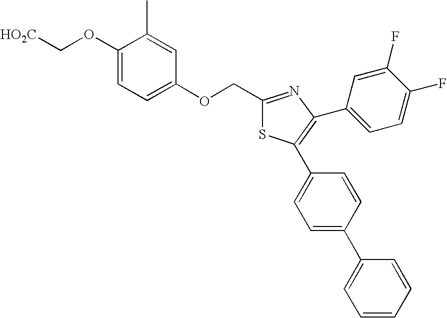 | ¹H-NMR (400 MHz, CD₃OD) δ =7.63-7.60 (m, 4H), 7.41-7.15 (m, 8H), 6.87 (d, J=2.8 Hz, 1H), 6.80-6.70 (m, 2H), 5.30 (s, 2H), 4.54 (s, 2H), 2.17 (s, 3H). MS calculated for C₃₁H₂₄F₂NO₄S (M + H⁺) 544.1, found 544.1. |
| K10 | 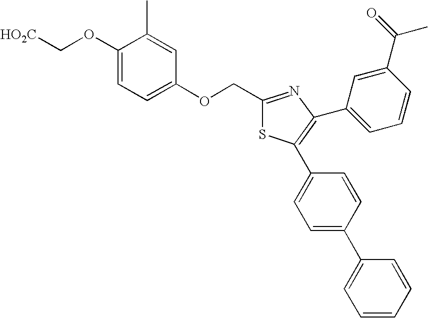 | ¹H-NMR (400 MHz, CD₃OD) δ =8.06 (s, 1H), 7.85 (d, J=7.6 hz, 1H), 7.69 (d, J=7.6 hz, 1H), 7.58-7.54 (m, 4H), 7.40-7.25 (m, 6H), 6.86 (d, J=2.4 Hz, 1H), 6.79-6.69 (m, 2H), 5.30 (s, 2H), 4.54 (s, 2H), 2.37 (s, 3H), 2.18 (s, 3H). MS calculated for C₃₃H₂₈NO₅S (M + H⁺) 550.2, found 550.2. |
| K11 | 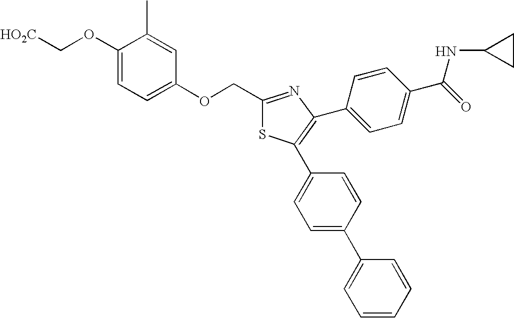 | ¹H-NMR (400 MHz, CD₃OD) δ =7.67 (d, J=8.4 Hz, 2H), 7.54-7.48 (m, 5H), 7.36-7.24 (m, 6H), 6.84 (d, J=2.8 Hz, 1H), 6.77-6.67 (m, 2H), 5.26 (s, 2H), 4.53 (s, 2H), 2.80-2.72 (m, 1H), 2.17 (s, 3H), 0.70-0.53 (m, 4H). MS calculated for C₃₅H₃₁N₂O₅S (M + H⁺) 591.2, found 591.2. |
| K12 | 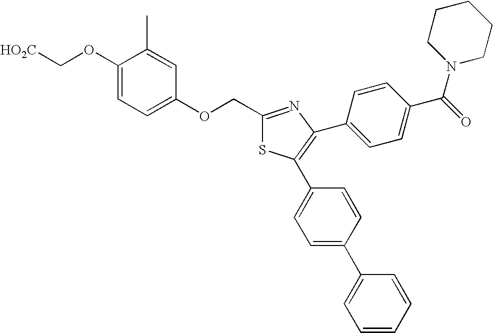 | ¹H-NMR (400 MHz, CD₃OD) δ =7.55-7.51 (m, 7H), 7.37-7.26 (m, 6H), 6.84 (d, J=2.8 Hz, 1H), 6.77-6.68 (m, 2H), 5.28 (s, 2H), 4.54 (s, 2H), 3.64-3.23 (m, 4H), 2.18 (s, 3H), 1.72-1.40 (m, 6H). MS calculated for C₃₇H₃₅N₂O₅S (M + H⁺) 619.2, found 619.2. |
| K13 | 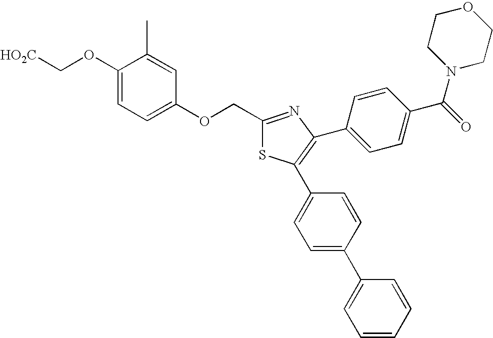 | ¹H-NMR (400 MHz, CD₃OD) δ =7.59-7.53 (m, 6H), 7.39-7.26 (m, 7H), 6.87 (d, J=2.4 Hz, 1H), 6.80-6.69 (m, 2H), 5.29 (s, 2H), 4.55 (s, 2H), 3.70-3.25 (m, 8H), 2.17 (s, 3H). MS calculated for C₃₆H₃₃N₂O₆S (M + H⁺) 621.2, found 621.2. |
| K14 | 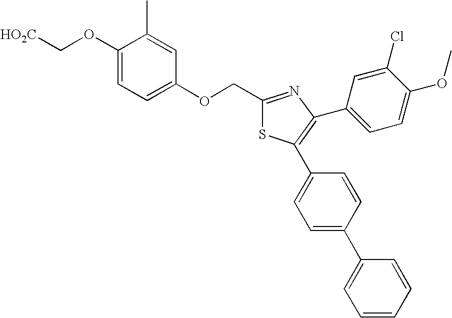 | ¹H-NMR (400 MHz, CD₃OD) δ =7.56 (d, J=8.0 Hz, 4H), 7.48 (d, J=1.6 Hz, 1H), 7.37-7.25 (m, 6H), 6.93 (d, J=8.4 Hz, 4H), 6.84 (d, J=2.8 Hz, 1H), 6.77-6.68 (m, 2H), 5.26 (s, 2H), 4.54 (s, 2H), 3.79 (s, 3H), 2.18 (s, 3H). MS calculated for C₃₂H₂₇ClNO₅S (M + H⁺) 572.1, found 572.1. |
| K15 | 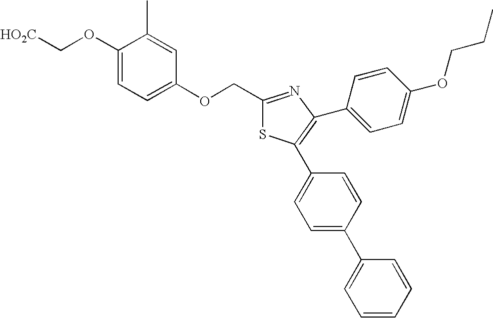 | ¹H-NMR (400 MHz, CD₃OD) δ =7.65-7.57 (m, 4H), 7.40-7.29 (m, 7H), 6.87 (d, J=2.4 Hz, 1H), 6.82-6.70 (m, 4H), 5.28 (s, 2H), 4.54 (s, 2H), 3.85 (t, J=6.6 Hz, 2H), 2.17 (s, 3H), 1.71-1.66 (m, 2H), 0.94 (t, J=7.0 Hz, 3H). MS calculated for C₃₄H₃₂NO₅S (M + H⁺) 566.2, found 566.1. |
| K16 | 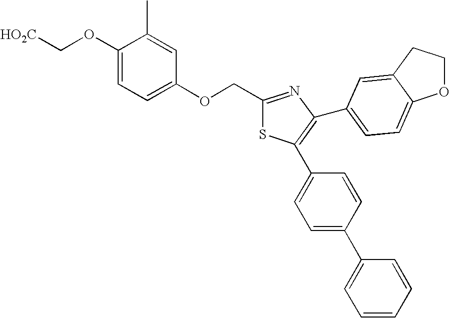 | ¹H-NMR (400 MHz, CD₃OD) δ =7.58-7.54 (m, 4H), 7.39-7.26 (m, 6H), 7.15 (d, J=8.4 Hz, 1H), 6.86 (d, J=2.8 Hz, 1H), 6.79-6.60 (m, 3H), 5.27 (s, 2H), 4.55 (s, 2H), 4.47 (t, J=8.8 Hz, 2H), 3.10 (t, J=8.4 Hz, 2H), 2.17 (s, 3H). MS calculated for C₃₃H₂₈NO₅S (M + H⁺) 550.2, found 550.1. |
| K17 | 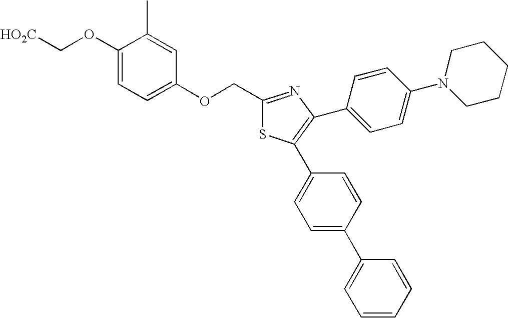 | ¹H-NMR (400 MHz, CD₃OD) δ =7.58-7.54 (m, 6H), 7.38-7.25 (m, 7H), 6.84 (d, J=2.8 Hz, 1H), 6.77-6.68 (m, 2H), 5.27 (s, 2H), 4.54 (s, 2H), 3.43 (t, J=5.4 Hz, 4H), 2.17 (s, 3H), 1.88-1.83 (m, 4H), 1.68-1.64 (m, 2H). MS calculated for C₃₆H₃₅N₂O₄S (M + H⁺) 591.2, found 591.2. |
| K18 | 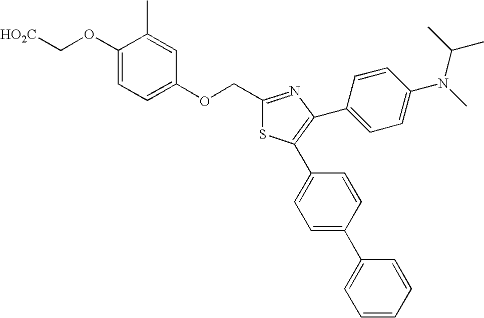 | MS calculated for C₃₅H₃₅N₂O₄S (M + H⁺) 579.2, found 579.2. |
| K19 | 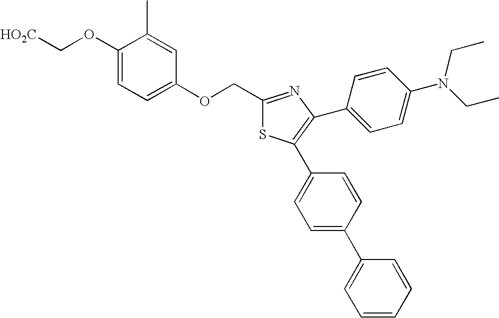 | MS calculated for C₃₅H₃₅N₂O₄S (M + H⁺) 579.2, found 579.2. |
| K20 | 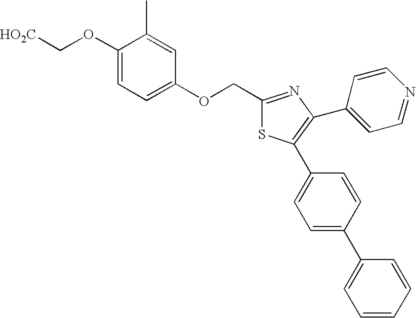 | ¹H-NMR (400 MHz, CD₃OD) δ =8.62 (d, J=6.8 Hz, 1H), 8.07 (d, J=6.4 Hz, 1H), 7.70-7.28 (m, 11H), 6.85 (d, J=2.8 Hz, 1H), 6.78-6.68 (m, 2H), 5.33 (s, 2H), 4.54 (s, 2H), 2.17 (s, 3H). MS calculated for C₃₀H₂₅N₂O₄S (M + H⁺) 509.2, found 509.1. |
| K21 | 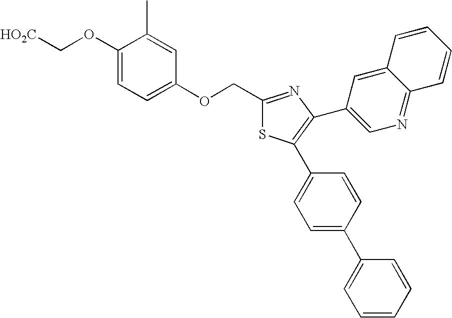 | ¹H-NMR (400 MHz, CD₃OD) δ =9.08 (s, 1H), 8.80 (s, 1H), 8.16-8.08 (m, 2H), 7.96 (t, J=7.6 Hz, 1H), 7.83-7.77 (m, 4H), 7.69-7.46 (m, 4H), 7.08 (d, J=2.4 Hz, 1H), 7.02-6.89 (m, 2H), 5.55 (s, 2H), 4.74 (s, 2H), 2.34 (s, 3H). MS calculated for C₃₄H₂₇N₂O₄S (M + H⁺) 559.2, found 559.2. |
| K22 | 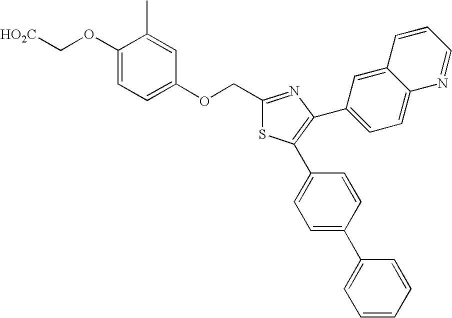 | ¹H-NMR (400 MHz, CD₃OD) δ =9.00 (d, J=4.0 Hz, 1H), 8.82-8.79 (m, 1H), 8.36 (s, 1H), 8.09-8.00 (m, 2H), 7.85-7.81 (m, 1H), 7.58-7.25 (m, 9H), 6.86 (d, J=3.2 Hz, 1H), 6.79-6.69 (m, 2H), 5.32 (s, 2H), 4.54 (s, 2H), 2.18 (s, 3H). MS calculated for C₃₄H₂₇N₂O₄S (M + H⁺) 559.2, found 559.1. |
| K23 | 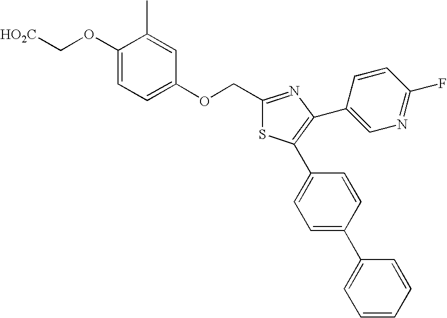 | ¹H-NMR (400 MHz, CD₃OD) δ =8.26 (s, 1H), 8.05-8.00 (m, 1H), 7.64-7.28 (m, 9H), 7.03 (dd, J=2.4 Hz, J=8.8 Hz, 1H), 6.88 (d, J=2.8 Hz, 1H), 6.81-6.70 (m, 2H), 5.32 (s, 2H), 4.54 (s, 2H), 2.17 (s, 3H). MS calculated for C₃₀H₂₄FN₂O₄S (M + H⁺) 527.1, found 527.1. |
| K24 | 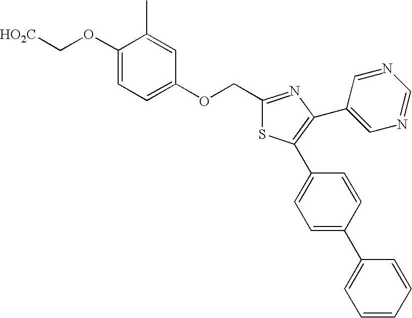 | ¹H-NMR (400 MHz, CD₃OD) δ =9.01 (s, 1H), 8.81 (s, 2H), 7.68-7.61 (m, 4H), 7.44-7.30 (m, 5H), 6.89 (d, J=2.4 Hz, 1H), 6.82-6.71 (m, 2H), 5.35 (s, 2H), 4.55 (s, 2H), 2.17 (s, 3H). MS calculated for C₂₉H₂₄N₃O₄S (M + H⁺) 510.1, found 510.1. |
| K25 | 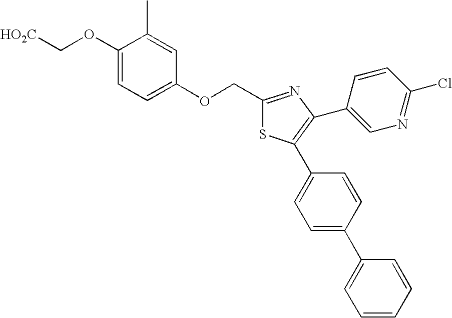 | ¹H-NMR (400 MHz, CD₃OD) δ =8.40 (d, J=2.0 Hz, 1H), 7.86 (dd, J=2.6 Hz, J=8.2 Hz, 1H), 7.61-7.55 (m, 4H), 7.37-7.25 (m, 6H), 6.83 (d, J=2.8 Hz, 1H), 6.77-6.67 (m, 2H), 5.29 (s, 2H), 4.48 (s, 2H), 2.17 (s, 3H). MS calculated for C₃₀H₂₄ClN₂O₄S (M + H⁺) 543.1, found 543.1. |
| K26 | 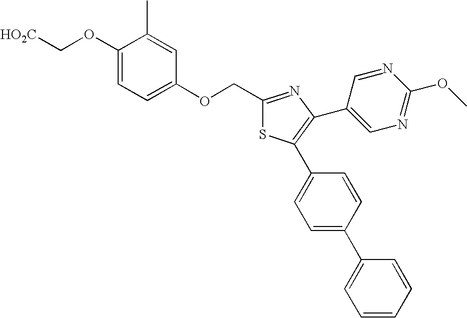 | ¹H-NMR (400MLHz, CD₃OD) δ =8.40 (d, J=2.0 Hz, 1H), 7.86 (dd, J=2.6 Hz, J=8.2 Hz, 1H), 7.61-7.55 (m, 4H), 7.37-7.25 (m, 6H), 6.83 (d, J=2.8 Hz, 1H), 6.77-6.67 (m, 2H), 5.29 (s, 2H), 4.48 (s, 2H), 2.17 (s, 3H). MS calculated for C₃₀H₂₆N₃O₅S (M + H⁺) 540.2, found 540.1. |
| K27 | 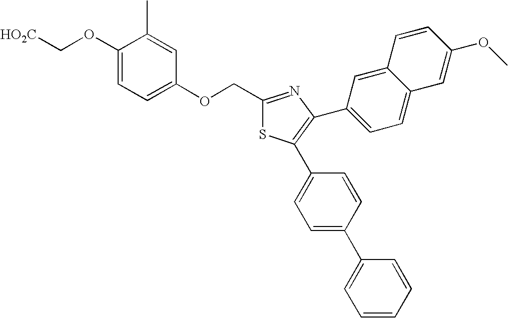 | ¹H-NMR (400 MHz, CD₃OD) δ =7.88 (s, 1H), 7.63-7.50 (m, 6H), 7.42-7.24 (m, 6H), 7.13 (d, J=2.8 Hz, 1H), 7.03 (dd, J=2.6 Hz, J=9.0 Hz, 1H), 6.86 (d, J=2.8 Hz, 1H), 6.77-6.69 (m, 2H), 5.29 (s, 2H), 4.54 (s, 2H), 3.81 (s, 3H), 2.18 (s, 3H). MS calculated for C₃₆H₃₀NO₅S (M + H⁺) 588.2, found 588.2. |
| K28 | 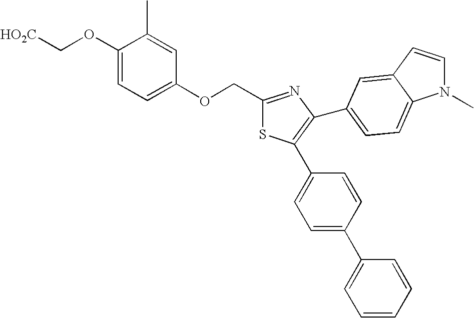 | ¹H-NMR (400 MHz, CD₃OD) δ =7.67 (s, 1H), 7.58-7.53 (m, 4H), 7.39-7.21 (m, 7H), 7.15 (s, 1H), 6.89 (d, J=2.4 Hz, 1H), 6.82-6.71 (m, 2H), 5.30 (s, 2H), 4.56 (s, 2H), 3.72 (s, 3H), 2.18 (s, 3H). MS calculated for C₃₄H₂₉N₂O₄S (M + H⁺) 561.2, found 561.2. |
| K29 | 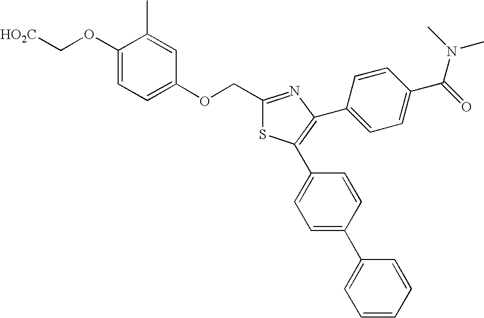 | ¹H-NMR (400 MHz, CD₃OD) δ =7.54-7.51 (m, 6H), 7.36-7.23 (m, 7H), 6.84 (d, J=2.8 Hz, 1H), 6.77-6.68 (m, 2H), 5.27 (s, 2H), 4.53 (s, 2H), 3.00 (s, 3H), 2.92 (s, 3H), 2.17 (s, 3H). MS calculated for C₃₄H₃₁N₂O₅S (M + H⁺) 579.2, found 579.2. |
| K30 | 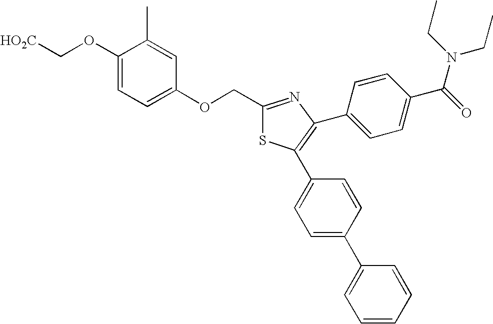 | ¹H-NMR (400 MHz, CD₃OD) δ =7.54-7.51 (m, 6H), 7.36-7.24 (m, 7H), 6.84 (d, J=2.8 Hz, 1H), 6.77-6.68 (m, 2H), 5.27 (s, 2H), 4.53 (s, 2H), 3.47-3.36 (m, 4H), 2.17 (s, 3H), 1.10-0.99 (m, 6H). MS calculated for C₃₄H₃₁N₂O₅S (M + H⁺) 607.2, found 607.2. |
| K31 | 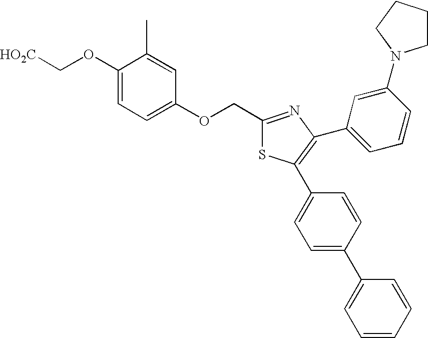 | ¹H-NMR (400 MHz, CD₃OD) δ =7.56 (d, J=8.0 Hz, 4H), 7.38-7.25 (m, 5H), 7.12 (t, J=8.0 Hz, 1H), 6.87-6.61 (m, 6H), 5.27 (s, 2H), 4.54 (s, 2H), 3.14-3.11 (m, 4H), 2.17 (s, 3H), 1.89-1.86 (m, 4H). MS calculated for C₃₅H₃₃N₂O₄S (M + H⁺) 577.2, found 577.2. |
| K32 | 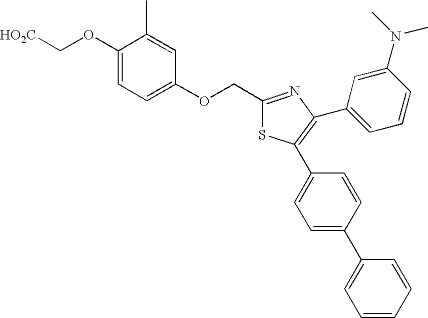 | ¹H-NMR (400 MHz, CD₃OD) δ =7.58-7.53 (m, 4H), 7.37-7.17 (m, 9H), 6.84 (d, J=2.8 Hz, 1H), 6.78-6.68 (m, 2H), 5.28 (s, 2H), 4.54 (s, 2H), 2.99 (s, 6H), 2.17 (s, 3H). MS calculated for C₃₃H₃₁N₂O₄S (M + H⁺) 551.2, found 551.2. |
| K33 | 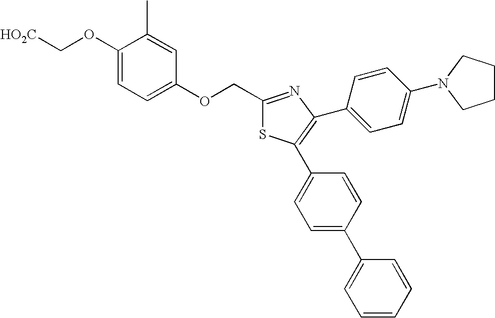 | ¹H-NMR (400 MHz, CD₃OD) δ =7.54-7.49 (m, 4H), 7.36-7.23 (m, 7H), 6.83 (d, J=3.2 Hz, 1H), 6.77-6.68 (m, 2H), 6.50 (d, J=8.4 Hz, 2H), 5.24 (s, 2H), 4.53 (s, 2H), 3.21-3.18 (m, 4H), 2.17 (s, 3H), 1.98-1.92 (m, 4H). MS calculated for C₃₅H₃₃N₂O₄S (M + H⁺) 577.2, found 577.2. |
| K34 | 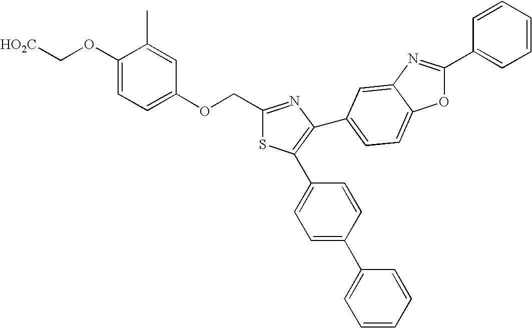 | ¹H-NMR (400 MHz, CD₃OD) δ =8.15 (d, J=7.2 Hz, 4H), 7.78-7.27 (m, 13H), 6.90-6.72 (m, 3H), 5.34 (s, 2H), 4.57 (s, 2H), 2.18 (s, 3H). MS calculated for C₃₈H₂₉N₂O₅S (M + H⁺) 625.2, found 625.2. |
| K35 | 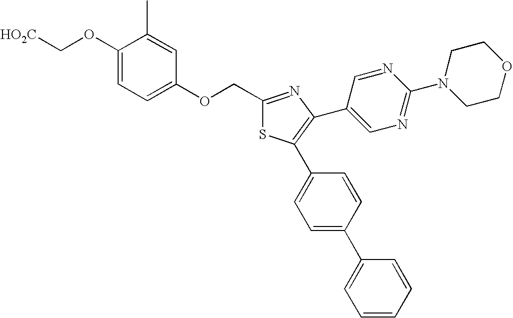 | ¹H-NMR (400 MHz, CDCl₃) δ =8.51 (s, 2H), 7.61-7.37 (m, 9H), 6.92-6.71 (m, 3H), 5.33 (s, 2H), 4.64 (s, 2H), 3.82 (m, 4H), 3.77 (m, 4H), 2.29 (s, 3H). MS calculated for C₃₃H₃₁N₄O₅S (M + H⁺) 595.2, found 595.2. |
| K36 | 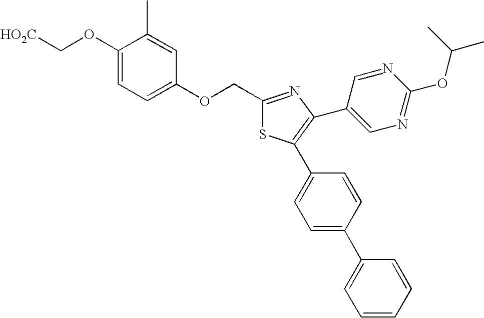 | ¹H-NMR (400 MHz, CDCl₃) δ =8.69 (s, 2H), 7.71-7.38 (m, 9H), 6.91-6.71 (m, 3H), 5.34 (s, 2H), 5.27 (m, 1H), 4.64 (s, 2H), 2.29 (s, 3H), 1.39 (d, J=6.2 Hz, 6H). MS calculated for C₃₂H₃₀N₃O₅S (M + H⁺) 568.2, found 568.2. |
| L2 | 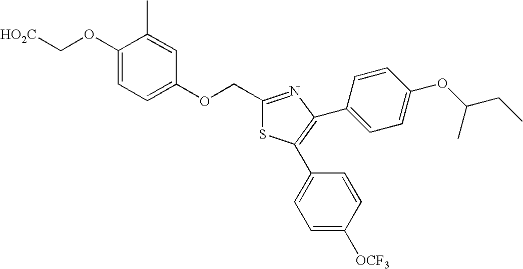 | ¹H-NMR (400 MHz, CD₃OD) δ =7.34 (d, J=8.8 Hz, 2H), 7.26 (d, J=8.8 Hz, 2H), 7.18 (d, J=8.0 Hz, 2H), 6.82 (d, J=2.8 Hz, 1H), 6.77-6.67 (m, 4H), 5.24 (s, 2H), 4.52 (s, 2H), 4.30-4.25 (m, 1H), 2.16 (s, 3H), 1.62-1.52 (m, 2H), 1.18 (d, J=6.0 Hz, 3H), 0.88 (t, J=7.4 Hz, 3H). MS calculated for C₃₀H₂₉F₃NO₆S (M + H⁺588.2, |
| # found 588.1. | | |
| L3 | 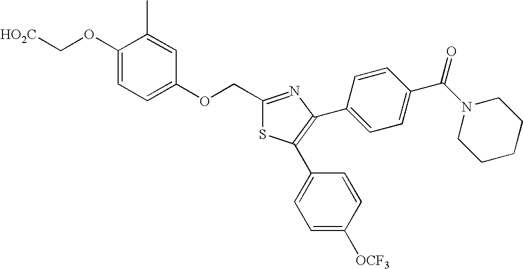 | ¹H-NMR (400 MHz, CD₃OD) δ =7.57 (d, J=8.4 Hz, 2H), 7.46 (d, J=8.8 Hz, 2H), 7.38 (d, J=8.4 Hz, 2H), 7.29 (d, J=8.0 Hz, 2H), 6.93 (d, J=2.8 Hz, 1H), 6.86-6.77 (m, 2H), 5.37 (s, 2H), 4.63 (s, 2H), 3.70 (bs, 2H), 3.39 (bs, 2H), 2.26 (s, 3H), 1.72-1.54 (m, 6H). MS calculated for C₃₂H₃₀F₃N₂O₆S (M + H⁺) 627.2, found 627.1. |
| L4 | 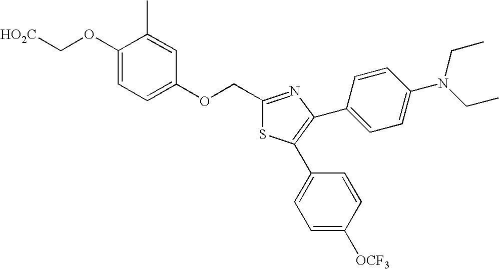 | ¹H-NMR (400 MHz, CD₃OD) δ =7.50 (d, J=8.4 Hz, 2H), 7.38 (d, J=8.4 Hz, 2H), 7.21 (d, J=8.0 Hz, 2H), 7.14 (bs, 2H), 6.83 (d, J=2.8 Hz, 1H), 6.76-6.67 (m, 2H), 5.26 (s, 2H), 4.53 (s, 2H), 3.51 (q, J=7.2 Hz, 4H), 2.16 (s, 3H), 1.05 (t, J=7.0 Hz, 6H). MS calculated for C₃₀H₃₀F₃N₂O₅S (M + H⁺) 587.2, found 587.2. |
| L5 | 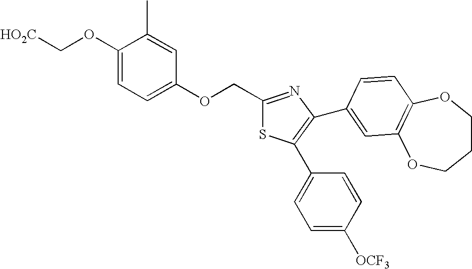 | ¹H-NMR (400 MHz, CD₃OD) δ =7.34 (d, J=8.8 Hz, 2H), 7.19 (d, J=8.0 Hz, 2H), 8.97 (d, J=2.0 Hz, 2H), 6.90-6.77 (m, 3H), 6.75-6.67 (m, 2H), 5.24 (s, 2H), 4.53 (s, 2H), 4.09-4.02 (q, 4H), 2.16 (s, 3H), 2.08-2.03 (m, 2H). MS calculated for C₂₉H₂₅F₃NO₇S (M + H⁺) 588.1, found 588.1. |
| L6 | 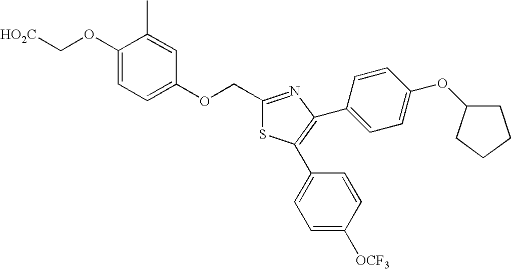 | ¹H-NMR (400 MHz, CD₃OD) δ =7.38 (d, J=8.8 Hz, 2H), 7.29 (d, J=8.8 Hz, 2H), 7.23 (d, J=8.8 Hz, 2H), 6.86 (d, J=2.8 Hz, 1H), 6.79-6.69 (m, 4H), 5.27 (s, 2H), 4.73 (m, 1H), 4.54 (s, 2H), 2.16 (s, 3H), 1.86-1.54 (m, 8H). MS calculated for C₃₁H₂₉F₃NO₆S (M + H⁺) 600.2, found 600.1. |
| L7 | 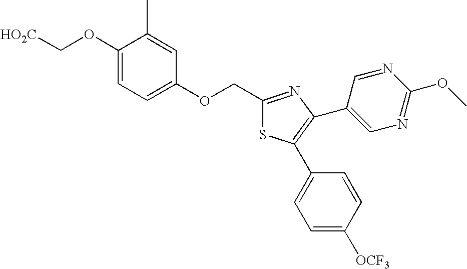 | ¹H-NMR (400 MHz, CD₃OD) δ =8.53 (s, 2H), 7.44 (d, J=8.8 Hz, 2H), 7.28 (d, J=8.8 Hz, 2H), 6.84 (d, J=2.8 Hz, 1H), 6.77-6.68 (m, 2H), 5.29 (s, 2H), 4.54 (s, 2H), 3.93 (s, 3H), 2.17 (s, 3H). MS calculated for C₂₅H₂₁F₃N₃O₆S (M + H⁺) 548.1, found 548.1. |
| L8 | 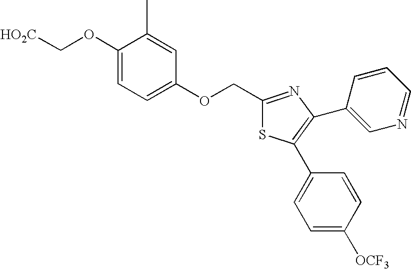 | ¹H-NMR (400 MHz, CD₃OD) δ =8.68 (s, 1H), 8.55 (d, J=5.2 Hz, 1H), 8.11 (dd, J=1.8 Hz, J=8.2 Hz, 1H), 7.60-7.56 (m, 1H), 7.47 (d, J=8.8 Hz, 2H), 7.32 (d, J=8.8 Hz, 2H), 6.88 (d, J=2.8 Hz, 1H), 6.81-6.70 (m, 2H), 5.34 (s, 2H), 4.56 (s, 2H), 2.16 (s, 3H). MS calculated for C₂₅H₂₀F₃N₂O₅S (M + H⁺) 517.1, found 516.9. |
| L9 | 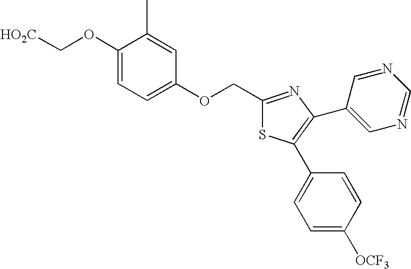 | ¹H-NMR (400 MHz, CD₃OD) δ =9.24 (s, 1H), 8.98 (s, 2H), 7.71 (d, J=8.8 Hz, 2H), 7.54 (d, J=8.8 Hz, 2H), 7.08 (d, J=2.8 Hz, 1H), 7.02-6.90 (m, 2H), 5.56 (s, 2H), 4.67 (s, 2H), 2.39 (s, 3H). MS calculated for C₂₄H₁₉F₃N₃O₅S (M + H⁺) 518.1, found 518.1. |
| L10 | 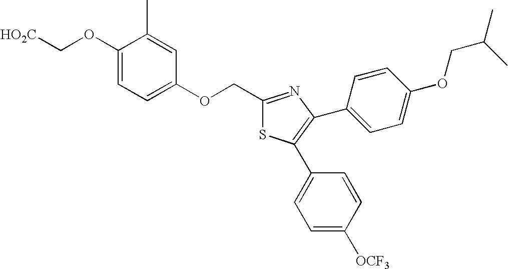 | ¹H-NMR (400 MHz, CD₃OD) δ =7.34 (d, J=8.8 Hz, 2H), 7.27 (d, J=8.8 Hz, 2H), 7.18 (d, J=8.0 Hz, 2H), 6.82 (d, J=2.8 Hz, 1H), 6.80-6.66 (m, 4H), 5.24 (s, 2H), 4.49 (s, 2H), 3.66 (d, J=6.4 Hz, 2H), 2.19 (s, 3H), 2.01-1.91 (m, 1H), 0.94 (s, 3H), 0.93 (s, 3H). MS calculated for C₃₀H₂₉F₃NO₆S (M + H⁺) 588.2, found 588.1. |
| L11 | 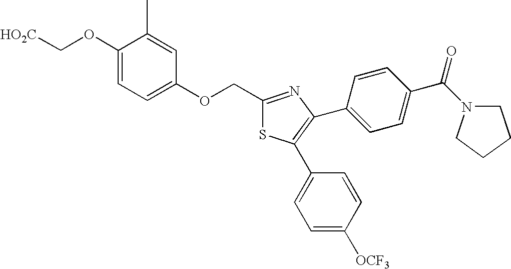 | ¹H-NMR (400 MHz, CD₃OD) δ =7.47-7.40 (m, 4H), 7.37 (d, J=8.8 Hz, 2H), 7.20 (d, J=8.0 Hz, 2H), 6.84 (d, J=2.8 Hz, 1H), 6.77-6.68 (m, 2H), 5.28 (s, 2H), 4.54 (s, 2H), 3.49 (t, J=7.0 Hz, 2H), 3.38 (t, J=6.4 Hz, 2H), 2.17 (s, 3H), 1.93-1.78 (m, 4H). MS calculated for C₃₁H₂₈F₃N₂O₆S (M + H⁺) 613.2, found 613.1. |
| L12 | 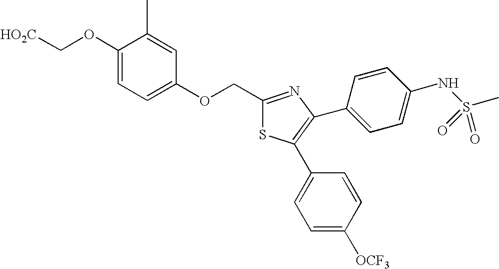 | ¹H-NMR (400 MHz, CD₃OD) δ =7.36-7.32 (m, 4H), 7.20 (d, J=8.8 Hz, 2H), 7.11 (d, J=8.8 Hz, 2H), 6.83 (d, J=2.8 Hz, 1H), 6.76-6.67 (m, 2H), 5.25 (s, 2H), 4.52 (s, 2H), 2.89 (s, 3H), 2.16 (s, 3H). MS calculated for C₂₇H₂₄F₃N₂O₇S₂(M + H⁺) 609.1, found 609.1. |
| L13 | 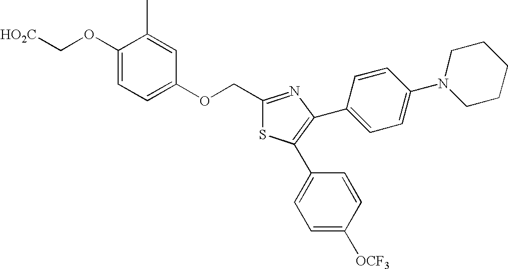 | ¹H-NMR (400 MHz, CD₃OD) δ =7.51 (d, J=8.8 Hz, 2H), 7.37 (d, J=8.8 Hz, 2H), 7.33 (d, J=8.8 Hz, 2H), 7.21 (d, J=8.0 Hz, 2H), 6.83 (d, J=2.8 Hz, 1H), 6.76-6.67 (m, 2H), 5.27 (s, 2H), 4.54 (s, 2H), 3.42 (t, J=5.4 Hz, 4H), 2.17 (s, 3H), 1.87-1.82 (m, 4H), 1.68-1.64 (m, 2H). MS calculated for C₃₁H₃₀F₃N₂O₅S (M + H⁺) |
| # 599.2, found 599.2. | | |
| L14 | 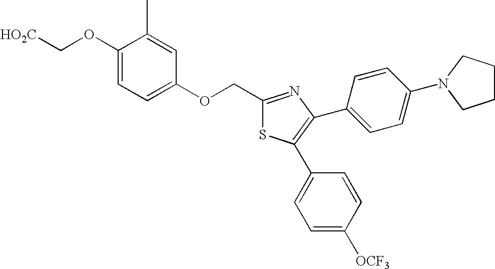 | ¹H-NMR (400 MHz, CD₃OD) δ =7.33 (d, J=8.8 Hz, 2H), 7.20 (d, J=8.8 Hz, 2H), 7.15 (d, J=8.0 Hz, 2H), 6.81 (d, J=2.8 Hz, 1H), 6.74-6.65 (m, 2H), 6.50 (d, J=8.8 Hz, 2H), 5.21 (s, 2H), 4.52 (s, 2H), 3.21 (t, J=5.4 Hz, 4H), 2.15 (s, 3H), 1.95-1.90 (m, 4H). MS calculated for C₃₀H₂₈F₃N₂O₅S (M + H⁺) 585.2, found 585.2. |
| L15 | 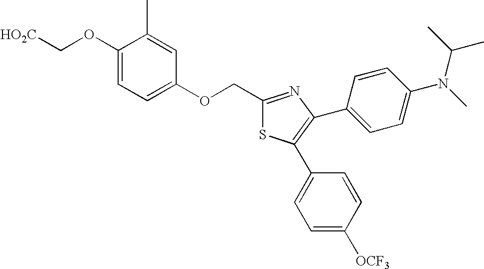 | ¹H-NMR (400 MHz, CD₃OD) δ =7.39-7.36 (m, 4H), 6.86-6.82 (m, 5H), 6.77-6.67 (m, 2H), 5.04 (s, 2H), 4.50 (s, 2H), 4.00-3.94 (m, 1H), 3.01 (s, 3H), 2.16 (s, 3H), 1.24 (s, 3H), 1.23 (s, 3H). MS calculated for C₃₀H₃₀F₃N₂O₅S (M + H⁺) 587.2, found 587.2. |
| L16 | 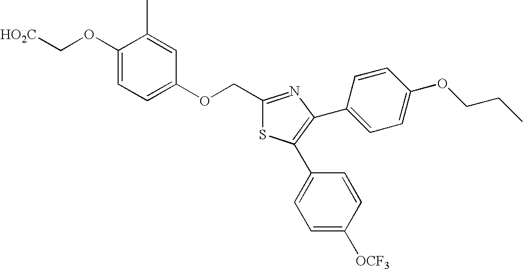 | MS calculated for C₂₉H₂₇F₃NO₆S (M + H⁺) 573.1, found 573.1. |
| L17 | 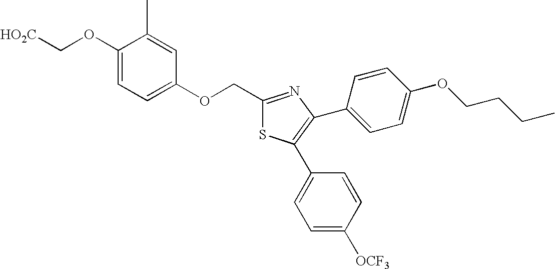 | MS calculated for C₃₀H₂₉F₃NO₆S (M + H⁺) 588.2, found 588.1. |
| L18 | 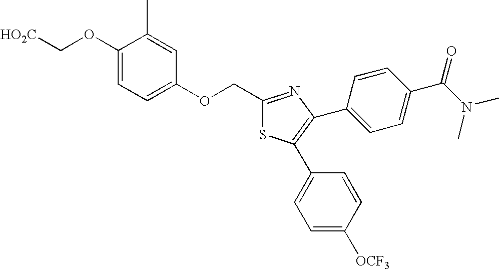 | ¹H-NMR (400 MHz, CD₃OD) δ =7.48 (d, J=8.4 Hz, 2H), 7.37-7.30 (m, 4H), 7.20 (d, J=8.8 Hz, 2H), 6.84 (d, J=2.8 Hz, 1H), 6.77-6.68 (m, 2H), 5.28 (s, 2H), 4.53 (s, 2H), 3.01 (s, 3H), 2.92 (s, 3H), 2.17 (s, 3H). MS calculated for C₂₉H₂₆F₃N₂O₆S (M + H⁺) 587.1, found 587.1. |
| L19 | 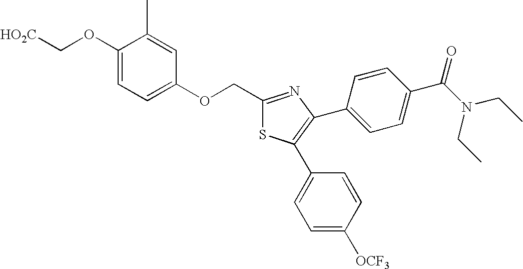 | ¹H-NMR (400 MHz, CD₃OD) δ =7.56 (d, J=8.0 Hz, 2H), 7.45-7.34 (m, 4H), 7.28 (d, J=8.8 Hz, 2H), 6.93 (d, J=2.8 Hz, 1H), 6.86-6.77 (m, 2H), 5.37 (s, 2H), 4.62 (s, 2H), 3.59-3.25 (m, 4H), 2.26 (s, 3H), 1.39-1.10 (m, 6H). MS calculated for C₃₁H₃₀F₃N₂O₆S (M + H⁺) 615.2, found 615.2. |
| L20 | 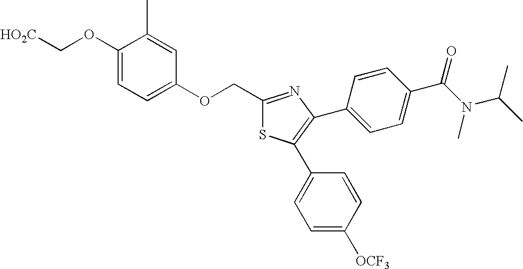 | ¹H-NMR (400 MHz, CD₃OD) δ =7.52 (d, J=8.0 Hz, 2H), 7.41-7.28 (m, 4H), 7.24 (d, J=8.8 Hz, 2H), 6.88 (d, J=3.2 Hz, 1H), 6.82-6.73 (m, 2H), 5.32 (s, 2H), 4.58 (s, 2H), 3.95-3.86 (m, 1H), 2.89-2.88 (m, 3H), 2.22 (s, 3H), 1.25-1.12 (m, 6H). MS calculated for C₃₁H₃₀F₃N₂O₆S (M + H⁺) 615.2, found 615.2. |
| L21 | 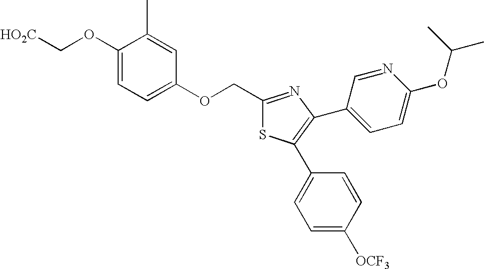 | ¹H-NMR (400 MHz, CDCl₃) δ =8.48 (m, 1H), 7.77 (m, 1H), 7.32-7.16 (m, 5H), 6.80-6.64 (m, 3H), 5.31 (m, 2H), 5.25 (s, 2H), 4.58 (s, 2H), 3.77 (m, 4H), 2.21 (s, 3H), 1.38 (d, J=6.1 Hz, 6H). MS calculated for C₂₈H₂₆F₃N₂O₆S (M + H⁺) 575.1, found 575.1. |
| L22 | 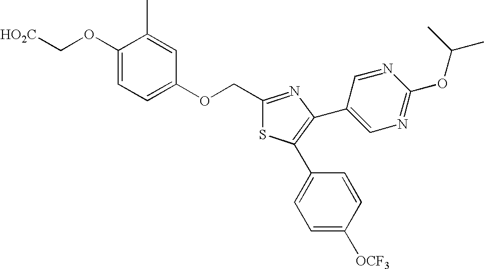 | MS calculated for C₂₇H₂₅F₃N₃O₆S (M + H⁺) 576.1, found 576.1. |
| L23 | 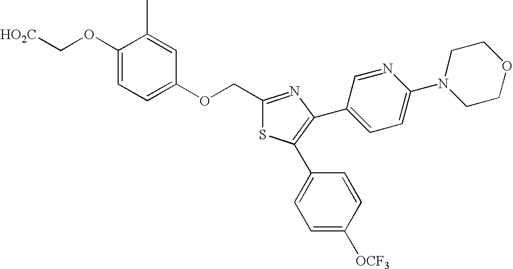 | ¹H-NMR (400 MHz, CDCl₃) δ =8.39 (d, J=2.1 Hz, 1H), 7.83 (m, 1H), 7.51-7.35 (m, 4H), 6.99-6.82 (m, 4H), 5.41 (s, 2H), 4.77 (s, 2H), 3.98 (m, 4H), 3.79 (m, 4H), 2.40 (s, 3H). MS calculated for C₂₉H₂₆F₃N₃O₆S (M + H⁺) 602.2, found 602.2. |
| L24 | 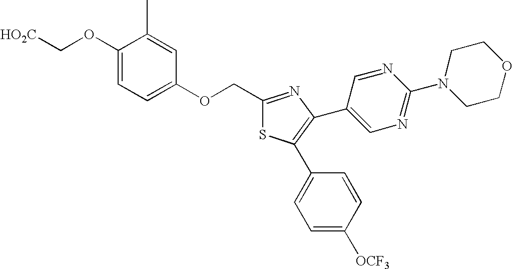 | ¹H-NMR (400 MHz, CDCl₃) δ =8.48 (s, 2H), 7.40 (d, J=8.4 Hz, 2H), 7.23 (d, J=8.4 Hz, 2H), 6.89 (s, 1H), 6.79 (d, J=7.3 Hz, 1H), 6.71 (d, J=7.3 Hz, 1H), 5.32 (s, 2H), 4.64 (s, 2H), 3.82 (m, 4H), 3.78 (m, 4H), 2.29 (s, 3H). MS calculated for C₂₈H₂₆F₃N₄O₆S (M + H⁺) 603.1, found 603.3. |
| M2 | 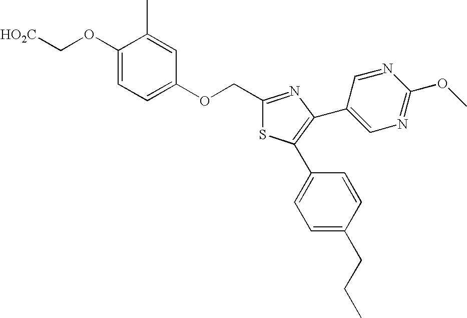 | ¹H-NMR (400 MHz, CD₃OD) δ =8.52 (s, 2H), 7.23-7.16 (m, 4H), 6.84 (d, J=2.8 Hz, 1H), 6.78-6.68 (m, 2H), 5.27 (s, 2H), 4.54 (s, 2H), 3.90 (s, 3H), 2.52 (t, J=7.6 Hz, 2H), 2.16 (s, 3H), 1.60-1.54 (m, 2H), 0.86 (t, J=7.4 Hz, 3H). MS calculated for C₂₇H₂₈N₃O₅S (M + H⁺) 506.2, found 506.2. |
| M3 | 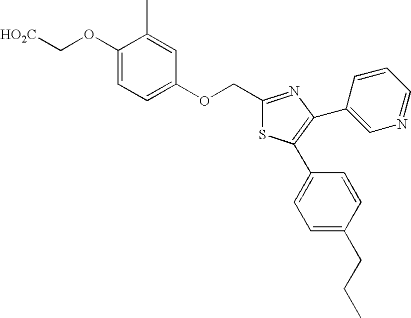 | ¹H-NMR (400 MHz, CD₃OD) δ =8.69 (bs, 1H), 8.51 (bs, 1H), 8.22 (d, J=8.0 Hz, 1H), 7.62 (bs, 1H), 7.23-7.16 (m, 4H), 6.83 (d, J=2.8 Hz, 1H), 6.76-6.67 (m, 2H), 5.28 (s, 2H), 4.53 (s, 2H), 2.54 (t, J=7.6 Hz, 2H), 2.16 (s, 3H), 1.62-1.52 (m, 2H), 0.87 (t, J=7.4 Hz, 3H). MS calculated for C₂₇H₂₇N₂O₄S (M + H⁺) 475.2, found 475.2. |
| M4 | 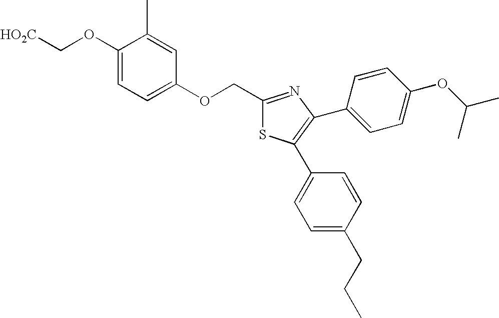 | ¹H-NMR (400 MHz, CD₃OD) δ =7.26 (d, J=8.8 Hz, 2H), 7.13-7.04 (m, 4H), 6.81 (d, J=2.8 Hz, 1H), 6.74-6.66 (m, 2H), 5.21 (s, 2H), 4.52 (s, 2H), 4.51-4.46 (m, 1H), 2.49 (t, J=7.6 Hz, 2H), 2.16 (s, 3H), 1.57-1.51 (m, 2H), 1.21 (d, J=6.0 Hz, 6H), 0.85 (t, J=7.4 Hz, 3H). MS calculated for C₃₁H₃₄NO₅S (M + H⁺) 532.2, found 532.2. |
| M5 | 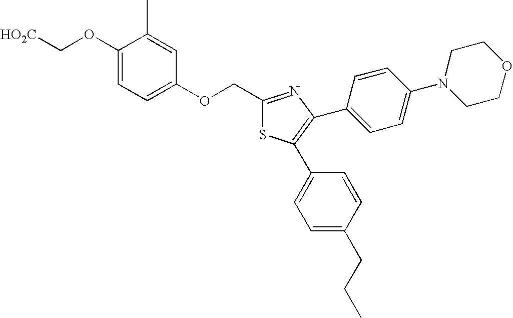 | ¹H-NMR (400 MHz, CD₃OD) δ =7.32 (d, J=8.8 Hz, 2H), 7.13-7.05 (m, 4H), 6.91 (d, J=8.4 Hz, 2H), 6.81 (d, J=2.8 Hz, 1H), 6.74-6.66 (m, 2H), 5.21 (s, 2H), 4.52 (s, 2H), 3.75 (t, J=4.6 Hz, 4H), 3.14 (bs, 4H), 2.49 (t, J=7.6 Hz, 2H), 2.16 (s, 3H), 1.57-1.50 (m, 2H), 0.85 (t, J=7.4 Hz, 3H). MS calculated for C₃₂H₃₅N₂O₅S (M + H⁺) |
| # 559.2, found 559.2. | | |
| M6 | 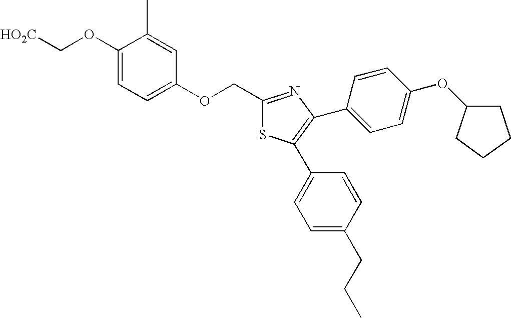 | ¹H-NMR (400 MHz, CD₃OD) δ =7.27 (d, J=8.4 Hz, 2H), 7.15-7.06 (m, 4H), 6.83 (d, J=2.4 Hz, 1H), 6.76-6.67 (m, 4H), 5.23 (s, 2H), 4.73-4.69 (m, 1H), 4.53 (s, 2H), 2.50 (t, J=7.6 Hz, 2H), 2.17 (s, 3H), 1.88-1.51 (m, 10H), 0.86 (t, J=7.4 Hz, 3H). MS calculated for C₃₃H₃₆NO₅S (M + H⁺) 558.2, found 558.2. |
| M7 | 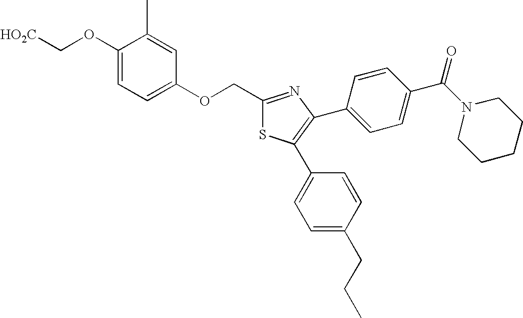 | ¹H-NMR (400 MHz, CD₃OD) δ =7.48 (d, J=8.0 Hz, 2H), 7.26 (d, J=8.4 Hz, 2H), 7.16-7.08 (m, 4H), 6.83 (d, J=2.8 Hz, 1H), 6.77-6.67 (m, 2H), 5.25 (s, 2H), 4.53 (s, 2H), 3.61 (bs, 2H), 3.30 (bs, 2H), 2.51 (t, J=7.6 Hz, 2H), 2.17 (s, 3H), 1.63-1.43 (m, 8H), 0.86 (t, J=7.4 Hz, 3H). MS calculated for C₃₄H₃₇N₂O₅S (M + H⁺) 585.2, |
| # found 585.2. | | |
| M8 | 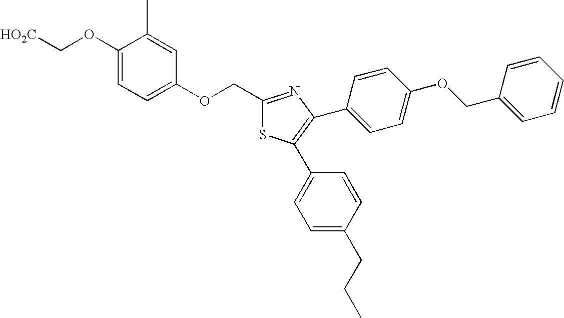 | ¹H-NMR (400 MHz, CD₃OD) δ =7.34-7.20 (m, 7H), 7.13-7.04 (m, 4H), 6.85-6.81 (m, 3H), 6.75-6.66 (m, 2H), 5.21 (s, 2H), 4.97 (s, 2H), 4.52 (s, 2H), 2.49 (t, J=7.6 Hz, 2H), 2.16 (s, 3H), 1.57-1.52 (m, 2H), 0.85 (t, J=7.4 Hz, 3H). MS calculated for C₃₅H₃₄NO₅S (M + H⁺) 580.2, found 580.2. |
| M9 | 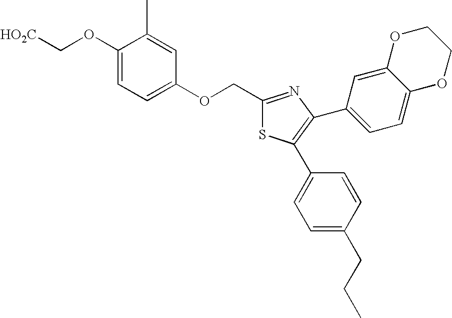 | ¹H-NMR (400 MHz, CD₃OD) δ =7.14-7.06 (m, 4H), 6.84-6.62 (m, 6H), 5.21 (s, 2H), 4.51 (s, 2H), 4.14-4.11 (m, 4H), 2.51 (t, J=7.6 Hz, 2H), 2.16 (s, 3H), 1.58-1.53 (m, 2H), 0.86 (t, J=7.4 Hz, 3H). MS calculated for C₃₀H₃₀NO₆S (M + H⁺) 532.1, found 532.1. |
| M10 | 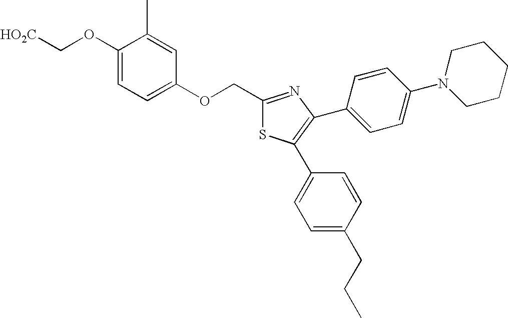 | ¹H-NMR (400 MHz, CD₃OD) δ =7.50 (d, J=8.8 Hz, 2H), 7.27 (d, J=8.8 Hz, 2H), 7.16-7.09 (m, 4H), 6.83 (d, J=2.4 Hz, 1H), 6.77-6.67 (m, 2H), 5.25 (s, 2H), 4.53 (s, 2H), 3.41-3.38 (m, 4H), 2.52 (t, J=7.6 Hz, 2H), 2.17 (s, 3H), 1.84-1.52 (m, 10H), 0.86 (t, J=7.2 Hz, 3H). MS calculated for C₃₃H₃₇N₂O₄S (M + H⁺) 557.2, found 557.2. |
| M11 | 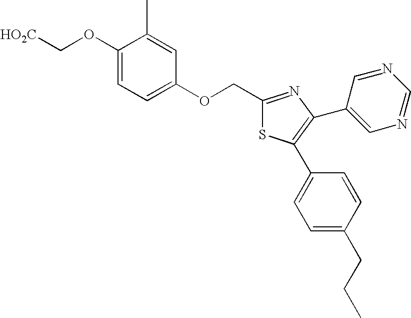 | ¹H-NMR (400 MHz, CD₃OD) δ =8.94 (s, 1H), 8.75 (s, 2H), 7.23-7.17 (m, 4H), 6.83 (d, J=2.8 Hz, 1H), 6.77-6.67 (m, 2H), 5.28 (s, 2H), 4.53 (s, 2H), 2.53 (t, J=7.6 Hz, 2H), 2.17 (s, 3H), 1.63-1.55 (m, 2H), 0.86 (t, J=7.2 Hz, 3H). MS calculated for C₂₆H₂₆N₃O₄S (M + H⁺) 476.1, found 476.1. |
| M12 | 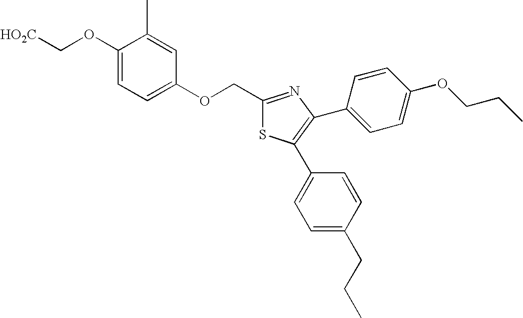 | ¹H-NMR (400 MHz, CDCl₃) δ =7.52-6.62 (m, 11H), 5.85 (s, 2H), 4.62 (s, 2H), 3.91 (t, J=6.5 Hz, 2H), 2.58 (m, 2H), 2.27 (s, 3H), 1.78 (m, 2H), 1.63 (m, 2H), 1.01 (t, J=7.4 Hz, 3H), 0.93 (t, J=7.3 Hz, 3H). MS calculated for C₃₁H₃₄NO₅S (M + H⁺) 532.2, found 532.2. |
| M13 | 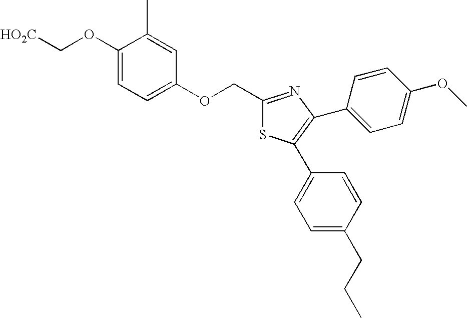 | ¹H-NMR (400 MHz, CDCl₃) δ =7.28-7.05 (m, 6H), 6.82 (d, J=2.8 Hz, 1H), 6.77-6.66 (m, 4H). 5.22 (s, 2H), 4.52 (s, 2H), 3.69 (s, 3H), 2.49 (t, J=7.6 Hz, 2H), 2.16 (s, 3H), 1.58-1.52 (m, 2H), 0.85 (t, J=7.4 Hz, 3H). MS calculated for C₂₉H₃₀NO₅S (M + H⁺) 504.2, found 504.2. |
| N2 | 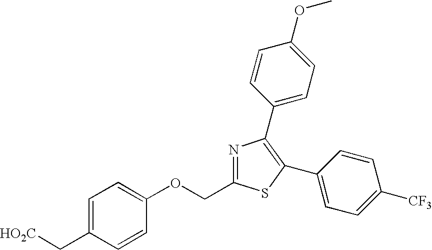 | ¹H-NMR (400 MHz, CDCl₃) δ =7.56 (d, J=8.2 Hz, 2H), 7.45 (d, J=8.2 Hz, 2H), 7.40 (d, J=8.8 Hz, 2H), 7.25 (d, J=10.0 Hz, 2H), 7.01 (d, J=8.7 Hz, 2H), 6.86 (d, J=8.8 Hz, 2H), 5.40 (s, 2H), 3.82 (s, 3H), 3.62 (s, 2H). MS calculated for C₂₆H₂₁F₃NO₄S (M + H⁺) 500.1, found 500.3. |
| N3 | 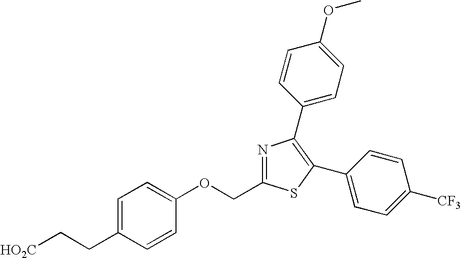 | ¹H-NMR (400 MHz, CDCl₃) δ =7.57 (d, J=8.2 Hz, 2H), 7.43 (d, J=8.2 Hz, 2H), 7.37 (d, J=8.8 Hz, 2H), 7.18 (d, J=8.6 Hz, 2H), 6.97 (d, J=8.6 Hz, 2H), 6.87 (d, J=8.8 Hz, 2H), 5.44 (s, 2H), 3.83 (s, 3H), 2.93 (t, J=7.6 Hz, 2H), 2.68 (t, J=7.6 Hz, 2H). MS calculated for C₂₇H₂₃F₃NO₄S (M + H⁺) 514.1, found 514.3. |
| N4 | 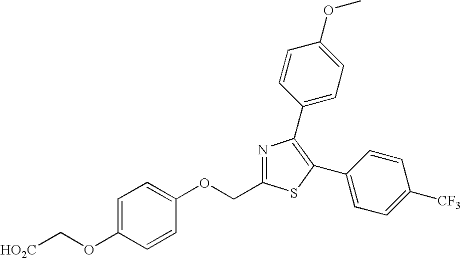 | ¹H-NMR (400 MHz, CDCl₃) δ =7.56 (d, J=8.2 Hz, 2H), 7.44 (d, J=8.2 Hz, 2H), 7.39 (d, J=8.8 Hz, 2H), 6.98 (d, J=9.1 Hz, 2H), 6.89 (d, J=9.1 Hz, 2H), 6.85 (d, J=8.8 Hz, 2H), 5.35 (s, 2H), 4.62 (s, 2H), 3.83 (s, 3H). MS calculated for C₂₆H₂₁F₃NO₅S (M + H⁺) 515.1, found 515.3. |
| N5 | 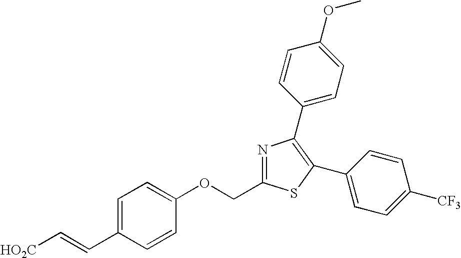 | ¹H-NMR (400 MHz, CDCl₃) δ =7.74 (d, J=15.9 Hz, 1H), 7.57 (d, J=8.8 Hz, 2H), 7.55 (d, J=8.8 Hz, 2H), 7.47-7.40 (m, 4H), 7.07 (d, J=8.8 Hz, 2H), 6.86 (d, J=8.8 Hz, 2H), 6.35 (d, J=15.9 Hz, 1H), 5.45 (s, 2H), 3.83 (s, 3H). MS calculated for C₂₇H₂₁F₃NO₄S (M + H⁺) 512.1, found 512.3. |
| N6 | 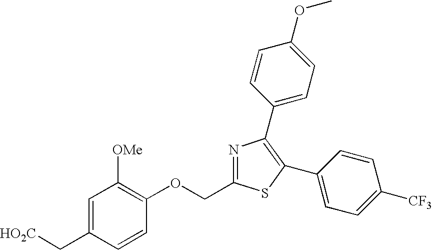 | ¹H-NMR (400 MHz, CDCl₃) δ =7.56 (d, J=8.2 Hz, 2H), 7.44 (d, J=8.2 Hz, 2H), 7.38 (d, J=8.6 Hz, 2H), 7.00 (d, J=8.1 Hz, 1H), 6.87-6.81 (m, 4H), 5.46 (s, 2H), 3.91 (s, 3H), 3.83 (s, 3H), 3.62 (s, 2H). MS calculated for C₂₇H₂₃F₃NO₅S (M + H⁺) 530.1, found 530.3. |
| N7 | 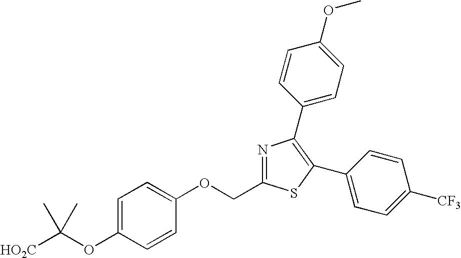 | ¹H-NMR (400 MHz, CDCl₃) δ =7.55 (d, J=8.2 Hz, 2H), 7.44 (d, J=8.2 Hz, 2H), 7.40 (d, J=8.8 Hz, 2H), 6.92 (s, 4H), 6.84 (d, J=8.8 Hz, 2H), 5.34 (s, 2H), 3.81 (s, 3H), 1.52 (s, 6H). MS calculated for C₂₈H₂₅F₃NO₅S (M + H⁺) 544.1, found 544.4. |
| N13 | 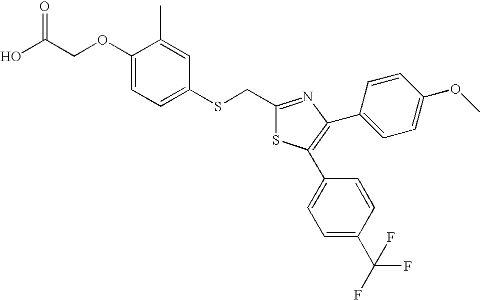 | ¹H-NMR (400 MHz, CDCl₃) δ =7.30 (d, J=8.4 Hz, 2H), 7.14 (d, J=8.0 Hz, 2H), 7.05 (m, 4H), 6.58 (d, J=8.8 Hz, 2H), 6.41 (d, J=8.4 Hz, 1H), 4.41 (s, 2H), 4.16 (s, 2H), 3.56 (s, 3H), 2.01 (s, 3H). MS calculated for C₂₇H₂₃F₃NO₄S₂(M + H⁺) 546.1, found 546.3. |
| N14 | 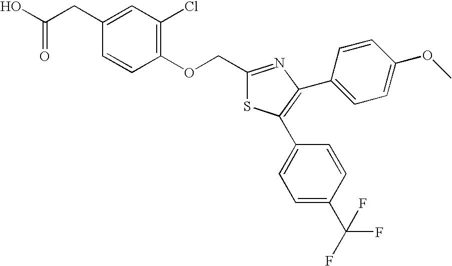 | ¹H-NMR (400 MHz, CDCl₃) δ =7.56 (d, J=8.4 Hz, 2H), 7.46 (d, J=8.0 Hz, 2H), 7.40 (d, J=8.8 Hz, 2H), 7.36 (d, J=2.0 Hz, 1H), 7.16 (dd, J=2.0, 8.4 Hz, 1H), 7.03 (d, J=8.4 Hz, 1H), 6.86 (d, J=8.8 Hz, 2H), 5.46 (s, 2H), 3.82 (s, 3H), 3.59 (s, 2H). MS calculated for C₂₆H₂₀ClF₃NO₄S (M + H⁺) 534.1, found 534.3. |
| N15 | 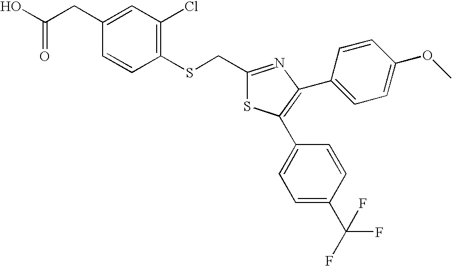 | ¹H-NMR (400 MHz, CDCl₃) δ =7.46 (d, J=8.0 Hz, 2H), 7.29 (m, 6H), 7.06 (dd, J=1.6, 8.0 Hz, 1H), 6.76 (d, J=8.8 Hz, 2H), 4.44 (s, 2H), 3.73 (s, 3H), 3.52 (s, 2H). MS calculated for C₂₆H₂₀ClF₃NO₃S₂ (M + H⁺) 550.0, found 550.3. |
| N20 | 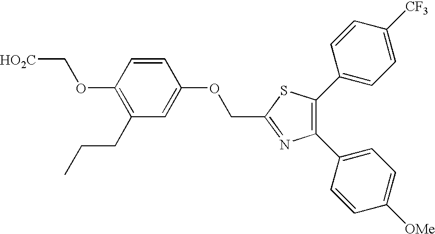 | ¹H-NMR (400 MHz, CDCl₃) δ =7.56 (d, J=8.4 Hz, 2H), 7.43 (d, J=8.0 Hz, 2H), 7.37 (d, J=8.4 Hz, 2H), 6.89 (d, J=3.0 Hz, 1H), 6.86 (d, J=8.8 Hz, 2H), 6.79 (dd, J=3.0, 9.0 Hz, 1H), 6.71 (d, J=9.0 Hz, 1H) 5.37 (s, 2H), 4.63 (s, 2H), 3.82 (s, 3H), 2.63 (t, J=7.6 Hz, 2H), 1.63 (m, 2H), 0.95 (t, J=7.4 Hz, 3H); ¹⁹F-NMR (376.5 MHz, CDCl₃) δ = −62.7. |
| # MS calculated for C₂₉H₂₇F₃NO₅S (M + H⁺) 558.2, found 558.2. | | |
| N21 | 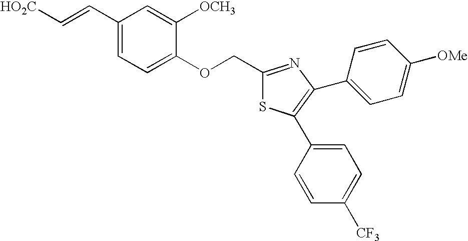 | ¹H-NMR (400 MHz, CDCl₃) δ =7.70 (d, J=15.6 Hz, 1H), 7.55 (d, J=8.4 Hz, 2H), 7.46 (d, J=8.0 Hz, 2H), 7.40 (d, J=8.4 Hz, 2H), 7.10 (m, 3H), 6.85 (d, J=8.8 Hz, 2H), 6.32 (d, J=16.0 Hz, 1H), 5.50 (s, 2H), 3.95 (s, 3H), 3.82 (s, 3H); ¹⁹F-NMR (376.5 MHz, CDCl₃) δ = −62.7. MS calculated for C₂₈H₂₃F₃NO₅S (M + H⁺) |
| # 542.1, found 542.1. | | |
| N22 | 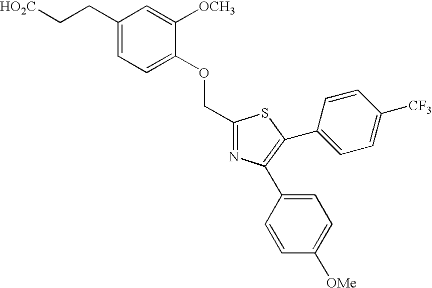 | ¹H-NMR (400 MHz, CDCl₃) δ =7.42 (d, J=8.0 Hz, 2H), 7.32 (d, J=8.4 Hz, 2H), 7.27 (d, J=8.4 Hz, 2H), 6.84 (d, J=8.0 Hz, 1H), 6.72 (d, J=8.8 Hz, 2H), 6.67 (d, J=2.0 Hz, 1H), 6.62 (dd, J=2.0, 8.0 Hz, 1H), 5.31 (s, 2H), 3.78 (s, 3H), 3.69 (s, 3H), 2.79 (t, J=7.8 Hz, 2H), 2.54 (t, J=7.8 Hz, 2H); ¹⁹F-NMR (376.5 MHz, CDCl₃) δ =−62.65. MS |
| # calculated for C₂₈H₂₃F₃NO₅S (M + H⁺) 544.1, found 544.1. | | |
| N23 | 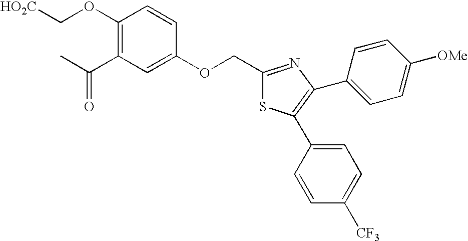 | ¹H-NMR (400 MHz, CDCl₃) δ =7.55 (d, J=8.4 Hz, 2H), 7.2 (m, 5H), 7.20 (m, 1H), 6.90 (m, 3H), 5.50 (s, 2H), 4.72 (s, 2H), 3.80 (s, 3H), 2.65 (s, 3H); ¹⁹F-NMR (376.5 MHz, CDCl₃) δ = −62.8. MS calculated for C₂₈H₂₃F₃NO₆S (M + H⁺) 558.1, found 558.0. |
| N24 | 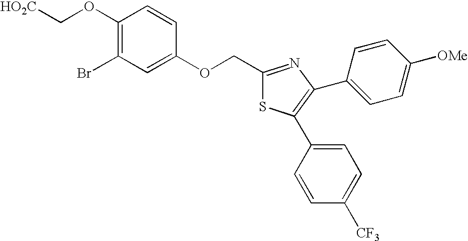 | ¹⁹F-NMR (376.5 MHz, CDCl₃) δ =−62.7. MS calculated for C₂₆H₁₉BrF₃NO₅S (M + H⁺) 594.1, found 594.0. |
| N25 | 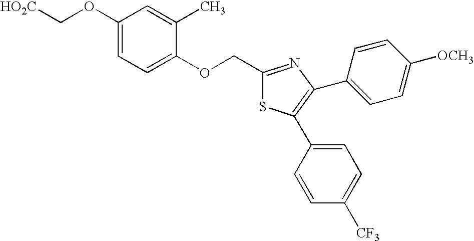 | ¹H-NMR (400 MHz, CDCl₃) δ =7.57 (d, J=8.4 Hz, 2H), 7.46 (d, J=8.0 Hz, 2H), 7.40 (d, J=8.4 Hz, 2H), 6.86 (d, J=8.8 Hz, 2H), 6.84 (m, 2H), 6.71 (dd, J=3.2, 8.8 Hz, 1H), 5.36 (s, 2H), 4.63 (s, 2H), 3.82 (s, 3H), 2.31 (s, 3H); ¹⁹F- NMR (376.5 MHz, CDCl₃) δ =−62.7. MS calculated for C₂₇H₂₃F₃NO₅S (M + H⁺) |
| # 530.1, found 530.1. | | |
| N26 | 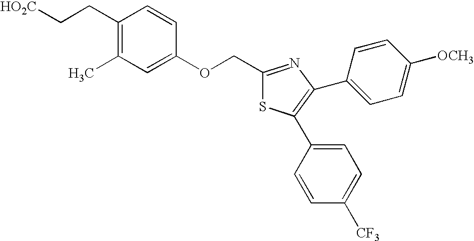 | ¹H-NMR (400 MHz, CD₃CN) δ =7.64 (d, J=8.0 Hz, 2H), 7.51 (d, J=8.0 Hz, 2H), 7.38 (d, J=8.8 Hz, 2H), 7.10 (d, J=8.4 Hz, 1H), 6.89 (d, J=2.8 Hz, 1H), 6.83 (d, J=8.8 Hz, 2H), 6.83 (dd, J=2.8, 8.4 Hz, 1H), 5.36 (s, 2H), 3.78 (s, 3H), 2.83 (t, J=7.2 Hz, 2H), 2.52 (t, J=7.2 Hz, 2H), 2.29 (s, 3H); ¹⁹F-NMR (376.5 MHz, CDCl₃) δ =−63.16. MS |
| # calculated for C₂₈H₂₅F₃NO₄S (M + H⁺) 528.1, found 528.2. | | |

By repeating the procedures described in the above examples, using appropriate starting materials, the following compounds of Formula I, as identified in Table 1, are obtained.

### Transcriptional Assay

Transfection assays are used to assess the ability of compounds of the invention to modulate the transcriptional activity of the PPARs. Briefly, expression vectors for chimeric proteins containing the DNA binding domain of yeast GAL4 fused to the ligand-binding domain (LBD) of either PPARδ, PPARα or PPARγ are introduced via transient transfection into mammalian cells, together with a reporter plasmid where the luciferase gene is under the control of a GAL4 binding site. Upon exposure to a PPAR modulator, PPAR transcriptional activity varies, and this can be monitored by changes in luciferase levels. If transfected cells are exposed to a PPAR agonist, PPAR-dependent transcriptional activity increases and luciferase levels rise.

293T human embryonic kidney cells (8×10⁶) are seeded in a 175 cm² flask a day prior to the start of the experiment in 10% FBS, 1% Penicillin/Streptomycin/Fungizome, DMEM Media. The cells are harvested by washing with PBS (30 ml) and then dissociating using trypsin (0.05%; 3 ml). The trypsin is inactivated by the addition of assay media (DMEM, CA-dextran fetal bovine serum (5%). The cells are spun down and resuspended to 170,000 cells/ml. A Transfection mixture of GAL4-PPAR LBD expression plasmid (1 μg), UAS-luciferase reporter plasmid (1 μg), Fugene (3:1 ratio; 6 μL) and serum-free media (200 μL) was prepared and incubated for 15-40 minutes at room temperature. Transfection mixtures are added to the cells to give 0.16M cells/mL, and cells (50 μl/well) are then plated into 384 white, solid-bottom, TC-treated plates. The cells are further incubated at 37° C., 5.0% CO₂ for 5-7 hours. A 12-point series of dilutions (3 fold serial dilutions) are prepared for each test compound in DMSO with a starting compound concentration of 10 μM. Test compound (500 nl) is added to each well of cells in the assay plate and the cells are incubated at 37° C., 5.0% CO₂ for 18-24 hours. The cell lysis/luciferase assay buffer, Bright-Glo™ (25%; 25 μl; Promega), is added to each well. After a further incubation for 5 minutes at room temperature, the luciferase activity is measured.

Raw luminescence values are normalized by dividing them by the value of the DMSO control present on each plate. Normalized data is analyzed and dose-response curves are fitted using Prizm graph fitting program. EC50 is defined as the concentration at which the compound elicits a response that is half way between the maximum and minimum values. Relative efficacy (or percent efficacy) is calculated by comparison of the response elicited by the compound with the maximum value obtained for a reference PPAR modulator.

Compounds of Formula I, in free form or in pharmaceutically acceptable salt form, exhibit valuable pharmacological properties, for example, as indicated by the in vitro tests described in this application. Compounds of the invention preferably have an EC50 for PPARδ of less than 1 μM, more preferably less than 500 nm, more preferably less than 100 nM. Compounds of the invention are at least 100-fold selective for PPARδ over PPARγ.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 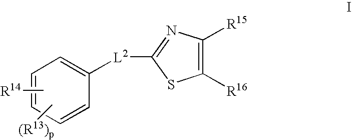
in which
p is an integer selected from 0 to 3;
L2 is selected from —XOX—, —XS(O)₀₋₂X— and —XS(O)₀₋₂XO—; wherein X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-substituted-C₁₋₆alkoxy;
R¹³ is selected from halo, C₁₋₆alkyl, C₁₋₆alkoxy, hydroxy-C₁₋₆alkyl, halo-substituted-C₁₋₆alkyl, halo-substituted-C₁₋₆alkoxy, C₆₋₁₀aryl, C₅₋₁₀heteroaryl, C₃₋₁₂cycloalkyl and C₃₋₈heterocycloalkyl; wherein any aryl, heteroaryl, cycloalkyl and heterocycloalkyl of R¹³ is optionally substituted with 1 to 3 radicals independently selected from halo, nitro, cyano, C₁₋₆alkyl, C₁₋₆alkoxy, hydroxy-C₁₋₆alkyl, halo-substituted-C₁₋₆alkyl and halo-substituted-C₁₋₆alkoxy;
R¹⁴ is selected from —XOXC(O)OR¹⁷ and —XC(O)OR¹⁷; wherein X is a bond or C₁₋₄alkylene; and R¹⁷ is selected from hydrogen and C₁₋₆alkyl;
R¹⁵ and R¹⁶ are independently selected from —R¹⁸ and —YR¹⁸; wherein Y is selected from C₁₋₆alkylene, C₂₋₆alkenylene, C₂₋₆alkynylene, —C(O)NR¹⁷— and —OX—; X is a bond or C₁₋₄alkylene; R¹⁷ is selected from hydrogen and C₁₋₆alkyl; and R¹⁸ is selected from C₃₋₁₂cycloalkyl, C₃₋₈heterocycloalkyl, C₆₋₁₀aryl and C₅₋₁₃heteroaryl; or R¹⁵ and R¹⁶ together with the atoms to which R¹⁵ and R¹⁶ are attached form fused bicyclic or tricyclic C₅₋₁₄heteroaryl;
wherein any aryl, heteroaryl, cycloalkyl and heterocycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl, C₅₋₁₃heteroaryl, —XS(O)₀₋₂R¹⁷, —XS(O)₀₋₂XR¹⁹, XNR¹⁷R¹⁷, —XNR¹⁷S(O)₀₋₂R¹⁷, —XNR¹⁷C(O)R¹⁷, —XC(O)NR¹⁷R¹⁷, —XNR¹⁷C(O)R¹⁹, —XC(O)NR¹⁷R¹⁹, —XC(O)R¹⁹, —XNR¹⁷XR¹⁹ and —XOXR¹⁹; wherein any aryl, heteroaryl, cycloalkyl or heterocycloalkyl substituent is further optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl and halo-substituted-C₁₋₆alkoxy; wherein X is a bond or C₁₋₄alkylene; R¹⁷ is selected from hydrogen and C₁₋₆alkyl; and R¹⁹ is selected from C₃₋₁₂cycloalkyl, C₃₋₈heterocycloalkyl, C₆₋₁₀aryl and C₅₋₁₀heteroaryl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy; and the pharmaceutically acceptable salts, hydrates, solvates, isomers and prodrugs thereof.

2. The compound of claim 1 in which:
p is an integer selected from 0 to 3;
L² is selected from —XOX—, —XS(O)₀₋₂X— and —XS(O)₀₋₂XO—; wherein X is independently selected from a bond and C₁₋₄alkylene; wherein any alkylene of L² can be optionally substituted by 1 to 3 radicals selected from halo, C₁₋₆alkyl, C₁₋₆alkoxy, halo-substituted-C₁₋₆alkyl and halo-substituted-C₁₋₆alkoxy;
R¹³ is C₁₋₆alkyl, C₁₋₆alkoxy and halogen; and
R¹⁴ is selected from —XOXC(O)OR¹⁷ and —XC(O)OR¹⁷; wherein X is a bond or C₁₋₄alkylene; and R¹⁷ is selected from hydrogen and C₁₋₆alkyl;
R¹⁵ and R¹⁶ are independently selected from —R¹⁸ and —YR¹⁸; wherein Y is selected from C₁₋₆alkylene, C₂₋₆alkenylene, —C(O)NR¹⁷— and —OX—; X is a bond or C₁₋₄alkylene; R¹⁷ is selected from hydrogen and C₁₋₆alkyl; and R¹⁸ is selected from C₆₋₁₀aryl, C₃₋₁₂cycloalkyl and C₅₋₁₃heteroaryl; or R¹⁵ and R¹⁶ together with the atoms to which R¹⁵ and R¹⁶ are attached form fused bicyclic or tricyclic C₅₋₁₄heteroaryl;
wherein any aryl, heteroaryl and cycloalkyl of R¹⁸, or the combination of R¹⁵ and R¹⁶, is optionally substituted with 1 to 3 radicals independently selected from halo, nitro, cyano, C₁₋₆alkyl, C₁₋₆alkoxy, C₁₋₆alkylthio, hydroxy-C₁₋₆alkyl, halo-substituted-C₁₋₆alkyl, halo-substituted-C₁₋₆alkoxy, C₃₋₁₂cycloalkyl, C₃₋₈heterocycloalkyl, C₆₋₁₀aryl optionally substituted with C₁₋₆alkoxy, C₅₋₁₃heteroaryl, —XS(O)₀₋₂R¹⁷, —XS(O)₀₋₂XR¹⁹, —XNR¹⁷R¹⁷, —XNR¹⁷S(O)₀₋₂R¹⁷, —XNR¹⁷C(O)R¹⁷, —XC(O)NR¹⁷R¹⁷, XNR¹⁷C(O)R¹⁹, —XC(O)NR¹⁷R¹⁹, —XC(O)R¹⁹, —XNR¹⁷R¹⁹ and —XOXR¹⁹; wherein X is a bond or C₁₋₄alkylene; R¹⁷ is selected from hydrogen and C₁₋₆alkyl; and R¹⁹ is selected from C₆₋₁₀aryl, C₅₋₁₀heteroaryl, C₃₋₈heterocycloalkyl and C₃₋₁₂cycloalkyl; wherein any aryl, heteroaryl, cycloalkyl or heterocycloalkyl of R¹⁹ is optionally substituted with 1 to 3 radicals independently selected from halo, nitro, cyano, C₁₋₆alkyl, C₁₋₆alkoxy, halo-substituted-C₁₋₆alkyl and halo-substituted-C₁₋₆alkoxy.

3. The compound of claim 1 of Formula Ia: 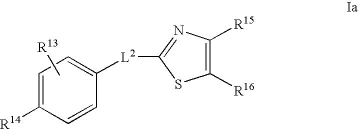
L² is selected from —S(O)₀₋₂(CH₂)₁₋₄O—, —O(CH₂)₁₋₄S(O)₀₋₂—, —CH₂S(O)₀₋₂—, —S(O)₀₋₂CH₂—, —S(O)₀₋₂—, —CH₂O— and —OCH₂—;
R¹³ is selected from C₁₋₆alkyl, C₁₋₆alkoxy and halo;
R¹⁴ is selected from —OCH₂C(O)OH and —CH₂C(O)OH;
R¹⁵ and R¹⁶ are independently selected from —R¹⁸ and —YR¹⁸; wherein Y is selected from C₁₋₆alkylene, C₂₋₆alkenylene, —C(O)NH— and —O(CH₂)₁₋₃—; and R¹⁸ is selected from phenyl, biphenyl, cyclohexyl, naphthyl, benzo[1,3]dioxol-5-yl, benzo[b]furanyl, pyridinyl, pyrimidinyl, dibenzo-furan-2-yl, furanyl, benzo[b]thiophene, thiophenyl, phenoxathiin-4-yl, benzoxazolyl, 3-oxo-3,4-dihydro-2H-benzo[1,4]oxazin-6-yl, 2-oxo-2,3-dihydro-benzooxazol-6-yl, 2,3-dihydro-benzo[1,4]dioxin-6-yl, benzoxazolyl, 3,4-dihydro-2H-benzo[b][1,4]dioxepin-7-yl and quinolinyl; or R¹⁵ and R¹⁶ together with the atoms to which R¹⁵ and R¹⁶ are attached form 4,5-dihydro-naphtho[1,2-d]thiazol-2-yl, 4H-chromeno[4,3-d]thiazol-2-yl, 5,6-dihydro-4H-3-thia-1-aza-benzo[e]azulen-2-yl, benzthiazolyl, benzoxazolyl and 1-oxa-3-aza-cyclopenta[a]naphthalen-2-yl;
wherein any aryl, heteroaryl, cycloalkyl and heterocycloalkyl of R¹⁵, R¹⁶ or the combination of R¹⁵ and R¹⁶, is optionally substituted with 1 to 3 radicals independently selected from halo, cyano, nitro, methyl, isopropyl, isopropyl-sulfanyl, isopropyloxy, hydroxy-methyl, methyl-sulfanyl, methoxy, ethoxy, pentafluoroethoxy, trifluoromethyl, trifluoromethoxy, trifluoromethyl-sulfonyl, morpholino, phenoxy, benzoxy, ethyl-sulfonyl, dimethylamino, methyl-sulfonyl-amino, ethyl-sulfonyl, propyl, vinyl, propyloxy, sec-butoxy, trifluoromethyl-sulfanyl, dimethyl-amino-carbonyl, diethyl-amino-carbonyl, methyl-carbonyl-amino, methyl-carbonyl, cyclopentyl-oxy, isopropyl-methylamino-carbonyl, cyclopropyl-amino-carbonyl, cyclohexyl, morpholino, piperidinyl, indolyl, pyrrolidinyl, pyrrolidinyl-carbonyl, 2,3-dihydro-benzofuran-5-yl piperidinyl-carbonyl, morpholino-carbonyl, isopropyl-methyl-amino, isopropyl-methyl-amino-carbonyl, diethyl-amino, and phenyl optionally substituted with methoxy.

4. The compound of claim 3 of Formula Ib: 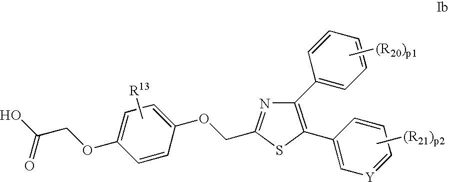
in which:
p1 and p2 are independently selected from 0, 1 and 2;
Y is selected from N and CH;
R¹³ is selected from C₁₋₆alkyl, C₁₋₆alkoxy and halo;
R²⁰ is selected from trifluoromethyl and trifluoromethoxy; and
R²¹ is selected from isopropyloxy and methoxy.

5. The compound of claim 4 selected from {4-[4-(4-isopropoxy-phenyl)-5-(4-trifluoromethoxy-phenyl)-thiazol-2-ylmethoxy]-2-methyl-phenoxy}-acetic acid; {4-[4-(4-isopropoxy-phenyl)-5-(4-trifluoromethyl-phenyl)-thiazol-2-ylmethoxy]-2-methyl-phenoxy}-acetic acid; and {4-[4-(6-methoxy-pyridin-3-yl)-5-(4-trifluoromethoxy-phenyl)-thiazol-2-ylmethoxy]-2-methyl-phenoxy}-acetic acid.

6. A method for treating a disease or disorder in an animal in which modulation of PPARδ activity can prevent, inhibit or ameliorate the pathology and/or symptomology of the disease, which method comprises administering to the animal a therapeutically effective amount of a compound of claim 1.

7. The method of claim 6 in which the disease or disorder is selected from the treatment of prophylaxis, dyslipidemia, hyperlipidemia, hypercholesteremia, atherosclerosis, atherogenesis, hypertriglyceridemia, heart failure, myocardial infarction, vascular diseases, cardiovascular diseases, hypertension, obesity, cachexia, inflammation, arthritis, cancer, anorexia, anorexia nervosa, bulimia, Alzheimer's disease, skin disorders, respiratory diseases, ophthalmic disorders, irritable bowel diseases, ulcerative colitis, Crohn's disease, type-1 diabetes, type-2 diabetes and Syndrome X.

8. The method of claim 6 in which the disease or disorder is selected from HIV wasting syndrome, long term critical illness, decreased muscle mass and/or muscle strength, decreased lean body mass, maintenance of muscle strength and function in the elderly, diminished muscle endurance and muscle function, and frailty in the elderly.

9. The use of a compound according to any of claims 1 to 5 in the manufacture of a medicament for treating a disease in an animal in which PPARδ activity contributes to the pathology and/or symptomology of the disease.

10. A pharmaceutical composition comprising a therapeutically effective amount of a compound of any of claim 1 to 5 in combination with one or more pharmaceutically acceptable excipients.

11. A pharmaceutical combination, especially a pharmaceutical composition, comprising: 1) a compound of any of claims 1 to 5 or a pharmaceutical acceptable salt thereof; and 2) at least one active ingredient selected from:
a) anti-diabetic agents such as insulin, insulin derivatives and mimetics; insulin secretagogues such as the sulfonylureas, e.g., Glipizide, glyburide and Amaryl;
insulinotropic sulfonylurea receptor ligands such as meglitinides, e.g., nateglinide and repaglinide; insulin sensitizer such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; dipeptidyl peptidase IV inhibitors such as DPP728, vildagliptin, MK-0431, saxagliptin, GSK3A; an AGE breaker; a thiazolidone derivative (glitazone) such as pioglitazone, rosiglitazone, or (R)-1-{4-[5-methyl-2-(4-trifluoromethyl-phenyl)-oxazol-4-ylmethoxy]-benzenesulfonyl}-2,3-dihydro-1H-indole-2-carboxylic acid, a non-glitazone type PPARγ agonist e.g. GI-262570;
b) hypolipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; squalene synthase inhibitors; FXR (farnesoid X receptor) and LXR (liver X receptor) ligands; cholestyramine; fibrates; nicotinic acid and aspirin;
c) an anti-obesity agent or appetite regulating agent such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine or cannabinoid receptor antagonists;
d) anti-hypertensive agents, e.g., loop diuretics such as ethacrynic acid, furosemide and torsemide; diuretics such as thiazide derivatives, chlorothiazide, hydrochlorothiazide, amiloride; angiotensin converting enzyme (ACE) inhibitors such as benazepril, captopril, enalapril, fosinopril, lisinopril, moexipril, perinodopril, quinapril, ramipril and trandolapril; inhibitors of the Na—K-ATPase membrane pump such as digoxin; neutralendopeptidase (NEP) inhibitors e.g. thiorphan, terteo-thiorphan, SQ29072; ECE inhibitors e.g. SLV306; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as aliskiren, terlakiren, ditekiren, RO 66-1132, RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists; and aldosterone synthase inhibitors;
e) a HDL increasing compound;
f) a cholesterol absorption modulator such as Zetia® and KT6-971;
g) Apo-A1 analogues and mimetics;
h) thrombin inhibitors such as Ximelagatran;
i) aldosterone inhibitors such as anastrazole, fadrazole, eplerenone;
j) Inhibitors of platelet aggregation such as aspirin, clopidogrel bisulfate;
k) estrogen, testosterone, a selective estrogen receptor modulator, a selective androgen receptor modulator;
l) a chemotherapeutic agent such as aromatase inhibitors e.g. femara, anti-estrogens, topoisomerase I inhibitors, topoisomerase II inhibitors, microtubule active agents, alkylating agents, antineoplastic antimetabolites, platin compounds, compounds decreasing the protein kinase activity such as a PDGF receptor tyrosine kinase inhibitor preferably Imatinib or 4-Methyl-N-[3-(4-methyl-imidazol-1-yl)-5-trifluoromethyl-phenyl]-3-(4-pyridin-3-yl-pyrimidin-2-ylamino)-benzamide; and
m) an agent interacting with a 5-HT₃ receptor and/or an agent interacting with 5-HT₄ receptor such as tegaserod, tegaserod hydrogen maleate, cisapride, cilansetron;
or, in each case a pharmaceutically acceptable salt thereof; and optionally a pharmaceutically acceptable carrier.

12. A pharmaceutical composition according to claim 10 or a combination according to claim 11, for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), impaired Glucose Tolerance (IGT), Impaired Fasting Glucose (IFG), and Syndrome-X.

13. A compound according to any of claims 1 to 5, or a pharmaceutical composition according to claim 10 or a combination according to claim 11, for use as a medicament.

14. Use of a compound according to any of claims 1 to 5, or a pharmaceutical composition according to claim 10 or a combination according to claim 11, for the manufacture of a medicament for the treatment or prevention of dyslipidemia, hyperlipidemia, hypercholesteremia, atherosclerosis, hypertriglyceridemia, heart failure, myocardial infarction, vascular diseases, cardiovascular diseases, hypertension, obesity, inflammation, arthritis, cancer, Alzheimer's disease, skin disorders, respiratory diseases, ophthalmic disorders, inflammatory bowel diseases, IBDs (irritable bowel disease), ulcerative colitis, Crohn's disease, conditions in which impaired glucose tolerance, hyperglycemia and insulin resistance are implicated, such as type-1 and type-2 diabetes, Impaired Glucose Metabolism (IGM), Impaired Glucose Tolerance (IGT), Impaired Fasting Glucose (IFG), and Syndrome-X.
